SEQUENCE LISTING (1556 sequences)

SEQ ID NO: 1 - DNA, 16 bp, Homo Sapiens
tacctgtttgtgtgcg

SEQ ID NO: 2 - DNA, 16 bp, Homo Sapiens
ccgagctccgctggtc

SEQ ID NO: 3 - DNA, 15 bp, Homo Sapiens
ggactcgcatgttcg

SEQ ID NO: 4 - DNA, 16 bp, Homo Sapiens
aacaaagttctctgtc

SEQ ID NO: 5 - DNA, 16 bp, Homo Sapiens
ccaggacgcacagttt

SEQ ID NO: 6 - DNA, 16 bp, Homo Sapiens
agtcggaggtgaggaa

SEQ ID NO: 7 - DNA, 16 bp, Homo Sapiens
ctgagcctgtgagtgc

SEQ ID NO: 8 - DNA, 15 bp, Homo Sapiens
gcgctggagaccatt

SEQ ID NO: 9 - DNA, 16 bp, Homo Sapiens
cagctctctcttcagg

SEQ ID NO: 10 - DNA, 16 bp, Homo Sapiens
gaggctgagcacgaat

SEQ ID NO: 11 - DNA, 15 bp, Homo Sapiens
gtagctacgatgagg

SEQ ID NO: 12 - DNA, 16 bp, Homo Sapiens
aaagaagcaagatgga

SEQ ID NO: 13 - DNA, 16 bp, Homo Sapiens
tcaaagagatgattga

SEQ ID NO: 14 - DNA, 14 bp, Homo Sapiens
catgcgctgtacat

SEQ ID NO: 15 - DNA, 16 bp, Homo Sapiens
acactacccaatgcct

SEQ ID NO: 16 - DNA, 16 bp, Homo Sapiens
aataatgtgatggaat

SEQ ID NO: 17 - DNA, 16 bp, Homo Sapiens
tgaagaactgttctac

SEQ ID NO: 18 - DNA, 16 bp, Homo Sapiens
gtggagagctactatt

SEQ ID NO: 19 - DNA, 15 bp, Homo Sapiens
aggcccaagtgtttt

SEQ ID NO: 20 - DNA, 15 bp, Homo Sapiens
tttgcagcagtggtg

SEQ ID NO: 21 - DNA, 16 bp, Homo Sapiens
taataatgctaataac

SEQ ID NO: 22 - DNA, 16 bp, Homo Sapiens
ggaggccagattgtag

SEQ ID NO: 23 - DNA, 16 bp, Homo Sapiens
gatctcctgggacact

SEQ ID NO: 24 - DNA, 16 bp, Homo Sapiens
cccttcgcctcttcct

SEQ ID NO: 25 - DNA, 16 bp, Homo Sapiens
atgtaccactactcaa

SEQ ID NO: 26 - DNA, 16 bp, Homo Sapiens
gaggagctggagatca

SEQ ID NO: 27 - DNA, 16 bp, Homo Sapiens
agtgtggacgtctctg

SEQ ID NO: 28 - DNA, 16 bp, Homo Sapiens
cgacttgtgcgttttc

SEQ ID NO: 29 - DNA, 16 bp, Homo Sapiens
agaacacgaatctccg

SEQ ID NO: 30 - DNA, 16 bp, Homo Sapiens
tgatgccacagtcgtc

SEQ ID NO: 31 - DNA, 16 bp, Homo Sapiens
gcaaaacagagcctcg

SEQ ID NO: 32 - DNA, 15 bp, Homo Sapiens
acgtcctgtcactcg

SEQ ID NO: 33 - DNA, 16 bp, Homo Sapiens
cctggaaaaggcaatc

SEQ ID NO: 34 - DNA, 16 bp, Homo Sapiens
cctcgctttatttttg

SEQ ID NO: 35 - DNA, 16 bp, Homo Sapiens
accgaagccacctttc

SEQ ID NO: 36 - DNA, 16 bp, Homo Sapiens
ctgtacctgtcataga

SEQ ID NO: 37 - DNA, 15 bp, Homo Sapiens
aacggtcgcactcaa

SEQ ID NO: 38 - DNA, 16 bp, Homo Sapiens
ctgcctcgatgaccta

SEQ ID NO: 39 - DNA, 16 bp, Homo Sapiens
acaggaacttcttggt

SEQ ID NO: 40 - DNA, 16 bp, Homo Sapiens
catcgaataccagaac

SEQ ID NO: 41 - DNA, 16 bp, Homo Sapiens
gagatgcaggtctcaa

SEQ ID NO: 42 - DNA, 16 bp, Homo Sapiens
gctgttgggaacgtaa

SEQ ID NO: 43 - DNA, 16 bp, Homo Sapiens
acttgagaaaacccag

SEQ ID NO: 44 - DNA, 16 bp, Homo Sapiens
tggaggatagaaagta

SEQ ID NO: 45 - DNA, 16 bp, Homo Sapiens
cgtgtacctgtcgtag

SEQ ID NO: 46 - DNA, 16 bp, Homo Sapiens
accactttccctctct

SEQ ID NO: 47 - DNA, 16 bp, Homo Sapiens
cagtaatgggaaatct

SEQ ID NO: 48 - DNA, 16 bp, Homo Sapiens
gaactccgcactttct

SEQ ID NO: 49 - DNA, 16 bp, Homo Sapiens
agaggagagaggaaaa

SEQ ID NO: 50 - DNA, 16 bp, Homo Sapiens
gcagctccgaggacca

SEQ ID NO: 51 - DNA, 14 bp, Homo Sapiens
caatcggcgtgtaa

SEQ ID NO: 52 - DNA, 16 bp, Homo Sapiens
tcttcttttcctctgg

SEQ ID NO: 53 - DNA, 16 bp, Homo Sapiens
ctctgctccggattcg

SEQ ID NO: 54 - DNA, 16 bp, Homo Sapiens
ggtgagcaggatccac

SEQ ID NO: 55 - DNA, 16 bp, Homo Sapiens
gtttagggtgtttgaa

SEQ ID NO: 56 - DNA, 16 bp, Homo Sapiens
ccggacgagcgcattt

SEQ ID NO: 57 - DNA, 16 bp, Homo Sapiens
tgacctggaaacttcg

SEQ ID NO: 58 - DNA, 16 bp, Homo Sapiens
cttttctctctggaca

SEQ ID NO: 59 - DNA, 15 bp, Homo Sapiens
acgcgtccgaaaatg

SEQ ID NO: 60 - DNA, 16 bp, Homo Sapiens
tcccgtgtaactccta

SEQ ID NO: 61 - DNA, 16 bp, Homo Sapiens
cccaggctgtttattt

SEQ ID NO: 62 - DNA, 16 bp, Homo Sapiens
aacattgcctatggag

SEQ ID NO: 63 - DNA, 16 bp, Homo Sapiens
gagcatagtaagcagt

SEQ ID NO: 64 - DNA, 16 bp, Homo Sapiens
caagcactgaaagata

SEQ ID NO: 65 - DNA, 16 bp, Homo Sapiens
tcccacagccactgtt

SEQ ID NO: 66 - DNA, 16 bp, Homo Sapiens
ttcctatatcctgtgt

SEQ ID NO: 67 - DNA, 16 bp, Homo Sapiens
cccactgtctatggtg

SEQ ID NO: 68 - DNA, 16 bp, Homo Sapiens
caggctgtatttcttc

SEQ ID NO: 69 - DNA, 16 bp, Homo Sapiens
taactagtcaagtact

SEQ ID NO: 70 - DNA, 16 bp, Homo Sapiens
ctttcatgactgcagc

SEQ ID NO: 71 - DNA, 16 bp, Homo Sapiens
agatcaaacaccctaa

SEQ ID NO: 72 - DNA, 16 bp, Homo Sapiens
gttttgtgcagtgagc

SEQ ID NO: 73 - DNA, 16 bp, Homo Sapiens
ctttttctttagacag

SEQ ID NO: 74 - DNA, 16 bp, Homo Sapiens
ttcccgtaggtcatga

SEQ ID NO: 75 - DNA, 16 bp, Homo Sapiens
cctcctggactacctg

SEQ ID NO: 76 - DNA, 16 bp, Homo Sapiens
acctgtggatgaagtt

SEQ ID NO: 77 - DNA, 16 bp, Homo Sapiens
ttggtgaaggtagctg

SEQ ID NO: 78 - DNA, 16 bp, Homo Sapiens
cttgatggagaacttg

SEQ ID NO: 79 - DNA, 16 bp, Homo Sapiens
ggtggtgtgatggtga

SEQ ID NO: 80 - DNA, 16 bp, Homo Sapiens
cgaagctcatgactgt

SEQ ID NO: 81 - DNA, 16 bp, Homo Sapiens
cggaagtccatctcct

SEQ ID NO: 82 - DNA, 16 bp, Homo Sapiens
gctcctgatctggtac

SEQ ID NO: 83 - DNA, 16 bp, Homo Sapiens
gtgaggatggctacag

SEQ ID NO: 84 - DNA, 16 bp, Homo Sapiens
ccatgtggagactcct

SEQ ID NO: 85 - DNA, 16 bp, Homo Sapiens
aaggtgcgttcgatga

SEQ ID NO: 86 - DNA, 16 bp, Homo Sapiens
acgcagacagaggctc

SEQ ID NO: 87 - DNA, 16 bp, Homo Sapiens
cacgttggtccacatc

SEQ ID NO: 88 - DNA, 15 bp, Homo Sapiens
accacccgcacgtct

SEQ ID NO: 89 - DNA, 16 bp, Homo Sapiens
aaatgttgaccaaagg

SEQ ID NO: 90 - DNA, 16 bp, Homo Sapiens
cttcttttagataccg

SEQ ID NO: 91 - DNA, 16 bp, Homo Sapiens
ggcagtctttactcac

SEQ ID NO: 92 - DNA, 16 bp, Homo Sapiens
aaatgcatttcctttc

SEQ ID NO: 93 - DNA, 16 bp, Homo Sapiens
aatgtgagcccttgag

SEQ ID NO: 94 - DNA, 16 bp, Homo Sapiens
tggctgtcagtatttg

SEQ ID NO: 95 - DNA, 16 bp, Homo Sapiens
ctcggaggaaggactt

SEQ ID NO: 96 - DNA, 16 bp, Homo Sapiens
tctgctctgcagcaaa

SEQ ID NO: 97 - DNA, 16 bp, Homo Sapiens
ccttggagatatcgat

SEQ ID NO: 98 - DNA, 16 bp, Homo Sapiens
gaacaggacgaactgg

SEQ ID NO: 99 - DNA, 16 bp, Homo Sapiens
agagtgagccacttct

SEQ ID NO: 100 - DNA, 16 bp, Homo Sapiens
tcttgtcttctccttt

SEQ ID NO: 101 - DNA, 16 bp, Homo Sapiens
ttgctcagcttgtact

SEQ ID NO: 102 - DNA, 16 bp, Homo Sapiens
gtgtagtgcttcaagg

SEQ ID NO: 103 - DNA, 15 bp, Homo Sapiens
acgctcaggttggag

SEQ ID NO: 104 - DNA, 16 bp, Homo Sapiens
atgagtgactgcctct

SEQ ID NO: 105 - DNA, 16 bp, Homo Sapiens
gcatagagcctacctg

SEQ ID NO: 106 - DNA, 16 bp, Homo Sapiens
gtgctagtggagaaca

SEQ ID NO: 107 - DNA, 16 bp, Homo Sapiens
ttcagggcaaagaagt

SEQ ID NO: 108 - DNA, 16 bp, Homo Sapiens
gttttccaatgcaacc

SEQ ID NO: 109 - DNA, 16 bp, Homo Sapiens
aaatcctaagagagaa

SEQ ID NO: 110 - DNA, 16 bp, Homo Sapiens
atgcttcctggtcttt

SEQ ID NO: 111 - DNA, 16 bp, Homo Sapiens
cacaggaagcagattc

SEQ ID NO: 112 - DNA, 16 bp, Homo Sapiens
tctgctggatttggtt

SEQ ID NO: 113 - DNA, 16 bp, Homo Sapiens
cccaaaagtccacctg

SEQ ID NO: 114 - DNA, 16 bp, Homo Sapiens
tccactgcatggttca

SEQ ID NO: 115 - DNA, 16 bp, Homo Sapiens
aagcagaagtaaaaca

SEQ ID NO: 116 - DNA, 16 bp, Homo Sapiens
tgaactgcagcattta

SEQ ID NO: 117 - DNA, 16 bp, Homo Sapiens
atgctgatactttatt

SEQ ID NO: 118 - DNA, 16 bp, Homo Sapiens
ctggaggcattattct

SEQ ID NO: 119 - DNA, 16 bp, Homo Sapiens
cagagcagcctaaaga

SEQ ID NO: 120 - DNA, 16 bp, Homo Sapiens
tggcagaaataataca

SEQ ID NO: 121 - DNA, 16 bp, Homo Sapiens
tcagcccatcttgaca

SEQ ID NO: 122 - DNA, 14 bp, Homo Sapiens
gtgcgttgcttgtt

SEQ ID NO: 123 - DNA, 16 bp, Homo Sapiens
tgagaggcatggctat

SEQ ID NO: 124 - DNA, 16 bp, Homo Sapiens
catcgagtcttcactg

SEQ ID NO: 125 - DNA, 16 bp, Homo Sapiens
tcagattccaaacaag

SEQ ID NO: 126 - DNA, 16 bp, Homo Sapiens
agacttacacacaaaa

SEQ ID NO: 127 - DNA, 16 bp, Homo Sapiens
cacctaggctaaaatg

SEQ ID NO: 128 - DNA, 16 bp, Homo Sapiens
agtattttctcacaga

SEQ ID NO: 129 - DNA, 16 bp, Homo Sapiens
tctgctagtgaatgag

SEQ ID NO: 130 - DNA, 16 bp, Homo Sapiens
acttactgtacctggt

SEQ ID NO: 131 - DNA, 16 bp, Homo Sapiens
tcaccttcagaagtca

SEQ ID NO: 132 - DNA, 16 bp, Homo Sapiens
ttgagatgaaaggatt

SEQ ID NO: 133 - DNA, 16 bp, Homo Sapiens
caccagtatagttcca

SEQ ID NO: 134 - DNA, 16 bp, Homo Sapiens
tctaactgatagaata

SEQ ID NO: 135 - DNA, 16 bp, Homo Sapiens
tggtttactttaagat

SEQ ID NO: 136 - DNA, 16 bp, Homo Sapiens
tctggaataattctga

SEQ ID NO: 137 - DNA, 16 bp, Homo Sapiens
gttctttgtttgtctt

SEQ ID NO: 138 - DNA, 16 bp, Homo Sapiens
aacaggaagcatactt

SEQ ID NO: 139 - DNA, 16 bp, Homo Sapiens
aagttcttgtgtttgt

SEQ ID NO: 140 - DNA, 16 bp, Homo Sapiens
atgcaggtggagggat

SEQ ID NO: 141 - DNA, 16 bp, Homo Sapiens
taccacagcaatgtgt

SEQ ID NO: 142 - DNA, 16 bp, Homo Sapiens
ttgagcatcccttgtg

SEQ ID NO: 143 - DNA, 16 bp, Homo Sapiens
ttttctgagtgctttt

SEQ ID NO: 144 - DNA, 16 bp, Homo Sapiens
aagcaaagttttaagg

SEQ ID NO: 145 - DNA, 16 bp, Homo Sapiens
cttaacacattgactt

SEQ ID NO: 146 - DNA, 16 bp, Homo Sapiens
cttgaagatttagcca

SEQ ID NO: 147 - DNA, 16 bp, Homo Sapiens
taaaaagtggcttagg

SEQ ID NO: 148 - DNA, 16 bp, Homo Sapiens
agaacaggatagaaag

SEQ ID NO: 149 - DNA, 16 bp, Homo Sapiens
tttctctcagtaagtg

SEQ ID NO: 150 - DNA, 16 bp, Homo Sapiens
aaaattagcaccctga

SEQ ID NO: 151 - DNA, 16 bp, Homo Sapiens
tatgtagaggctgttg

SEQ ID NO: 152 - DNA, 16 bp, Homo Sapiens
ctgaagttctttatct

SEQ ID NO: 153 - DNA, 16 bp, Homo Sapiens
ctggtgtacttgatag

SEQ ID NO: 154 - DNA, 16 bp, Homo Sapiens
tgttgtcatcttataa

SEQ ID NO: 155 - DNA, 16 bp, Homo Sapiens
caaactattgggtgga

SEQ ID NO: 156 - DNA, 16 bp, Homo Sapiens
tgtgtagaaagcagat

SEQ ID NO: 157 - DNA, 16 bp, Homo Sapiens
tttgtcagagtcagag

SEQ ID NO: 158 - DNA, 16 bp, Homo Sapiens
gatcctaaacgtaaga

SEQ ID NO: 159 - DNA, 16 bp, Homo Sapiens
gctttctggctggatt

SEQ ID NO: 160 - DNA, 16 bp, Homo Sapiens
taccttttctcttgat

SEQ ID NO: 161 - DNA, 16 bp, Homo Sapiens
tgcatttgaagaagga

SEQ ID NO: 162 - DNA, 16 bp, Homo Sapiens
caaagtatgagataaa

SEQ ID NO: 163 - DNA, 16 bp, Homo Sapiens
ttcttcaatttttgtt

SEQ ID NO: 164 - DNA, 16 bp, Homo Sapiens
atttacctagtaatgg

SEQ ID NO: 165 - DNA, 16 bp, Homo Sapiens
tttaggccttgcagaa

SEQ ID NO: 166 - DNA, 16 bp, Homo Sapiens
actgcatattcctcca

SEQ ID NO: 167 - DNA, 16 bp, Homo Sapiens
cagtagaagttgctgg

SEQ ID NO: 168 - DNA, 16 bp, Homo Sapiens
atgatttcatgtagtt

SEQ ID NO: 169 - DNA, 16 bp, Homo Sapiens
atttgaaaacaagcaa

SEQ ID NO: 170 - DNA, 16 bp, Homo Sapiens
cactcagttaactggt

SEQ ID NO: 171 - DNA, 16 bp, Homo Sapiens
tgttaaagttcatggc

SEQ ID NO: 172 - DNA, 16 bp, Homo Sapiens
cataagaagcgtttac

SEQ ID NO: 173 - DNA, 16 bp, Homo Sapiens
acagagatgaatttct

SEQ ID NO: 174 - DNA, 16 bp, Homo Sapiens
gaatatcacacttcta

SEQ ID NO: 175 - DNA, 16 bp, Homo Sapiens
ccacacaggagaatat

SEQ ID NO: 176 - DNA, 16 bp, Homo Sapiens
acaagctgtctcctct

SEQ ID NO: 177 - DNA, 16 bp, Homo Sapiens
aatatgaagtcttcat

SEQ ID NO: 178 - DNA, 16 bp, Homo Sapiens
tagctacactgcgcgt

SEQ ID NO: 179 - DNA, 16 bp, Homo Sapiens
ttggtgatagacatgt

SEQ ID NO: 180 - DNA, 16 bp, Homo Sapiens
acaacattccatgatg

SEQ ID NO: 181 - DNA, 16 bp, Homo Sapiens
cttttgaacagggcaa

SEQ ID NO: 182 - DNA, 16 bp, Homo Sapiens
ctcctttacttcatat

SEQ ID NO: 183 - DNA, 16 bp, Homo Sapiens
cctcactgaaaccttt

SEQ ID NO: 184 - DNA, 16 bp, Homo Sapiens
accaacactgagcaca

SEQ ID NO: 185 - DNA, 16 bp, Homo Sapiens
ggacaagtgaatttgc

SEQ ID NO: 186 - DNA, 16 bp, Homo Sapiens
aaaggctgaatgaaag

SEQ ID NO: 187 - DNA, 16 bp, Homo Sapiens
tgtttttggagaagca

SEQ ID NO: 188 - DNA, 16 bp, Homo Sapiens
attctcgcctctattg

SEQ ID NO: 189 - DNA, 16 bp, Homo Sapiens
tggaaaaatagcctca

SEQ ID NO: 190 - DNA, 16 bp, Homo Sapiens
atgaagacctcacagt

SEQ ID NO: 191 - DNA, 16 bp, Homo Sapiens
aagaaaaagtcaggat

SEQ ID NO: 192 - DNA, 16 bp, Homo Sapiens
tactcaggttaaaatg

SEQ ID NO: 193 - DNA, 16 bp, Homo Sapiens
ctcaatgatatggaga

SEQ ID NO: 194 - DNA, 16 bp, Homo Sapiens
atttctttgtacagga

SEQ ID NO: 195 - DNA, 16 bp, Homo Sapiens
atgacttgtcacaatg

SEQ ID NO: 196 - DNA, 16 bp, Homo Sapiens
cgagtgatgattggga

SEQ ID NO: 197 - DNA, 16 bp, Homo Sapiens
atttttggattactta

SEQ ID NO: 198 - DNA, 16 bp, Homo Sapiens
gctgcttttccagggt

SEQ ID NO: 199 - DNA, 16 bp, Homo Sapiens
tttcgacaaaagtcac

SEQ ID NO: 200 - DNA, 16 bp, Homo Sapiens
acaagagagtagatac

SEQ ID NO: 201 - DNA, 16 bp, Homo Sapiens
aggccctcagttcaag

SEQ ID NO: 202 - DNA, 16 bp, Homo Sapiens
tcccttcacaggtggt

SEQ ID NO: 203 - DNA, 16 bp, Homo Sapiens
ggagaaactcccagat

SEQ ID NO: 204 - DNA, 16 bp, Homo Sapiens
ccagtcagatcaggat

SEQ ID NO: 205 - DNA, 16 bp, Homo Sapiens
gaacaatatgaattat

SEQ ID NO: 206 - DNA, 16 bp, Homo Sapiens
ctgccaggatgtaaga

SEQ ID NO: 207 - DNA, 16 bp, Homo Sapiens
gatgcatctgttacta

SEQ ID NO: 208 - DNA, 16 bp, Homo Sapiens
actccctctaggatca

SEQ ID NO: 209 - DNA, 16 bp, Homo Sapiens
tcataacccacagatc

SEQ ID NO: 210 - DNA, 16 bp, Homo Sapiens
gaaaaatgccaacata

SEQ ID NO: 211 - DNA, 16 bp, Homo Sapiens
tgttcctggtcctgac

SEQ ID NO: 212 - DNA, 16 bp, Homo Sapiens
tcagtgactgtgatca

SEQ ID NO: 213 - DNA, 16 bp, Homo Sapiens
tgaaaagagagtggaa

SEQ ID NO: 214 - DNA, 16 bp, Homo Sapiens
gctgcaagtcagttga

SEQ ID NO: 215 - DNA, 16 bp, Homo Sapiens
aagaacagcacgtaca

SEQ ID NO: 216 - DNA, 16 bp, Homo Sapiens
tgaactcttccctcca

SEQ ID NO: 217 - DNA, 16 bp, Homo Sapiens
tcttgagggccacaaa

SEQ ID NO: 218 - DNA, 16 bp, Homo Sapiens
attgtagggtctccct

SEQ ID NO: 219 - DNA, 16 bp, Homo Sapiens
atcgaagcgctacctg

SEQ ID NO: 220 - DNA, 16 bp, Homo Sapiens
ccaacgcaccgaatag

SEQ ID NO: 221 - DNA, 16 bp, Homo Sapiens
acctggtcttctagga

SEQ ID NO: 222 - DNA, 16 bp, Homo Sapiens
gttttcgtggttcaca

SEQ ID NO: 223 - DNA, 16 bp, Homo Sapiens
ccacataaggttctca

SEQ ID NO: 224 - DNA, 16 bp, Homo Sapiens
ctggcagactatgtta

SEQ ID NO: 225 - DNA, 16 bp, Homo Sapiens
tacaggaatagccaca

SEQ ID NO: 226 - DNA, 16 bp, Homo Sapiens
ctgtgtagtaccttca

SEQ ID NO: 227 - DNA, 16 bp, Homo Sapiens
accatgactttgaggt

SEQ ID NO: 228 - DNA, 16 bp, Homo Sapiens
ggacatcttcccacta

SEQ ID NO: 229 - DNA, 16 bp, Homo Sapiens
ccatggaaccagacag

SEQ ID NO: 230 - DNA, 16 bp, Homo Sapiens
catcctcttcctcagg

SEQ ID NO: 231 - DNA, 16 bp, Homo Sapiens
aagttgttttgttttg

SEQ ID NO: 232 - DNA, 16 bp, Homo Sapiens
ttgagctgaggtcttc

SEQ ID NO: 233 - DNA, 16 bp, Homo Sapiens
aacgtttcgattataa

SEQ ID NO: 234 - DNA, 16 bp, Homo Sapiens
agactgtaagtggttt

SEQ ID NO: 235 - DNA, 16 bp, Homo Sapiens
aataattttcccacta

SEQ ID NO: 236 - DNA, 16 bp, Homo Sapiens
agggttgttgatgtcc

SEQ ID NO: 237 - DNA, 16 bp, Homo Sapiens
agggtcaaagatattt

SEQ ID NO: 238 - DNA, 16 bp, Homo Sapiens
ctcctcacttctggac

SEQ ID NO: 239 - DNA, 16 bp, Homo Sapiens
aggattcgatgaatca

SEQ ID NO: 240 - DNA, 16 bp, Homo Sapiens
caccaagcatcactgg

SEQ ID NO: 241 - DNA, 16 bp, Homo Sapiens
taaggtcaatgacgca

SEQ ID NO: 242 - DNA, 16 bp, Homo Sapiens
acaaccttgcggaatt

SEQ ID NO: 243 - DNA, 16 bp, Homo Sapiens
ttttgtcctactcctt

SEQ ID NO: 244 - DNA, 16 bp, Homo Sapiens
ttcagtgtttctccat

SEQ ID NO: 245 - DNA, 16 bp, Homo Sapiens
ttggaggaggtcaggc

SEQ ID NO: 246 - DNA, 16 bp, Homo Sapiens
agcccatctgggaaac

SEQ ID NO: 247 - DNA, 16 bp, Homo Sapiens
acatccggatgtagga

SEQ ID NO: 248 - DNA, 16 bp, Homo Sapiens
cctgagagcagcttca

SEQ ID NO: 249 - DNA, 16 bp, Homo Sapiens
tctcaccttctccatc

SEQ ID NO: 250 - DNA, 16 bp, Homo Sapiens
gtcctacgcttccaaa

SEQ ID NO: 251 - DNA, 16 bp, Homo Sapiens
cggctttgtccaagag

SEQ ID NO: 252 - DNA, 16 bp, Homo Sapiens
tgggcagaagggcaca

SEQ ID NO: 253 - DNA, 16 bp, Homo Sapiens
ggtgtgttctggaagt

SEQ ID NO: 254 - DNA, 16 bp, Homo Sapiens
atagtggccatcttcc

SEQ ID NO: 255 - DNA, 16 bp, Homo Sapiens
atgactgtcctgtaga

SEQ ID NO: 256 - DNA, 16 bp, Homo Sapiens
ccgtgacccaaagtac

SEQ ID NO: 257 - DNA, 16 bp, Homo Sapiens
atcaaatcttaaaagc

SEQ ID NO: 258 - DNA, 16 bp, Homo Sapiens
tctccactcagcgtct

SEQ ID NO: 259 - DNA, 16 bp, Homo Sapiens
gctgcgcttctactta

SEQ ID NO: 260 - DNA, 16 bp, Homo Sapiens
gggtggtgttgtgttt

SEQ ID NO: 261 - DNA, 16 bp, Homo Sapiens
gaggagcctggacagt

SEQ ID NO: 262 - DNA, 16 bp, Homo Sapiens
gaagactcagaggaga

SEQ ID NO: 263 - DNA, 16 bp, Homo Sapiens
aagtcctctcaagtct

SEQ ID NO: 264 - DNA, 16 bp, Homo Sapiens
tatccaattctgtttc

SEQ ID NO: 265 - DNA, 16 bp, Homo Sapiens
ttcatatgatgaaggg

SEQ ID NO: 266 - DNA, 16 bp, Homo Sapiens
agatacccacgtatgt

SEQ ID NO: 267 - DNA, 16 bp, Homo Sapiens
ctgatgacctgaagga

SEQ ID NO: 268 - DNA, 16 bp, Homo Sapiens
gactgtaattgatctt

SEQ ID NO: 269 - DNA, 16 bp, Homo Sapiens
gacccaaacatcatcc

SEQ ID NO: 270 - DNA, 16 bp, Homo Sapiens
gctggaggaagaatta

SEQ ID NO: 271 - DNA, 16 bp, Homo Sapiens
gagaagagatacgaag

SEQ ID NO: 272 - DNA, 16 bp, Homo Sapiens
gtggtaggtcttgtag

SEQ ID NO: 273 - DNA, 16 bp, Homo Sapiens
gtgccctcatttacct

SEQ ID NO: 274 - DNA, 16 bp, Homo Sapiens
cacgacagcgatgaga

SEQ ID NO: 275 - DNA, 16 bp, Homo Sapiens
ctccatatgtagttcg

SEQ ID NO: 276 - DNA, 16 bp, Homo Sapiens
atgttgatgtgtcttg

SEQ ID NO: 277 - DNA, 16 bp, Homo Sapiens
caccaacttatgccaa

SEQ ID NO: 278 - DNA, 16 bp, Homo Sapiens
ctgaatattgagctca

SEQ ID NO: 279 - DNA, 16 bp, Homo Sapiens
ccagcttcaaaagctc

SEQ ID NO: 280 - DNA, 16 bp, Homo Sapiens
cttttacactcctatt

SEQ ID NO: 281 - DNA, 16 bp, Homo Sapiens
agcatgaaatagtgta

SEQ ID NO: 282 - DNA, 16 bp, Homo Sapiens
gctttaaatcctcgaa

SEQ ID NO: 283 - DNA, 16 bp, Homo Sapiens
ttgggcacttttgaag

SEQ ID NO: 284 - DNA, 16 bp, Homo Sapiens
aaagtcaccaaccttt

SEQ ID NO: 285 - DNA, 16 bp, Homo Sapiens
tgtcctcattgccctc

SEQ ID NO: 286 - DNA, 16 bp, Homo Sapiens
agtccggttttatttg

SEQ ID NO: 287 - DNA, 16 bp, Homo Sapiens
aatgtgtcttcacttt

SEQ ID NO: 288 - DNA, 16 bp, Homo Sapiens
tgggcacagatgattt

SEQ ID NO: 289 - DNA, 16 bp, Homo Sapiens
ggcaaatacagctttg

SEQ ID NO: 290 - DNA, 16 bp, Homo Sapiens
ctccaagatgggatac

SEQ ID NO: 291 - DNA, 16 bp, Homo Sapiens
ggagatgtgataattt

SEQ ID NO: 292 - DNA, 16 bp, Homo Sapiens
gacttactgcagctgt

SEQ ID NO: 293 - DNA, 16 bp, Homo Sapiens
gtcactgactgctgtg

SEQ ID NO: 294 - DNA, 16 bp, Homo Sapiens
acattccggcaagaga

SEQ ID NO: 295 - DNA, 16 bp, Homo Sapiens
agttatttggaatttt

SEQ ID NO: 296 - DNA, 16 bp, Homo Sapiens
ctgtatgcactcagag

SEQ ID NO: 297 - DNA, 16 bp, Homo Sapiens
catgaacatttttctc

SEQ ID NO: 298 - DNA, 16 bp, Homo Sapiens
cagaccagggtgttgt

SEQ ID NO: 299 - DNA, 16 bp, Homo Sapiens
acacccagtggagaag

SEQ ID NO: 300 - DNA, 16 bp, Homo Sapiens
ccagggaccttacctt

SEQ ID NO: 301 - DNA, 16 bp, Homo Sapiens
gtcttccttctctctc

SEQ ID NO: 302 - DNA, 16 bp, Homo Sapiens
gaaactcacatcgagg

SEQ ID NO: 303 - DNA, 15 bp, Homo Sapiens
cctacgtgatggcca

SEQ ID NO: 304 - DNA, 16 bp, Homo Sapiens
ctttgtgttcccggac

SEQ ID NO: 305 - DNA, 16 bp, Homo Sapiens
ggaacgtactggtgaa

SEQ ID NO: 306 - DNA, 16 bp, Homo Sapiens
ctaaagccacctcctt

SEQ ID NO: 307 - DNA, 16 bp, Homo Sapiens
agagtgagttaacttt

SEQ ID NO: 308 - DNA, 16 bp, Homo Sapiens
actctggtgggtatag

SEQ ID NO: 309 - DNA, 16 bp, Homo Sapiens
agaaacgcatccagca

SEQ ID NO: 310 - DNA, 16 bp, Homo Sapiens
agcgacaatgaaaaac

SEQ ID NO: 311 - DNA, 15 bp, Homo Sapiens
gggtatgtggctaca

SEQ ID NO: 312 - DNA, 16 bp, Homo Sapiens
ctcacaccgctgtgtt

SEQ ID NO: 313 - DNA, 15 bp, Homo Sapiens
ccctgaccctggctt

SEQ ID NO: 314 - DNA, 16 bp, Homo Sapiens
acttccggatcttctg

SEQ ID NO: 315 - DNA, 15 bp, Homo Sapiens
ggatctggcgctttt

SEQ ID NO: 316 - DNA, 16 bp, Homo Sapiens
actgcctccagctctt

SEQ ID NO: 317 - DNA, 16 bp, Homo Sapiens
catctggatccctgat

SEQ ID NO: 318 - DNA, 16 bp, Homo Sapiens
ctgtcctcctagcagg

SEQ ID NO: 319 - DNA, 16 bp, Homo Sapiens
cataccctctcagcgt

SEQ ID NO: 320 - DNA, 16 bp, Homo Sapiens
atagggcataagctgt

SEQ ID NO: 321 - DNA, 16 bp, Homo Sapiens
aggtctacatgggtgc

SEQ ID NO: 322 - DNA, 16 bp, Homo Sapiens
gcccgaagtctgtaat

SEQ ID NO: 323 - DNA, 16 bp, Homo Sapiens
cacacagttggaggac

SEQ ID NO: 324 - DNA, 16 bp, Homo Sapiens
tcacacaccataactc

SEQ ID NO: 325 - DNA, 16 bp, Homo Sapiens
cactgtacaagctcta

SEQ ID NO: 326 - DNA, 16 bp, Homo Sapiens
aaagaggagcaggttg

SEQ ID NO: 327 - DNA, 16 bp, Homo Sapiens
gtcacagtggattcga

SEQ ID NO: 328 - DNA, 16 bp, Homo Sapiens
atgacgaagatggcaa

SEQ ID NO: 329 - DNA, 16 bp, Homo Sapiens
acacacgtaacataaa

SEQ ID NO: 330 - DNA, 16 bp, Homo Sapiens
gggttccagctggaaa

SEQ ID NO: 331 - DNA, 16 bp, Homo Sapiens
caccaagtatcagtat

SEQ ID NO: 332 - DNA, 16 bp, Homo Sapiens
caactggattcttttt

SEQ ID NO: 333 - DNA, 16 bp, Homo Sapiens
ccattggtagctggtg

SEQ ID NO: 334 - DNA, 16 bp, Homo Sapiens
atttttatctacttcc

SEQ ID NO: 335 - DNA, 16 bp, Homo Sapiens
tcctctcatcctgtct

SEQ ID NO: 336 - DNA, 16 bp, Homo Sapiens
tattggcacttatata

SEQ ID NO: 337 - DNA, 16 bp, Homo Sapiens
agagctaaggaagctt

SEQ ID NO: 338 - DNA, 16 bp, Homo Sapiens
aatcctatgcaaaaat

SEQ ID NO: 339 - DNA, 16 bp, Homo Sapiens
accttgaggaacatgg

SEQ ID NO: 340 - DNA, 16 bp, Homo Sapiens
atagcagctgcttatc

SEQ ID NO: 341 - DNA, 16 bp, Homo Sapiens
aaaccctacagatacc

SEQ ID NO: 342 - DNA, 16 bp, Homo Sapiens
atgtatccagatgatg

SEQ ID NO: 343 - DNA, 16 bp, Homo Sapiens
cttacatttgaccatg

SEQ ID NO: 344 - DNA, 16 bp, Homo Sapiens
atgacctttggaggaa

SEQ ID NO: 345 - DNA, 16 bp, Homo Sapiens
ccgatctggatcagca

SEQ ID NO: 346 - DNA, 16 bp, Homo Sapiens
acatttcagggtcctg

SEQ ID NO: 347 - DNA, 16 bp, Homo Sapiens
agagtgttgtccagtt

SEQ ID NO: 348 - DNA, 16 bp, Homo Sapiens
tgcttatcctcaagca

SEQ ID NO: 349 - DNA, 16 bp, Homo Sapiens
acaaaaatttaatact

SEQ ID NO: 350 - DNA, 16 bp, Homo Sapiens
ggcacaggatcattga

SEQ ID NO: 351 - DNA, 16 bp, Homo Sapiens
ttttcttctacttcca

SEQ ID NO: 352 - DNA, 16 bp, Homo Sapiens
tgagcttgtttgctga

SEQ ID NO: 353 - DNA, 16 bp, Homo Sapiens
aatcaaatagggaagg

SEQ ID NO: 354 - DNA, 16 bp, Homo Sapiens
gaccttacgtcagtga

SEQ ID NO: 355 - DNA, 16 bp, Homo Sapiens
aatgtaacaaatatga

SEQ ID NO: 356 - DNA, 16 bp, Homo Sapiens
ggaaactttggacttc

SEQ ID NO: 357 - DNA, 16 bp, Homo Sapiens
aagcccttagagatca

SEQ ID NO: 358 - DNA, 16 bp, Homo Sapiens
ctttgcagttatgatg

SEQ ID NO: 359 - DNA, 16 bp, Homo Sapiens
gggagttccaattctc

SEQ ID NO: 360 - DNA, 16 bp, Homo Sapiens
ctttttagattttgtg

SEQ ID NO: 361 - DNA, 16 bp, Homo Sapiens
tctgaaacataccatt

SEQ ID NO: 362 - DNA, 16 bp, Homo Sapiens
ttatccaaaagaattt

SEQ ID NO: 363 - DNA, 16 bp, Homo Sapiens
acattaacgctgactt

SEQ ID NO: 364 - DNA, 16 bp, Homo Sapiens
aacagctctagacaac

SEQ ID NO: 365 - DNA, 16 bp, Homo Sapiens
acattcaggaggaagt

SEQ ID NO: 366 - DNA, 16 bp, Homo Sapiens
catggaaaccttttag

SEQ ID NO: 367 - DNA, 16 bp, Homo Sapiens
tacattgattccattt

SEQ ID NO: 368 - DNA, 16 bp, Homo Sapiens
ttcctcaatgtttcca

SEQ ID NO: 369 - DNA, 16 bp, Homo Sapiens
agccctatttctactc

SEQ ID NO: 370 - DNA, 16 bp, Homo Sapiens
gatcctgaagaaagag

SEQ ID NO: 371 - DNA, 16 bp, Homo Sapiens
gtctccatcatcatca

SEQ ID NO: 372 - DNA, 16 bp, Homo Sapiens
ttattgcttctcctgt

SEQ ID NO: 373 - DNA, 16 bp, Homo Sapiens
ttatgttaaccaacct

SEQ ID NO: 374 - DNA, 16 bp, Homo Sapiens
caatcgtcagaaaatt

SEQ ID NO: 375 - DNA, 16 bp, Homo Sapiens
tatatcgtctacacaa

SEQ ID NO: 376 - DNA, 16 bp, Homo Sapiens
aacacaaagctggtgt

SEQ ID NO: 377 - DNA, 16 bp, Homo Sapiens
ctctccaatgtgacta

SEQ ID NO: 378 - DNA, 16 bp, Homo Sapiens
caagcatcagagtgct

SEQ ID NO: 379 - DNA, 16 bp, Homo Sapiens
gtaaacactgtcctgt

SEQ ID NO: 380 - DNA, 16 bp, Homo Sapiens
ggaattgcattcacac

SEQ ID NO: 381 - DNA, 16 bp, Homo Sapiens
catcaggagagcattt

SEQ ID NO: 382 - DNA, 16 bp, Homo Sapiens
gcatatgattttatgg

SEQ ID NO: 383 - DNA, 16 bp, Homo Sapiens
aaccctcctgccatca

SEQ ID NO: 384 - DNA, 16 bp, Homo Sapiens
ctctgcagagttgtta

SEQ ID NO: 385 - DNA, 16 bp, Homo Sapiens
caaattcaccaataat

SEQ ID NO: 386 - DNA, 16 bp, Homo Sapiens
ggagaatgtatacaca

SEQ ID NO: 387 - DNA, 16 bp, Homo Sapiens
tctctgcattccacac

SEQ ID NO: 388 - DNA, 16 bp, Homo Sapiens
tcttaagtgtttttcc

SEQ ID NO: 389 - DNA, 16 bp, Homo Sapiens
tgccaagtgaaatagt

SEQ ID NO: 390 - DNA, 16 bp, Homo Sapiens
acatcagacagcacag

SEQ ID NO: 391 - DNA, 16 bp, Homo Sapiens
caattttgaaccttac

SEQ ID NO: 392 - DNA, 16 bp, Homo Sapiens
ctatgtgctttcattc

SEQ ID NO: 393 - DNA, 16 bp, Homo Sapiens
atctttacctctttag

SEQ ID NO: 394 - DNA, 16 bp, Homo Sapiens
accagagaaattgctt

SEQ ID NO: 395 - DNA, 16 bp, Homo Sapiens
cacctgaaacattttt

SEQ ID NO: 396 - DNA, 16 bp, Homo Sapiens
gtaccatgttcagcaa

SEQ ID NO: 397 - DNA, 16 bp, Homo Sapiens
actatcatcagactga

SEQ ID NO: 398 - DNA, 16 bp, Homo Sapiens
gatgaggactcctcag

SEQ ID NO: 399 - DNA, 16 bp, Homo Sapiens
cctcctctaccacacg

SEQ ID NO: 400 - DNA, 16 bp, Homo Sapiens
gctggcttttggaaat

SEQ ID NO: 401 - DNA, 16 bp, Homo Sapiens
tccttgcttctcagat

SEQ ID NO: 402 - DNA, 16 bp, Homo Sapiens
ggacaatgtgatgaag

SEQ ID NO: 403 - DNA, 16 bp, Homo Sapiens
aggccttgggactgat

SEQ ID NO: 404 - DNA, 16 bp, Homo Sapiens
aagatgatcgggaagc

SEQ ID NO: 405 - DNA, 16 bp, Homo Sapiens
gtgttactgccatcga

SEQ ID NO: 406 - DNA, 16 bp, Homo Sapiens
gatttagcagccagaa

SEQ ID NO: 407 - DNA, 16 bp, Homo Sapiens
caatcaaactgcagag

SEQ ID NO: 408 - DNA, 16 bp, Homo Sapiens
ctggtgtcagagatgg

SEQ ID NO: 409 - DNA, 16 bp, Homo Sapiens
gacatggccaagagaa

SEQ ID NO: 410 - DNA, 16 bp, Homo Sapiens
ataacaacacgcctct

SEQ ID NO: 411 - DNA, 16 bp, Homo Sapiens
cagaagtcgatggcat

SEQ ID NO: 412 - DNA, 16 bp, Homo Sapiens
agctgaccaaacgtat

SEQ ID NO: 413 - DNA, 16 bp, Homo Sapiens
cggcacaggatgactg

SEQ ID NO: 414 - DNA, 16 bp, Homo Sapiens
tcctgtgatctgcaat

SEQ ID NO: 415 - DNA, 16 bp, Homo Sapiens
gcgtcctcaaaagtta

SEQ ID NO: 416 - DNA, 16 bp, Homo Sapiens
ccacaatcattcctgt

SEQ ID NO: 417 - DNA, 16 bp, Homo Sapiens
ctgccctatataattg

SEQ ID NO: 418 - DNA, 16 bp, Homo Sapiens
tatattgttctcctgt

SEQ ID NO: 419 - DNA, 16 bp, Homo Sapiens
agattcagaaagtcct

SEQ ID NO: 420 - DNA, 16 bp, Homo Sapiens
ttgtctgcaaggttta

SEQ ID NO: 421 - DNA, 16 bp, Homo Sapiens
acgtctcctccgacca

SEQ ID NO: 422 - DNA, 16 bp, Homo Sapiens
tttattggtctctcat

SEQ ID NO: 423 - DNA, 16 bp, Homo Sapiens
aaacttatgggagaaa

SEQ ID NO: 424 - DNA, 16 bp, Homo Sapiens
catcaatcacacgtac

SEQ ID NO: 425 - DNA, 16 bp, Homo Sapiens
gacccgtattcattct

SEQ ID NO: 426 - DNA, 16 bp, Homo Sapiens
aggattgttaaataac

SEQ ID NO: 427 - DNA, 16 bp, Homo Sapiens
atgttctgaaagctta

SEQ ID NO: 428 - DNA, 16 bp, Homo Sapiens
caacactgtcaagttt

SEQ ID NO: 429 - DNA, 16 bp, Homo Sapiens
cctgtgacattcacca

SEQ ID NO: 430 - DNA, 16 bp, Homo Sapiens
caataggacagtgctt

SEQ ID NO: 431 - DNA, 16 bp, Homo Sapiens
cgacacaacacaaaat

SEQ ID NO: 432 - DNA, 16 bp, Homo Sapiens
gggaaagtggtgaaga

SEQ ID NO: 433 - DNA, 16 bp, Homo Sapiens
tctctgtccaagtcct

SEQ ID NO: 434 - DNA, 16 bp, Homo Sapiens
tgtgtatgcctataat

SEQ ID NO: 435 - DNA, 16 bp, Homo Sapiens
tgggttacctgacagt

SEQ ID NO: 436 - DNA, 16 bp, Homo Sapiens
ctttctttgacagaaa

SEQ ID NO: 437 - DNA, 16 bp, Homo Sapiens
ctaaattttctcttgg

SEQ ID NO: 438 - DNA, 16 bp, Homo Sapiens
aagcaatttaggtatg

SEQ ID NO: 439 - DNA, 16 bp, Homo Sapiens
agtcagttaggaatag

SEQ ID NO: 440 - DNA, 16 bp, Homo Sapiens
caattggtgtttgtct

SEQ ID NO: 441 - DNA, 16 bp, Homo Sapiens
ttacctacgatggtaa

SEQ ID NO: 442 - DNA, 16 bp, Homo Sapiens
ttcaacaaacagaact

SEQ ID NO: 443 - DNA, 15 bp, Homo Sapiens
cctgaccgacgttga

SEQ ID NO: 444 - DNA, 16 bp, Homo Sapiens
gtaccggaagatgatg

SEQ ID NO: 445 - DNA, 16 bp, Homo Sapiens
ctgggattgcagattg

SEQ ID NO: 446 - DNA, 16 bp, Homo Sapiens
gatgtcacgttctcaa

SEQ ID NO: 447 - DNA, 16 bp, Homo Sapiens
accagttcagagtgga

SEQ ID NO: 448 - DNA, 16 bp, Homo Sapiens
tcatgttcctatatgg

SEQ ID NO: 449 - DNA, 16 bp, Homo Sapiens
tcactttcaggaattc

SEQ ID NO: 450 - DNA, 16 bp, Homo Sapiens
ggtggcggttacttac

SEQ ID NO: 451 - DNA, 16 bp, Homo Sapiens
ttagattggcaattat

SEQ ID NO: 452 - DNA, 16 bp, Homo Sapiens
actttgtccacctgga

SEQ ID NO: 453 - DNA, 16 bp, Homo Sapiens
ggatgatacatggtgg

SEQ ID NO: 454 - DNA, 16 bp, Homo Sapiens
tctcccacaatgaagg

SEQ ID NO: 455 - DNA, 14 bp, Homo Sapiens
tggtacgtccgcaa

SEQ ID NO: 456 - DNA, 16 bp, Homo Sapiens
tggtgaagcttccagc

SEQ ID NO: 457 - DNA, 16 bp, Homo Sapiens
gaagagcccacaggtg

SEQ ID NO: 458 - DNA, 16 bp, Homo Sapiens
tccttgctagggttct

SEQ ID NO: 459 - DNA, 16 bp, Homo Sapiens
tactggtggatgtcct

SEQ ID NO: 460 - DNA, 16 bp, Homo Sapiens
gttggacatcctggat

SEQ ID NO: 461 - DNA, 16 bp, Homo Sapiens
aggctcacctctatag

SEQ ID NO: 462 - DNA, 16 bp, Homo Sapiens
gcgatgacggaatata

SEQ ID NO: 463 - DNA, 16 bp, Homo Sapiens
ttgtactgcagagaca

SEQ ID NO: 464 - DNA, 16 bp, Homo Sapiens
accaagcccatcacca

SEQ ID NO: 465 - DNA, 16 bp, Homo Sapiens
aggcgtgggatgtttt

SEQ ID NO: 466 - DNA, 16 bp, Homo Sapiens
gaccactattatctct

SEQ ID NO: 467 - DNA, 16 bp, Homo Sapiens
gaggttccttaagatc

SEQ ID NO: 468 - DNA, 16 bp, Homo Sapiens
gttgagctctgcaggt

SEQ ID NO: 469 - DNA, 16 bp, Homo Sapiens
cctagacagcaccgta

SEQ ID NO: 470 - DNA, 16 bp, Homo Sapiens
ggataaagacctggtc

SEQ ID NO: 471 - DNA, 16 bp, Homo Sapiens
ttctggtgggaccatt

SEQ ID NO: 472 - DNA, 16 bp, Homo Sapiens
tctggatctcttcatg

SEQ ID NO: 473 - DNA, 16 bp, Homo Sapiens
aagagaacacacttac

SEQ ID NO: 474 - DNA, 16 bp, Homo Sapiens
gacattcctatgtcct

SEQ ID NO: 475 - DNA, 16 bp, Homo Sapiens
ctctgtaaattctacc

SEQ ID NO: 476 - DNA, 16 bp, Homo Sapiens
ctcggctttcatttga

SEQ ID NO: 477 - DNA, 16 bp, Homo Sapiens
taactctaataggaag

SEQ ID NO: 478 - DNA, 16 bp, Homo Sapiens
aatactaatgccagga

SEQ ID NO: 479 - DNA, 16 bp, Homo Sapiens
actgaggtatcgcctc

SEQ ID NO: 480 - DNA, 16 bp, Homo Sapiens
cacatcatccttggtt

SEQ ID NO: 481 - DNA, 16 bp, Homo Sapiens
gtggatgcttcctttt

SEQ ID NO: 482 - DNA, 16 bp, Homo Sapiens
ctccagtgaggaagca

SEQ ID NO: 483 - DNA, 16 bp, Homo Sapiens
caaacctgctgagcat

SEQ ID NO: 484 - DNA, 16 bp, Homo Sapiens
atcagtgttttgcttc

SEQ ID NO: 485 - DNA, 16 bp, Homo Sapiens
gctgacactggacatc

SEQ ID NO: 486 - DNA, 16 bp, Homo Sapiens
catctcatctgttaca

SEQ ID NO: 487 - DNA, 16 bp, Homo Sapiens
tgagagctcgatgctc

SEQ ID NO: 488 - DNA, 16 bp, Homo Sapiens
gagggtaagttgtata

SEQ ID NO: 489 - DNA, 16 bp, Homo Sapiens
ttttgcacagccaaga

SEQ ID NO: 490 - DNA, 16 bp, Homo Sapiens
aatgatcgttttcttc

SEQ ID NO: 491 - DNA, 16 bp, Homo Sapiens
gtgctcaaaaatttct

SEQ ID NO: 492 - DNA, 16 bp, Homo Sapiens
attgggtaatgttata

SEQ ID NO: 493 - DNA, 16 bp, Homo Sapiens
attaatttttgcttca

SEQ ID NO: 494 - DNA, 16 bp, Homo Sapiens
acccagagatacccag

SEQ ID NO: 495 - DNA, 16 bp, Homo Sapiens
catccatccaggaaaa

SEQ ID NO: 496 - DNA, 16 bp, Homo Sapiens
cattcattctgcttat

SEQ ID NO: 497 - DNA, 16 bp, Homo Sapiens
ctgtagcaaaaccaga

SEQ ID NO: 498 - DNA, 16 bp, Homo Sapiens
aatcatctcacctctg

SEQ ID NO: 499 - DNA, 16 bp, Homo Sapiens
tggatgtgcagacact

SEQ ID NO: 500 - DNA, 16 bp, Homo Sapiens
cttgcccacatcgttg

SEQ ID NO: 501 - DNA, 16 bp, Homo Sapiens
cagaaacccatgtatg

SEQ ID NO: 502 - DNA, 16 bp, Homo Sapiens
accaaaactcagcctg

SEQ ID NO: 503 - DNA, 16 bp, Homo Sapiens
agttgtgctttttgct

SEQ ID NO: 504 - DNA, 16 bp, Homo Sapiens
caagtagattcacaat

SEQ ID NO: 505 - DNA, 16 bp, Homo Sapiens
ttctttctaacctttt

SEQ ID NO: 506 - DNA, 16 bp, Homo Sapiens
gctttgaacaaataaa

SEQ ID NO: 507 - DNA, 16 bp, Homo Sapiens
actcatggtcggatca

SEQ ID NO: 508 - DNA, 16 bp, Homo Sapiens
aaactaaaaatccttt

SEQ ID NO: 509 - DNA, 16 bp, Homo Sapiens
tgttcaattttgttga

SEQ ID NO: 510 - DNA, 16 bp, Homo Sapiens
gacgtcactttcaaac

SEQ ID NO: 511 - DNA, 16 bp, Homo Sapiens
ggtcctatgggatttt

SEQ ID NO: 512 - DNA, 16 bp, Homo Sapiens
agcagtgttaatcaca

SEQ ID NO: 513 - DNA, 16 bp, Homo Sapiens
tgctcatcttttcttt

SEQ ID NO: 514 - DNA, 16 bp, Homo Sapiens
aaatttgttacctgta

SEQ ID NO: 515 - DNA, 16 bp, Homo Sapiens
tcacacagccaggagt

SEQ ID NO: 516 - DNA, 16 bp, Homo Sapiens
tgcaacagactttaaa

SEQ ID NO: 517 - DNA, 16 bp, Homo Sapiens
tgattttgcagaaaac

SEQ ID NO: 518 - DNA, 16 bp, Homo Sapiens
tctagaacagtagaca

SEQ ID NO: 519 - DNA, 16 bp, Homo Sapiens
tactggtccctcattg

SEQ ID NO: 520 - DNA, 16 bp, Homo Sapiens
taatccagactgtgtt

SEQ ID NO: 521 - DNA, 16 bp, Homo Sapiens
tctattgttggatcat

SEQ ID NO: 522 - DNA, 16 bp, Homo Sapiens
ataaggcctgctgaaa

SEQ ID NO: 523 - DNA, 16 bp, Homo Sapiens
ggctcattaagatgac

SEQ ID NO: 524 - DNA, 16 bp, Homo Sapiens
tatcactccttgaagc

SEQ ID NO: 525 - DNA, 16 bp, Homo Sapiens
caatcatactgctgac

SEQ ID NO: 526 - DNA, 16 bp, Homo Sapiens
aaccggtcctttacag

SEQ ID NO: 527 - DNA, 16 bp, Homo Sapiens
cacaaagcaagccaga

SEQ ID NO: 528 - DNA, 16 bp, Homo Sapiens
cgtaaaaatgctggag

SEQ ID NO: 529 - DNA, 16 bp, Homo Sapiens
tgtaataacaagtatt

SEQ ID NO: 530 - DNA, 16 bp, Homo Sapiens
tttttaaagtacatgt

SEQ ID NO: 531 - DNA, 16 bp, Homo Sapiens
gtaagtgcccgaagtg

SEQ ID NO: 532 - DNA, 16 bp, Homo Sapiens
acccactgaggtatat

SEQ ID NO: 533 - DNA, 16 bp, Homo Sapiens
gtgctaaccaagttct

SEQ ID NO: 534 - DNA, 16 bp, Homo Sapiens
ggttaaataaaatgcc

SEQ ID NO: 535 - DNA, 16 bp, Homo Sapiens
tgttccataatgaagt

SEQ ID NO: 536 - DNA, 16 bp, Homo Sapiens
caggagcgagaggaca

SEQ ID NO: 537 - DNA, 16 bp, Homo Sapiens
gtggtcctaccataca

SEQ ID NO: 538 - DNA, 16 bp, Homo Sapiens
agcaggcctattttga

SEQ ID NO: 539 - DNA, 16 bp, Homo Sapiens
tttctaactctctttg

SEQ ID NO: 540 - DNA, 16 bp, Homo Sapiens
tacagtttcttgcagc

SEQ ID NO: 541 - DNA, 16 bp, Homo Sapiens
cacgggtaataatttt

SEQ ID NO: 542 - DNA, 16 bp, Homo Sapiens
ctttgcacctgttttg

SEQ ID NO: 543 - DNA, 16 bp, Homo Sapiens
atacagctgattgcca

SEQ ID NO: 544 - DNA, 16 bp, Homo Sapiens
tctgctttggtccatc

SEQ ID NO: 545 - DNA, 16 bp, Homo Sapiens
aagtctgacccttttt

SEQ ID NO: 546 - DNA, 16 bp, Homo Sapiens
cctgtagtgtgcagga

SEQ ID NO: 547 - DNA, 16 bp, Homo Sapiens
tttgtgaccattccgg

SEQ ID NO: 548 - DNA, 16 bp, Homo Sapiens
ttctacctgaagagca

SEQ ID NO: 549 - DNA, 16 bp, Homo Sapiens
tgtcagcctcaaagaa

SEQ ID NO: 550 - DNA, 16 bp, Homo Sapiens
catccctattggcagg

SEQ ID NO: 551 - DNA, 16 bp, Homo Sapiens
aagtaacagtgtctac

SEQ ID NO: 552 - DNA, 16 bp, Homo Sapiens
ccgaagacttcaccta

SEQ ID NO: 553 - DNA, 16 bp, Homo Sapiens
tgttactctgtgccag

SEQ ID NO: 554 - DNA, 14 bp, Homo Sapiens
gacatcactcagga

SEQ ID NO: 555 - DNA, 16 bp, Homo Sapiens
ttattttatacctcac

SEQ ID NO: 556 - DNA, 16 bp, Homo Sapiens
tcctttgtgtcattcc

SEQ ID NO: 557 - DNA, 16 bp, Homo Sapiens
agaactgagtactctg

SEQ ID NO: 558 - DNA, 16 bp, Homo Sapiens
agaaagcctttttcgc

SEQ ID NO: 559 - DNA, 16 bp, Homo Sapiens
gttcttgtctttcctt

SEQ ID NO: 560 - DNA, 16 bp, Homo Sapiens
tggatttgattgacat

SEQ ID NO: 561 - DNA, 16 bp, Homo Sapiens
gctcatcatctgggac

SEQ ID NO: 562 - DNA, 16 bp, Homo Sapiens
aaccaatacaaccctc

SEQ ID NO: 563 - DNA, 16 bp, Homo Sapiens
ggcctcgatcttgtag

SEQ ID NO: 564 - DNA, 16 bp, Homo Sapiens
tacctggagattgaca

SEQ ID NO: 565 - DNA, 16 bp, Homo Sapiens
tgtttagtgatgaaaa

SEQ ID NO: 566 - DNA, 16 bp, Homo Sapiens
atacctactaagtgct

SEQ ID NO: 567 - DNA, 16 bp, Homo Sapiens
attctccaggcgtttc

SEQ ID NO: 568 - DNA, 16 bp, Homo Sapiens
tatcctcagagtggca

SEQ ID NO: 569 - DNA, 16 bp, Homo Sapiens
tacacagaggaagcct

SEQ ID NO: 570 - DNA, 16 bp, Homo Sapiens
gattcttacagaaaac

SEQ ID NO: 571 - DNA, 16 bp, Homo Sapiens
acctctatggtgggat

SEQ ID NO: 572 - DNA, 16 bp, Homo Sapiens
gttcttgctggtgtga

SEQ ID NO: 573 - DNA, 16 bp, Homo Sapiens
aaacatggtgggattt

SEQ ID NO: 574 - DNA, 16 bp, Homo Sapiens
tctttgggtcctaggt

SEQ ID NO: 575 - DNA, 16 bp, Homo Sapiens
ctgtcaacctgcatga

SEQ ID NO: 576 - DNA, 16 bp, Homo Sapiens
tcttttccacatcagt

SEQ ID NO: 577 - DNA, 16 bp, Homo Sapiens
ttttggccaacaatgt

SEQ ID NO: 578 - DNA, 16 bp, Homo Sapiens
caaggagattcttagc

SEQ ID NO: 579 - DNA, 16 bp, Homo Sapiens
tgtctgccaggaaact

SEQ ID NO: 580 - DNA, 16 bp, Homo Sapiens
atgacaaccaggacaa

SEQ ID NO: 581 - DNA, 16 bp, Homo Sapiens
ttacctgtcctggtca

SEQ ID NO: 582 - DNA, 16 bp, Homo Sapiens
actcccatcttgagtc

SEQ ID NO: 583 - DNA, 16 bp, Homo Sapiens
aaaaacaagctctcat

SEQ ID NO: 584 - DNA, 16 bp, Homo Sapiens
tgtccagtgaaaatcc

SEQ ID NO: 585 - DNA, 16 bp, Homo Sapiens
gtctgcaggacaattc

SEQ ID NO: 586 - DNA, 16 bp, Homo Sapiens
atgcaaatagttgacc

SEQ ID NO: 587 - DNA, 16 bp, Homo Sapiens
aacaatggtgactaca

SEQ ID NO: 588 - DNA, 16 bp, Homo Sapiens
cttatatgaggctgga

SEQ ID NO: 589 - DNA, 16 bp, Homo Sapiens
aaattgtgaagatctg

SEQ ID NO: 590 - DNA, 16 bp, Homo Sapiens
ctttagagattaaagt

SEQ ID NO: 591 - DNA, 16 bp, Homo Sapiens
tatatcattaagcaat

SEQ ID NO: 592 - DNA, 16 bp, Homo Sapiens
ttctaacattttgttt

SEQ ID NO: 593 - DNA, 16 bp, Homo Sapiens
gtagaatgtttactac

SEQ ID NO: 594 - DNA, 16 bp, Homo Sapiens
tcatcttgaagaagtt

SEQ ID NO: 595 - DNA, 16 bp, Homo Sapiens
tgatgaaacaagacga

SEQ ID NO: 596 - DNA, 16 bp, Homo Sapiens
aggatattgtatcata

SEQ ID NO: 597 - DNA, 16 bp, Homo Sapiens
caaatctacagagttc

SEQ ID NO: 598 - DNA, 16 bp, Homo Sapiens
catatcaaattcacac

SEQ ID NO: 599 - DNA, 16 bp, Homo Sapiens
gagcaatgtatgtcta

SEQ ID NO: 600 - DNA, 16 bp, Homo Sapiens
caggtagaagactgca

SEQ ID NO: 601 - DNA, 16 bp, Homo Sapiens
tgatctgggtaatagt

SEQ ID NO: 602 - DNA, 16 bp, Homo Sapiens
cagaggatagcaacat

SEQ ID NO: 603 - DNA, 16 bp, Homo Sapiens
ctacaccatatatgaa

SEQ ID NO: 604 - DNA, 16 bp, Homo Sapiens
catttgactttacctt

SEQ ID NO: 605 - DNA, 16 bp, Homo Sapiens
tatgttcgaacaggta

SEQ ID NO: 606 - DNA, 16 bp, Homo Sapiens
ctaaacactaatataa

SEQ ID NO: 607 - DNA, 16 bp, Homo Sapiens
gtcttcgtgatttgta

SEQ ID NO: 608 - DNA, 16 bp, Homo Sapiens
cgaggaagatcaggaa

SEQ ID NO: 609 - DNA, 16 bp, Homo Sapiens
agaaaagtgttttgaa

SEQ ID NO: 610 - DNA, 16 bp, Homo Sapiens
tttccagatactagag

SEQ ID NO: 611 - DNA, 16 bp, Homo Sapiens
aatctttggccagtac

SEQ ID NO: 612 - DNA, 16 bp, Homo Sapiens
agagagaaggtttgac

SEQ ID NO: 613 - DNA, 16 bp, Homo Sapiens
gccaattggtctgtat

SEQ ID NO: 614 - DNA, 16 bp, Homo Sapiens
ccttttccatagagaa

SEQ ID NO: 615 - DNA, 16 bp, Homo Sapiens
gagacaatgaattaag

SEQ ID NO: 616 - DNA, 16 bp, Homo Sapiens
agaatctccattttag

SEQ ID NO: 617 - DNA, 16 bp, Homo Sapiens
atggctcattcacaac

SEQ ID NO: 618 - DNA, 16 bp, Homo Sapiens
taattacagtccagaa

SEQ ID NO: 619 - DNA, 16 bp, Homo Sapiens
gattcttttagatctg

SEQ ID NO: 620 - DNA, 16 bp, Homo Sapiens
tttccattgcctcgac

SEQ ID NO: 621 - DNA, 16 bp, Homo Sapiens
gctcattaacttaact

SEQ ID NO: 622 - DNA, 16 bp, Homo Sapiens
gtatatacactgggct

SEQ ID NO: 623 - DNA, 16 bp, Homo Sapiens
ttgtagatatgatgca

SEQ ID NO: 624 - DNA, 16 bp, Homo Sapiens
accattacttgtccat

SEQ ID NO: 625 - DNA, 16 bp, Homo Sapiens
ctctaattttgtgaca

SEQ ID NO: 626 - DNA, 16 bp, Homo Sapiens
tgctgtcgaatagcta

SEQ ID NO: 627 - DNA, 16 bp, Homo Sapiens
tgccaatctcttcata

SEQ ID NO: 628 - DNA, 16 bp, Homo Sapiens
cttgctcagttttatc

SEQ ID NO: 629 - DNA, 16 bp, Homo Sapiens
gctttggagtatttca

SEQ ID NO: 630 - DNA, 16 bp, Homo Sapiens
tgagctttcattttct

SEQ ID NO: 631 - DNA, 16 bp, Homo Sapiens
tccatgtgttctgagc

SEQ ID NO: 632 - DNA, 16 bp, Homo Sapiens
aggttcccagctgttc

SEQ ID NO: 633 - DNA, 16 bp, Homo Sapiens
tcacaaagtttttgct

SEQ ID NO: 634 - DNA, 16 bp, Homo Sapiens
atcggaatcaagctca

SEQ ID NO: 635 - DNA, 16 bp, Homo Sapiens
aagctgaacacgcaaa

SEQ ID NO: 636 - DNA, 16 bp, Homo Sapiens
taacgtgaagtatgtc

SEQ ID NO: 637 - DNA, 16 bp, Homo Sapiens
gtagaagcaatctgat

SEQ ID NO: 638 - DNA, 16 bp, Homo Sapiens
tcatcttttgctgaga

SEQ ID NO: 639 - DNA, 16 bp, Homo Sapiens
gggtgtcctgtgtcac

SEQ ID NO: 640 - DNA, 16 bp, Homo Sapiens
aaacgcagattaccat

SEQ ID NO: 641 - DNA, 16 bp, Homo Sapiens
cagtgcatatactttc

SEQ ID NO: 642 - DNA, 16 bp, Homo Sapiens
ggattttaacaaggca

SEQ ID NO: 643 - DNA, 16 bp, Homo Sapiens
gacatgacagccatca

SEQ ID NO: 644 - DNA, 16 bp, Homo Sapiens
tctaagagagtgacag

SEQ ID NO: 645 - DNA, 16 bp, Homo Sapiens
tatttctttccttaac

SEQ ID NO: 646 - DNA, 16 bp, Homo Sapiens
aatcaaagcattctta

SEQ ID NO: 647 - DNA, 16 bp, Homo Sapiens
atgttagctcattttt

SEQ ID NO: 648 - DNA, 16 bp, Homo Sapiens
agcatacaaataagaa

SEQ ID NO: 649 - DNA, 16 bp, Homo Sapiens
attcaggcaatgtttg

SEQ ID NO: 650 - DNA, 16 bp, Homo Sapiens
ctctgcaattaaattt

SEQ ID NO: 651 - DNA, 16 bp, Homo Sapiens
attctgaggttatctt

SEQ ID NO: 652 - DNA, 16 bp, Homo Sapiens
caacatgattgtcatc

SEQ ID NO: 653 - DNA, 16 bp, Homo Sapiens
aatgatatgtgcatat

SEQ ID NO: 654 - DNA, 16 bp, Homo Sapiens
aggaagaggaaaggaa

SEQ ID NO: 655 - DNA, 16 bp, Homo Sapiens
tctgtccaccagggag

SEQ ID NO: 656 - DNA, 16 bp, Homo Sapiens
tggaatagtttcaaac

SEQ ID NO: 657 - DNA, 16 bp, Homo Sapiens
aagttcatgtactttg

SEQ ID NO: 658 - DNA, 16 bp, Homo Sapiens
ttttggatatttctcc

SEQ ID NO: 659 - DNA, 16 bp, Homo Sapiens
tagagcgtgcagataa

SEQ ID NO: 660 - DNA, 16 bp, Homo Sapiens
caaaatgtttaattta

SEQ ID NO: 661 - DNA, 16 bp, Homo Sapiens
atcacttcactggctt

SEQ ID NO: 662 - DNA, 16 bp, Homo Sapiens
tcctttgatgccctca

SEQ ID NO: 663 - DNA, 16 bp, Homo Sapiens
cctattacctcaatca

SEQ ID NO: 664 - DNA, 16 bp, Homo Sapiens
cttcacctttaacacc

SEQ ID NO: 665 - DNA, 16 bp, Homo Sapiens
aggcttgagtttgaag

SEQ ID NO: 666 - DNA, 16 bp, Homo Sapiens
taccaatactccatcc

SEQ ID NO: 667 - DNA, 16 bp, Homo Sapiens
ggaaaactttctttca

SEQ ID NO: 668 - DNA, 16 bp, Homo Sapiens
ttagctaataaaaatg

SEQ ID NO: 669 - DNA, 16 bp, Homo Sapiens
tttacagaaacagctg

SEQ ID NO: 670 - DNA, 16 bp, Homo Sapiens
gttctttacagagaac

SEQ ID NO: 671 - DNA, 16 bp, Homo Sapiens
atgtaaaggataattg

SEQ ID NO: 672 - DNA, 16 bp, Homo Sapiens
tctgaagagttttatc

SEQ ID NO: 673 - DNA, 16 bp, Homo Sapiens
tcattgcttaacacat

SEQ ID NO: 674 - DNA, 16 bp, Homo Sapiens
cttacgttaaaatagg

SEQ ID NO: 675 - DNA, 16 bp, Homo Sapiens
cagtgaatccaaaaga

SEQ ID NO: 676 - DNA, 16 bp, Homo Sapiens
aattacaatgaattca

SEQ ID NO: 677 - DNA, 16 bp, Homo Sapiens
aattgtgattttctaa

SEQ ID NO: 678 - DNA, 16 bp, Homo Sapiens
tttttaacttactgat

SEQ ID NO: 679 - DNA, 16 bp, Homo Sapiens
tatcaaagcagaaggc

SEQ ID NO: 680 - DNA, 16 bp, Homo Sapiens
tatgcacatgaatgaa

SEQ ID NO: 681 - DNA, 16 bp, Homo Sapiens
gagaaggaccaactga

SEQ ID NO: 682 - DNA, 16 bp, Homo Sapiens
tctatttgcagtttga

SEQ ID NO: 683 - DNA, 16 bp, Homo Sapiens
gtacaaccttgaagtg

SEQ ID NO: 684 - DNA, 16 bp, Homo Sapiens
tttacacgcgtagttg

SEQ ID NO: 685 - DNA, 16 bp, Homo Sapiens
tgaacgccttctgtct

SEQ ID NO: 686 - DNA, 16 bp, Homo Sapiens
ggtgaagtgcttgatt

SEQ ID NO: 687 - DNA, 16 bp, Homo Sapiens
attatgatgtgttcca

SEQ ID NO: 688 - DNA, 16 bp, Homo Sapiens
atggaaaattacctac

SEQ ID NO: 689 - DNA, 16 bp, Homo Sapiens
tactgttcttcctcag

SEQ ID NO: 690 - DNA, 16 bp, Homo Sapiens
ccctggtggaagcata

SEQ ID NO: 691 - DNA, 16 bp, Homo Sapiens
ggctgtgtgggacgtg

SEQ ID NO: 692 - DNA, 15 bp, Homo Sapiens
gcatcgaagacacgc

SEQ ID NO: 693 - DNA, 16 bp, Homo Sapiens
tctgccacctgcagat

SEQ ID NO: 694 - DNA, 16 bp, Homo Sapiens
tccttgcctccactca

SEQ ID NO: 695 - DNA, 14 bp, Homo Sapiens
agtgggctacgtct

SEQ ID NO: 696 - DNA, 14 bp, Homo Sapiens
tcgggctcgcagaa

SEQ ID NO: 697 - DNA, 14 bp, Homo Sapiens
tgcgacgagctgtg

SEQ ID NO: 698 - DNA, 16 bp, Homo Sapiens
cagctgaggagatggg

SEQ ID NO: 699 - DNA, 16 bp, Homo Sapiens
cctgacctggtatggt

SEQ ID NO: 700 - DNA, 16 bp, Homo Sapiens
cttcaggacgttgaac

SEQ ID NO: 701 - DNA, 16 bp, Homo Sapiens
aaccacccacatgtca

SEQ ID NO: 702 - DNA, 16 bp, Homo Sapiens
gggagaacagggctgt

SEQ ID NO: 703 - DNA, 16 bp, Homo Sapiens
tcgttcatcgggactt

SEQ ID NO: 704 - DNA, 16 bp, Homo Sapiens
ggctcctcttcacgta

SEQ ID NO: 705 - DNA, 16 bp, Homo Sapiens
cttcctagagagttag

SEQ ID NO: 706 - DNA, 16 bp, Homo Sapiens
cacacttacacatcac

SEQ ID NO: 707 - DNA, 16 bp, Homo Sapiens
ttacacacacgcaaaa

SEQ ID NO: 708 - DNA, 16 bp, Homo Sapiens
ttcccagtccactata

SEQ ID NO: 709 - DNA, 16 bp, Homo Sapiens
gatgagaacgcaatgc

SEQ ID NO: 710 - DNA, 16 bp, Homo Sapiens
ggtgatcagttatgaa

SEQ ID NO: 711 - DNA, 16 bp, Homo Sapiens
ttccataaaggcttta

SEQ ID NO: 712 - DNA, 16 bp, Homo Sapiens
tggttttgtaggtctt

SEQ ID NO: 713 - DNA, 15 bp, Homo Sapiens
aagcctggcacatac

SEQ ID NO: 714 - DNA, 16 bp, Homo Sapiens
tggtcctgtctgtcct

SEQ ID NO: 715 - DNA, 16 bp, Homo Sapiens
agtgcaagtgaaagcc

SEQ ID NO: 716 - DNA, 16 bp, Homo Sapiens
aaccttaaatgtctct

SEQ ID NO: 717 - DNA, 16 bp, Homo Sapiens
ccttcaagctgcccta

SEQ ID NO: 718 - DNA, 16 bp, Homo Sapiens
tatacaatcaatacct

SEQ ID NO: 719 - DNA, 16 bp, Homo Sapiens
ctaaggttgcacatag

SEQ ID NO: 720 - DNA, 16 bp, Homo Sapiens
agcttctctgtctaag

SEQ ID NO: 721 - DNA, 16 bp, Homo Sapiens
aaaggtctttgatttg

SEQ ID NO: 722 - DNA, 16 bp, Homo Sapiens
ctattccacctactga

SEQ ID NO: 723 - DNA, 16 bp, Homo Sapiens
acccaagacagagcat

SEQ ID NO: 724 - DNA, 16 bp, Homo Sapiens
gtaaaagacctcagtc

SEQ ID NO: 725 - DNA, 16 bp, Homo Sapiens
gacccttataatgagc

SEQ ID NO: 726 - DNA, 16 bp, Homo Sapiens
ctattttcttcctctc

SEQ ID NO: 727 - DNA, 16 bp, Homo Sapiens
gctgtgatccatgaga

SEQ ID NO: 728 - DNA, 16 bp, Homo Sapiens
ctgccttgtaccattc

SEQ ID NO: 729 - DNA, 16 bp, Homo Sapiens
gcagaacatcaagttc

SEQ ID NO: 730 - DNA, 16 bp, Homo Sapiens
tcagcctctgtgaaga

SEQ ID NO: 731 - DNA, 16 bp, Homo Sapiens
ggtttgtggtcctcac

SEQ ID NO: 732 - DNA, 16 bp, Homo Sapiens
tgccacagtgaggaca

SEQ ID NO: 733 - DNA, 16 bp, Homo Sapiens
gtaacccaccttctgt

SEQ ID NO: 734 - DNA, 15 bp, Homo Sapiens
agcaccaggccgatt

SEQ ID NO: 735 - DNA, 16 bp, Homo Sapiens
cctccacaggcatttt

SEQ ID NO: 736 - DNA, 16 bp, Homo Sapiens
cactcacagcacatag

SEQ ID NO: 737 - DNA, 16 bp, Homo Sapiens
ccctgactgtgagatc

SEQ ID NO: 738 - DNA, 16 bp, Homo Sapiens
gtacgcctccagatga

SEQ ID NO: 739 - DNA, 16 bp, Homo Sapiens
cccttcccaagatttg

SEQ ID NO: 740 - DNA, 16 bp, Homo Sapiens
aggccttggcaatcat

SEQ ID NO: 741 - DNA, 16 bp, Homo Sapiens
atgagccctcagctga

SEQ ID NO: 742 - DNA, 16 bp, Homo Sapiens
aagcttgaactctcat

SEQ ID NO: 743 - DNA, 16 bp, Homo Sapiens
gtctctcctcctgtca

SEQ ID NO: 744 - DNA, 16 bp, Homo Sapiens
acctccttcttcctct

SEQ ID NO: 745 - DNA, 15 bp, Homo Sapiens
ggtctccaacccatt

SEQ ID NO: 746 - DNA, 16 bp, Homo Sapiens
ggtgcgggagtgaata

SEQ ID NO: 747 - DNA, 16 bp, Homo Sapiens
acaaagtctgtcaacc

SEQ ID NO: 748 - DNA, 16 bp, Homo Sapiens
tgcagcaataccattg

SEQ ID NO: 749 - DNA, 16 bp, Homo Sapiens
atcaccacgggtctgt

SEQ ID NO: 750 - DNA, 16 bp, Homo Sapiens
aggctcccacctttca

SEQ ID NO: 751 - DNA, 16 bp, Homo Sapiens
gtggtgtagttggtca

SEQ ID NO: 752 - DNA, 16 bp, Homo Sapiens
gattcaaaagatcctg

SEQ ID NO: 753 - DNA, 16 bp, Homo Sapiens
ctgtgggaatgaacaa

SEQ ID NO: 754 - DNA, 15 bp, Homo Sapiens
tgctgcaccaggttg

SEQ ID NO: 755 - DNA, 16 bp, Homo Sapiens
gagttccaaggcctca

SEQ ID NO: 756 - DNA, 16 bp, Homo Sapiens
aacttgaaccatcttt

SEQ ID NO: 757 - DNA, 16 bp, Homo Sapiens
ttagtacctgaagggt

SEQ ID NO: 758 - DNA, 16 bp, Homo Sapiens
gcagttatgcctcaga

SEQ ID NO: 759 - DNA, 16 bp, Homo Sapiens
ttcttgcggagattct

SEQ ID NO: 760 - DNA, 16 bp, Homo Sapiens
cttactgcctcttgct

SEQ ID NO: 761 - DNA, 16 bp, Homo Sapiens
gagtcttccagtgtga

SEQ ID NO: 762 - DNA, 16 bp, Homo Sapiens
atcttgggcctgtgtt

SEQ ID NO: 763 - DNA, 16 bp, Homo Sapiens
agggcaccaccacact

SEQ ID NO: 764 - DNA, 16 bp, Homo Sapiens
tctgattcctcactga

SEQ ID NO: 765 - DNA, 16 bp, Homo Sapiens
gctcaccatcgctatc

SEQ ID NO: 766 - DNA, 16 bp, Homo Sapiens
tgtgggttgattccac

SEQ ID NO: 767 - DNA, 16 bp, Homo Sapiens
gcattgaagtctcatg

SEQ ID NO: 768 - DNA, 16 bp, Homo Sapiens
tctgtcccttcccaga

SEQ ID NO: 769 - DNA, 16 bp, Homo Sapiens
ttctgggagcttcatc

SEQ ID NO: 770 - DNA, 16 bp, Homo Sapiens
ctgctcttttcaccca

SEQ ID NO: 771 - DNA, 16 bp, Homo Sapiens
cctcaaaaacatgtca

SEQ ID NO: 772 - DNA, 16 bp, Homo Sapiens
atgcttttgaagaacg

SEQ ID NO: 773 - DNA, 16 bp, Homo Sapiens
caaccttctcaagaca

SEQ ID NO: 774 - DNA, 16 bp, Homo Sapiens
ccagccaattttgagt

SEQ ID NO: 775 - DNA, 16 bp, Homo Sapiens
catttgcgatcacctg

SEQ ID NO: 776 - DNA, 16 bp, Homo Sapiens
tcatcttcttaaagat

SEQ ID NO: 777 - DNA, 16 bp, Homo Sapiens
gtatcccaagttcact

SEQ ID NO: 778 - DNA, 16 bp, Homo Sapiens
ttactctaatataacc

SEQ ID NO: 779 - DNA, 16 bp, Homo Sapiens
gaccacggtctctaga

SEQ ID NO: 780 - DNA, 16 bp, Homo Sapiens
agagagtgggtgtggt

SEQ ID NO: 781 - DNA, 16 bp, Homo Sapiens
aggcaaacaaacctgg

SEQ ID NO: 782 - DNA, 16 bp, Homo Sapiens
gcaaaataatcagctc

SEQ ID NO: 783 - DNA, 16 bp, Homo Sapiens
tctgaaagtgaactcc

SEQ ID NO: 784 - DNA, 16 bp, Homo Sapiens
ttcgccctctcctact

SEQ ID NO: 785 - DNA, 16 bp, Homo Sapiens
catgaaatagttgtcc

SEQ ID NO: 786 - DNA, 16 bp, Homo Sapiens
ttatgcccgagactaa

SEQ ID NO: 787 - DNA, 16 bp, Homo Sapiens
gcttgtcccgataggt

SEQ ID NO: 788 - DNA, 16 bp, Homo Sapiens
gtgtgatattggcaaa

SEQ ID NO: 789 - DNA, 23 bp, Homo Sapiens
ccactctcaccttctccatctct

SEQ ID NO: 790 - DNA, 22 bp, Homo Sapiens
caaggtcctacgcttccaaaag

SEQ ID NO: 791 - DNA, 19 bp, Homo Sapiens
gggaaacggtcgcactcaa

SEQ ID NO: 792 - DNA, 20 bp, Homo Sapiens
ccgtctgcctcgatgaccta

SEQ ID NO: 793 - DNA, 28 bp, Homo Sapiens
gatataaaaagtggcttaggaggagctt

SEQ ID NO: 794 - DNA, 30 bp, Homo Sapiens
aggaagaacaggatagaaagactgcttata

SEQ ID NO: 795 - DNA, 22 bp, Homo Sapiens
ctcacagaagtcgatggcatca

SEQ ID NO: 796 - DNA, 22 bp, Homo Sapiens
cacaagctgaccaaacgtatcc

SEQ ID NO: 797 - DNA, 22 bp, Homo Sapiens
catgtactggtccctcattgca

SEQ ID NO: 798 - DNA, 28 bp, Homo Sapiens
gtaataatccagactgtgtttctccctt

SEQ ID NO: 799 - DNA, 21 bp, Homo Sapiens
ctcccataccctctcagcgta

SEQ ID NO: 800 - DNA, 21 bp, Homo Sapiens
agccatagggcataagctgtg

SEQ ID NO: 801 - DNA, 31 bp, Homo Sapiens
gcattcagattccaaacaaggaaaatatttg

SEQ ID NO: 802 - DNA, 33 bp, Homo Sapiens
gttaagacttacacacaaaagtaatatcacaac

SEQ ID NO: 803 - DNA, 28 bp, Homo Sapiens
atgatctgctagtgaatgagataagtca

SEQ ID NO: 804 - DNA, 24 bp, Homo Sapiens
acctacttactgtacctggtgaca

SEQ ID NO: 805 - DNA, 29 bp, Homo Sapiens
gcaaacagagatgaatttctgactaaacc

SEQ ID NO: 806 - DNA, 30 bp, Homo Sapiens
gactgaatatcacacttctaaaaggtacgt

SEQ ID NO: 807 - DNA, 18 bp, Homo Sapiens
gctgagtgggctacgtct

SEQ ID NO: 808 - DNA, 18 bp, Homo Sapiens
gtcttcgggctcgcagaa

SEQ ID NO: 809 - DNA, 22 bp, Homo Sapiens
gggtacattcaggaggaagtgc

SEQ ID NO: 810 - DNA, 30 bp, Homo Sapiens
aacacatggaaaccttttagaaactgtttt

SEQ ID NO: 811 - DNA, 22 bp, Homo Sapiens
ccatccctgactgtgagatcaa

SEQ ID NO: 812 - DNA, 20 bp, Homo Sapiens
ccaggtacgcctccagatga

SEQ ID NO: 813 - DNA, 24 bp, Homo Sapiens
aaaagggagttccaattctcacgt

SEQ ID NO: 814 - DNA, 30 bp, Homo Sapiens
ttttctttttagattttgtggtggatgcaa

SEQ ID NO: 815 - DNA, 22 bp, Homo Sapiens
gtctctcggaggaaggacttga

SEQ ID NO: 816 - DNA, 22 bp, Homo Sapiens
cctctctgctctgcagcaaatt

SEQ ID NO: 817 - DNA, 24 bp, Homo Sapiens
caccaagcagaagtaaaacacctc

SEQ ID NO: 818 - DNA, 23 bp, Homo Sapiens
cctctgaactgcagcatttactg

SEQ ID NO: 819 - DNA, 30 bp, Homo Sapiens
agttgttcttgtctttccttttcaagtttt

SEQ ID NO: 820 - DNA, 29 bp, Homo Sapiens
gacatggatttgattgacatactttggag

SEQ ID NO: 821 - DNA, 30 bp, Homo Sapiens
ccagatgctgatactttattacattttgcc

SEQ ID NO: 822 - DNA, 29 bp, Homo Sapiens
tgaactggaggcattattcttaattccac

SEQ ID NO: 823 - DNA, 27 bp, Homo Sapiens
actattttggccaacaatgtctcaaac

SEQ ID NO: 824 - DNA, 22 bp, Homo Sapiens
gctccaaggagattcttagcca

SEQ ID NO: 825 - DNA, 20 bp, Homo Sapiens
agggcatctggatccctgat

SEQ ID NO: 826 - DNA, 21 bp, Homo Sapiens
cttcctgtcctcctagcagga

SEQ ID NO: 827 - DNA, 25 bp, Homo Sapiens
cccaagaactgagtactctgtacct

SEQ ID NO: 828 - DNA, 23 bp, Homo Sapiens
caagagaaagcctttttcgctca

SEQ ID NO: 829 - DNA, 19 bp, Homo Sapiens
gccagggtatgtggctaca

SEQ ID NO: 830 - DNA, 20 bp, Homo Sapiens
acttctcacaccgctgtgtt

SEQ ID NO: 831 - DNA, 22 bp, Homo Sapiens
cacctcagcccatcttgacaaa

SEQ ID NO: 832 - DNA, 18 bp, Homo Sapiens
caccgtgcgttgcttgtt

SEQ ID NO: 833 - DNA, 29 bp, Homo Sapiens
tgatggagatgtgataatttcaggaaaca

SEQ ID NO: 834 - DNA, 22 bp, Homo Sapiens
cggtgacttactgcagctgttt

SEQ ID NO: 835 - DNA, 22 bp, Homo Sapiens
agggtgagaggcatggctatta

SEQ ID NO: 836 - DNA, 22 bp, Homo Sapiens
gctccatcgagtcttcactgtg

SEQ ID NO: 837 - DNA, 22 bp, Homo Sapiens
catggctgcgcttctacttact

SEQ ID NO: 838 - DNA, 24 bp, Homo Sapiens
ctatgggtggtgttgtgttttgtg

SEQ ID NO: 839 - DNA, 30 bp, Homo Sapiens
ccgctacattgattccatttgtaataaacc

SEQ ID NO: 840 - DNA, 27 bp, Homo Sapiens
ccatttcctcaatgtttccagataagg

SEQ ID NO: 841 - DNA, 22 bp, Homo Sapiens
agaagaggagcctggacagtta

SEQ ID NO: 842 - DNA, 28 bp, Homo Sapiens
aaaggaagactcagaggagagagataag

SEQ ID NO: 843 - DNA, 27 bp, Homo Sapiens
catgttatgttaaccaacctccctagt

SEQ ID NO: 844 - DNA, 30 bp, Homo Sapiens
gttccaatcgtcagaaaattttggaaagaa

SEQ ID NO: 845 - DNA, 28 bp, Homo Sapiens
cagaaggattcgatgaatcacaaaatgg

SEQ ID NO: 846 - DNA, 23 bp, Homo Sapiens
agaacaccaagcatcactggatg

SEQ ID NO: 847 - DNA, 22 bp, Homo Sapiens
actcccgatctggatcagcata

SEQ ID NO: 848 - DNA, 24 bp, Homo Sapiens
tttcacatttcagggtcctgacaa

SEQ ID NO: 849 - DNA, 22 bp, Homo Sapiens
cctcacctctatggtgggatca

SEQ ID NO: 850 - DNA, 23 bp, Homo Sapiens
acaggttcttgctggtgtgaaat

SEQ ID NO: 851 - DNA, 22 bp, Homo Sapiens
gaagctgtcaacctgcatgaag

SEQ ID NO: 852 - DNA, 27 bp, Homo Sapiens
tgaatcttttccacatcagtggtgatc

SEQ ID NO: 853 - DNA, 27 bp, Homo Sapiens
gcagaagaaaaagtcaggatgttttca

SEQ ID NO: 854 - DNA, 28 bp, Homo Sapiens
gccctactcaggttaaaatgatgttttg

SEQ ID NO: 855 - DNA, 22 bp, Homo Sapiens
tgcatgtctgccaggaaacttt

SEQ ID NO: 856 - DNA, 27 bp, Homo Sapiens
ccaaatgacaaccaggacaataagtga

SEQ ID NO: 857 - DNA, 22 bp, Homo Sapiens
ctgggacatggccaagagaagt

SEQ ID NO: 858 - DNA, 23 bp, Homo Sapiens
gaggataacaacacgcctctctt

SEQ ID NO: 859 - DNA, 22 bp, Homo Sapiens
aatggctgacactggacatctt

SEQ ID NO: 860 - DNA, 25 bp, Homo Sapiens
ggagcatctcatctgttacagcttc

SEQ ID NO: 861 - DNA, 23 bp, Homo Sapiens
cattcggcacaggatgactgtta

SEQ ID NO: 862 - DNA, 24 bp, Homo Sapiens
ctcctcctgtgatctgcaatctag

SEQ ID NO: 863 - DNA, 28 bp, Homo Sapiens
atcctgtaataacaagtatttcgccgaa

SEQ ID NO: 864 - DNA, 30 bp, Homo Sapiens
cacctttttaaagtacatgtttttccacca

SEQ ID NO: 865 - DNA, 25 bp, Homo Sapiens
cctgaaacttatgggagaaacagga

SEQ ID NO: 866 - DNA, 22 bp, Homo Sapiens
ggtccatcaatcacacgtacca

SEQ ID NO: 867 - DNA, 22 bp, Homo Sapiens
gctggtggtcctaccatacatg

SEQ ID NO: 868 - DNA, 23 bp, Homo Sapiens
tcagagcaggcctattttgaagg

SEQ ID NO: 869 - DNA, 23 bp, Homo Sapiens
gatggacccgtattcattctcca

SEQ ID NO: 870 - DNA, 27 bp, Homo Sapiens
tgctaggattgttaaataaccgccttt

SEQ ID NO: 871 - DNA, 27 bp, Homo Sapiens
gatgagtcagttaggaataggcagttc

SEQ ID NO: 872 - DNA, 23 bp, Homo Sapiens
gcaacaattggtgtttgtctcct

SEQ ID NO: 873 - DNA, 21 bp, Homo Sapiens
acactcttgagggccacaaag

SEQ ID NO: 874 - DNA, 24 bp, Homo Sapiens
tgtgattgtagggtctcccttgat

SEQ ID NO: 875 - DNA, 24 bp, Homo Sapiens
gagaaccagttcagagtggactac

SEQ ID NO: 876 - DNA, 27 bp, Homo Sapiens
tcactcatgttcctatatggacactgt

SEQ ID NO: 877 - DNA, 29 bp, Homo Sapiens
acagctacaccatatatgaatggagaaac

SEQ ID NO: 878 - DNA, 30 bp, Homo Sapiens
tcagcatttgactttaccttatcaatgtct

SEQ ID NO: 879 - DNA, 27 bp, Homo Sapiens
gggtacacacgtaacataaatctgatg

SEQ ID NO: 880 - DNA, 24 bp, Homo Sapiens
gattgggttccagctggaaagtta

SEQ ID NO: 881 - DNA, 22 bp, Homo Sapiens
gccaggaacgtactggtgaaaa

SEQ ID NO: 882 - DNA, 24 bp, Homo Sapiens
tgacctaaagccacctccttactt

SEQ ID NO: 883 - DNA, 26 bp, Homo Sapiens
tactaccttgaggaacatggtatggt

SEQ ID NO: 884 - DNA, 26 bp, Homo Sapiens
ctgtatagcagctgcttatcatcagg

SEQ ID NO: 885 - DNA, 20 bp, Homo Sapiens
gcacggcctcgatcttgtag

SEQ ID NO: 886 - DNA, 23 bp, Homo Sapiens
cgtctacctggagattgacaacc

SEQ ID NO: 887 - DNA, 25 bp, Homo Sapiens
gggtgtacaaccttgaagtgtatgt

SEQ ID NO: 888 - DNA, 25 bp, Homo Sapiens
agaatttacacgcgtagttgaacct

SEQ ID NO: 889 - DNA, 26 bp, Homo Sapiens
tatctctgaaagtgaactccctgcta

SEQ ID NO: 890 - DNA, 22 bp, Homo Sapiens
gaggttcgccctctcctactta

SEQ ID NO: 891 - DNA, 27 bp, Homo Sapiens
agtagagcaatgtatgtctatcctcca

SEQ ID NO: 892 - DNA, 28 bp, Homo Sapiens
gacacaggtagaagactgcactatagta

SEQ ID NO: 893 - DNA, 27 bp, Homo Sapiens
tcttgtatcccaagttcactgatttcc

SEQ ID NO: 894 - DNA, 33 bp, Homo Sapiens
atgcttactctaatataaccctctatttagtca

SEQ ID NO: 895 - DNA, 27 bp, Homo Sapiens
agaggctttggagtatttcatgaaaca

SEQ ID NO: 896 - DNA, 30 bp, Homo Sapiens
agagtgagctttcattttctcagttatctt

SEQ ID NO: 897 - DNA, 20 bp, Homo Sapiens
gcctcacgttggtccacatc

SEQ ID NO: 898 - DNA, 19 bp, Homo Sapiens
tctcaccacccgcacgtct

SEQ ID NO: 899 - DNA, 28 bp, Homo Sapiens
tcagtgaagaactgttctaccagatact

SEQ ID NO: 900 - DNA, 30 bp, Homo Sapiens
attagtggagagctactattttcagaaacg

SEQ ID NO: 901 - DNA, 28 bp, Homo Sapiens
gaatcagagcagcctaaagaatcaaatg

SEQ ID NO: 902 - DNA, 30 bp, Homo Sapiens
ggcatggcagaaataatacattcttctagt

SEQ ID NO: 903 - DNA, 21 bp, Homo Sapiens
ctcacggctttgtccaagaga

SEQ ID NO: 904 - DNA, 23 bp, Homo Sapiens
gtgatgggcagaagggcacaaag

SEQ ID NO: 905 - DNA, 33 bp, Homo Sapiens
agatatttttggattacttactcaagttggtca

SEQ ID NO: 906 - DNA, 23 bp, Homo Sapiens
gaaagctgcttttccagggtttc

SEQ ID NO: 907 - DNA, 24 bp, Homo Sapiens
gtgccctattacctcaatcatcct

SEQ ID NO: 908 - DNA, 22 bp, Homo Sapiens
acgccttcacctttaacacctc

SEQ ID NO: 909 - DNA, 21 bp, Homo Sapiens
ctctgtcactgactgctgtga

SEQ ID NO: 910 - DNA, 20 bp, Homo Sapiens
gctgacattccggcaagaga

SEQ ID NO: 911 - DNA, 23 bp, Homo Sapiens
ctagtgttcctggtcctgacttg

SEQ ID NO: 912 - DNA, 23 bp, Homo Sapiens
ggtgtcagtgactgtgatcacag

SEQ ID NO: 913 - DNA, 32 bp, Homo Sapiens
cctagtagaatgtttactaccaaatggaatga

SEQ ID NO: 914 - DNA, 28 bp, Homo Sapiens
agattcatcttgaagaagttgatggagg

SEQ ID NO: 915 - DNA, 28 bp, Homo Sapiens
cacttttacagaaacagctgttataccc

SEQ ID NO: 916 - DNA, 33 bp, Homo Sapiens
tcatgttctttacagagaacttcaataattctt

SEQ ID NO: 917 - DNA, 22 bp, Homo Sapiens
gcatgccaattggtctgtatcc

SEQ ID NO: 918 - DNA, 30 bp, Homo Sapiens
ggatccttttccatagagaaagtatctacc

SEQ ID NO: 919 - DNA, 19 bp, Homo Sapiens
aggcaagcctggcacatac

SEQ ID NO: 920 - DNA, 21 bp, Homo Sapiens
tccatggtcctgtctgtcctt

SEQ ID NO: 921 - DNA, 27 bp, Homo Sapiens
gcacgattcttttagatctgagatgca

SEQ ID NO: 922 - DNA, 22 bp, Homo Sapiens
agcttttccattgcctcgactt

SEQ ID NO: 923 - DNA, 26 bp, Homo Sapiens
agacagagctaaggaagcttaaagtg

SEQ ID NO: 924 - DNA, 28 bp, Homo Sapiens
ggtcaatcctatgcaaaaatctttcacc

SEQ ID NO: 925 - DNA, 29 bp, Homo Sapiens
gatctatgttcgaacaggtatctaccatg

SEQ ID NO: 926 - DNA, 33 bp, Homo Sapiens
actgctaaacactaatataacctttggaaatat

SEQ ID NO: 927 - DNA, 28 bp, Homo Sapiens
cttggttctttgtttgtcttaattgcag

SEQ ID NO: 928 - DNA, 30 bp, Homo Sapiens
gcaaaacaggaagcatacttactaaacttt

SEQ ID NO: 929 - DNA, 27 bp, Homo Sapiens
ggagtatatcgtctacacaattggaca

SEQ ID NO: 930 - DNA, 22 bp, Homo Sapiens
ctcaaacacaaagctggtgtgt

SEQ ID NO: 931 - DNA, 25 bp, Homo Sapiens
tctctcctctcatcctgtctcctta

SEQ ID NO: 932 - DNA, 27 bp, Homo Sapiens
tgcctattggcacttatatagatacgc

SEQ ID NO: 933 - DNA, 29 bp, Homo Sapiens
tattatgacttgtcacaatgtcaccacat

SEQ ID NO: 934 - DNA, 25 bp, Homo Sapiens
gactcgagtgatgattgggagattc

SEQ ID NO: 935 - DNA, 19 bp, Homo Sapiens
cgtgcctgccaatggtgat

SEQ ID NO: 936 - DNA, 25 bp, Homo Sapiens
ctgaagaagatgtggaaaagtccca

SEQ ID NO: 937 - DNA, 33 bp, Homo Sapiens
aattcctcaaaaacatgtcagtgtgattttatt

SEQ ID NO: 938 - DNA, 29 bp, Homo Sapiens
gttgatgcttttgaagaacgacataaaag

SEQ ID NO: 939 - DNA, 22 bp, Homo Sapiens
gaagtgggttacctgacagtgt

SEQ ID NO: 940 - DNA, 26 bp, Homo Sapiens
gctcctttctttgacagaaaaagcag

SEQ ID NO: 941 - DNA, 23 bp, Homo Sapiens
ccatccagcttcaaaagctcttc

SEQ ID NO: 942 - DNA, 27 bp, Homo Sapiens
ccctcttttacactcctattgatctgg

SEQ ID NO: 943 - DNA, 28 bp, Homo Sapiens
tttcccttggagatatcgatctgttaga

SEQ ID NO: 944 - DNA, 22 bp, Homo Sapiens
ctctgaacaggacgaactggat

SEQ ID NO: 945 - DNA, 22 bp, Homo Sapiens
gacggtggtgtagttggtcata

SEQ ID NO: 946 - DNA, 25 bp, Homo Sapiens
gggagattcaaaagatcctggagtt

SEQ ID NO: 947 - DNA, 26 bp, Homo Sapiens
tgcttttctaactctctttgactgca

SEQ ID NO: 948 - DNA, 25 bp, Homo Sapiens
tacatacagtttcttgcagccaagt

SEQ ID NO: 949 - DNA, 18 bp, Homo Sapiens
actgtgcgacgagctgtg

SEQ ID NO: 950 - DNA, 21 bp, Homo Sapiens
atctcagctgaggagatgggt

SEQ ID NO: 951 - DNA, 22 bp, Homo Sapiens
ccttgagttccaaggcctcatt

SEQ ID NO: 952 - DNA, 30 bp, Homo Sapiens
ttgtaacttgaaccatcttttaactcaggt

SEQ ID NO: 953 - DNA, 23 bp, Homo Sapiens
gttcttcagggcaaagaagtcca

SEQ ID NO: 954 - DNA, 24 bp, Homo Sapiens
ctctgttttccaatgcaaccagtt

SEQ ID NO: 955 - DNA, 26 bp, Homo Sapiens
tttcctgtagcaaaaccagaaatcct

SEQ ID NO: 956 - DNA, 28 bp, Homo Sapiens
aaataatcatctcacctctgctcagttc

SEQ ID NO: 957 - DNA, 29 bp, Homo Sapiens
gatagaggttccttaagatctcgatttcc

SEQ ID NO: 958 - DNA, 22 bp, Homo Sapiens
gaaggttgagctctgcaggtat

SEQ ID NO: 959 - DNA, 28 bp, Homo Sapiens
tttcagcatgaaatagtgtatcagtggt

SEQ ID NO: 960 - DNA, 25 bp, Homo Sapiens
cctggctttaaatcctcgaacacaa

SEQ ID NO: 961 - DNA, 23 bp, Homo Sapiens
tggtttctggtgggaccattatg

SEQ ID NO: 962 - DNA, 22 bp, Homo Sapiens
gtcctctggatctcttcatgca

SEQ ID NO: 963 - DNA, 26 bp, Homo Sapiens
agggcttttgttttcttccctttaga

SEQ ID NO: 964 - DNA, 22 bp, Homo Sapiens
gccattgtgcttgaatgcacta

SEQ ID NO: 965 - DNA, 22 bp, Homo Sapiens
ctctgtcacagtggattcgaga

SEQ ID NO: 966 - DNA, 25 bp, Homo Sapiens
caacatgacgaagatggcaaacttc

SEQ ID NO: 967 - DNA, 24 bp, Homo Sapiens
gttgcttttgaacagggcaaaatc

SEQ ID NO: 968 - DNA, 29 bp, Homo Sapiens
ttccctcctttacttcatatcacttacct

SEQ ID NO: 969 - DNA, 23 bp, Homo Sapiens
aaaaaggcaaacaaacctggcta

SEQ ID NO: 970 - DNA, 28 bp, Homo Sapiens
cccagcaaaataatcagctctcattttc

SEQ ID NO: 971 - DNA, 22 bp, Homo Sapiens
tctgcgtgtacctgtcgtagta

SEQ ID NO: 972 - DNA, 24 bp, Homo Sapiens
cctgaccactttccctctcttttg

SEQ ID NO: 973 - DNA, 25 bp, Homo Sapiens
gggacatgaaatagttgtcctagca

SEQ ID NO: 974 - DNA, 27 bp, Homo Sapiens
aacattatgcccgagactaacaaaaga

SEQ ID NO: 975 - DNA, 22 bp, Homo Sapiens
gactgatgagaacgcaatgcaa

SEQ ID NO: 976 - DNA, 27 bp, Homo Sapiens
ttagggtgatcagttatgaagaaggga

SEQ ID NO: 977 - DNA, 21 bp, Homo Sapiens
ctcttcccacagccactgttt

SEQ ID NO: 978 - DNA, 28 bp, Homo Sapiens
tcagttcctatatcctgtgtctgtgaat

SEQ ID NO: 979 - DNA, 23 bp, Homo Sapiens
caaatacacagaggaagccttcg

SEQ ID NO: 980 - DNA, 29 bp, Homo Sapiens
ccaggattcttacagaaaacaagtggtta

SEQ ID NO: 981 - DNA, 30 bp, Homo Sapiens
tcactgttccataatgaagttaatgtctcc

SEQ ID NO: 982 - DNA, 22 bp, Homo Sapiens
ttcccaggagcgagaggacatt

SEQ ID NO: 983 - DNA, 23 bp, Homo Sapiens
tatgttggaggaggtcaggctta

SEQ ID NO: 984 - DNA, 20 bp, Homo Sapiens
cgtgagcccatctgggaaac

SEQ ID NO: 985 - DNA, 27 bp, Homo Sapiens
tttttgatgaaacaagacgactttgtg

SEQ ID NO: 986 - DNA, 33 bp, Homo Sapiens
gaataggatattgtatcataccaatttctcgat

SEQ ID NO: 987 - DNA, 30 bp, Homo Sapiens
tgagttttctgagtgcttttatcagaatga

SEQ ID NO: 988 - DNA, 29 bp, Homo Sapiens
cctcaagcaaagttttaaggcaatttact

SEQ ID NO: 989 - DNA, 22 bp, Homo Sapiens
ctgtcctccacaggcatttttg

SEQ ID NO: 990 - DNA, 22 bp, Homo Sapiens
ccctcactcacagcacatagtc

SEQ ID NO: 991 - DNA, 22 bp, Homo Sapiens
gtgaggcagtctttactcacct

SEQ ID NO: 992 - DNA, 26 bp, Homo Sapiens
taggaaatgcatttcctttcttccca

SEQ ID NO: 993 - DNA, 29 bp, Homo Sapiens
cccttcattgcttaacacattttcctatt

SEQ ID NO: 994 - DNA, 31 bp, Homo Sapiens
atggcttacgttaaaataggaaatcagattt

SEQ ID NO: 995 - DNA, 28 bp, Homo Sapiens
cttctgttcaattttgttgagcttctga

SEQ ID NO: 996 - DNA, 22 bp, Homo Sapiens
accagacgtcactttcaaacgt

SEQ ID NO: 997 - DNA, 31 bp, Homo Sapiens
gctagagacaatgaattaagggaaaatgaca

SEQ ID NO: 998 - DNA, 28 bp, Homo Sapiens
acagagaatctccattttagcacttacc

SEQ ID NO: 999 - DNA, 19 bp, Homo Sapiens
ctccacgctcaggttggag

SEQ ID NO: 1000 - DNA, 21 bp, Homo Sapiens
ccacatgagtgactgcctctc

SEQ ID NO: 1001 - DNA, 19 bp, Homo Sapiens
gaagcctacgtgatggcca

SEQ ID NO: 1002 - DNA, 22 bp, Homo Sapiens
ttgtctttgtgttcccggacat

SEQ ID NO: 1003 - DNA, 22 bp, Homo Sapiens
gggaaatgtgagcccttgagat

SEQ ID NO: 1004 - DNA, 22 bp, Homo Sapiens
cctgtggctgtcagtatttgga

SEQ ID NO: 1005 - DNA, 24 bp, Homo Sapiens
aatgtgtcagcctcaaagaaaagc

SEQ ID NO: 1006 - DNA, 24 bp, Homo Sapiens
ctgtcatccctattggcaggttac

SEQ ID NO: 1007 - DNA, 21 bp, Homo Sapiens
ggagtccatgtgttctgagct

SEQ ID NO: 1008 - DNA, 21 bp, Homo Sapiens
gtgaaggttcccagctgttct

SEQ ID NO: 1009 - DNA, 22 bp, Homo Sapiens
tcactttgtgaccattccggtt

SEQ ID NO: 1010 - DNA, 24 bp, Homo Sapiens
cctcttctacctgaagagcaagtc

SEQ ID NO: 1011 - DNA, 28 bp, Homo Sapiens
gttatgattttgcagaaaacagatctgt

SEQ ID NO: 1012 - DNA, 28 bp, Homo Sapiens
gccttctagaacagtagacacaaaacag

SEQ ID NO: 1013 - DNA, 26 bp, Homo Sapiens
ttgcaagtcctctcaagtctaatagc

SEQ ID NO: 1014 - DNA, 30 bp, Homo Sapiens
agattatccaattctgtttctttccttcca

SEQ ID NO: 1015 - DNA, 25 bp, Homo Sapiens
ctgttaaggtcaatgacgcagagta

SEQ ID NO: 1016 - DNA, 23 bp, Homo Sapiens
cctcacaaccttgcggaattttg

SEQ ID NO: 1017 - DNA, 27 bp, Homo Sapiens
ccacatgactgtcctgtagattaagag

SEQ ID NO: 1018 - DNA, 22 bp, Homo Sapiens
ttcaccgtgacccaaagtactg

SEQ ID NO: 1019 - DNA, 28 bp, Homo Sapiens
gctatcaaagagatgattgagaactggt

SEQ ID NO: 1020 - DNA, 18 bp, Homo Sapiens
tgggcatgcgctgtacat

SEQ ID NO: 1021 - DNA, 33 bp, Homo Sapiens
atgcaataattttcccactatcattgattattt

SEQ ID NO: 1022 - DNA, 20 bp, Homo Sapiens
cccgagggttgttgatgtcc

SEQ ID NO: 1023 - DNA, 22 bp, Homo Sapiens
ccagactgaggtatcgcctcat

SEQ ID NO: 1024 - DNA, 22 bp, Homo Sapiens
cacccacatcatccttggttca

SEQ ID NO: 1025 - DNA, 23 bp, Homo Sapiens
tgctcccaggctgtttatttgaa

SEQ ID NO: 1026 - DNA, 25 bp, Homo Sapiens
tgagaacattgcctatggagacaac

SEQ ID NO: 1027 - DNA, 21 bp, Homo Sapiens
ctgggtgccctcatttacctt

SEQ ID NO: 1028 - DNA, 20 bp, Homo Sapiens
gtgccacgacagcgatgaga

SEQ ID NO: 1029 - DNA, 26 bp, Homo Sapiens
ctgaagagtgttgtccagttaatggt

SEQ ID NO: 1030 - DNA, 24 bp, Homo Sapiens
tccttgcttatcctcaagcaacag

SEQ ID NO: 1031 - DNA, 23 bp, Homo Sapiens
attttcacacagccaggagtctt

SEQ ID NO: 1032 - DNA, 29 bp, Homo Sapiens
ccaatgcaacagactttaaagaagttgtg

SEQ ID NO: 1033 - DNA, 24 bp, Homo Sapiens
cactgtgttactgccatcgactta

SEQ ID NO: 1034 - DNA, 26 bp, Homo Sapiens
tcgagatttagcagccagaaatgttt

SEQ ID NO: 1035 - DNA, 22 bp, Homo Sapiens
tgggtgtttttggagaagcaca

SEQ ID NO: 1036 - DNA, 25 bp, Homo Sapiens
gtagattctcgcctctattgagctg

SEQ ID NO: 1037 - DNA, 28 bp, Homo Sapiens
cattcaccaacttatgccaattctcttg

SEQ ID NO: 1038 - DNA, 29 bp, Homo Sapiens
ctttctgaatattgagctcatcagtgaga

SEQ ID NO: 1039 - DNA, 31 bp, Homo Sapiens
aaaaatgatcttgacaaagcaaataaagaca

SEQ ID NO: 1040 - DNA, 29 bp, Homo Sapiens
agctgtactcctagaattaaacacacatc

SEQ ID NO: 1041 - DNA, 24 bp, Homo Sapiens
ttaccatttgcgatcacctggatt

SEQ ID NO: 1042 - DNA, 33 bp, Homo Sapiens
ctgctcatcttcttaaagatttgatttttctcc

SEQ ID NO: 1043 - DNA, 24 bp, Homo Sapiens
cccacaccaagtatcagtatggag

SEQ ID NO: 1044 - DNA, 25 bp, Homo Sapiens
tcaccaactggattctttttccctt

SEQ ID NO: 1045 - DNA, 25 bp, Homo Sapiens
aaatattctccaggcgtttcttcca

SEQ ID NO: 1046 - DNA, 24 bp, Homo Sapiens
tctgtatcctcagagtggcattct

SEQ ID NO: 1047 - DNA, 21 bp, Homo Sapiens
gtcggagatgcaggtctcaag

SEQ ID NO: 1048 - DNA, 21 bp, Homo Sapiens
ccaggctgttgggaacgtaag

SEQ ID NO: 1049 - DNA, 25 bp, Homo Sapiens
acccttccataaaggctttaacaca

SEQ ID NO: 1050 - DNA, 25 bp, Homo Sapiens
tgtttggttttgtaggtcttgtgga

SEQ ID NO: 1051 - DNA, 30 bp, Homo Sapiens
actaagccctatttctactctttctactgt

SEQ ID NO: 1052 - DNA, 28 bp, Homo Sapiens
agacgatcctgaagaaagagaagaaaag

SEQ ID NO: 1053 - DNA, 26 bp, Homo Sapiens
ataacgacacaacacaaaatagccgt

SEQ ID NO: 1054 - DNA, 24 bp, Homo Sapiens
ccacgggaaagtggtgaagatatg

SEQ ID NO: 1055 - DNA, 24 bp, Homo Sapiens
agggacaaagtctgtcaaccaaat

SEQ ID NO: 1056 - DNA, 22 bp, Homo Sapiens
gacctgcagcaataccattgac

SEQ ID NO: 1057 - DNA, 25 bp, Homo Sapiens
agtaggatgatacatggtggtgtct

SEQ ID NO: 1058 - DNA, 22 bp, Homo Sapiens
ctggtctcccacaatgaaggtc

SEQ ID NO: 1059 - DNA, 22 bp, Homo Sapiens
ccagtctctgcattccacactt

SEQ ID NO: 1060 - DNA, 29 bp, Homo Sapiens
atcatcttaagtgtttttccagtgtctga

SEQ ID NO: 1061 - DNA, 30 bp, Homo Sapiens
gagaaatatgaagtcttcatggatgtttgc

SEQ ID NO: 1062 - DNA, 24 bp, Homo Sapiens
gaagtagctacactgcgcgtataa

SEQ ID NO: 1063 - DNA, 25 bp, Homo Sapiens
ggcccaaattcaccaataatagagg

SEQ ID NO: 1064 - DNA, 30 bp, Homo Sapiens
gactggagaatgtatacacaccttatatgg

SEQ ID NO: 1065 - DNA, 23 bp, Homo Sapiens
cgcagtgctaaccaagttctttc

SEQ ID NO: 1066 - DNA, 30 bp, Homo Sapiens
ccatggttaaataaaatgccacttactgtt

SEQ ID NO: 1067 - DNA, 22 bp, Homo Sapiens
gagaatcgaagcgctacctgat

SEQ ID NO: 1068 - DNA, 21 bp, Homo Sapiens
ctgcccaacgcaccgaatagt

SEQ ID NO: 1069 - DNA, 25 bp, Homo Sapiens
cagccacgggtaataatttttgtcc

SEQ ID NO: 1070 - DNA, 22 bp, Homo Sapiens
gcagctttgcacctgttttgtt

SEQ ID NO: 1071 - DNA, 30 bp, Homo Sapiens
ttgcacaaaaatttaatactgacccatgaa

SEQ ID NO: 1072 - DNA, 24 bp, Homo Sapiens
cattggcacaggatcattgatgtc

SEQ ID NO: 1073 - DNA, 25 bp, Homo Sapiens
tttttaccacagcaatgtgtgttct

SEQ ID NO: 1074 - DNA, 22 bp, Homo Sapiens
gtccttgagcatcccttgtgtt

SEQ ID NO: 1075 - DNA, 21 bp, Homo Sapiens
caagcccactgtctatggtgt

SEQ ID NO: 1076 - DNA, 23 bp, Homo Sapiens
ccgtcaggctgtatttcttccac

SEQ ID NO: 1077 - DNA, 26 bp, Homo Sapiens
cttccaaatctacagagttccctgtt

SEQ ID NO: 1078 - DNA, 28 bp, Homo Sapiens
taaccatatcaaattcacacactggcat

SEQ ID NO: 1079 - DNA, 30 bp, Homo Sapiens
aactctaaattttctcttggaaactcccat

SEQ ID NO: 1080 - DNA, 27 bp, Homo Sapiens
tctgaagcaatttaggtatgaaagcca

SEQ ID NO: 1081 - DNA, 23 bp, Homo Sapiens
ttacagaaacgcatccagcaaga

SEQ ID NO: 1082 - DNA, 29 bp, Homo Sapiens
caatagcgacaatgaaaaactccaagatc

SEQ ID NO: 1083 - DNA, 26 bp, Homo Sapiens
cagctctgaaacataccattgttcaa

SEQ ID NO: 1084 - DNA, 30 bp, Homo Sapiens
acctttatccaaaagaattttctcctgtgt

SEQ ID NO: 1085 - DNA, 20 bp, Homo Sapiens
tatgggctgtgtgggacgtg

SEQ ID NO: 1086 - DNA, 19 bp, Homo Sapiens
gtctgcatcgaagacacgc

SEQ ID NO: 1087 - DNA, 28 bp, Homo Sapiens
tatgcaattttgaaccttaccctcttct

SEQ ID NO: 1088 - DNA, 26 bp, Homo Sapiens
cactctatgtgctttcattcctggaa

SEQ ID NO: 1089 - DNA, 20 bp, Homo Sapiens
cttgtctgccacctgcagat

SEQ ID NO: 1090 - DNA, 21 bp, Homo Sapiens
catctccttgcctccactcac

SEQ ID NO: 1091 - DNA, 25 bp, Homo Sapiens
taacaaccctcctgccatcatattg

SEQ ID NO: 1092 - DNA, 22 bp, Homo Sapiens
ctccctctgcagagttgttagc

SEQ ID NO: 1093 - DNA, 22 bp, Homo Sapiens
tcaagtgacacctcacctctct

SEQ ID NO: 1094 - DNA, 21 bp, Homo Sapiens
gaaggaagtgtgccaggcata

SEQ ID NO: 1095 - DNA, 26 bp, Homo Sapiens
gattcatcaggagagcatttaaggga

SEQ ID NO: 1096 - DNA, 29 bp, Homo Sapiens
tggagcatatgattttatggtaaaggtgt

SEQ ID NO: 1097 - DNA, 20 bp, Homo Sapiens
gccagtaacccaccttctgt

SEQ ID NO: 1098 - DNA, 19 bp, Homo Sapiens
gatgagcaccaggccgatt

SEQ ID NO: 1099 - DNA, 26 bp, Homo Sapiens
atggctctgtaaattctacccgtttt

SEQ ID NO: 1100 - DNA, 23 bp, Homo Sapiens
acaactcggctttcatttgaacc

SEQ ID NO: 1101 - DNA, 20 bp, Homo Sapiens
cccagtaccattcctcgact

SEQ ID NO: 1102 - DNA, 20 bp, Homo Sapiens
gctctgggcagaatgggttg

SEQ ID NO: 1103 - DNA, 19 bp, Homo Sapiens
gcgggtagctacgatgagg

SEQ ID NO: 1104 - DNA, 24 bp, Homo Sapiens
cccaaaagaagcaagatggaagtc

SEQ ID NO: 1105 - DNA, 20 bp, Homo Sapiens
ctctacgtctcctccgacca

SEQ ID NO: 1106 - DNA, 30 bp, Homo Sapiens
cttatttattggtctctcattctcccatcc

SEQ ID NO: 1107 - DNA, 20 bp, Homo Sapiens
gagcagggccaacgttagaa

SEQ ID NO: 1108 - DNA, 22 bp, Homo Sapiens
ccagccaataggagcagagatg

SEQ ID NO: 1109 - DNA, 24 bp, Homo Sapiens
caaggacccaaacatcatccatct

SEQ ID NO: 1110 - DNA, 23 bp, Homo Sapiens
catcgctggaggaagaattaggg

SEQ ID NO: 1111 - DNA, 33 bp, Homo Sapiens
cagatatttctttccttaactaaagtactcaga

SEQ ID NO: 1112 - DNA, 33 bp, Homo Sapiens
agaaaatcaaagcattcttaccttactacatca

SEQ ID NO: 1113 - DNA, 24 bp, Homo Sapiens
gctggagaagagatacgaagaacc

SEQ ID NO: 1114 - DNA, 23 bp, Homo Sapiens
gtgagtggtaggtcttgtaggga

SEQ ID NO: 1115 - DNA, 30 bp, Homo Sapiens
ataactggtgtacttgataggcatttgaat

SEQ ID NO: 1116 - DNA, 30 bp, Homo Sapiens
gatctgttgtcatcttataaatctcccaga

SEQ ID NO: 1117 - DNA, 27 bp, Homo Sapiens
ttgaaagagaacacacttactctccac

SEQ ID NO: 1118 - DNA, 24 bp, Homo Sapiens
ctgagacattcctatgtcctgctc

SEQ ID NO: 1119 - DNA, 25 bp, Homo Sapiens
ggttccacataaggttctcatgaga

SEQ ID NO: 1120 - DNA, 33 bp, Homo Sapiens
tggactggcagactatgttaatctttttatttt

SEQ ID NO: 1121 - DNA, 22 bp, Homo Sapiens
cttgcctagacagcaccgtaat

SEQ ID NO: 1122 - DNA, 23 bp, Homo Sapiens
aggaggataaagacctggtccat

SEQ ID NO: 1123 - DNA, 22 bp, Homo Sapiens
acaacacacagttggaggactt

SEQ ID NO: 1124 - DNA, 22 bp, Homo Sapiens
cccatcacacaccataactcca

SEQ ID NO: 1125 - DNA, 30 bp, Homo Sapiens
agacttagtacctgaagggtgaaatattct

SEQ ID NO: 1126 - DNA, 23 bp, Homo Sapiens
gggtgcagttatgcctcagattc

SEQ ID NO: 1127 - DNA, 26 bp, Homo Sapiens
tgaaaacaatggtgactacatggaca

SEQ ID NO: 1128 - DNA, 28 bp, Homo Sapiens
tcttcttatatgaggctggacgatcata

SEQ ID NO: 1129 - DNA, 22 bp, Homo Sapiens
gaccgagaaggaccaactgatc

SEQ ID NO: 1130 - DNA, 30 bp, Homo Sapiens
aaaatctatttgcagtttgaatggtcaaca

SEQ ID NO: 1131 - DNA, 26 bp, Homo Sapiens
tggtctcaatgatatggagatggtga

SEQ ID NO: 1132 - DNA, 27 bp, Homo Sapiens
tcacatttctttgtacaggaaaacacg

SEQ ID NO: 1133 - DNA, 23 bp, Homo Sapiens
gttgaagctgaacacgcaaaaga

SEQ ID NO: 1134 - DNA, 27 bp, Homo Sapiens
tcagtaacgtgaagtatgtcatgttgg

SEQ ID NO: 1135 - DNA, 22 bp, Homo Sapiens
cccagacatgacagccatcatc

SEQ ID NO: 1136 - DNA, 28 bp, Homo Sapiens
acgttctaagagagtgacagaaaggtaa

SEQ ID NO: 1137 - DNA, 23 bp, Homo Sapiens
ctcacctgtgacattcaccatga

SEQ ID NO: 1138 - DNA, 25 bp, Homo Sapiens
ccaacaataggacagtgcttattgg

SEQ ID NO: 1139 - DNA, 30 bp, Homo Sapiens
caggttattttatacctcacctcattgtca

SEQ ID NO: 1140 - DNA, 30 bp, Homo Sapiens
gttttcctttgtgtcattcccttttatcag

SEQ ID NO: 1141 - DNA, 22 bp, Homo Sapiens
ccactccttgcttctcagatga

SEQ ID NO: 1142 - DNA, 26 bp, Homo Sapiens
cagaggacaatgtgatgaagatagca

SEQ ID NO: 1143 - DNA, 22 bp, Homo Sapiens
gcctggctcattaagatgacct

SEQ ID NO: 1144 - DNA, 25 bp, Homo Sapiens
tctctatcactccttgaagccatca

SEQ ID NO: 1145 - DNA, 22 bp, Homo Sapiens
agagaggccttgggactgatac

SEQ ID NO: 1146 - DNA, 26 bp, Homo Sapiens
gatgaagatgatcgggaagcataaga

SEQ ID NO: 1147 - DNA, 22 bp, Homo Sapiens
aggcaaacatggtgggattttg

SEQ ID NO: 1148 - DNA, 29 bp, Homo Sapiens
tttctctttgggtcctaggtattatgaga

SEQ ID NO: 1149 - DNA, 29 bp, Homo Sapiens
tactcaaactattgggtggatttgtttgt

SEQ ID NO: 1150 - DNA, 28 bp, Homo Sapiens
aacatgtgtagaaagcagatttctccat

SEQ ID NO: 1151 - DNA, 20 bp, Homo Sapiens
ctctccaggacgcacagttt

SEQ ID NO: 1152 - DNA, 20 bp, Homo Sapiens
actcagtcggaggtgaggaa

SEQ ID NO: 1153 - DNA, 29 bp, Homo Sapiens
tgcacagtgaatccaaaagaaagtatact

SEQ ID NO: 1154 - DNA, 30 bp, Homo Sapiens
cacgaattacaatgaattcaagttacctgt

SEQ ID NO: 1155 - DNA, 21 bp, Homo Sapiens
cgagcagctctctcttcagga

SEQ ID NO: 1156 - DNA, 21 bp, Homo Sapiens
ctacgaggctgagcacgaata

SEQ ID NO: 1157 - DNA, 26 bp, Homo Sapiens
ggtttcataacccacagatccatttc

SEQ ID NO: 1158 - DNA, 27 bp, Homo Sapiens
ctcagaaaaatgccaacatacctgatg

SEQ ID NO: 1159 - DNA, 26 bp, Homo Sapiens
aaaaatgtaccactactcaactgtgg

SEQ ID NO: 1160 - DNA, 21 bp, Homo Sapiens
agaggaggagctggagatcag

SEQ ID NO: 1161 - DNA, 28 bp, Homo Sapiens
cttagagcatagtaagcagtagggagta

SEQ ID NO: 1162 - DNA, 27 bp, Homo Sapiens
tggacaagcactgaaagataagaaaga

SEQ ID NO: 1163 - DNA, 24 bp, Homo Sapiens
agttagtgtggacgtctctgtaca

SEQ ID NO: 1164 - DNA, 20 bp, Homo Sapiens
atggcgacttgtgcgttttc

SEQ ID NO: 1165 - DNA, 28 bp, Homo Sapiens
agtttgccaagtgaaatagtacactagg

SEQ ID NO: 1166 - DNA, 28 bp, Homo Sapiens
gcatacatcagacagcacagaattgata

SEQ ID NO: 1167 - DNA, 22 bp, Homo Sapiens
aatccctggaaaaggcaatcga

SEQ ID NO: 1168 - DNA, 23 bp, Homo Sapiens
ccctcctcgctttatttttggga

SEQ ID NO: 1169 - DNA, 22 bp, Homo Sapiens
tgttcctcctctaccacacgat

SEQ ID NO: 1170 - DNA, 24 bp, Homo Sapiens
gcaagctggcttttggaaatgaat

SEQ ID NO: 1171 - DNA, 27 bp, Homo Sapiens
taacacttgagaaaacccaggctaaaa

SEQ ID NO: 1172 - DNA, 25 bp, Homo Sapiens
ttgctggaggatagaaagtaagtgc

SEQ ID NO: 1173 - DNA, 29 bp, Homo Sapiens
ggaaaaattgtgaagatctgtgactttgg

SEQ ID NO: 1174 - DNA, 30 bp, Homo Sapiens
ctgactttagagattaaagtgaaggaggat

SEQ ID NO: 1175 - DNA, 22 bp, Homo Sapiens
gacacccaaaagtccacctgaa

SEQ ID NO: 1176 - DNA, 22 bp, Homo Sapiens
ccattccactgcatggttcact

SEQ ID NO: 1177 - DNA, 22 bp, Homo Sapiens
tcatagggcaccaccacactat

SEQ ID NO: 1178 - DNA, 23 bp, Homo Sapiens
ggcctctgattcctcactgattg

SEQ ID NO: 1179 - DNA, 30 bp, Homo Sapiens
ttccttcttcaatttttgttgtttccatgt

SEQ ID NO: 1180 - DNA, 29 bp, Homo Sapiens
tgcaatttacctagtaatgggttgtaaca

SEQ ID NO: 1181 - DNA, 22 bp, Homo Sapiens
ccctttcttgcggagattctct

SEQ ID NO: 1182 - DNA, 24 bp, Homo Sapiens
tttccttactgcctcttgcttctc

SEQ ID NO: 1183 - DNA, 24 bp, Homo Sapiens
gtcttgcatttgaagaaggaagcc

SEQ ID NO: 1184 - DNA, 33 bp, Homo Sapiens
aacccaaagtatgagataaatactgtcataaat

SEQ ID NO: 1185 - DNA, 29 bp, Homo Sapiens
ttcagatgcatctgttactatcttttgct

SEQ ID NO: 1186 - DNA, 22 bp, Homo Sapiens
tgccactccctctaggatcaaa

SEQ ID NO: 1187 - DNA, 25 bp, Homo Sapiens
ccatgtatgaagtacagtggaaggt

SEQ ID NO: 1188 - DNA, 26 bp, Homo Sapiens
ccctgtttcatactgaccaaaactca

SEQ ID NO: 1189 - DNA, 20 bp, Homo Sapiens
cgccaggctcacctctatag

SEQ ID NO: 1190 - DNA, 23 bp, Homo Sapiens
aggagcgatgacggaatataagc

SEQ ID NO: 1191 - DNA, 30 bp, Homo Sapiens
tgatgctttctggctggatttaaattatct

SEQ ID NO: 1192 - DNA, 30 bp, Homo Sapiens
ccattaccttttctcttgatcatccatact

SEQ ID NO: 1193 - DNA, 22 bp, Homo Sapiens
cctggagtcttccagtgtgatg

SEQ ID NO: 1194 - DNA, 22 bp, Homo Sapiens
cctcatcttgggcctgtgttat

SEQ ID NO: 1195 - DNA, 22 bp, Homo Sapiens
ggtagctcatcatctgggacag

SEQ ID NO: 1196 - DNA, 21 bp, Homo Sapiens
gccgaaccaatacaaccctct

SEQ ID NO: 1197 - DNA, 29 bp, Homo Sapiens
ctagattatgatgtgttccatgtatggca

SEQ ID NO: 1198 - DNA, 30 bp, Homo Sapiens
tactatggaaaattacctacctcctgaaca

SEQ ID NO: 1199 - DNA, 29 bp, Homo Sapiens
tacctctattgttggatcatattcgtcca

SEQ ID NO: 1200 - DNA, 30 bp, Homo Sapiens
tattataaggcctgctgaaaatgactgaat

SEQ ID NO: 1201 - DNA, 22 bp, Homo Sapiens
gccgaagtctgaccctttttgt

SEQ ID NO: 1202 - DNA, 22 bp, Homo Sapiens
ggtacctgtagtgtgcaggaaa

SEQ ID NO: 1203 - DNA, 23 bp, Homo Sapiens
cttcctaaggttgcacataggca

SEQ ID NO: 1204 - DNA, 26 bp, Homo Sapiens
gcccagcttctctgtctaagtagtaa

SEQ ID NO: 1205 - DNA, 28 bp, Homo Sapiens
gggaagaaaagtgttttgaaatgtgttt

SEQ ID NO: 1206 - DNA, 30 bp, Homo Sapiens
catttttccagatactagagtgtctgtgta

SEQ ID NO: 1207 - DNA, 33 bp, Homo Sapiens
tcttattctgaggttatctttttaccacagttg

SEQ ID NO: 1208 - DNA, 24 bp, Homo Sapiens
gctgcaacatgattgtcatcttca

SEQ ID NO: 1209 - DNA, 21 bp, Homo Sapiens
gtcaggtggtgtgatggtgat

SEQ ID NO: 1210 - DNA, 21 bp, Homo Sapiens
ggagcgaagctcatgactgtc

SEQ ID NO: 1211 - DNA, 19 bp, Homo Sapiens
gcttggatctggcgctttt

SEQ ID NO: 1212 - DNA, 20 bp, Homo Sapiens
aaacactgcctccagctctt

SEQ ID NO: 1213 - DNA, 20 bp, Homo Sapiens
atggaaggtgcgttcgatga

SEQ ID NO: 1214 - DNA, 21 bp, Homo Sapiens
atgcacgcagacagaggctct

SEQ ID NO: 1215 - DNA, 20 bp, Homo Sapiens
agctgctcaccatcgctatc

SEQ ID NO: 1216 - DNA, 20 bp, Homo Sapiens
cagctgtgggttgattccac

SEQ ID NO: 1217 - DNA, 26 bp, Homo Sapiens
cctgaatcaaatagggaaggaaagga

SEQ ID NO: 1218 - DNA, 22 bp, Homo Sapiens
tacggaccttacgtcagtgact

SEQ ID NO: 1219 - DNA, 27 bp, Homo Sapiens
agcaaatgtgtcttcactttttcatga

SEQ ID NO: 1220 - DNA, 22 bp, Homo Sapiens
ctgctgggcacagatgattttg

SEQ ID NO: 1221 - DNA, 28 bp, Homo Sapiens
gattcaatcaaactgcagagtatttggg

SEQ ID NO: 1222 - DNA, 24 bp, Homo Sapiens
tgatctggtgtcagagatggagat

SEQ ID NO: 1223 - DNA, 25 bp, Homo Sapiens
gtgtcagtaatgggaaatctgcaag

SEQ ID NO: 1224 - DNA, 23 bp, Homo Sapiens
ccaagaactccgcactttctctc

SEQ ID NO: 1225 - DNA, 30 bp, Homo Sapiens
cacatgtttagtgatgaaaaatttctccct

SEQ ID NO: 1226 - DNA, 30 bp, Homo Sapiens
taacatacctactaagtgctgtccactaat

SEQ ID NO: 1227 - DNA, 20 bp, Homo Sapiens
ggagatccgctgggacaaat

SEQ ID NO: 1228 - DNA, 22 bp, Homo Sapiens
ggctagaccaaaccgcaattct

SEQ ID NO: 1229 - DNA, 22 bp, Homo Sapiens
tttgtgaacgccttctgtctga

SEQ ID NO: 1230 - DNA, 29 bp, Homo Sapiens
agaaggtgaagtgcttgattttcttactt

SEQ ID NO: 1231 - DNA, 21 bp, Homo Sapiens
gggatgacctggaaacttcgg

SEQ ID NO: 1232 - DNA, 24 bp, Homo Sapiens
caaacttttctctctggacactcg

SEQ ID NO: 1233 - DNA, 33 bp, Homo Sapiens
tcataattgtgattttctaaaatagcaggctct

SEQ ID NO: 1234 - DNA, 33 bp, Homo Sapiens
attgtttttaacttactgatttaagcatggatt

SEQ ID NO: 1235 - DNA, 19 bp, Homo Sapiens
cggaacgcgtccgaaaatg

SEQ ID NO: 1236 - DNA, 23 bp, Homo Sapiens
gcactcccgtgtaactcctatga

SEQ ID NO: 1237 - DNA, 22 bp, Homo Sapiens
ggttccattggtagctggtgat

SEQ ID NO: 1238 - DNA, 29 bp, Homo Sapiens
gcccatttttatctacttccatcttgtca

SEQ ID NO: 1239 - DNA, 19 bp, Homo Sapiens
catgggactcgcatgttcg

SEQ ID NO: 1240 - DNA, 23 bp, Homo Sapiens
gccaaacaaagttctctgtcacc

SEQ ID NO: 1241 - DNA, 24 bp, Homo Sapiens
gttgcagcaattcactgtaaagct

SEQ ID NO: 1242 - DNA, 26 bp, Homo Sapiens
acctttttgtctctggtccttacttc

SEQ ID NO: 1243 - DNA, 20 bp, Homo Sapiens
gtctctgagcctgtgagtgc

SEQ ID NO: 1244 - DNA, 19 bp, Homo Sapiens
cagagcgctggagaccatt

SEQ ID NO: 1245 - DNA, 29 bp, Homo Sapiens
caccttctttctaaccttttcttatgtgc

SEQ ID NO: 1246 - DNA, 29 bp, Homo Sapiens
tcctgctttgaacaaataaatgaatcacg

SEQ ID NO: 1247 - DNA, 22 bp, Homo Sapiens
ttgaagaacacgaatctccgca

SEQ ID NO: 1248 - DNA, 20 bp, Homo Sapiens
aggatgatgccacagtcgtc

SEQ ID NO: 1249 - DNA, 24 bp, Homo Sapiens
gctcaagttcttgtgtttgtgtgt

SEQ ID NO: 1250 - DNA, 23 bp, Homo Sapiens
ccatatgcaggtggagggatttg

SEQ ID NO: 1251 - DNA, 20 bp, Homo Sapiens
gagagaccgaagccaccttt

SEQ ID NO: 1252 - DNA, 19 bp, Homo Sapiens
tagagccgcagcatgtgtt

SEQ ID NO: 1253 - DNA, 22 bp, Homo Sapiens
taggacactacccaatgcctca

SEQ ID NO: 1254 - DNA, 33 bp, Homo Sapiens
ccaaaataatgtgatggaatgataaaccaagat

SEQ ID NO: 1255 - DNA, 20 bp, Homo Sapiens
gtgctacctgtttgtgtgcg

SEQ ID NO: 1256 - DNA, 21 bp, Homo Sapiens
taatccgagctccgctggtca

SEQ ID NO: 1257 - DNA, 26 bp, Homo Sapiens
taacgtcttccttctctctctgtcat

SEQ ID NO: 1258 - DNA, 25 bp, Homo Sapiens
agcagaaactcacatcgaggatttc

SEQ ID NO: 1259 - DNA, 26 bp, Homo Sapiens
gtggtgatctgggtaatagtttctcc

SEQ ID NO: 1260 - DNA, 26 bp, Homo Sapiens
tgttcagaggatagcaacatacttcg

SEQ ID NO: 1261 - DNA, 29 bp, Homo Sapiens
aatctacaggaatagccacatacagaatg

SEQ ID NO: 1262 - DNA, 28 bp, Homo Sapiens
ctttctgtgtagtaccttcatgaaaacg

SEQ ID NO: 1263 - DNA, 24 bp, Homo Sapiens
tatggtctgcaggacaattcatgg

SEQ ID NO: 1264 - DNA, 30 bp, Homo Sapiens
tcttatgcaaatagttgaccaaatctccat

SEQ ID NO: 1265 - DNA, 22 bp, Homo Sapiens
cccagcgtcctcaaaagttaca

SEQ ID NO: 1266 - DNA, 22 bp, Homo Sapiens
ccctccacaatcattcctgtgt

SEQ ID NO: 1267 - DNA, 22 bp, Homo Sapiens
ccacttttgcacagccaagaac

SEQ ID NO: 1268 - DNA, 29 bp, Homo Sapiens
tgagaatgatcgttttcttcctctgttag

SEQ ID NO: 1269 - DNA, 22 bp, Homo Sapiens
ccaggcattgaagtctcatgga

SEQ ID NO: 1270 - DNA, 24 bp, Homo Sapiens
atcttctgtcccttcccagaaaac

SEQ ID NO: 1271 - DNA, 22 bp, Homo Sapiens
gcagttgggcacttttgaagat

SEQ ID NO: 1272 - DNA, 28 bp, Homo Sapiens
aatcaaagtcaccaacctttaagaagga

SEQ ID NO: 1273 - DNA, 22 bp, Homo Sapiens
ggcattctgggagcttcatctg

SEQ ID NO: 1274 - DNA, 23 bp, Homo Sapiens
ctgactgctcttttcacccatct

SEQ ID NO: 1275 - DNA, 23 bp, Homo Sapiens
ggccagggtcaaagatatttgga

SEQ ID NO: 1276 - DNA, 29 bp, Homo Sapiens
acttctcctcacttctggacttctttata

SEQ ID NO: 1277 - DNA, 19 bp, Homo Sapiens
agaagccttccggcacaag

SEQ ID NO: 1278 - DNA, 21 bp, Homo Sapiens
cttaccgtggaccttactggg

SEQ ID NO: 1279 - DNA, 23 bp, Homo Sapiens
gtatggtgtgttctggaagtcca

SEQ ID NO: 1280 - DNA, 21 bp, Homo Sapiens
cgtgatagtggccatcttcct

SEQ ID NO: 1281 - DNA, 18 bp, Homo Sapiens
cacctggtacgtccgcaa

SEQ ID NO: 1282 - DNA, 20 bp, Homo Sapiens
gggatggtgaagcttccagc

SEQ ID NO: 1283 - DNA, 30 bp, Homo Sapiens
ttttcttaacacattgactttttggttcgt

SEQ ID NO: 1284 - DNA, 30 bp, Homo Sapiens
gtatcttgaagatttagccattccaaaacc

SEQ ID NO: 1285 - DNA, 20 bp, Homo Sapiens
cgaccggaagtccatctcct

SEQ ID NO: 1286 - DNA, 22 bp, Homo Sapiens
tggagctcctgatctggtacag

SEQ ID NO: 1287 - DNA, 21 bp, Homo Sapiens
gaatgcaaaacagagcctcgt

SEQ ID NO: 1288 - DNA, 19 bp, Homo Sapiens
ccagacgtcctgtcactcg

SEQ ID NO: 1289 - DNA, 29 bp, Homo Sapiens
gagtaaatgttgaccaaagggagaaaatg

SEQ ID NO: 1290 - DNA, 30 bp, Homo Sapiens
gcttcttcttttagataccggataatgact

SEQ ID NO: 1291 - DNA, 23 bp, Homo Sapiens
tgaccaccagtatagttccagga

SEQ ID NO: 1292 - DNA, 30 bp, Homo Sapiens
accctctaactgatagaataagagccattt

SEQ ID NO: 1293 - DNA, 23 bp, Homo Sapiens
ttgacagaacgggaagccctcat

SEQ ID NO: 1294 - DNA, 25 bp, Homo Sapiens
cctgacagacaataaaaggcagctt

SEQ ID NO: 1295 - DNA, 20 bp, Homo Sapiens
cagctcgttcatcgggactt

SEQ ID NO: 1296 - DNA, 20 bp, Homo Sapiens
acctggctcctcttcacgta

SEQ ID NO: 1297 - DNA, 26 bp, Homo Sapiens
agtgaaaaacaagctctcatgtctga

SEQ ID NO: 1298 - DNA, 25 bp, Homo Sapiens
catgtgtccagtgaaaatcctcact

SEQ ID NO: 1299 - DNA, 24 bp, Homo Sapiens
ctcaagagtgagccacttcttacc

SEQ ID NO: 1300 - DNA, 23 bp, Homo Sapiens
ctcctcttgtcttctcctttgca

SEQ ID NO: 1301 - DNA, 23 bp, Homo Sapiens
ccttactcatggtcggatcacaa

SEQ ID NO: 1302 - DNA, 28 bp, Homo Sapiens
gttgaaactaaaaatcctttgcaggact

SEQ ID NO: 1303 - DNA, 22 bp, Homo Sapiens
gagcttgctcagcttgtactca

SEQ ID NO: 1304 - DNA, 21 bp, Homo Sapiens
gcctgtgtagtgcttcaaggg

SEQ ID NO: 1305 - DNA, 21 bp, Homo Sapiens
caacatcaccacgggtctgta

SEQ ID NO: 1306 - DNA, 21 bp, Homo Sapiens
gatgaggctcccacctttcag

SEQ ID NO: 1307 - DNA, 27 bp, Homo Sapiens
cccattttcttctacttccatcttgga

SEQ ID NO: 1308 - DNA, 28 bp, Homo Sapiens
gttttgagcttgtttgctgaatgttaac

SEQ ID NO: 1309 - DNA, 21 bp, Homo Sapiens
cgtcctgggattgcagattgg

SEQ ID NO: 1310 - DNA, 23 bp, Homo Sapiens
gatggatgtcacgttctcaaagc

SEQ ID NO: 1311 - DNA, 30 bp, Homo Sapiens
cctcaatgtaacaaatatgacagtaaccct

SEQ ID NO: 1312 - DNA, 28 bp, Homo Sapiens
agatggaaactttggacttcaagaactt

SEQ ID NO: 1313 - DNA, 27 bp, Homo Sapiens
cttaaaaggtctttgatttgcgtcagt

SEQ ID NO: 1314 - DNA, 24 bp, Homo Sapiens
ggagctattccacctactgatcct

SEQ ID NO: 1315 - DNA, 24 bp, Homo Sapiens
tttgaatctttggccagtacctca

SEQ ID NO: 1316 - DNA, 29 bp, Homo Sapiens
cataagagagaaggtttgactgccataaa

SEQ ID NO: 1317 - DNA, 22 bp, Homo Sapiens
gaacctgatgacctgaaggagt

SEQ ID NO: 1318 - DNA, 27 bp, Homo Sapiens
tcccgactgtaattgatcttgtacatg

SEQ ID NO: 1319 - DNA, 27 bp, Homo Sapiens
gtccagagtgagttaactttttccaac

SEQ ID NO: 1320 - DNA, 26 bp, Homo Sapiens
catcactctggtgggtatagattctg

SEQ ID NO: 1321 - DNA, 22 bp, Homo Sapiens
ctggcccttcccaagatttgat

SEQ ID NO: 1322 - DNA, 22 bp, Homo Sapiens
gagaaggccttggcaatcatct

SEQ ID NO: 1323 - DNA, 28 bp, Homo Sapiens
aaaagtagaagcaatctgatgaactcca

SEQ ID NO: 1324 - DNA, 24 bp, Homo Sapiens
actctcatcttttgctgagaagca

SEQ ID NO: 1325 - DNA, 19 bp, Homo Sapiens
caatccctgaccctggctt

SEQ ID NO: 1326 - DNA, 23 bp, Homo Sapiens
gtgtacttccggatcttctgctg

SEQ ID NO: 1327 - DNA, 30 bp, Homo Sapiens
tttttactgttcttcctcagacattcaaac

SEQ ID NO: 1328 - DNA, 22 bp, Homo Sapiens
cctaccctggtggaagcatact

SEQ ID NO: 1329 - DNA, 21 bp, Homo Sapiens
ggaacctcctggactacctga

SEQ ID NO: 1330 - DNA, 27 bp, Homo Sapiens
ccctacctgtggatgaagtttttcttc

SEQ ID NO: 1331 - DNA, 31 bp, Homo Sapiens
ttggaagttgttttgttttgctaaaacaaag

SEQ ID NO: 1332 - DNA, 25 bp, Homo Sapiens
ggatttgagctgaggtcttctgatg

SEQ ID NO: 1333 - DNA, 23 bp, Homo Sapiens
cagacactgtacaagctctacga

SEQ ID NO: 1334 - DNA, 25 bp, Homo Sapiens
gaataaagaggagcaggttgaggaa

SEQ ID NO: 1335 - DNA, 21 bp, Homo Sapiens
gctgctgtgggaatgaacaaa

SEQ ID NO: 1336 - DNA, 19 bp, Homo Sapiens
gcaatgctgcaccaggttg

SEQ ID NO: 1337 - DNA, 25 bp, Homo Sapiens
actcctccatatgtagttcgctttg

SEQ ID NO: 1338 - DNA, 27 bp, Homo Sapiens
gaaaatgttgatgtgtcttgcataggt

SEQ ID NO: 1339 - DNA, 30 bp, Homo Sapiens
aaaagctcattaacttaactgacattctca

SEQ ID NO: 1340 - DNA, 30 bp, Homo Sapiens
atctgtatatacactgggcttctaaacaac

SEQ ID NO: 1341 - DNA, 25 bp, Homo Sapiens
tggtaggcttgagtttgaagaaaca

SEQ ID NO: 1342 - DNA, 25 bp, Homo Sapiens
tccttaccaatactccatccacaga

SEQ ID NO: 1343 - DNA, 24 bp, Homo Sapiens
gcatcaaccttctcaagacaacgt

SEQ ID NO: 1344 - DNA, 30 bp, Homo Sapiens
gcacccagccaattttgagtatttttaaaa

SEQ ID NO: 1345 - DNA, 31 bp, Homo Sapiens
tgacatgtaaaggataattgtcagtgacttt

SEQ ID NO: 1346 - DNA, 33 bp, Homo Sapiens
tcagtctgaagagttttatcatgatccaaaaat

SEQ ID NO: 1347 - DNA, 31 bp, Homo Sapiens
aaagattcaggcaatgtttgttagtattagt

SEQ ID NO: 1348 - DNA, 25 bp, Homo Sapiens
ctacctctgcaattaaatttggcgg

SEQ ID NO: 1349 - DNA, 23 bp, Homo Sapiens
tcctacctggtcttctaggaagc

SEQ ID NO: 1350 - DNA, 22 bp, Homo Sapiens
gagggttttcgtggttcacatc

SEQ ID NO: 1351 - DNA, 27 bp, Homo Sapiens
ctttgtcttcgtgatttgtaggagtca

SEQ ID NO: 1352 - DNA, 22 bp, Homo Sapiens
agcacgaggaagatcaggaatg

SEQ ID NO: 1353 - DNA, 22 bp, Homo Sapiens
cgtgaagaacagcacgtacaca

SEQ ID NO: 1354 - DNA, 27 bp, Homo Sapiens
agaatgaactcttccctccaaaagaag

SEQ ID NO: 1355 - DNA, 24 bp, Homo Sapiens
ctgccagtgcatatactttctgga

SEQ ID NO: 1356 - DNA, 25 bp, Homo Sapiens
cactggattttaacaaggcatgtga

SEQ ID NO: 1357 - DNA, 30 bp, Homo Sapiens
ctcaagttatttggaattttgaagaggtga

SEQ ID NO: 1358 - DNA, 23 bp, Homo Sapiens
ggcactgtatgcactcagagttc

SEQ ID NO: 1359 - DNA, 23 bp, Homo Sapiens
agatgcatagagcctacctgtca

SEQ ID NO: 1360 - DNA, 24 bp, Homo Sapiens
cttggtgctagtggagaacaaaac

SEQ ID NO: 1361 - DNA, 19 bp, Homo Sapiens
tcctgctcgtcgtcctgtg

SEQ ID NO: 1362 - DNA, 20 bp, Homo Sapiens
cttcctcaccgacgaggaag

SEQ ID NO: 1363 - DNA, 22 bp, Homo Sapiens
cagcatcacttcactggcttct

SEQ ID NO: 1364 - DNA, 27 bp, Homo Sapiens
ttgatcctttgatgccctcattatcaa

SEQ ID NO: 1365 - DNA, 27 bp, Homo Sapiens
tgcttactttgaaatggatgttcaggt

SEQ ID NO: 1366 - DNA, 22 bp, Homo Sapiens
tcctgtggacattggagagttg

SEQ ID NO: 1367 - DNA, 25 bp, Homo Sapiens
catccatccatccaggaaaatcaga

SEQ ID NO: 1368 - DNA, 29 bp, Homo Sapiens
gatccattcattctgcttattctcattcg

SEQ ID NO: 1369 - DNA, 27 bp, Homo Sapiens
gttttatcaaagcagaaggcaacttga

SEQ ID NO: 1370 - DNA, 30 bp, Homo Sapiens
gccatatgcacatgaatgaatttcttcaat

SEQ ID NO: 1371 - DNA, 21 bp, Homo Sapiens
gacatgagagctcgatgctca

SEQ ID NO: 1372 - DNA, 23 bp, Homo Sapiens
cccggagggtaagttgtatagtg

SEQ ID NO: 1373 - DNA, 26 bp, Homo Sapiens
catgcatgaacatttttctccacctt

SEQ ID NO: 1374 - DNA, 22 bp, Homo Sapiens
cttccagaccagggtgttgttt

SEQ ID NO: 1375 - DNA, 30 bp, Homo Sapiens
taaggtgctcaaaaatttcttcatctcact

SEQ ID NO: 1376 - DNA, 30 bp, Homo Sapiens
agttattgggtaatgttatatgctgtgctt

SEQ ID NO: 1377 - DNA, 22 bp, Homo Sapiens
cgagggcaaatacagctttggt

SEQ ID NO: 1378 - DNA, 24 bp, Homo Sapiens
gactctccaagatgggatactcca

SEQ ID NO: 1379 - DNA, 30 bp, Homo Sapiens
gtttgtaaacactgtcctgttttgatatcc

SEQ ID NO: 1380 - DNA, 24 bp, Homo Sapiens
acagggaattgcattcacacgtta

SEQ ID NO: 1381 - DNA, 24 bp, Homo Sapiens
ttcacctcactgaaacctttgtgt

SEQ ID NO: 1382 - DNA, 22 bp, Homo Sapiens
gtccaccaacactgagcacagt

SEQ ID NO: 1383 - DNA, 30 bp, Homo Sapiens
gataatctttacctctttagggagcaatga

SEQ ID NO: 1384 - DNA, 22 bp, Homo Sapiens
gtggaccagagaaattgcttgc

SEQ ID NO: 1385 - DNA, 22 bp, Homo Sapiens
ccatcctgacctggtatggtca

SEQ ID NO: 1386 - DNA, 22 bp, Homo Sapiens
cctgcttcaggacgttgaactc

SEQ ID NO: 1387 - DNA, 24 bp, Homo Sapiens
gttatgtcctcattgccctcaaca

SEQ ID NO: 1388 - DNA, 28 bp, Homo Sapiens
cttcagtccggttttatttgcatcatag

SEQ ID NO: 1389 - DNA, 23 bp, Homo Sapiens
ctccaccatgactttgaggttga

SEQ ID NO: 1390 - DNA, 24 bp, Homo Sapiens
acaaggacatcttcccactaatgc

SEQ ID NO: 1391 - DNA, 25 bp, Homo Sapiens
cccacaatcatactgctgacataca

SEQ ID NO: 1392 - DNA, 26 bp, Homo Sapiens
gatgaaccggtcctttacagatgaaa

SEQ ID NO: 1393 - DNA, 22 bp, Homo Sapiens
atggccatggaaccagacagaa

SEQ ID NO: 1394 - DNA, 23 bp, Homo Sapiens
tccacatcctcttcctcaggatt

SEQ ID NO: 1395 - DNA, 22 bp, Homo Sapiens
gttcgcacaaagcaagccagat

SEQ ID NO: 1396 - DNA, 24 bp, Homo Sapiens
gtccgtaaaaatgctggagacatc

SEQ ID NO: 1397 - DNA, 22 bp, Homo Sapiens
cccagctgtgatccatgagaac

SEQ ID NO: 1398 - DNA, 22 bp, Homo Sapiens
ccgactgccttgtaccattcat

SEQ ID NO: 1399 - DNA, 22 bp, Homo Sapiens
gcttgtaagtgcccgaagtgta

SEQ ID NO: 1400 - DNA, 30 bp, Homo Sapiens
cacaacccactgaggtatatgtataggtat

SEQ ID NO: 1401 - DNA, 31 bp, Homo Sapiens
tcaaatgttagctcatttttgttaatggtgg

SEQ ID NO: 1402 - DNA, 33 bp, Homo Sapiens
tgcaagcatacaaataagaaaacatacttacag

SEQ ID NO: 1403 - DNA, 21 bp, Homo Sapiens
ctcaatgagccctcagctgat

SEQ ID NO: 1404 - DNA, 25 bp, Homo Sapiens
ccagaagcttgaactctcatacctg

SEQ ID NO: 1405 - DNA, 33 bp, Homo Sapiens
gcatttcctgtgaaataatactggtatgtattt

SEQ ID NO: 1406 - DNA, 27 bp, Homo Sapiens
gggaactcaaagtacatgaacttgtct

SEQ ID NO: 1407 - DNA, 30 bp, Homo Sapiens
tttttcacaaagtttttgcttcaaatgtct

SEQ ID NO: 1408 - DNA, 22 bp, Homo Sapiens
cctcatcggaatcaagctcagt

SEQ ID NO: 1409 - DNA, 22 bp, Homo Sapiens
ctttgcttgtcccgataggtca

SEQ ID NO: 1410 - DNA, 26 bp, Homo Sapiens
ggcagtgtgatattggcaaaaatagg

SEQ ID NO: 1411 - DNA, 22 bp, Homo Sapiens
ccacttggtgaaggtagctgat

SEQ ID NO: 1412 - DNA, 26 bp, Homo Sapiens
cggacttgatggagaacttgttgtag

SEQ ID NO: 1413 - DNA, 26 bp, Homo Sapiens
cagctttcgacaaaagtcacaaaatg

SEQ ID NO: 1414 - DNA, 33 bp, Homo Sapiens
ttaaacaagagagtagatacgtcagtttctaga

SEQ ID NO: 1415 - DNA, 29 bp, Homo Sapiens
ttagatggctcattcacaactatctttcc

SEQ ID NO: 1416 - DNA, 28 bp, Homo Sapiens
tgggtaattacagtccagaagttccata

SEQ ID NO: 1417 - DNA, 22 bp, Homo Sapiens
gagcacaggaacttcttggtgt

SEQ ID NO: 1418 - DNA, 22 bp, Homo Sapiens
acggcatcgaataccagaacat

SEQ ID NO: 1419 - DNA, 26 bp, Homo Sapiens
aggcaaatcctaagagagaacaactg

SEQ ID NO: 1420 - DNA, 29 bp, Homo Sapiens
cataatgcttcctggtctttaggatttct

SEQ ID NO: 1421 - DNA, 23 bp, Homo Sapiens
cccactctccaatgtgactaggt

SEQ ID NO: 1422 - DNA, 22 bp, Homo Sapiens
ccaacaagcatcagagtgctgt

SEQ ID NO: 1423 - DNA, 27 bp, Homo Sapiens
gaaaaagcccttagagatcatgctaga

SEQ ID NO: 1424 - DNA, 27 bp, Homo Sapiens
gtctctttgcagttatgatggttaacg

SEQ ID NO: 1425 - DNA, 28 bp, Homo Sapiens
atgtcacctgaaacatttttagccattc

SEQ ID NO: 1426 - DNA, 23 bp, Homo Sapiens
gcttgtaccatgttcagcaacac

SEQ ID NO: 1427 - DNA, 26 bp, Homo Sapiens
gacaacattaacgctgacttgatcac

SEQ ID NO: 1428 - DNA, 26 bp, Homo Sapiens
cagaaacagctctagacaacaaacct

SEQ ID NO: 1429 - DNA, 20 bp, Homo Sapiens
ctgagggtgtcctgtgtcac

SEQ ID NO: 1430 - DNA, 24 bp, Homo Sapiens
catgaaacgcagattaccatgcag

SEQ ID NO: 1431 - DNA, 24 bp, Homo Sapiens
tggcctgccctatataattggaga

SEQ ID NO: 1432 - DNA, 26 bp, Homo Sapiens
ccgttatattgttctcctgtgtctgt

SEQ ID NO: 1433 - DNA, 20 bp, Homo Sapiens
gggagtgaggatggctacag

SEQ ID NO: 1434 - DNA, 21 bp, Homo Sapiens
ccttccatgtggagactcctg

SEQ ID NO: 1435 - DNA, 23 bp, Homo Sapiens
aaggcagtagaagttgctggaaa

SEQ ID NO: 1436 - DNA, 32 bp, Homo Sapiens
tccgatgatttcatgtagttttcaattctttg

SEQ ID NO: 1437 - DNA, 29 bp, Homo Sapiens
agcatgccaatctcttcataaatcttttc

SEQ ID NO: 1438 - DNA, 26 bp, Homo Sapiens
gcctcttgctcagttttatctaaggc

SEQ ID NO: 1439 - DNA, 25 bp, Homo Sapiens
cggaatttgaaaacaagcaagctct

SEQ ID NO: 1440 - DNA, 29 bp, Homo Sapiens
cactcactcagttaactggtgaacataaa

SEQ ID NO: 1441 - DNA, 22 bp, Homo Sapiens
ggtcatacagctgattgccaca

SEQ ID NO: 1442 - DNA, 22 bp, Homo Sapiens
gaggtctgctttggtccatctt

SEQ ID NO: 1443 - DNA, 25 bp, Homo Sapiens
gaatggagaaactcccagattccat

SEQ ID NO: 1444 - DNA, 26 bp, Homo Sapiens
taagccagtcagatcaggattctgat

SEQ ID NO: 1445 - DNA, 20 bp, Homo Sapiens
ggtcaaccacccacatgtca

SEQ ID NO: 1446 - DNA, 21 bp, Homo Sapiens
aagagggagaacagggctgta

SEQ ID NO: 1447 - DNA, 32 bp, Homo Sapiens
aaaggaacaatatgaattatactgtgagatgg

SEQ ID NO: 1448 - DNA, 23 bp, Homo Sapiens
gtacctgccaggatgtaagacag

SEQ ID NO: 1449 - DNA, 28 bp, Homo Sapiens
ctttagattcagaaagtcctcaccttga

SEQ ID NO: 1450 - DNA, 25 bp, Homo Sapiens
gagtttgtctgcaaggtttacagtg

SEQ ID NO: 1451 - DNA, 24 bp, Homo Sapiens
tcacaaaccctacagatacccaga

SEQ ID NO: 1452 - DNA, 22 bp, Homo Sapiens
gggcatgtatccagatgatgga

SEQ ID NO: 1453 - DNA, 30 bp, Homo Sapiens
tgtgatgttctgaaagcttaattctacctt

SEQ ID NO: 1454 - DNA, 21 bp, Homo Sapiens
cggccaacactgtcaagtttc

SEQ ID NO: 1455 - DNA, 28 bp, Homo Sapiens
atctggaaaactttctttcagtgataca

SEQ ID NO: 1456 - DNA, 33 bp, Homo Sapiens
acctttagctaataaaaatgtgatccaagaaac

SEQ ID NO: 1457 - DNA, 23 bp, Homo Sapiens
ggagcacctaggctaaaatgtca

SEQ ID NO: 1458 - DNA, 29 bp, Homo Sapiens
caccagtattttctcacagaaagaatgtc

SEQ ID NO: 1459 - DNA, 31 bp, Homo Sapiens
gtttaacctttctactgttttctttgtctga

SEQ ID NO: 1460 - DNA, 29 bp, Homo Sapiens
atctgttccagaatcaagattctgagatg

SEQ ID NO: 1461 - DNA, 26 bp, Homo Sapiens
cagtcttacatttgaccatgaccatg

SEQ ID NO: 1462 - DNA, 24 bp, Homo Sapiens
actgatgacctttggaggaaaacc

SEQ ID NO: 1463 - DNA, 27 bp, Homo Sapiens
cctccttcctagagagttagagtaact

SEQ ID NO: 1464 - DNA, 24 bp, Homo Sapiens
cacccacacttacacatcactttg

SEQ ID NO: 1465 - DNA, 22 bp, Homo Sapiens
ccagttacctgtcctggtcatt

SEQ ID NO: 1466 - DNA, 26 bp, Homo Sapiens
ggaaactcccatcttgagtcataagg

SEQ ID NO: 1467 - DNA, 22 bp, Homo Sapiens
tttttctgtccaccagggagta

SEQ ID NO: 1468 - DNA, 31 bp, Homo Sapiens
acattggaatagtttcaaacatcatcttgtg

SEQ ID NO: 1469 - DNA, 22 bp, Homo Sapiens
agacgacacaggaagcagattc

SEQ ID NO: 1470 - DNA, 24 bp, Homo Sapiens
cagtctgctggatttggttctagg

SEQ ID NO: 1471 - DNA, 27 bp, Homo Sapiens
aagatcaccttcagaagtcacagaatg

SEQ ID NO: 1472 - DNA, 25 bp, Homo Sapiens
ctggttgagatgaaaggattccact

SEQ ID NO: 1473 - DNA, 23 bp, Homo Sapiens
tggaccacacaggagaatatgga

SEQ ID NO: 1474 - DNA, 24 bp, Homo Sapiens
cttaacaagctgtctcctctcctt

SEQ ID NO: 1475 - DNA, 28 bp, Homo Sapiens
gttctgttaaagttcatggcttttgtgt

SEQ ID NO: 1476 - DNA, 30 bp, Homo Sapiens
tttacataagaagcgtttacgatcctcttt

SEQ ID NO: 1477 - DNA, 24 bp, Homo Sapiens
aggtgcagaacatcaagttcaaca

SEQ ID NO: 1478 - DNA, 21 bp, Homo Sapiens
gtgctcagcctctgtgaagag

SEQ ID NO: 1479 - DNA, 22 bp, Homo Sapiens
cagaaggtctacatgggtgctt

SEQ ID NO: 1480 - DNA, 23 bp, Homo Sapiens
gccagcccgaagtctgtaatttt

SEQ ID NO: 1481 - DNA, 26 bp, Homo Sapiens
tctatatgtagaggctgttggaagct

SEQ ID NO: 1482 - DNA, 30 bp, Homo Sapiens
tccactgaagttctttatcttcaaataact

SEQ ID NO: 1483 - DNA, 30 bp, Homo Sapiens
tgctttagattggcaattattactgtttcg

SEQ ID NO: 1484 - DNA, 23 bp, Homo Sapiens
gttgactttgtccacctggaact

SEQ ID NO: 1485 - DNA, 29 bp, Homo Sapiens
aaggctttttctttagacagttgtttgtt

SEQ ID NO: 1486 - DNA, 22 bp, Homo Sapiens
gaggttcccgtaggtcatgaac

SEQ ID NO: 1487 - DNA, 22 bp, Homo Sapiens
ctgcgacccttataatgagcct

SEQ ID NO: 1488 - DNA, 27 bp, Homo Sapiens
gcaactattttcttcctctcttccaca

SEQ ID NO: 1489 - DNA, 23 bp, Homo Sapiens
ggcacggttgaatgtaaggctta

SEQ ID NO: 1490 - DNA, 28 bp, Homo Sapiens
actgatatggtagacagagcctaaacat

SEQ ID NO: 1491 - DNA, 27 bp, Homo Sapiens
aggcttcatatgatgaagggtaatgtg

SEQ ID NO: 1492 - DNA, 25 bp, Homo Sapiens
taggagatacccacgtatgtaccac

SEQ ID NO: 1493 - DNA, 24 bp, Homo Sapiens
ccactccatcgagatttcactgta

SEQ ID NO: 1494 - DNA, 29 bp, Homo Sapiens
tcataatgcttgctctgataggaaaatga

SEQ ID NO: 1495 - DNA, 30 bp, Homo Sapiens
aaaaatcaaatcttaaaagcttcttggtgt

SEQ ID NO: 1496 - DNA, 23 bp, Homo Sapiens
tctttctccactcagcgtctttg

SEQ ID NO: 1497 - DNA, 22 bp, Homo Sapiens
gattgaagagcccacaggtgat

SEQ ID NO: 1498 - DNA, 22 bp, Homo Sapiens
ctcctccttgctagggttcttc

SEQ ID NO: 1499 - DNA, 29 bp, Homo Sapiens
cagaaacgtttcgattataaagatcagca

SEQ ID NO: 1500 - DNA, 28 bp, Homo Sapiens
aaaaagactgtaagtggtttctcaggaa

SEQ ID NO: 1501 - DNA, 27 bp, Homo Sapiens
ggacttggtgatagacatgtacagaat

SEQ ID NO: 1502 - DNA, 30 bp, Homo Sapiens
gcaaacaacattccatgatgaccaaatatt

SEQ ID NO: 1503 - DNA, 22 bp, Homo Sapiens
ctgcacatccggatgtaggatc

SEQ ID NO: 1504 - DNA, 22 bp, Homo Sapiens
gaaccctgagagcagcttcaat

SEQ ID NO: 1505 - DNA, 28 bp, Homo Sapiens
ttctttgtagatatgatgcagccattga

SEQ ID NO: 1506 - DNA, 28 bp, Homo Sapiens
gaaaaccattacttgtccatcgtctttc

SEQ ID NO: 1507 - DNA, 30 bp, Homo Sapiens
agaaaattgactaacctgtgtttctttaca

SEQ ID NO: 1508 - DNA, 23 bp, Homo Sapiens
cctttggaagtggacccagaaac

SEQ ID NO: 1509 - DNA, 30 bp, Homo Sapiens
ccattttctctcagtaagtgtttatgatgc

SEQ ID NO: 1510 - DNA, 28 bp, Homo Sapiens
atttaaaattagcaccctgagaagctct

SEQ ID NO: 1511 - DNA, 22 bp, Homo Sapiens
gaacaggccctcagttcaagat

SEQ ID NO: 1512 - DNA, 23 bp, Homo Sapiens
actctcccttcacaggtggtatt

SEQ ID NO: 1513 - DNA, 24 bp, Homo Sapiens
tttgtttgtcagagtcagagcact

SEQ ID NO: 1514 - DNA, 28 bp, Homo Sapiens
tctagatcctaaacgtaagaagcaacac

SEQ ID NO: 1515 - DNA, 23 bp, Homo Sapiens
gtgacaaacctgctgagcattag

SEQ ID NO: 1516 - DNA, 29 bp, Homo Sapiens
tgaaatcagtgttttgcttctctaggtac

SEQ ID NO: 1517 - DNA, 24 bp, Homo Sapiens
cctgtttaggccttgcagaatttg

SEQ ID NO: 1518 - DNA, 22 bp, Homo Sapiens
tcccactgcatattcctccatg

SEQ ID NO: 1519 - DNA, 24 bp, Homo Sapiens
gcatagaggagagaggaaaagtgg

SEQ ID NO: 1520 - DNA, 20 bp, Homo Sapiens
attggcagctccgaggacca

SEQ ID NO: 1521 - DNA, 23 bp, Homo Sapiens
tggtggacaagtgaatttgctca

SEQ ID NO: 1522 - DNA, 30 bp, Homo Sapiens
ttctaaaggctgaatgaaagggtaattcat

SEQ ID NO: 1523 - DNA, 18 bp, Homo Sapiens
ctgccaatcggcgtgtaa

SEQ ID NO: 1524 - DNA, 22 bp, Homo Sapiens
ctcctcttcttttcctctggct

SEQ ID NO: 1525 - DNA, 33 bp, Homo Sapiens
tctttgctcatcttttctttatgttttcgaatt

SEQ ID NO: 1526 - DNA, 30 bp, Homo Sapiens
aatgaaatttgttacctgtacacatgaagc

SEQ ID NO: 1527 - DNA, 27 bp, Homo Sapiens
gttcttttgtcctactccttctttcca

SEQ ID NO: 1528 - DNA, 28 bp, Homo Sapiens
ttacttcagtgtttctccatcatcacag

SEQ ID NO: 1529 - DNA, 26 bp, Homo Sapiens
aaaatctctgtccaagtcctgtgaaa

SEQ ID NO: 1530 - DNA, 29 bp, Homo Sapiens
gctttgtgtatgcctataattgaaactgt

SEQ ID NO: 1531 - DNA, 22 bp, Homo Sapiens
tcttacacccagtggagaagct

SEQ ID NO: 1532 - DNA, 23 bp, Homo Sapiens
tgtgccagggaccttaccttata

SEQ ID NO: 1533 - DNA, 26 bp, Homo Sapiens
tgcattacctacgatggtaaccaaag

SEQ ID NO: 1534 - DNA, 30 bp, Homo Sapiens
cctattcaacaaacagaactatgatacgga

SEQ ID NO: 1535 - DNA, 28 bp, Homo Sapiens
gcattaactagtcaagtacttacccact

SEQ ID NO: 1536 - DNA, 25 bp, Homo Sapiens
atctctttcatgactgcagcttctt

SEQ ID NO: 1537 - DNA, 27 bp, Homo Sapiens
gtgttcactttcaggaattctatgagc

SEQ ID NO: 1538 - DNA, 22 bp, Homo Sapiens
gttgggtggcggttacttacta

SEQ ID NO: 1539 - DNA, 25 bp, Homo Sapiens
aaagagatcaaacaccctaacctgg

SEQ ID NO: 1540 - DNA, 20 bp, Homo Sapiens
cgaggttttgtgcagtgagc

SEQ ID NO: 1541 - DNA, 27 bp, Homo Sapiens
gcctctctaattttgtgacatttgagc

SEQ ID NO: 1542 - DNA, 22 bp, Homo Sapiens
ggcatgctgtcgaatagctaga

SEQ ID NO: 1543 - DNA, 24 bp, Homo Sapiens
ctcctgaaaagagagtggaagtgt

SEQ ID NO: 1544 - DNA, 26 bp, Homo Sapiens
agttgctgcaagtcagttgaaaaatc

SEQ ID NO: 1545 - DNA, 24 bp, Homo Sapiens
gggtgtggatgcttccttttaaac

SEQ ID NO: 1546 - DNA, 23 bp, Homo Sapiens
tgtactccagtgaggaagcagaa

SEQ ID NO: 1547 - DNA, 24 bp, Homo Sapiens
gatcgtctccatcatcatcatcgt

SEQ ID NO: 1548 - DNA, 27 bp, Homo Sapiens
gacattattgcttctcctgtgtgtttc

SEQ ID NO: 1549 - DNA, 30 bp, Homo Sapiens
catcattaatttttgcttcacagaagacca

SEQ ID NO: 1550 - DNA, 30 bp, Homo Sapiens
tattacccagagatacccagaaaagagatt

SEQ ID NO: 1551 - DNA, 33 bp, Homo Sapiens
tttgtggtttactttaagattacaaattcagaa

SEQ ID NO: 1552 - DNA, 32 bp, Homo Sapiens
gctttctggaataattctgacttatatgcttc

SEQ ID NO: 1553 - DNA, 29 bp, Homo Sapiens
tgctactatcatcagactgatcaaaatcg

SEQ ID NO: 1554 - DNA, 24 bp, Homo Sapiens
ggtagatgaggactcctcaggaaa

SEQ ID NO: 1555 - DNA, 20 bp, Homo Sapiens
ccacgaccacggtctctaga

SEQ ID NO: 1556 - DNA, 21 bp, Homo Sapiens
gttgagagagtgggtgtggtt

(19) United States
(12) Patent Application Publication
(10) Pub. No.: US 2020/0048697 A1
(43) Pub. Date: Feb. 13, 2020

(54) Compositions and methods for detection of genomic variance and DNA methylation status

(71) Applicant: Singlera Genomics, Inc., La Jolla, CA (US)

(72) Inventor: Rui LIU, Shanghai (CN)

(73) Assignee: Singlera Genomics, Inc., La Jolla, CA (US)

(21) Appl. No.: 16/605,201

(22) PCT Filed: Apr. 18, 2018
(86) PCT No.: PCT/US2018/028185

§ 371 Date: Oct. 14, 2019

Related U.S. Application Data
(60) Provisional application No. 62/487,422, filed on Apr. 19, 2017.

(51) Int. Cl. C12Q 1/6858; C12Q 1/6806; C12Q 1/683; C12Q 1/686; C12Q 1/6886

(52) U.S. Cl. CPC C12Q 1/6858; C12Q 1/6806; C12Q 1/683; C12Q 2600/156; C12Q 1/6886; C12Q 2600/154; C12Q 1/686

(57) ABSTRACT

In one aspect, provided herein is an integrated method for simultaneous detection of both a genomic variance and quantification of a DNA methylation state/status on one or more (e.g., hundreds of thousands of) targets, without splitting the limited materials for two different workflows. The present disclosure relates to compositions, kus, devices, and methods for conducting genetic arid genomic analysis, for example, by polynucleotide sequencing in particular aspects, provided herein are compositions, kits, and methods for constructing libraries for simultaneous detection of genomic variants and DNA methylation status on limited DNA inputs, such as circulating polynucleotide fragments in the body of a subject, including circulating tumor DNA.

### CROSS-REFERENCE TO RELATED APPLICATIONS

This application claims benefit of priority to U.S. Provisional Application Ser. No. 62/487,422, filed on Apr. 19, 2017, the content of which is incorporated by reference its entirety for all purposes. In some aspect, the present disclosure relates to U.S. provisional application Ser. No. 62/487,423, filed on Apr. 19, 2017, and U.S. Provisional Application Ser. No. 62/657,544, filed Apr. 13, 2018, the contents of both applications are incorporated by reference in their entireties for all purposes.

### TECHNICAL FIELD

The present disclosure relates to compositions, kits, devices, and methods for conducting genetic and genomic analysis, for example, by polynucleotide sequencing. In particular aspects, provided herein are compositions, kits, and methods for constructing libraries for simultaneous detection of genomic variants and DNA methylation status on limited DNA inputs, such as circulating polynucleotide fragments in the body of a subject, including circulating tumor DNA.

### BACKGROUND

In the following discussion, certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Mammalian (including human) cells typically have DNA methylation at CpG di-nucleotides. The status of CpG methylation in general can be determined with at least four mechanisms, (i) sodium bisulfite treatment to convert the modification status into different genetic codes; (ii) affinity enrichment by antibodies or methyl-CpG binding proteins; (iii) digestion by methyl-sensitive restriction enzymes; (iv) direct sequencing by nano-pores or PacBio polymerase real-time monitoring. Depending on the number of targets per assay, the methylation information can be read out by gel electrophoresis, real-time quantitative PCR, Sanger sequencing, microarray, second-generation sequencing, or mass spectrometry. Notably, while genome-wide measurements provide very rich information for discovery purposes, many clinical assays focus on limited number of most informative and reliable markers, and use PCR, hybridization-based enrichment, or padlock capture to enrich assay targets specifically. Laird (2010), “Principles and challenges of genome-wide DNA methylation analysis,” Nat Rev Genet 11: 191-203; and Plongthongkum et al. (2014), “Advances in the profiling of DNA modifications: cytosine methylation and beyond,” Nat Rev Genet 15: 647-661. In general, bisulfite-based methods provide absolute quantification at the single-base resolution, both are highly desirable features. Yet the chemical treatment is harsh and tends to lead to material losses, which can compromise the assay sensitivity on low-input samples.

Methods for detecting and quantifying germline or somatic genetic variants have evolved over the past three decades. While Sanger sequencing and real-time quantitative PCR based methods have been routinely implemented in clinical labs, several targeted sequencing methods based on next-generation sequencing have started to be implemented as clinical tests. Rehm (2013). “Disease-targeted sequencing: a cornerstone in the clinic,” Nat Rev Genet 14: 295-300. These tests typically use hybridization capture methods, multiplexed PCR, or circularization capture using padlock probes or selectors. These methods differ in scalability, uniformity, library conversion efficiency, and assay cost.

Many clinical samples contain limited amounts of DNA molecules, which can often be degraded or fragmented. For multiple diagnostic purposes, it will be beneficial to obtain multi-layer of information for making accurate and specific prediction of disease status or disease types. There is a growing need for assays that can efficiently read out both genomics and epigenetics information from very limited amount of DNA materials, and can be easily deployed and robustly implemented in clinical laboratories. The present disclosure addresses this and other related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, provided herein is a method for analyzing a first target polynucleotide sequence and a methylation status of a second target polynucleotide sequence in a sample, comprising contacting a sample containing or suspected of containing a polynucleotide with a methylation-sensitive restriction enzyme (MSRE). In one aspect, the MSRE selectively cleaves the polynucleotide at a residue when it is unmethylated or selectively cleaves the polynucleotide at the residue when it is methylated.

In another aspect, the method comprises subjecting an MSRE-treated sample to polynucleotide amplification, using a mixture of: i) a first primer set for amplifying a first target polynucleotide sequence in the sample, and ii) a second primer set for analyzing a methylation status of a second target polynucleotide sequence in the sample.

In any of the preceding embodiments, the methylation status can be of a residue in the second target polynucleotide sequence, and one primer of the second primer set can hybridize to the uncleaved second target polynucleotide sequence and together with another primer in the set, can amplify the uncleaved sequence but not the second target polynucleotide sequence cleaved at the residue by the MSRE.

In any of the preceding embodiments, the method can further comprise sequencing the amplified polynucleotides.

In any of the preceding embodiments, the first target polynucleotide sequence can be analyzed using sequencing reads from the amplified first target polynucleotide sequence.

In any of the preceding embodiments, the methylation status of the residue of the second target polynucleotide sequence can be analyzed by comparing the observed number of sequencing reads (Nₒ) from the amplified second target polynucleotide sequence to a reference number.

In yet another aspect, provided herein is a method for analyzing a first target polynucleotide sequence and a methylation status of a second target polynucleotide sequence in a sample. In one embodiment, the method comprises: (1) contacting a sample comprising a polynucleotide with a methylation-sensitive restriction enzyme (MSRE), and the MSRE selectively cleaves the polynucleotide at a residue when it is unmethylated or selectively cleaves the polynucleotide at the residue when it is methylated; (2) subjecting the sample from step (1) to polynucleotide amplification, using a mixture of: i) a first primer set for amplifying a first target polynucleotide sequence in the sample, and ii) a second primer set for analyzing a methylation status of a second target polynucleotide sequence in the sample, and the methylation status is of a residue in the second target polynucleotide sequence, and one primer of the second primer set hybridizes to the uncleaved second target polynudeotide sequence and together with another primer in the set, amplifies the uncleaved sequence but not the second target polynucleotide sequence cleaved at the residue by the MSRE; and (3) sequencing polynucleotides amplified in step (2), and the first target polynucleotide sequence is analyzed using sequencing reads from the amplified first target polynucleotide sequence, and the methylation status of the residue of the second target polynucleotide sequence is analyzed by comparing the observed number of sequencing reads (No) from the amplified second target polynucleotide sequence to a reference number.

In any of the preceding embodiments, the MSRE can cleave the polynucleotide at a residue when it is unmethylated and not cleave at the residue when it is methylated.

In any of the preceding embodiments, the method can further comprise amplification and sequencing of a polynucleotide from a sample that is not contacted with the MSRE.

In any of the preceding embodiments, the MSRE can be selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and any combination thereof.

In any of the preceding embodiments, the first target polynucleotide sequence can comprise a genetic or epigenetic information, such as a mutation, a single nucleotide polymorphism (SNP), a copy number variation (CNV), a DNA modification such as DNA methylation, and/or a histone modification. In one embodiment, the mutation comprises a point mutation, an insertion, a deletion, an indel, an inversion, a truncation, a fusion, a translocation, an amplification, or any combination thereof. In any of the preceding embodiments, the genetic or epigenetic information can be associated with a condition or disease in a subject or a population, such as a cancer-related mutation.

In any of the preceding embodiments, the second target polynucleotide sequence can comprise one or more CpG sites within the recognition site of the MSRE. In one embodiment, at each CpG site the cytosine (C) comprises a 5-methyl moiety or a 5-hydrogen moiety.

In any of the preceding embodiments, the second target polynucleotide sequence can comprise a regulatory sequence for a gene, such as a promoter region, an enhancer region, an insulator region, a silencer region, a 5′UTR region, a 3′UTR region, or a splice control region, and one or more CpG sites are located within the regulatory sequence. In one aspect, the gene is associated with a condition or disease in a subject or a population, such as a gene overexpressed, underexpressed, constitutively active, silenced, or ectopically expressed in a cancer or neoplasia.

In any of the preceding embodiments, the sample is can be a biological sample. In one aspect, the biological sample is from a subject having or suspected of having a disease or condition, such as a cancer or neoplasia.

In any of the preceding embodiments, the sample can comprise circulating tumor DNA (ctDNA), such as a blood, serum, plasma, or body fluid sample, or any combination thereof.

In any of the preceding embodiments, the polynucleotide in the sample can be or comprise a double-stranded sequence.

In any of the preceding embodiments, the polynucleotide in the sample can be or comprise a single-stranded sequence.

In any of the preceding embodiments, the method can comprise converting the single-stranded sequence to a double-stranded sequence based on sequence complementarity, for example, by primer extension.

In any of the preceding embodiments, the first and second target polynucleotide sequences can be on the same molecule or on different molecules, for example, two different DNA fragments, in the sample.

In any of the preceding embodiments, the first and second target polynucleotide sequences can be on the same gene.

In any of the preceding embodiments, the first target polynucleotide sequence can be in a coding region of a gene whereas the second target polynucleotide sequence can be in a non-coding and/or regulatory region of or for the same gene.

In any of the preceding embodiments, the first and second target polynucleotide sequences can be on different genes. In one aspect, the genes function in the same biological pathway or network.

In any of the preceding embodiments, the first and second target polynucleotide sequences can be on the same or different chromosomes, or on the same or different extrachromosomal DNA molecules (such as mitochondria DNA), or one on a chromosome and the other on an extrachromosomal DNA molecule.

In any of the preceding embodiments, the amplification step can comprise a polymerase chain reaction (PCR), reverse-transcription PCR amplification, allele-specific PCR (ASPCR), single-base extension (SBE), allele specific primer extension (ASPE), strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), the use of Q Beta replicase, nick translation, or loop-mediated isothermal amplification (LAMP), or any combination thereof.

In any of the preceding embodiments, allele-specific PCR (ASPCR) can be used to amplify the first target polynucleotide sequence, and the first set of primers comprise at least two allele-specific primers and a common primer. In one aspect, the ASPCR uses a DNA polymerase without a 3′ to 5′ exonuclease activity. In another aspect, at least one of the at least two allele-specific primers is specific for a cancer mutation.

In any of the preceding embodiments, the second set of primers can comprise a common primer and at least two primers each for a different CpG site in the second target polynucleotide sequence.

In any of the preceding embodiments, the method can further comprise purifying polynucleotides from an MSRE-treated sample, purifying polynucleotides from the sample from the amplification step, and/or purifying polynucleotides before, during, and/or after the sequencing step.

In any of the preceding embodiments, the sequencing step can comprise attaching a sequencing adapter and/or a sample-specific barcode to each polynucleotide. In one aspect, the attaching step is performed using a polymerase chain reaction (PCR).

In any of the preceding embodiments, the sequencing can be a high-throughput sequencing, a digital sequencing, or a next-generating sequencing (NGS) such as Illumina (Solexa) sequencing, Roche 454 sequencing, Ion torrent: Proton/PGM sequencing, and SOLiD sequencing.

In any of the preceding embodiments, the reference number can be predetermined (for example, based on literature) or determined in parallel as the analysis of the first and second target polynucleotide sequences. In one aspect, the reference number is an expected number of sequencing reads (Nₑ) based on a control locus and/or a reference sample, with or without a control reaction using an isoschizomer of the MSRS that is methylation insensitive.

In any of the preceding embodiments, the sample can be a tumor sample and the reference sample can be from a normal tissue adjacent to the tumor.

In any of the preceding embodiments, the methylation status at the residue in the second target polynucleotide sequence can be a qualitative or quantitative readout, for example, as indicated by the methylation level mC=Nₒ/Nₑ.

In any of the preceding embodiments, the first primer set and/or the second primer set can comprise one or more primers listed in Table 1 and/or Table 2, in any suitable combination.

In any of the preceding embodiments, the first primer set can comprise one or more primers for a gene selected from the group consisting of ABCB1, CYP2C19, CYP2C8, CYP2D6, CYP3A4, CYP3A5, DPYD, GSTP1, MTHFR, NQO1, RHEB, SULT1A1, UGT1A1, MPL, JAK1, NRAS, DDR2, PTEN, FGFR2, HRAS, ATM, CBL, KRAS, ERBB3, CDK4, HNF1A, FLT3, RB1, AKT1, IDH2, CDH1, TR53, ERBB2, STAT3, SMAD4, STK11, GNA11, JAK3, PPP2R1A, RET, DNMT3A, ALK, NFE2L2, SF3B1, PIK3CA, ERBB4, GNAS, U2AF1, SLC19A1, SMARCB1, CHEK2, VHL, RAF1, CTNNB1, PDGFRA, KIT, KDR, FBXW7, APC, NEUROG1, CSF1R, NPM1, TPMT, EGFR, MET, SMO, BRAF, EZH2, FGFR1, JAK2, CDKN2A, PAX5, PTCH1, ABL1, NOTCH1, ARAF, MED12, BTK, and any combination thereof.

In any of the preceding embodiments, the one or more primers from the first primer set can comprise, consist essentially of, or consist of a sequence set forth in SEQ ID NOs: 61-788, or any combination thereof.

In any of the preceding embodiments, the second primer set can comprise one or more primers for a gene selected from the group consisting of NDRG4, SEPT, MLH1, WTN5A, AGTR1, BMP3, SFRP2, NEUROG1, TFPI2, SDC2, and any combination thereof.

In any of the preceding embodiments, the one or more primers from the second primer set can comprise, consist essentially of, or consist of a sequence set forth in SEQ ID NOs: 1-60, or any combination thereof.

In any of the preceding embodiments, the amplification can be multiplexed.

In any of the preceding embodiments, the analysis of the first target polynucleotide sequence and the analysis of the methylation status of the second target polynucleotide sequence can be conducted simultaneously in a single reaction.

In any of the preceding embodiments, the polynucleotide concentration in the sample can be less than about 0.1 ng/mL, less than about 1 ng/mL, less than about 3 ng/mL, less than about 5 ng/mL, less than about 10 ng/mL, less than about 20 ng/mL, or less than about 100 ng/mL.

In any of the preceding embodiments, the method can be used for the diagnosis and/or prognosis of a disease or condition in a subject, predicting the responsiveness of a subject to a treatment, identifying a pharmacogenetics marker for the disease/condition or treatment, and/or screening a population for a genetic information. In one aspect, the disease or condition is a cancer or neoplasia, and the treatment is a cancer or neoplasia treatment.

In another aspect, disclosed herein is a kit, comprising: a methylation-sensitive restriction enzyme (MSRE), and the MSRE selectively cleaves at a residue when it is unmethylated or selectively cleaves at the residue when it is methylated; a first primer set for amplifying a first target polynucleotide sequence in a sample; and/or a second primer set for analyzing a methylation status of a second target polynucleotide sequence in the sample, and the methylation status is of a residue in the second target polynucleotide sequence, and one primer of the second primer set hybridizes to the uncleaved second target polynucleotide sequence and together with another primer in the set, amplifies the uncleaved sequence but not the second target polynucleotide sequence cleaved at the residue by the MSRE. In one embodiment, the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and any combination thereof.

In any of the preceding embodiments, the first set of primers can comprise at least two allele-specific primers and a common primer.

In any of the preceding embodiments, the kit can comprise a DNA polymerase without a 3′ to 5′ exonuclease activity.

In any of the preceding embodiments, the second set of primers of the kit can comprise a common primer and at least two primers each for a different CpG site in the second target polynucleotide sequence.

In any of the preceding embodiments, the kit can further comprise an agent for purifying polynucleotides from a sample.

In any of the preceding embodiments, the kit can further comprise an agent for sequencing, such as a sequencing adapter and/or a sample-specific barcode.

In any of the preceding embodiments, the first and second sets of primers can be mixed, for example, in one vial within the kit, or the first and second sets of primers can be in separate vials and the kit can further comprise an instruction to mix all or a subset of the primers.

In any of the preceding embodiments, the first primer set and/or the second primer set of the kit can comprise one or more primers listed in Table 1 and/or Table 2, in any suitable combination.

In any of the preceding embodiments, the first primer set of the kit can comprise one or more primers for a gene selected from the group consisting of ABCB1, CYP2C19, CYP2C8, CYP2D6, CYP3A4, CYP3A5, DPYD, GSTP1, MTHFR, NQO1, RHEB, SULT1A1, UGT1A1, MPL, JAK1, NRAS, DDR2, PTEN, FGFR2, HRAS, ATM, CBL, KRAS, ERBB3, CDK4, HNF1A, FLT3, RB1, AKT1, IDH2, CDH1, TR53, ERBB2, STAT3, SMAD4, STK11, GNA11, JAK3, PPP2R1A, RET, DNMT3A, ALK, NFE2L2, SF3B1, PIK3CA, ERBB4, GNAS, U2AF1, SLC19A1, SMARCB1, CHEK2, VHL, RAF1, CTNNB1, PDGFRA, KIT, KDR, FBXW7, APC, NEUROG1, CSF1R, NPM1, TPMT, EGFR, MET, SMO, BRAF, EZH2, FGFR1, JAK2, CDKN2A, PAX5, PTCH1, ABL1, NOTCH1, ARAF, MED12, BTK, and any combination thereof.

In any of the preceding embodiments, the first primer set of the kit can comprise, consist essentially of, or consist of a sequence set forth in SEQ ID NOs: 61-788, or any combination thereof.

In any of the preceding embodiments, the second primer set of the kit can comprise one or more primers for a gene selected from the group consisting of NDRG4, SEPT, MLH1, WTN5A, AGTR1, BMP3, SFRP2, NEUROG1, TFPI2, SDC2, and any combination thereof.

In any of the preceding embodiments, the second primer set of the kit can comprise, consist essentially of, or consist of a sequence set forth in SEQ ID NOs: 1-60, or any combination thereof.

In any of the preceding embodiments, the kit can further comprise an instruction of comparing an observed number of sequencing reads to a reference number. In one embodiment, the kit further comprises a reference sample and/or information of a control locus.

In any of the preceding embodiments, the kit can further comprise separate vials for one or more components and/or instructions for using the kit.

### BRIEF DESCRIPTION OF THE DRAWINGS

FIG. 1 is an overview of the MSA-Seq (methylation specific amplification sequencing) method, according to one aspect of the present disclosure.

FIG. 2 shows validation of analytical performance with synthetic DNA mixtures (1%, 5%, 10%, 20%, 50%) of fragmented genomic DNA from the cancer cell line HCT116, which is methylated at the 24 CpG sites, with genomic DNA from NA12878 that is unmethylated at all these sites. MSA-seq was performed on these mixtures in triplicates.

FIG. 3 shows MSMC-Seq quantified CpG methylation for tumor clustering. MSMC stands for Multiple Sequentially Markovian Coalescent, a method for clustering multiple genome sequences, and in this instance, MSMC performs unbiased heretical clustering of tumor subgroups based on quantified CpG methylation.

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press: A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

### A. DEFINITIONS

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, the singular forms “a,” “an,” and “the” include the plural reference unless the context clearly dictates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z”

The term “average” as used herein refers to either a mean or a median, or any value used to approximate the mean or the median, unless the context clearly indicates otherwise.

A “subject” as used herein refers to an organism, or a part or component of the organism, to which the provided compositions, methods, kits, devices, and systems can be administered or applied. For example, the subject can be a mammal or a cell, a tissue, an organ, or a part of the mammal. As used herein, “mammal” refers to any of the mammalian class of species, preferably human (including humans, human subjects, or human patients). Mammals include, but are not limited to, farm animals, sport animals, pets, primates, horses, dogs, cats, and rodents such as mice and rats.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from animals and plants and processed samples derived therefrom.

The terms “polynucleotide,” “oligonucleotide,” “nucleic acid” and “nucleic acid molecule” are used interchangeably herein to refer to a polymeric form of nucleotides of any length, and comprise ribonucleotides, deoxyribonucleotides, and analogs or mixtures thereof. The terms include triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid,” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, inter-nucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA:DNA hybrids can exhibit higher stability in some environments. The terms “polynucleotide,” “oligonucleotide,” “nucleic acid” and “nucleic acid molecule” can comprise any suitable length, such as at least 5, 6, 7, 8, 9, 10, 20, 30, 40, 50, 100, 200, 300, 400, 500, 1,000 or more nucleotides.

It will be appreciated that, as used herein, the terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

The terms “complementary” and “substantially complementary” include the hybridization or base pairing or the formation of a duplex between nucleotides or nucleic acids, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%. In one aspect, two complementary sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein, for a reference sequence, the reverse complementary sequence is the complementary sequence of the reference sequence in the reverse order. For example, for 5′-ATCG-3′, the complementary sequence is 3′-TAGC-5′, and the reverse-complementary sequence is 5′-CGAT-3′.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, EDTA) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a polymerase, for example, a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically. As used herein, ligations are usually carried out enzymatically to form a phosphodiester linkage between a 5′ carbon terminal nucleotide of one oligonucleotide with a 3′ carbon of another nucleotide.

“Amplification,” as used herein, generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

“Sequence determination” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid.

The term “Sequencing,” “High throughput sequencing,” or “next generation sequencing” includes sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Mass.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edulcgi-bin/hgGateway) or the NCBI dbSNP website (ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations.

It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome. Sequencing technologies are available for SNP sequencing, such as the BeadArray platform (GOLDENGATE™ assay) (Illumina, Inc., San Diego, Calif.) (see Fan, et al., Cold Spring Symp. Quant. Biol., 68:69-78 (2003)), may be employed.

In some embodiments, the term “methylation state” or “methylation status” refers to the presence or absence of 5-methylcytosine (“5-mC” or “5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated,” and “hemi-methylated.” The term “hemi-methylation” or “hemimethylation” refers to the methylation state of a double stranded DNA wherein only one strand thereof is methylated. The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

“Multiplexing” or “multiplex assay” herein may refer to an assay or other analytical method in which the presence and/or amount of multiple targets, e.g., multiple nucleic acid sequences, can be assayed simultaneously by using more than one markers, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, “disease or disorder” refers to a pathological condition in an organism resulting from, e.g., infection or genetic defect, and characterized by identifiable symptoms.

### B. GENETIC VARIANT DETECTION

Mutant DNA molecules offer unique advantages over cancer-associated biomarkers because they are specific. Though mutations occur in individual normal cells at a low rate (about 10⁻⁹ to 10⁻¹⁰ mutations/bp/generation), such mutations represent such a tiny fraction of the total normal DNA that they are orders of magnitude below the detection limit of certain art methods. Several studies have shown that mutant DNA can be detected in stool, urine, and blood of CRC patients (Osborn and Ahlquist, Stool screening for colorectal cancer: molecular approaches, Gastroenterology 2005; 128:192-206).

Based on the sequencing results, detection of mutant DNA (including tumor-associated mutations) in a patient can be made, and diagnosis of a disease such as cancer and predictions regarding tumor recurrence can be made. Based on the predictions, treatment and surveillance decisions can be made. For example, circulating tumor DNA which indicates a future recurrence, can lead to additional or more aggressive therapies as well as additional or more sophisticated imaging and monitoring. Circulating DNA refers to DNA that is ectopic to a tumor.

Samples which can be analyzed include blood and stool. Blood samples may be for example a fraction of blood, such as serum or plasma. Similarly stool can be fractionated to purify DNA from other components. Tumor samples are used to identify a somatically mutated gene in the tumor that can be used as a marker of tumor in other locations in the body. Thus, as an example, a particular somatic mutation in a tumor can be identified by any standard means known in the art. Typical means include direct sequencing of tumor DNA, using allele-specific probes, allele-specific amplification, primer extension, etc. Once the somatic mutation is identified, it can be used in other compartments of the body to distinguish tumor derived DNA from DNA derived from other cells of the body. Somatic mutations are confirmed by determining that they do not occur in normal tissues of the body of the same patient. Types of tumors which can be diagnosed and/or monitored in this fashion are virtually unlimited. Any tumor which sheds cells and/or DNA into the blood or stool or other bodily fluid can be used. Such tumors include, in addition to colorectal tumors, tumors of the breast, lung, kidney, liver, pancreas, stomach, brain, head and neck, lymphatics, ovaries, uterus, bone, blood, etc.

In one aspect, highly parallel next-generation sequencing methods are used to analyze a target sequence in sample, in order to detect a genetic variant associated with a disease or condition, such as cancer. Such sequencing methods can be carried out, for example, using a one pass sequencing method or using paired-end sequencing. Next generation sequencing methods include, but are not limited to, hybridization-based methods, such as disclosed in Drmanac, U.S. Pat. Nos. 6,864,052; 6,309,824; and 6,401,267; and Drmanac et al, U.S. patent publication 2005/0191656, and sequencing by synthesis methods, e.g., Nyren et al., U.S. Pat. No. 6,210,891; Ronaghi, U.S. Pat. No. 6,828,100; Ronaghi et al. (1998), Science, 281: 363-365; Balasubramanian, U.S. Pat. No. 6,833,246; Quake, U.S. Pat. No. 6,911,345; Li et al., Proc. Natl. Acad. Sci., 100: 414-419 (2003); Smith et al., PCT publication WO 2006/074351; use of reversible extension terminators, e.g., Turner, U.S. Pat. No. 6,833,246 and Turner, U.S. Pat. No. 6,833,246 and ligation-based methods. e.g., Shendure et al. (2005), Science, 309: 1728-1739, Macevicz, U.S. Pat. No. 6,306,597; Soddart et al, PNAS USA. 2009 Apr. 20; Xiao et al., Nat Methods. 2009 March; 6(3): 199-201, all of which references are incorporated by reference herein for all purposes.

For Illumina sequencing, on each end, these constructs have flow cell binding sites, P5 and P7, which allow the library fragment to attach to the flow cell surface. The P5 and P7 regions of single-stranded library fragments anneal to their complementary oligos on the flowcell surface. The flow cell oligos act as primers and a strand complementary to the library fragment is synthesized. Then, the original strand is washed away, leaving behind fragment copies that are covalently bonded to the flowcell surface in a mixture of orientations. Copies of each fragment are then generated by bridge amplification, creating clusters. Then, the P5 region is cleaved, resulting in clusters containing only fragments which are attached by the P7 region. This ensures that all copies are sequenced in the same direction. The sequencing primer anneals to the P5 end of the fragment, and begins the sequencing by synthesis process. Index reads are performed when a sample is barcoded. When Read 1 is finished, everything from Read 1 is removed and an index primer is added, which anneals at the P7 end of the fragment and sequences the barcode. Then, everything is stripped from the template, which forms clusters by bridge amplification as in Read 1. This leaves behind fragment copies that are covalently bonded to the flowcell surface in a mixture of orientations. This time, P7 is cut instead of P5, resulting in clusters containing only fragments which are attached by the P5 region. This ensures that all copies are sequenced in the same direction (opposite Read 1). The sequencing primer anneals to the P7 region and sequences the other end of the template.

Next-generation sequencing platforms, such as MiSeq (Illumina Inc., San Diego, Calif.), can also be used for highly multiplexed assay readout. A variety of statistical tools, such as the Proportion test, multiple comparison corrections based on False Discovery Rates (see Benjamini and Hochberg, 1995, Journal of the Royal Statistical Society Series B (Methodological) 57, 289-300), and Bonferroni corrections for multiple testing, can be used to analyze assay results. In addition, approaches developed for the analysis of differential expression from RNA-Seq data can be used to reduce variance for each target sequence and increase overall power in the analysis. See Smyth, 2004, Stat Appl. Genet. Mol. Biol. 3, Article 3.

In any of the preceding embodiments, the method can be used for the diagnosis and/or prognosis of a disease or condition in a subject, predicting the responsiveness of a subject to a treatment, identifying a pharmacogenetics marker for the disease/condition or treatment, and/or screening a population for a genetic information. In one aspect, the disease or condition is a cancer or neoplasia, and the treatment is a cancer or neoplasia treatment.

In some embodiments, the nucleic acid molecule of interest disclosed herein is a cell-free DNA, such as cell-free fetal DNA (also referred to as “cfDNA”) or ctDNA. cfDNA circulates in the body, such as in the blood, of a pregnant mother, and represents the fetal genome, while ctDNA circulates in the body, such as in the blood, of a cancer patient, and is generally pre-fragmented. In other embodiments, the nucleic acid molecule of interest disclosed herein is an ancient and/or damaged DNA, for example, due to storage under damaging conditions such as in formalin-fixed samples, or partially digested samples.

As cancer cells die, they release DNA into the bloodstream. This DNA, known as ctDNA, is highly fragmented, with an average length of approximately 150 base pairs. Once the white blood cells are removed, ctDNA generally comprises a very small fraction of the remaining plasma DNA, for example, ctDNA may constitute less than about 10% of the plasma DNA. Generally, this percentage is less than about 1%, for example, less than about 0.5% or less than about 0.01%. Additionally, the total amount of plasma DNA is generally very low, for example, at about 10 ng/mL of plasma.

A DNA sample can be contacted with primers that result in specific amplification of a mutant sequence, if the mutant sequence is present in the sample. “Specific amplification” means that the primers amplify a specific mutant sequence and not other mutant sequences or the wild-type sequence. Allele-specific amplification-based methods or extension-based methods are described in WO 93/22456 and U.S. Pat. Nos. 4,851,331; 5,137,806; 5,595,890; and 5,639,611, all of which are specifically incorporated herein by reference for their teachings regarding same. While methods such as ligase chain reaction, strand displacement assay, and various transcription-based amplification methods can be used (see, e.g., review by Abramson and Myers, Current Opinion in Biotechnology 4:41-47 (1993)), PCR and/or sequencing methods can be used.

Multiple allele-specific primers, such as multiple mutant alleles or various combinations of wild-type and mutant alleles, can be employed simultaneously in a single amplification and/or sequencing reaction. Amplification products can be distinguished by different labels or size.

### C. DNA METHYLATION AND ANALYSIS

DNA methylation was first the discovered epigenetic mark. Epigenetics is the study of changes in gene expression or cellular phenotype caused by mechanisms other than changes in the underlying DNA sequence. Methylation predominately involves the addition of a methyl group to the carbon-5 position of cytosine residues of the dinucleotide CpG and is associated with repression or inhibition of transcriptional activity.

DNA methylation may affect the transcription of genes in two ways. First, the methylation of DNA itself may physically impede the binding of transcriptional proteins to the gene and, second and likely more important, methylated DNA may be bound by proteins known as methyl-CpG-binding domain proteins (MBDs). MBD proteins then recruit additional proteins to the locus, such as histone deacetylases and other chromatin remodeling proteins that can modify histones, thereby forming compact, inactive chromatin, termed heterochromatin. This link between DNA methylation and chromatin structure is very important. In particular, loss of methyl-CpG-binding protein 2 (MeCP2) has been implicated in Rett syndrome; and methyl-CpG-binding domain protein 2 (MBD2) mediates the transcriptional silencing of hypermethylated genes in cancer.

DNA methylation is an important regulator of gene transcription and a large body of evidence has demonstrated that genes with high levels of 5-methylcytosine in their promoter region are transcriptionally silent, and that DNA methylation gradually accumulates upon long-term gene silencing. DNA methylation is essential during embryonic development and in somatic cells patterns of DNA methylation are generally transmitted to daughter cells with a high fidelity. Aberrant DNA methylation patterns—hypermethylation and hypomethylation compared to normal tissue—have been associated with a large number of human malignancies. Hypermethylation typically occurs at CpG islands in the promoter region and is associated with gene inactivation. Global hypomethylation has also been implicated in the development and progression of cancer through different mechanisms.

The detection of methylated DNA, therefore, can be useful in the diagnosis of certain cancers and, for example, for following treatment efficacy. For example, WO1998056952A1 discloses a cancer diagnostic method based upon DNA methylation differences at specific CpG sites, and the method comprises bisulfite treatment of DNA, followed by methylation-sensitive single nucleotide primer extension (Ms-SNuPE) for determination of strand-specific methylation status at cytosine residues. U.S. Pat. No. 8,541,207 B2 discloses methods for analyzing the methylation state of DNA with a gene array. WO2005123942A2 discloses a method for analysis methylation patterns in DNA and identifying aberrantly methylated genes in disease tissue. Other method for detection of cytosine methylation are disclosed in WO2005071106A1, WO2003074730A1, EP1342794A1, EP1461458A2, EP1360317A2, U.S. Pat. No. 7,524,629 B2, WO2000070090A1, WO2000026401A1, US20060134650A1, and U.S. Pat. No. 7,247,428 B2. All of the patent documents in this paragraph are incorporated by reference for all purposes.

One example of a cancer wherein bisulfite sequencing has proven useful is for the screening of colorectal cancer wherein the detection of methylated Septin 9 (mS9) is used as a biomarker. Other examples of target sequences for bisulfite conversion are esophageal squamous cell carcinoma (Baba et al., Surg. Today, 2013), breast cancer (Dagdemir et al., In vivo, 2013, 27(1): 1-9), prostate cancer (Willard and Koochekpour, Am. J. Cancer Res. 2012, 2(6):620-657), non-Hodgkin's lymphomas (Yin et al., Front Genet., 2012, 3:233), oral cancers (Gasche and Goel, Future Onocol., 2012, 8(11):1407-1425), etc. One of ordinary skill in the art will appreciate that the methods of the present invention are applicable to and easily adapted to the improved detection of these and other cancers known to be manifested at least in part by hypermethylation or hypomethylation of target gene sequences. Likewise, other medical conditions known to those of skill line art that wherein hypermethylation and/or hypomethylation are part of the known etiology will have improved detection, for diagnosis and/or prognosis and/or as companion diagnostics, with the application of the methods disclosed herein.

Bisulfite conversion is the use of bisulfite reagents to treat DNA to determine its pattern of methylation. The treatment of DNA with bisulfite converts cytosine residues to uracil but leaves 5-methylcytosine residues unaffected. Thus, bisulfite treatment introduces specific changes in the DNA sequence that depend on the methylation status of the individual cytosine residues. Various analyses can be performed on the altered sequence to retrieve this information, for example, in order to differentiate between single nucleotide polymorphisms (SNP) resulting from the bisulfite conversion. U.S. Pat. Nos. 7,620,386, 9,365,902, and U.S. Patent Application Publication 2006/0134643, all of which are incorporated herein by reference, exemplify methods known to one of ordinary skill in the art with regard to detecting sequences altered due to bisulfite conversion. However, one consequence of bisulfite conversion is that the double-stranded conformation of the original target is disrupted due to loss of sequence complementarity. In addition, bisulfite conversion is a harsh treatment that tends to lead to material losses, which can compromise the assay sensitivity on low-input samples, such cell-free DNA, including circulating tumor DNA (also referred to as “cell-free tumor DNA,” or “ctDNA”).

### D. SIMULTANEOUS DETECTION OF GENETIC VARIANTS AND DNA METHYLATION ON LIMITED SAMPLE INPUT

Simultaneous detection of genetic variants and DNA methylation is difficult for the first- and second-generation sequencing, especially when the input DNA amount is low and that limited input needs to be further divided for two separate work flows, one for genetic variant detection and the other for DNA methylation analysis.

Flusberg et al. (2010) in “Direct detection of DNA methylation during single-molecule, real-time sequencing,” Nat. Methods 7: 461-465, and Manrao et al. (2012) in “Reading DNA at single-nucleotide resolution with a mutant MspA nanopore and phi29 DNA polymerase,” Nat. Biotechnol 30: 349-353, attempted to combine third generation sequencing with DNA methylation analysis. However, their detection accuracy was low, and far from being adequate for routine clinical tests.

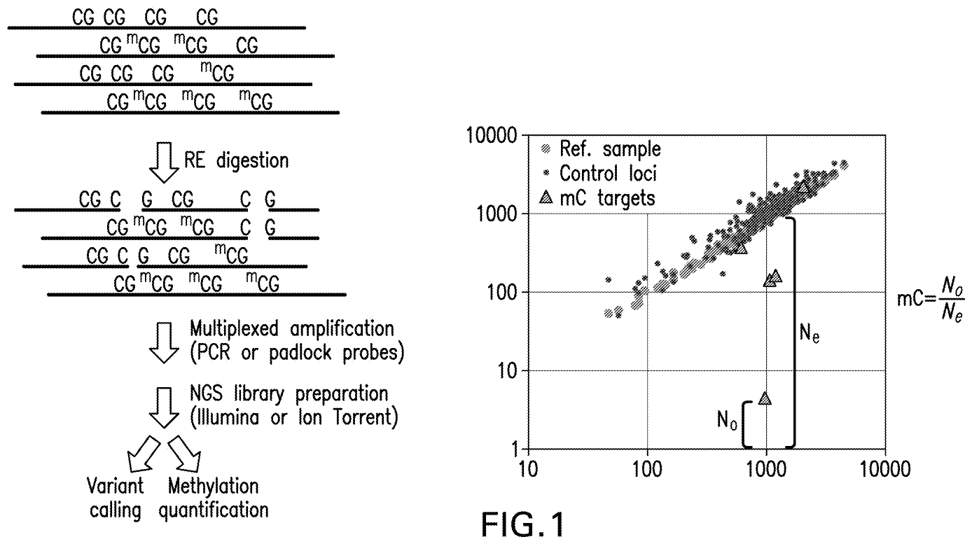

In one aspect, disclosed herein is a method (MSA-seq) for efficient quantification of DNA methylation status of multiple CpG sites, and simultaneous detection and quantification of genetic variants at multiple targets. In some embodiments, the input DNAs, such as ctDNA, are first digested with methylation-sensitivity restriction enzymes, such as HapII and/or SalI, followed by multiplexed amplification of assayed targets and next-generation sequencing (FIG. 1, left panel). The methylation levels of the target CpG sites are inferred by the relative read depth, whereas the genetic variants are called from the raw sequencing reads (FIG. 1, right panel). In one aspect, the majority of genetic variants are accessible with a single-reaction assay. The variants in the ctDNA can be interrogated using various methods, including next generation sequencing discussed above.

In some embodiments, for a minority of variants that locate too close to the restriction enzyme recognition sites, a second multiplexed amplification reaction is performed on the undigested input DNA, for a separate sequencing library.

While methylation sensitive restriction enzyme digestion has been adopted for multiple methylation assays, including several NGS-based methods, such as Methyl-seq, MCA-seq, HELP-seq and MSCC, MSA-seq is unique in that genomic fragments containing the targeted CpG sites were extracted from the remaining genomic fragments by multiplexed amplification with at least one defined end, and the methylation levels are correlated with the amplifiable fragments. For a review of methods for methylation analysis, see Laird (2010), “Principles and challenges of genome-wide DNA methylation analysis,” Nat Rev Genet 11: 191-203.

In one aspect, the present method does not rely on adaptor ligation with the digested ends. The number of targeted CpG sites per assay is highly flexible, in the range from one to tens of thousands. The methylation levels can be quantitated by normalization using the read depth information of internal control loci that do not contain the digestion sites, without requiring a second control reaction using methyl-insensitive restriction enzymes. In another method, the present method does not involve bisulfite conversion, which can result in >90% loss of DNA molecules. The combination of these features leads to high scalability, superior sensitivity and low input requirements which are particularly relevant to liquid biopsies.

In one aspect of the present disclosure, target capture can be implemented with at least three different methods, including multiplexed PCR (Qiagen Multiplexed PCR, Thermo Fisher AmpliSeq), padlock capture (Roche Heat-Seq), and selector capture (Agilent HaloPlex). In some embodiments, primers or probes targeting short genomic intervals (40-200 bp including the oligo annealing regions) covering the CpG sites of interests are designed. A separate set of primers or probes is also designed for the genetic variants (mutations) of interest. Typically a larger fraction of target sequence in the second set do not contain restriction enzyme recognition sites, hence their sequencing read depth can be used as the internal controls for the calculation of CpG methylation levels. In rare situation where all targets in the second set can be digested by the restriction enzyme(s), additional amplicons will be designed as non-digested internal controls. The relative read depth (mean and variance) for all amplicons in an assay is first determined by multiplexed amplification and sequencing on the non-digested DNA fragments that mimic the fragment size distribution of real samples. In one aspect, this only needs to be done once for each type of clinical samples. For each clinical sample of interest, the methylation of each target CpG site is determined by calculating the ratio of observed read depth over expected read depth after regression normalization. In one aspect, genetic variants are called by routine variant calling procedures, including read mapping, local alignment, variant calling and/or filtering.

In one aspect, the present method has a number of immediate clinical applications. One of such applications is non-invasive screening, early detection, or monitor of tumors on patients' plasma, stool, urine or other types of biofluids. Another application is non-invasive prenatal screening of fetal aneuploidy, such as trisomy 21 Down's syndrome.

In one aspect, provided herein is a method for analyzing a first target polynucleotide sequence and a methylation status of a second target polynucleotide sequence in a sample, comprising contacting a sample containing or suspected of containing a polynucleotide with a methylation-sensitive restriction enzyme (MSRE). In one aspect, the MSRE selectively cleaves the polynucleotide at a residue when it is unmethylated or selectively cleaves the polynucleotide at the residue when it is methylated. In any of the preceding embodiments, the MSRE can be selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and any combination thereof.

In one aspect, disclosed herein is a method for analyzing a first target set of polynucleotide sequence for sequence changes and a second target set of polynucleotide sequence for methylation status in a sample, comprising: 1) contacting a sample comprising a polynucleotide with an MSRE, wherein the MSRE selectively cleaves the polynucleotide at a residue when it is unmethylated or selectively cleaves the polynucleotide at the residue when it is methylated; 2) subjecting the sample from step 1) to polynucleotide amplification, using a mixture of: i) a first primer set for amplifying a first target set of polynucleotide sequence in the sample, and ii) a second primer set for analyzing a methylation status of a second target set of polynucleotide sequence in the sample, wherein the methylation status is of a residue in the second target set of polynucleotide sequence, and one primer of the second primer set hybridizes to the uncleaved second target polynucleotide sequence and together with another primer in the set, amplifies the uncleaved sequence but not the second target polynucleotide sequence cleaved at the residue by the MSRE; and 3) sequencing analysis polynucleotides amplified in step 2), wherein the first target set of polynucleotide sequence is analyzed using sequencing reads from the amplified first target set of polynucleotide sequence, and the methylation status of the residue of the second target polynucleotide sequence is analyzed by comparing the observed number of sequencing reads (Nₒ) from the amplified second target set of polynucleotide sequence to an expected reference number (Nₑ).

In one embodiment, the first target set of polynucleotide sequence is analyzed using sequencing reads from the amplified first target set of polynucleotide sequence, as compared to a reference sequence, for example, a wild-type sequence and/or a human sequence for the target sequence. The comparison can be done by sequence alignment.

In another embodiment, the first target set of polynucleotide sequence is analyzed using without comparing sequencing reads from the amplified first target set of polynucleotide sequence to a reference sequence. For example, by aligning all the sequencing reads to obtain a consensus sequence so it is possible to tell which variants are the minority alleles. In one aspect, the minority allele comprises a mutation.

In one embodiment, a sample contacted with an MSRE can be analyzed by constructing a single-stranded library by ligation, as disclosed in U.S. Provisional Application No. ______, entitled “Compositions and Methods for Library Construction and Sequence Analysis,” filed Apr. 19, 2017 (Attorney Docket No. 737993000200), which is incorporated herein by reference in its entirety for all purposes. In one aspect, the MSRE treatment is before the dephosphorylation and/or the denaturing step of the single-stranded ligation method. In one embodiment, a method comprising ligating a set of adaptors to a library of single-stranded polynucleotides is provided, and in the method, an MSRE-treated sample is denatured to create the library of single-stranded polynucleotides, and the ligation is catalyzed by a single-stranded DNA (ssDNA) ligase, each single-stranded polynucleotide is blocked at the 5′ end to prevent ligation at the 5′ end, each adaptor comprises a unique molecular identifier (UMI) sequence that earmarks the single-stranded polynucleotide to which the adaptor is ligated, each adaptor is blocked at the 3′ end to prevent ligation at the 3′ end, and the 5′ end of the adaptor is ligated to the 3′ end of the single-stranded polynucleotide by the ssDNA ligase to form a linear ligation product, thereby obtaining a library of linear, single-stranded ligation products. In any of the preceding embodiments, the method can further comprise converting the library of linear, single-stranded ligation products into a library of linear, double-stranded ligation products. In one aspect, the conversion uses a primer or a set of primers each comprising a sequence that is reverse-complement to the adaptor and/or hybridizable to the adaptor. In any of the preceding embodiments, the method can further comprise amplifying and/or purifying the library of linear, double-stranded ligation products. In any of the preceding embodiments, the method herein can comprise amplifying the library of linear, double-stranded ligation products, e.g., by a polymerase chain reaction (PCR), using a primer or a set of primers each comprising a sequence that is reverse-complement to the adaptor and/or hybridizable to the adaptor, a primer hybridizable to the target sequence (e.g., an EGFR gene sequence), thereby obtaining an amplified library of linear, double-stranded ligation products comprising sequence information of the target sequence. In any of the preceding embodiments, the method can further comprise sequencing the amplified library of linear, double-stranded ligation products. Thus, the methylation status and/or genetic variant analysis of one or more target sequences can be performed using semi-targeted amplification of the single-stranded library.

The target sequence(s) for methylation analysis and/or the target sequence(s) for variant detection can be on the same molecule or on different molecules, for example, two different DNA fragments, in the sample. In one aspect, the target polynucleotide sequences can be on the same gene. In another aspect, the target polynucleotide sequences can be in a coding region of a gene whereas the second target polynucleotide sequence can be in a non-coding and/or regulatory region of or for the same gene. In another aspect, the target polynucleotide sequences can be on different genes. In one aspect, the genes function in the same biological pathway or network. In another aspect, the target polynucleotide sequences can be on the same or different chromosomes (for example, as shown in Table 3) or on the same or different extrachromosomal DNA molecules (such as mitochondria DNA), or one on a chromosome and the other on an extrachromosomal DNA molecule.

In summary, one aspect of the present disclosure is an integrated method for simultaneous detection of both a genomic variance and quantification of a DNA methylation state/status on one or more (e.g., hundreds of thousands of) targets, without splitting the limited materials for two different workflows.

### E. KITS

Disclosed in another aspect herein is a kit, comprising: a first primer set for amplifying a first target polynucleotide sequence in a sample; and/or a second primer set for analyzing a methylation status of a second target polynucleotide sequence in the sample, and the methylation status is of a residue in the second target polynucleotide sequence, and one primer of the second primer set hybridizes to the uncleaved second target polynucleotide sequence and together with another primer in the set, amplifies the uncleaved sequence but not the second target polynucleotide sequence cleaved at the residue by the MSRE. In one embodiment, the kit further comprises an MSRE, and the MSRE selectively cleaves at a residue when it is unmethylated or selectively cleaves at the residue when it is methylated. In one embodiment, the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and any combination thereof.

In any of the preceding embodiments, the first primer set of the kit can comprise one or more primers for a gene selected from the group consisting of ABCB1, CYP2C19, CYP2C8, CYP2D6, CYP3A4, CYP3A5, DPYD, GSTP1, MTHFR, NQO1, RHEB, SULT1A1, UGT1A1, MPL, JAK1, NRAS, DDR2, PTEN, FGFR2, HRAS, ATM, CBL, KRAS, ERBB3, CDK4, HNF1A, FLT3, RB1, AKT1, IDH2, CDH1, TR53, ERBB2, STAT3, SMAD4, STK11, GNA11, JAK3, PPP2R1A, RET, DNMT3A, ALK, NFE2L2, SF3B1, PIK3CA, ERBB4, GNAS, U2AF1, SLC19A1, SMARCB1, CHEK2, VHL, RAF1, CTNNB1, PDGFRA, KIT, KDR, FBXW7, APC, NEUROG1, CSF1R, NPM1, TPMT, EGFR, MET, SMO, BRAF, EZH2, FGFR1, JAK2, CDKN2A, PAX5, PTCH1, ABL1, NOTCH1, ARAF, MED12, BTK, and any combination thereof.

In any of the preceding embodiments, the first primer set of the kit can comprise, consist essentially of, or consist of a sequence set forth in SEQ ID NOs: 61-788, or any combination thereof.

In any of the preceding embodiments, the second primer set of the kit can comprise one or more primers for a gene selected from the group consisting of NDRG4, SEPT, MLH1, WTN5A, AGTR1, BMP3, SFRP2, NEUROG1, TFPI2, SDC2, and any combination thereof.

In any of the preceding embodiments, the second primer set of the kit can comprise, consist essentially of, or consist of a sequence set forth in SEQ ID NOs: 1-60, or any combination thereof.

Diagnostic kits based on the kit components described above are also provided, and they can be used to diagnose a disease or condition in a subject, for example, cancer. In another aspect, the kit can be used to predict individual's response to a drug, therapy, treatment, or a combination thereof. Such test kits can include devices and instructions that a subject can use to obtain a sample, e.g., of ctDNA, without the aid of a health care provider.

For use in the applications described or suggested above, kits or articles of manufacture are also provided. Such kits may comprise at least one reagent specific for genotyping a marker for a disease or condition, and may further include instructions for carrying out a method described herein.

In some embodiments, provided herein are compositions and kits comprising primers and primer pairs, which allow the specific amplification of the polynucleotides or of any specific parts thereof, and probes that selectively or specifically hybridize to nucleic acid molecules or to any part thereof for the purpose of detection, either qualitatively or quantitatively. Probes may be labeled with a detectable marker, such as, for example, a radioisotope, fluorescent compound, bioluminescent compound, a chemiluminescent compound, metal chelator or enzyme. Such probes and primers can be used to detect the presence of polynucleotides in a sample and as a means for detecting cell expressing proteins encoded by the polynucleotides. As will be understood by the skilled artisan, a great many different primers and probes may be prepared based on the sequences provided herein and used effectively to amplify, clone and/or determine the presence and/or levels of polynucleotides, such as genomic DNAs, mtDNAs, and fragments thereof.

In some embodiments, the kit may additionally comprise reagents for detecting presence of polypeptides. Such reagents may be antibodies or other binding molecules that specifically bind to a polypeptide. In some embodiments, such antibodies or binding molecules may be capable of distinguishing a structural variation to the polypeptide as a result of polymorphism, and thus may be used for genotyping. The antibodies or binding molecules may be labeled with a detectable marker, such as, for example, a radioisotope, fluorescent compound, bioluminescent compound, a chemiluminescent compound, metal chelator or enzyme. Other reagents for performing binding assays, such as ELISA, may be included in the kit.

In some embodiments, the kits comprise reagents for genotyping at least two, at least three, at least five, at least ten, or more markers. The markers may be a polynucleotide marker (such as a cancer-associated mutation or SNP) or a polypeptide marker (such as overexpression or a post-translational modification, including hyper- or hypo-phosphorylation, of a protein) or any combination thereof. In some embodiments, the kits may further comprise a surface or substrate (such as a microarray) for capture probes for detecting of amplified nucleic acids.

The kits may further comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a biomarker. The kit may also have containers containing nucleotide(s) for amplification of the target nucleic acid sequence and/or a container comprising a reporter-means bound to a reporter molecule, such as an enzymatic, florescent, or radioisotope label.

The kit typically comprises the container(s) described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The kit can further comprise a set of instructions and materials for preparing a tissue or cell or body fluid sample and preparing nucleic acids (such as ctDNA) from the sample.

### H. FURTHER EXEMPLARY EMBODIMENTS

In any of the preceding embodiments, the ssDNA ligase can be a Thermus bacteriophage RNA ligase such as a bacteriophage TS2126 RNA ligase (e.g., CircLigase™ and CircLigase II™), or an archaebacterium RNA ligase such as Methanobacterium thermoautotrophicum RNA ligase 1. In other aspects, the ssDNA ligase is an RNA ligase, such as a T4 RNA ligase, e.g., T4 RNA ligase I, e.g., New England Biosciences, M0204S, T4 RNA ligase 2, e.g., New England Biosciences, M0239S, T4 RNA ligase 2 truncated, e.g., New England Biosciences, M0242S, T4 RNA ligase 2 truncated KQ, e.g., M0373S, or T4 RNA ligase 2 truncated K227Q, e.g., New England Biosciences, M0351S. In any of the preceding embodiments, the ssDNA ligase can also be a thermostable 5′ App DNA/RNA ligase, e.g., New England Biosciences, M0319S, or T4 DNA ligase, e.g., New England Biosciences, M0202S.

In some embodiments, the present methods comprise ligating a set of adaptors to a library of single-stranded polynucleotides using a single-stranded DNA (ssDNA) ligase. Any suitable ssDNA ligase, including the ones disclosed herein, can be used. The adaptors can be used at any suitable level or concentration, e.g., from about 1 μM to about 100 μM such as about 1 μM, 10 μM, 20 μM, 30 μM, 40 μM, 50 μM, 60 μM, 70 μM, 80 μM, 90 μM, or 100 μM. or any subrange thereof. The adapter can comprise or begin with any suitable sequences or bases. For example, the adapter sequence can begin with all 2 bp combinations of bases.

In some embodiments, the ligation reaction can be conducted in the presence of a crowding agent. In one aspect, the crowding agent comprises a polyethylene glycol (PEG), such as PEG 4000, PEG 6000, or PEG 8000, Dextran, and/or Ficoll. The crowding agent, e.g., PEG, can be used at any suitable level or concentration. For example, the crowding agent, e.g., PEG, can be used at a level or concentration from about 0% (w/v) to about 25% (w/v), e.g., at about 0% (w/v), 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), 15% (w/v), 16% (w/v), 17% (w/v), 18% (w/v), 19% (w/v), 20% (w/v), 21% (w/v), 22% (w/v), 23% (w/v), 24% (w/v), or 25% (w/v), or any subrange thereof.

In some embodiments, the ligation reaction can be conducted for any suitable length of time. For example, the ligation reaction can be conducted for a time from about 2 to about 16 hours, %, e.g., for about 2 hours, 3 hours, 4 hours, 5 hours, 6 hours, 7 hours, 8 hours, 9 hours, 10 hours, 11 hours, 12 hours, 13 hours, 14 hours, 15 hours, or 16 hours, or any subrange thereof.

In some embodiments, the ssDNA ligase in the ligation reaction can be used in any suitable volume. For example, the ssDNA ligase in the ligation reaction can be used at a volume from about 0.5 μl to about 2 μl, %, e.g., at about 0.5 μl, 0.6 μl, 0.7 μl, 0.8 μl, 0.9 μl 1 μl, 1.1 μl, 1.2 μl, 1.3 μl, 1.4 μl, 1.5 μl, 1.6 μl, 1.7 μl, 1.8 μl, 1.9 μl, or 2 μl, or any subrange thereof.

In some embodiments, the ligation reaction can be conducted in the presence of a ligation enhancer, e.g., betaine. The ligation enhancer, e.g., betaine, can be used at any suitable volume, e.g., from about 0 μl to about 1 μl, e.g., at about 0 μl, 0.1 μl, 0.2 μl, 0.3 μl, 0.4 μl, 0.5 μl, 0.6 μl, 0.7 μl, 0.8 μl, 0.9 μl, 1 μl, or any subrange thereof.

In some embodiments, the ligation reaction can be conducted using a T4 RNA ligase I, e.g., the T4 RNA ligase I from New England Biosciences, M0204S, in the following exemplary reaction mix (20 μl): 1×Reaction Buffer (50 mM Tris-HCl, pH 7.5, 10 mM MgCl2, 1 mM DTT), 25% (wt/vol) PEG 8000, 1 mM hexamine cobalt chloride (optional), 1 μl (10 units) T4 RNA Ligase, and 1 mM ATP. The reaction can be incubated at 25° C. for 16 hours. The reaction can be stopped by adding 40 μl of 10 mM Tris-HCl pH 8.0, 2.5 mM EDTA.

In some embodiments, the ligation reaction can be conducted using a Thermostable 5′ App DNA/RNA ligase, e.g., the Thermostable 5′ App DNA/RNA ligase from New England Biosciences, M0319S, in the following exemplary reaction mix (20 μl): ssDNA/RNA Substrate 20 pmol (1 pmol/ul), 5′ App DNA Oligonucleotide 40 pmol (2 pmol/μl), 10×NEBuffer 1 (2 μl), 50 mM MnCl₂ (for ssDNA ligation only) (2 μl), Thermostable 5′ App DNA/RNA Ligase (2 μl (40 pmol)), and Nuclease-free Water (to 20 μl). The reaction can be incubated at 65° C. for 1 hour. The reaction can be stopped by heating at 90° C. for 3 minutes.

In some embodiments, the ligation reaction can be conducted using a T4 RNA ligase 2, e.g., the T4 RNA ligase 2 from New England Biosciences, M0239S, in the following exemplary reaction mix (20 μl): T4 RNA ligase buffer (2 μl), enzyme (1 μl), PEG (10 μl), DNA (1 μl), Adapter (2 μl), and water (4 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated, e.g., the T4 RNA ligase 2 Truncated from New England Biosciences, M0242S, in the following exemplary reaction mix (20 μl): T4 RNA ligase buffer (2 μl), enzyme (1 μl), PEG (10 μl), DNA (1 μl), Adapter (2 μl), and water (4 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated K227Q, e.g., the T4 RNA ligase 2 Truncated K227Q from New England Biosciences, M0351S, in the following exemplary reaction mix (20 μl): T4 RNA ligase buffer (2 μl), enzyme (1 μl), PEG (10 μl), DNA (1 μl), Adenylated Adapter (0.72 μl), and water (5.28 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated KQ, e.g., the T4 RNA ligase 2 Truncated KQ from New England Biosciences, M0373S, in the following exemplary reaction mix (20 μl): T4 RNA ligase buffer (2 μl), enzyme (1 μl), PEG (10 μl), DNA (1 μl), Adenylated Adapter (0.72 μl), and water (5.28 μl). The reaction can be incubated at 25° C. for 16 hours. The reaction can be stopped by heating at 65° C. for 20 minutes.

In some embodiments, the ligation reaction can be conducted using a T4 DNA ligase, e.g., the T4 DNA ligase from New England Biosciences, M0202S, in the following exemplary reaction mix (20 μl): T4 RNA ligase buffer (2 μl), enzyme (1 μl), PEG (10 μl), DNA (1 μl), Adenylated Adapter (0.72 μl), and water (5.28 μl). The reaction can be incubated at 16° C. for 16 hours. The reaction can be stopped by heating at 65 C for 10 minutes.

The second strand synthesis step can be conducted using any suitable enzyme. For example, the second strand synthesis step can be conducted using Bst polymerase, e.g., New England Biosciences, M0275S or Klenow fragment (3′->5′ exo-), e.g., New England Biosciences, M0212S.

In some embodiments, the second strand synthesis step can be conducted using Bst polymerase, e.g., New England Biosciences, M0275S, in the following exemplary reaction mix (10 μl): water (1.5 μl), primer (0.5 μl), dNTP (1 μl), ThermoPol Reaction buffer (5 μl), and Bst (2 μl). The reaction can be incubated at 62° C. for 2 minutes and at 65° C. for 30 minutes. After the reaction, the double stranded DNA molecules can be further purified.

In some embodiments, the second strand synthesis step can be conducted using Klenow fragment (3′->5′ exo-), e.g., New England Biosciences, M0212S, in the following exemplary reaction mix (10 μl): water (0.5 μl), primer (0.5 μl), dNTP (1 μl), NEB buffer (2 μl), and exo- (3 μl). The reaction can be incubated at 37° C. for 5 minutes and at 75° C. for 20 minutes. After the reaction, the double stranded DNA molecules can be further purified.

After the second strand synthesis, but before the first or semi-targeted PCR, the double stranded DNA can be purified. The double stranded DNA can be purified using any suitable technique or procedure. For example, the double stranded DNA can be purified using any of the following kits: Zymo clean and concentrator, Zymo research, D4103; Qiaquick, Qiagen, 28104; Zymo ssDNA purification kit, Zymo Research, D7010; Zymo Oligo purification kit, Zymo Research, D4060; and AmpureXP beads, Beckman Coulter, A63882: 1.2×-4× bead ratio.

The first or semi-targeted PCR can be conducted using any suitable enzyme or reaction conditions. For example, the polynucleotides or DNA strands can be annealed at a temperature ranging from about 52° C. to about 72° C., e.g., at about 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., or 72° C., or any subrange thereof. The first or semi-targeted PCR can be conducted for any suitable rounds of cycles. For example, the first or semi-targeted PCR can be conducted for 10-40 cycles, e.g., for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 cycles. The primer pool can be used at any suitable concentration. For example, the primer pool can be used at a concentration ranging from about 5 nM to about 200 nM, e.g., at about 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, or 200 nM, or any subrange thereof.

The first or semi-targeted PCR can be conducted using any suitable temperature cycle conditions. For example, the first or semi-targeted PCR can be conducted using any of the following cycle conditions: 95° C. 3 minutes, (95° C. 15 seconds, 62° C. 30 seconds, 72° C. 90 seconds) ×3 or ×5; or (95° C. 15 seconds, 72° C. 90 seconds) ×23 or ×21, 72C 1 minute, 4° C. forever.

In some embodiments, the first or semi-targeted PCR can be conducted using KAPA SYBR FAST, e.g., KAPA biosciences, KK4600, in the following exemplary reaction mix (50 μl): DNA (2 μl), KAPASYBR (25 μl), Primer Pool (26 nM each) (10 μl), Aprimer (100 uM) (0.4 μl), and water (12.6 μl). The first or semi-targeted PCR can be conducted using any of the following cycle conditions: 95° C. 30 seconds, (95° C. 10 seconds, 50-56° C. 45 seconds, 72° C. 35 seconds) ×40.

In some embodiments, the first or semi-targeted PCR can be conducted using KAPA HiFi, e.g., KAPA Biosciences, KK2601, in the following exemplary reaction mix (50 μl): DNA (15 μl), KAPAHiFi (25 μl), Primer Pool (26 nM each) (10 μl), and Aprimer (100 uM) (0.4 μl). The first or semi-targeted PCR can be conducted using any of the following cycle conditions: 95° C. 3 minutes, (98° C. 20 seconds, 53-54° C. 15 seconds, 72° C. 35 seconds) ×15, 72° C. 2 minutes, 4° C. forever.

Bisulfite conversion can be conducted using any suitable techniques, procedures or reagents. In some embodiments, bisulfite conversion can be conducted using any of the following kits and procedures provided in the kit: EpiMark Bisulfite Conversion Kit, New England Biosciences, E3318S; EZ DNA Methylation Kit, Zymo Research, D5001; MethylCode Bisulfite Conversion Kit, Thermo Fisher Scientific, MECOV50; EZ DNA Methylation Gold Kit, Zymo Research, D5005; EZ DNA Methylation Direct Kit, Zymo Research, D5020; EZ DNA Methylation Lightning Kit, Zymo Research, D5030T; EpiJET Bisulfite Conversion Kit, Thermo Fisher Scientific, K1461; or EpiTect Bisulfite Kit, Qiagen, 59104.

In some embodiments, DNA molecules can be prepared using the procedures illustrated in Example 4, including the steps for constructing single-stranded polynucleotide, conversion of single-stranded polynucleotide library to double-stranded polynucleotide library, semi-targeted amplification of double-stranded polynucleotide library, and construction of sequence library. Such DNA molecules can further be analyzed for methylation status using any suitable methods or procedures.

### I. EXAMPLES

### Example 1

In this example, 24 CpG sites that overlap with the HpaII recognition motif in the promoters of ten genes (AGTR1, BMP3, MLH1, NDRG4, NEUROG1, SDC2, SEPT, SFRP2, TFPI2, WNT5A) were selected. An AmpliSeq customized primer set was designed to cover these methylation targets, as well as 370 genomic regions that are commonly mutated in cancers.

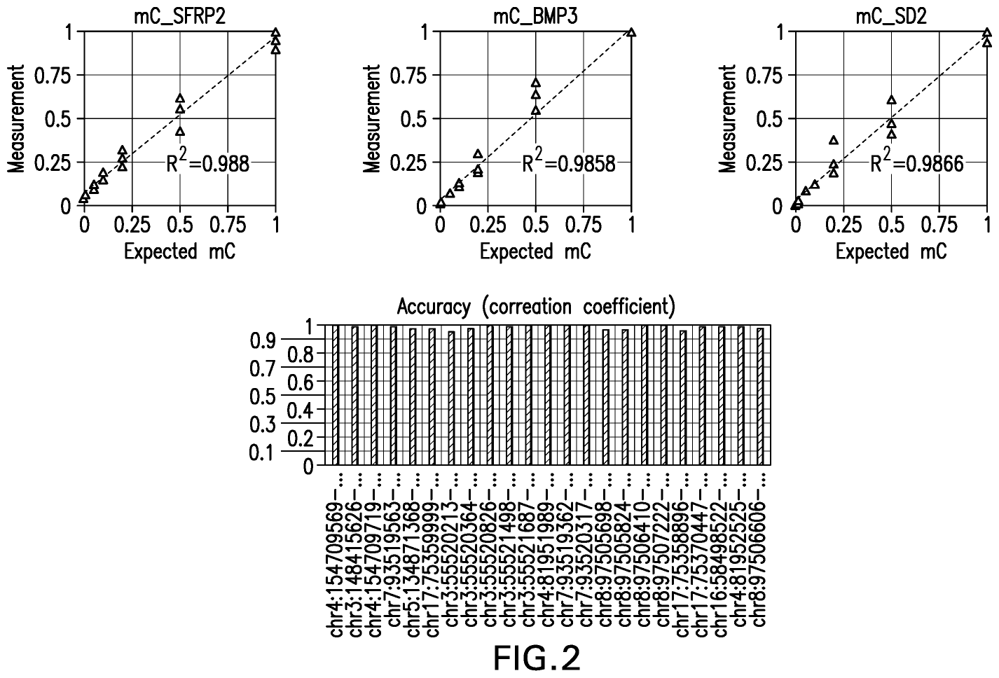

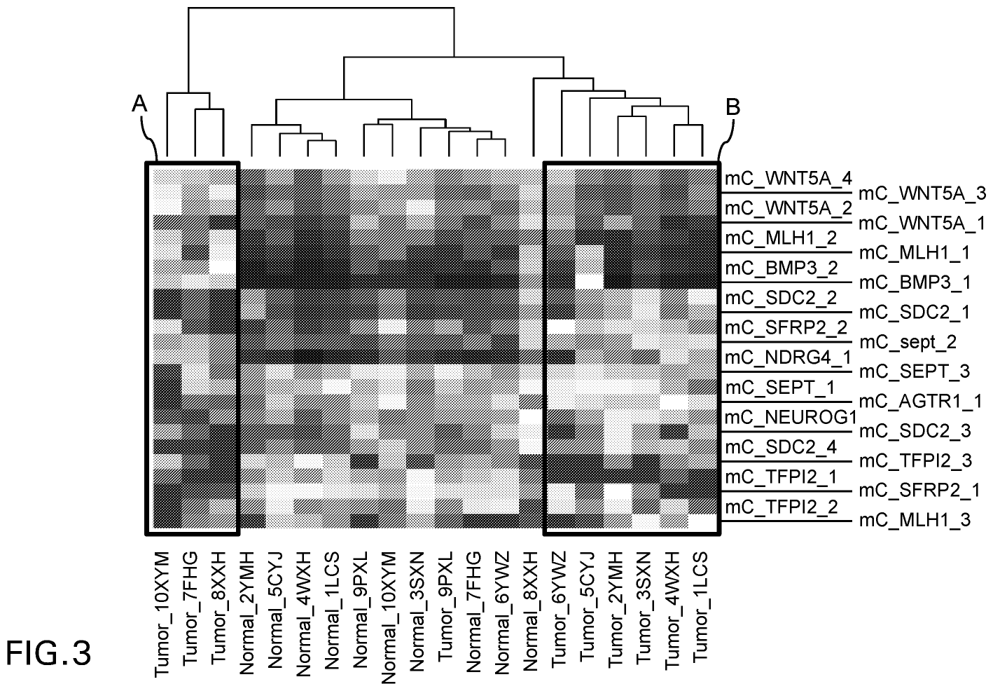

Mixtures (1%, 5%, 10%, 20%, 50%) were created of fragmented genomic DNA from the cancer cell line HCT116, which is methylated at the 24 CpG sites, with genomic DNA from NA12878 that is unmethylated at all these sites. MSA-seq was performed on these mixtures in triplicates. The methylation measurements have high correlation (average correlation coefficient R=0.983) and linearity with the expected values (FIG. 2). FIG. 3 shows MSMC-Seq quantified CpG methylation for tumor clustering. This method of unbiased hierarchical clustering of tumor samples separates these tumor samples into 3 groups based on methylation biomarker level/status: Group A, Group B, and the group in between A and B.

Exemplary primer pairs used are listed in Table 1 below.

TABLE 1
Exemplary primer pairs.

| Gene | Forward Primer | SEQ ID NO | Reverse Primer | SEQ ID NO |
|---|---|---|---|---|
| mC_NDRG4 | TACCTGTTTGTGTGCG | SEQ ID NO: 1 | CCGAGCTCCGCTGGTC | SEQ ID NO: 2 |
| mC_SEPT | GGACTCGCATGTTCG | SEQ ID NO: 3 | AACAAAGTTCTCTGTC | SEQ ID NO: 4 |
| mC_SEPT | CCAGGACGCACAGTTT | SEQ ID NO: 5 | AGTCGGAGGTGAGGAA | SEQ ID NO: 6 |
| mC_SEPT | CTGAGCCTGTGAGTGC | SEQ ID NO: 7 | GCGCTGGAGACCATT | SEQ ID NO: 8 |
| mC_MLH1 | CAGCTCTCTCTTCAGG | SEQ ID NO: 9 | GAGGCTGAGCACGAAT | SEQ ID NO: 10 |
| mC_MLH1 | GTAGCTACGATGAGG | SEQ ID NO: 11 | AAAGAAGCAAGATGGA | SEQ ID NO: 12 |
| mC_MLH1 | TCAAAGAGATGATTGA | SEQ ID NO: 13 | CATGCGCTGTACAT | SEQ ID NO: 14 |
| mC_MLH1 | ACACTACCCAATGCCT | SEQ ID NO: 15 | AATAATGTGATGGAAT | SEQ ID NO: 16 |
| mC_MLH1 | TGAAGAACTGTTCTAC | SEQ ID NO: 17 | GTGGAGAGCTACTATT | SEQ ID NO: 18 |
| mC_WNT5A | AGGCCCAAGTGTTTT | SEQ ID NO: 19 | TTTGCAGCAGTGGTG | SEQ ID NO: 20 |
| mC_WNT5A | TAATAATGCTAATAAC | SEQ ID NO: 21 | GGAGGCCAGATTGTAG | SEQ ID NO: 22 |
| mC_WNT5A | GATCTCCTGGGACACT | SEQ ID NO: 23 | CCCTTCGCCTCTTCCT | SEQ ID NO: 24 |
| mC_WNT5A | ATGTACCACTACTCAA | SEQ ID NO: 25 | GAGGAGCTGGAGATCA | SEQ ID NO: 26 |
| mC_WNT5A | AGTGTGGACGTCTCTG | SEQ ID NO: 27 | CGACTTGTGCGTTTTC | SEQ ID NO: 28 |
| mC_AGTR1 | AGAACACGAATCTCCG | SEQ ID NO: 29 | TGATGCCACAGTCGTC | SEQ ID NO: 30 |
| mC_AGTR1 | GCAAAACAGAGCCTCG | SEQ ID NO: 31 | ACGTCCTGTCACTCG | SEQ ID NO: 32 |
| mC_BMP3 | CCTGGAAAAGGCAATC | SEQ ID NO: 33 | CCTCGCTTTATTTTTG | SEQ ID NO: 34 |
| mC_BMP3 | ACCGAAGCCACCTTTC | SEQ ID NO: 35 | CTGTACCTGTCATAGA | SEQ ID NO: 36 |
| mC_SFRP2 | AACGGTCGCACTCAA | SEQ ID NO: 37 | CTGCCTCGATGACCTA | SEQ ID NO: 38 |
| mC_SFRP2 | ACAGGAACTTCTTGGT | SEQ ID NO: 39 | CATCGAATACCAGAAC | SEQ ID NO: 40 |
| mC_NEUROG1 | GAGATGCAGGTCTCAA | SEQ ID NO: 41 | GCTGTTGGGAACGTAA | SEQ ID NO: 42 |
| mC_TFPI2 | ACTTGAGAAAACCCAG | SEQ ID NO: 43 | TGGAGGATAGAAAGTA | SEQ ID NO: 44 |
| mC_TFPI2 | CGTGTACCTGTCGTAG | SEQ ID NO: 45 | ACCACTTTCCCTCTCT | SEQ ID NO: 46 |
| mC_TFPI2 | CAGTAATGGGAAATCT | SEQ ID NO: 47 | GAACTCCGCACTTTCT | SEQ ID NO: 48 |
| mC_SDC2 | AGAGGAGAGAGGAAAA | SEQ ID NO: 49 | GCAGCTCCGAGGACCA | SEQ ID NO: 50 |
| mC_SDC2 | CAATCGGCGTGTAA | SEQ ID NO: 51 | TCTTCTTTTCCTCTGG | SEQ ID NO: 52 |
| mC_SDC2 | CTCTGCTCCGGATTCG | SEQ ID NO: 53 | GGTGAGCAGGATCCAC | SEQ ID NO: 54 |
| mC_SDC2 | GTTTAGGGTGTTTGAA | SEQ ID NO: 55 | CCGGACGAGCGCATTT | SEQ ID NO: 56 |
| mC_SDC2 | TGACCTGGAAACTTCG | SEQ ID NO: 57 | CTTTTCTCTCTGGACA | SEQ ID NO: 58 |
| mC_SDC2 | ACGCGTCCGAAAATG | SEQ ID NO: 59 | TCCCGTGTAACTCCTA | SEQ ID NO: 60 |
| ACCB1 | CCCAGGCTGTTTATTT | SEQ ID NO: 61 | AACATTGCCTATGGAG | SEQ ID NO: 62 |
| ABCB1 | GAGCATAGTAAGCAGT | SEQ ID NO: 63 | CAAGCACTGAAAGATA | SEQ ID NO: 64 |
| ABCB1 | TCCCACAGCCACTGTT | SEQ ID NO: 65 | TTCCTATATCCTGTGT | SEQ ID NO: 66 |
| ABL1 | CCCACTGTCTATGGTG | SEQ ID NO: 67 | CAGGCTGTATTTCTTC | SEQ ID NO: 68 |
| ABL1 | TAACTAGTCAAGTACT | SEQ ID NO: 69 | CTTTCATGACTGCAGC | SEQ ID NO: 70 |
| ABL1 | AGATCAAACACCCTAA | SEQ ID NO: 71 | GTTTTGTGCAGTGAGC | SEQ ID NO: 72 |
| ABL1 | CTTTTTCTTTAGACAG | SEQ ID NO: 73 | TTCCCGTAGGTCATGA | SEQ ID NO: 74 |
| ABL1 | CCTCCTGGACTACCTG | SEQ ID NO: 75 | ACCTGTGGATGAAGTT | SEQ ID NO: 76 |
| ABL1 | TTGGTGAAGGTAGCTG | SEQ ID NO: 77 | CTTGATGGAGAACTTG | SEQ ID NO: 78 |
| AKT1 | GGTGGTGTGATGGTGA | SEQ ID NO: 79 | CGAAGCTCATGACTGT | SEQ ID NO: 80 |
| AKT1 | CGGAAGTCCATCTCCT | SEQ ID NO: 81 | GCTCCTGATCTGGTAC | SEQ ID NO: 82 |
| AKT1 | GTGAGGATGGCTACAG | SEQ ID NO: 83 | CCATGTGGAGACTCCT | SEQ ID NO: 84 |
| AKT1 | AAGGTGCGTTCGATGA | SEQ ID NO: 85 | ACGCAGACAGAGGCTC | SEQ ID NO: 86 |
| AKT1 | CACGTTGGTCCACATC | SEQ ID NO: 87 | ACCACCCGCACGTCT | SEQ ID NO: 88 |
| ALK | AAATGTTGACCAAAGG | SEQ ID NO: 89 | CTTCTTTTAGATACCG | SEQ ID NO: 90 |
| ALK | GGCAGTCTTTACTCAC | SEQ ID NO: 91 | AAATGCATTTCCTTTC | SEQ ID NO: 92 |
| ALK | AATGTGAGCCCTTGAG | SEQ ID NO: 93 | TGGCTGTCAGTATTTG | SEQ ID NO: 94 |
| ALK | CTCGGAGGAAGGACTT | SEQ ID NO: 95 | TCTGCTCTGCAGCAAA | SEQ ID NO: 96 |
| ALK | CCTTGGAGATATCGAT | SEQ ID NO: 97 | GAACAGGACGAACTGG | SEQ ID NO: 98 |
| ALK | AGAGTGAGCCACTTCT | SEQ ID NO: 99 | TCTTGTCTTCTCCTTT | SEQ ID NO: 100 |
| ALK | TTGCTCAGCTTGTACT | SEQ ID NO: 100 | GTGTAGTGCTTCAAGG | SEQ ID NO: 102 |
| ALK | ACGCTCAGGTTGGAG | SEQ ID NO: 103 | ATGAGTGACTGCCTCT | SEQ ID NO: 104 |
| ALK | GCATAGAGCCTACCTG | SEQ ID NO: 105 | GTGCTAGTGGAGAACA | SEQ ID NO: 106 |
| ALK | TTCAGGGCAAAGAAGT | SEQ ID NO: 107 | GTTTTCCAATGCAACC | SEQ ID NO: 108 |
| APC | AAATCCTAAGAGAGAA | SEQ ID NO: 109 | ATGCTTCCTGGTCTTT | SEQ ID NO: 110 |
| APC | CACAGGAAGCAGATTC | SEQ ID NO: 111 | TCTGCTGGATTTGGTT | SEQ ID NO: 112 |
| APC | CCCAAAAGTCCACCTG | SEQ ID NO: 113 | TCCACTGCATGGTTCA | SEQ ID NO: 114 |
| APC | AAGCAGAAGTAAAACA | SEQ ID NO: 115 | TGAACTGCAGCATTTA | SEQ ID NO: 116 |
| APC | ATGCTGATACTTTATT | SEQ ID NO: 117 | CTGGAGGCATTATTCT | SEQ ID NO: 118 |
| APC | CAGAGCAGCCTAAAGA | SEQ ID NO: 119 | TGGCAGAAATAATACA | SEQ ID NO: 120 |
| ARAF | TCAGCCCATCTTGACA | SEQ ID NO: 121 | GTGCGTTGCTTGTT | SEQ ID NO: 122 |
| ARAF | TGAGAGGCATGGCTAT | SEQ ID NO: 123 | CATCGAGTCTTCACTG | SEQ ID NO: 124 |
| ATM | TCAGATTCCAAACAAG | SEQ ID NO: 125 | AGACTTACACACAAAA | SEQ ID NO: 126 |
| ATM | CACCTAGGCTAAAATG | SEQ ID NO: 127 | AGTATTTTCTCACAGA | SEQ ID NO: 128 |
| ATM | TCTGCTAGTGAATGAG | SEQ ID NO: 129 | ACTTACTGTACCTGGT | SEQ ID NO: 130 |
| ATM | TCACCTTCAGAAGTCA | SEQ ID NO: 131 | TTGAGATGAAAGGATT | SEQ ID NO: 132 |
| ATM | CACCAGTATAGTTCCA | SEQ ID NO: 133 | TCTAACTGATAGAATA | SEQ ID NO: 134 |
| ATM | TGGTTTACTTTAAGAT | SEQ ID NO: 135 | TCTGGAATAATTCTGA | SEQ ID NO: 136 |
| ATM | GTTCTTTGTTTGTCTT | SEQ ID NO: 137 | AACAGGAAGCATACTT | SEQ ID NO: 138 |
| ATM | AAGTTCTTGTGTTTGT | SEQ ID NO: 139 | ATGCAGGTGGAGGGAT | SEQ ID NO: 140 |
| ATM | TACCACAGCAATGTGT | SEQ ID NO: 141 | TTGAGCATCCCTTGTG | SEQ ID NO: 142 |
| ATM | TTTTCTGAGTGCTTTT | SEQ ID NO: 143 | AAGCAAAGTTTTAAGG | SEQ ID NO: 144 |
| ATM | CTTAACACATTGACTT | SEQ ID NO: 145 | CTTGAAGATTTAGCCA | SEQ ID NO: 146 |
| ATM | TAAAAAGTGGCTTAGG | SEQ ID NO: 147 | AGAACAGGATAGAAAG | SEQ ID NO: 148 |
| ATM | TTTCTCTCAGTAAGTG | SEQ ID NO: 149 | AAAATTAGCACCCTGA | SEQ ID NO: 150 |
| ATM | TATGTAGAGGCTGTTG | SEQ ID NO: 151 | CTGAAGTTCTTTATCT | SEQ ID NO: 152 |
| ATM | CTGGTGTACTTGATAG | SEQ ID NO: 153 | TGTTGTCATCTTATAA | SEQ ID NO: 154 |
| ATM | CAAACTATTGGGTGGA | SEQ ID NO: 155 | TGTGTAGAAAGCAGAT | SEQ ID NO: 156 |
| ATM | TTTGTCAGAGTCAGAG | SEQ ID NO: 157 | GATCCTAAACGTAAGA | SEQ ID NO: 158 |
| ATM | GCTTTCTGGCTGGATT | SEQ ID NO: 159 | TACCTTTTCTCTTGAT | SEQ ID NO: 160 |
| ATM | TGCATTTGAAGAAGGA | SEQ ID NO: 161 | CAAAGTATGAGATAAA | SEQ ID NO: 162 |
| ATM | TTCTTCAATTTTTGTT | SEQ ID NO: 163 | ATTTACCTAGTAATGG | SEQ ID NO: 164 |
| ATM | TTTAGGCCTTGCAGAA | SEQ ID NO: 165 | ACTGCATATTCCTCCA | SEQ ID NO: 166 |
| ATM | CAGTAGAAGTTGCTGG | SEQ ID NO: 167 | ATGATTTCATGTAGTT | SEQ ID NO: 168 |
| ATM | ATTTGAAAACAAGCAA | SEQ ID NO: 169 | CACTCAGTTAACTGGT | SEQ ID NO: 170 |
| ATM | TGTTAAAGTTCATGGC | SEQ ID NO: 171 | CATAAGAAGCGTTTAC | SEQ ID NO: 172 |
| ATM | ACAGAGATGAATTTCT | SEQ ID NO: 173 | GAATATCACACTTCTA | SEQ ID NO: 174 |
| ATM | CCACACAGGAGAATAT | SEQ ID NO: 175 | ACAAGCTGTCTCCTCT | SEQ ID NO: 176 |
| ATM | AATATGAAGTCTTCAT | SEQ ID NO: 177 | TAGCTACACTGCGCGT | SEQ ID NO: 178 |
| ATM | TTGGTGATAGACATGT | SEQ ID NO: 179 | ACAACATTCCATGATG | SEQ ID NO: 180 |
| ATM | CTTTTGAACAGGGCAA | SEQ ID NO: 181 | CTCCTTTACTTCATAT | SEQ ID NO: 182 |
| ATM | CCTCACTGAAACCTTT | SEQ ID NO: 183 | ACCAACACTGAGCACA | SEQ ID NO: 184 |
| ATM | GGACAAGTGAATTTGC | SEQ ID NO: 185 | AAAGGCTGAATGAAAG | SEQ ID NO: 186 |
| BRAF | TGTTTTTGGAGAAGCA | SEQ ID NO: 187 | ATTCTCGCCTCTATTG | SEQ ID NO: 188 |
| BRAF | TGGAAAAATAGCCTCA | SEQ ID NO: 189 | ATGAAGACCTCACAGT | SEQ ID NO: 190 |
| BRAF | AAGAAAAAGTCAGGAT | SEQ ID NO: 191 | TACTCAGGTTAAAATG | SEQ ID NO: 192 |
| BRAF | CTCAATGATATGGAGA | SEQ ID NO: 193 | ATTTCTTTGTACAGGA | SEQ ID NO: 194 |
| BRAF | ATGACTTGTCACAATG | SEQ ID NO: 195 | CGAGTGATGATTGGGA | SEQ ID NO: 196 |
| BRAF | ATTTTTGGATTACTTA | SEQ ID NO: 197 | GCTGCTTTTCCAGGGT | SEQ ID NO: 198 |
| BRAF | TTTCGACAAAAGTCAC | SEQ ID NO: 199 | ACAAGAGAGTAGATAC | SEQ ID NO: 200 |
| BTK | AGGCCCTCAGTTCAAG | SEQ ID NO: 201 | TCCCTTCACAGGTGGT | SEQ ID NO: 202 |
| CBL | GGAGAAACTCCCAGAT | SEQ ID NO: 203 | CCAGTCAGATCAGGAT | SEQ ID NO: 204 |
| CBL | GAACAATATGAATTAT | SEQ ID NO: 205 | CTGCCAGGATGTAAGA | SEQ ID NO: 206 |
| CBL | GATGCATCTGTTACTA | SEQ ID NO: 207 | ACTCCCTCTAGGATCA | SEQ ID NO: 208 |
| CDH1 | TCATAACCCACAGATC | SEQ ID NO: 209 | GAAAAATGCCAACATA | SEQ ID NO: 210 |
| CDH1 | TGTTCCTGGTCCTGAC | SEQ ID NO: 211 | TCAGTGACTGTGATCA | SEQ ID NO: 212 |
| CDH1 | TGAAAAGAGAGTGGAA | SEQ ID NO: 213 | GCTGCAAGTCAGTTGA | SEQ ID NO: 214 |
| CDH1 | AAGAACAGCACGTACA | SEQ ID NO: 215 | TGAACTCTTCCCTCCA | SEQ ID NO: 216 |
| CDK4 | TCTTGAGGGCCACAAA | SEQ ID NO: 217 | ATTGTAGGGTCTCCCT | SEQ ID NO: 218 |
| CDKN2A | ATCGAAGCGCTACCTG | SEQ ID NO: 219 | CCAACGCACCGAATAG | SEQ ID NO: 220 |
| CDKN2A | ACCTGGTCTTCTAGGA | SEQ ID NO: 221 | GTTTTCGTGGTTCACA | SEQ ID NO: 222 |
| CHEK2 | CCACATAAGGTTCTCA | SEQ ID NO: 223 | CTGGCAGACTATGTTA | SEQ ID NO: 224 |
| CHEK2 | TACAGGAATAGCCACA | SEQ ID NO: 225 | CTGTGTAGTACCTTCA | SEQ ID NO: 226 |
| CSF1R | ACCATGACTTTGAGGT | SEQ ID NO: 227 | GGACATCTTCCCACTA | SEQ ID NO: 228 |
| CTNNB1 | CCATGGAACCAGACAG | SEQ ID NO: 229 | CATCCTCTTCCTCAGG | SEQ ID NO: 230 |
| CYP2C19 | AAGTTGTTTTGTTTTG | SEQ ID NO: 231 | TTGAGCTGAGGTCTTC | SEQ ID NO: 232 |
| CYP2C19 | AACGTTTCGATTATAA | SEQ ID NO: 233 | AGACTGTAAGTGGTTT | SEQ ID NO: 234 |
| CYP2C19 | AATAATTTTCCCACTA | SEQ ID NO: 235 | AGGGTTGTTGATGTCC | SEQ ID NO: 236 |
| CYP2C8 | AGGGTCAAAGATATTT | SEQ ID NO: 237 | CTCCTCACTTCTGGAC | SEQ ID NO: 238 |
| CYP2C8 | AGGATTCGATGAATCA | SEQ ID NO: 239 | CACCAAGCATCACTGG | SEQ ID NO: 240 |
| CYP2C8 | TAAGGTCAATGACGCA | SEQ ID NO: 241 | ACAACCTTGCGGAATT | SEQ ID NO: 242 |
| CYP2C8 | TTTTGTCCTACTCCTT | SEQ ID NO: 243 | TTCAGTGTTTCTCCAT | SEQ ID NO: 244 |
| CYP2D6 | TTGGAGGAGGTCAGGC | SEQ ID NO: 245 | AGCCCATCTGGGAAAC | SEQ ID NO: 246 |
| CYP2D6 | ACATCCGGATGTAGGA | SEQ ID NO: 247 | CCTGAGAGCAGCTTCA | SEQ ID NO: 248 |
| CYP2D6 | TCTCACCTTCTCCATC | SEQ ID NO: 249 | GTCCTACGCTTCCAAA | SEQ ID NO: 250 |
| CYP2D6 | CGGCTTTGTCCAAGAG | SEQ ID NO: 251 | TGGGCAGAAGGGCACA | SEQ ID NO: 252 |
| CYP2D6 | GGTGTGTTCTGGAAGT | SEQ ID NO: 253 | ATAGTGGCCATCTTCC | SEQ ID NO: 254 |
| CYP3A4 | ATGACTGTCCTGTAGA | SEQ ID NO: 255 | CCGTGACCCAAAGTAC | SEQ ID NO: 256 |
| CYP3A4 | ATCAAATCTTAAAAGC | SEQ ID NO: 257 | TCTCCACTCAGCGTCT | SEQ ID NO: 258 |
| CYP3A4 | GCTGCGCTTCTACTTA | SEQ ID NO: 259 | GGGTGGTGTTGTGTTT | SEQ ID NO: 260 |
| CYP3A4 | GAGGAGCCTGGACAGT | SEQ ID NO: 261 | GAAGACTCAGAGGAGA | SEQ ID NO: 262 |
| CYP3A5 | AAGTCCTCTCAAGTCT | SEQ ID NO: 263 | TATCCAATTCTGTTTC | SEQ ID NO: 264 |
| CYP3A5 | TTCATATGATGAAGGG | SEQ ID NO: 265 | AGATACCCACGTATGT | SEQ ID NO: 266 |
| DDR2 | CTGATGACCTGAAGGA | SEQ ID NO: 267 | GACTGTAATTGATCTT | SEQ ID NO: 268 |
| DDR2 | GACCCAAACATCATCC | SEQ ID NO: 269 | GCTGGAGGAAGAATTA | SEQ ID NO: 270 |
| DDR2 | GAGAAGAGATACGAAG | SEQ ID NO: 271 | GTGGTAGGTCTTGTAG | SEQ ID NO: 272 |
| DNMT3A | GTGCCCTCATTTACCT | SEQ ID NO: 273 | CACGACAGCGATGAGA | SEQ ID NO: 274 |
| DPYD | CTCCATATGTAGTTCG | SEQ ID NO: 275 | ATGTTGATGTGTCTTG | SEQ ID NO: 276 |
| DPYD | CACCAACTTATGCCAA | SEQ ID NO: 277 | CTGAATATTGAGCTCA | SEQ ID NO: 278 |
| DPYD | CCAGCTTCAAAAGCTC | SEQ ID NO: 279 | CTTTTACACTCCTATT | SEQ ID NO: 280 |
| DPYD | AGCATGAAATAGTGTA | SEQ ID NO: 281 | GCTTTAAATCCTCGAA | SEQ ID NO: 282 |
| EGFR | TTGGGCACTTTTGAAG | SEQ ID NO: 283 | AAAGTCACCAACCTTT | SEQ ID NO: 284 |
| EGFR | TGTCCTCATTGCCCTC | SEQ ID NO: 285 | AGTCCGGTTTTATTTG | SEQ ID NO: 286 |
| EGFR | AATGTGTCTTCACTTT | SEQ ID NO: 287 | TGGGCACAGATGATTT | SEQ ID NO: 288 |
| EGFR | GGCAAATACAGCTTTG | SEQ ID NO: 289 | CTCCAAGATGGGATAC | SEQ ID NO: 290 |
| EGFR | GGAGATGTGATAATTT | SEQ ID NO: 291 | GACTTACTGCAGCTGT | SEQ ID NO: 292 |
| EGFR | GTCACTGACTGCTGTG | SEQ ID NO: 293 | ACATTCCGGCAAGAGA | SEQ ID NO: 294 |
| EGFR | AGTTATTTGGAATTTT | SEQ ID NO: 295 | CTGTATGCACTCAGAG | SEQ ID NO: 296 |
| EGFR | CATGAACATTTTTCTC | SEQ ID NO: 297 | CAGACCAGGGTGTTGT | SEQ ID NO: 298 |
| EGFR | ACACCCAGTGGAGAAG | SEQ ID NO: 299 | CCAGGGACCTTACCTT | SEQ ID NO: 300 |
| EGFR | GTCTTCCTTCTCTCTC | SEQ ID NO: 301 | GAAACTCACATCGAGG | SEQ ID NO: 302 |
| EGFR | CCTACGTGATGGCCA | SEQ ID NO: 303 | CTTTGTGTTCCCGGAC | SEQ ID NO: 304 |
| EGFR | GGAACGTACTGGTGAA | SEQ ID NO: 305 | CTAAAGCCACCTCCTT | SEQ ID NO: 306 |
| EGFR | AGAGTGAGTTAACTTT | SEQ ID NO: 307 | ACTCTGGTGGGTATAG | SEQ ID NO: 308 |
| EGFR | AGAAACGCATCCAGCA | SEQ ID NO: 309 | AGCGACAATGAAAAAC | SEQ ID NO: 310 |
| ERBB2 | GGGTATGTGGCTACA | SEQ ID NO: 311 | CTCACACCGCTGTGTT | SEQ ID NO: 312 |
| ERBB2 | CCCTGACCCTGGCTT | SEQ ID NO: 313 | ACTTCCGGATCTTCTG | SEQ ID NO: 314 |
| ERBB2 | GGATCTGGCGCTTTT | SEQ ID NO: 315 | ACTGCCTCCAGCTCTT | SEQ ID NO: 316 |
| ERBB2 | CATCTGGATCCCTGAT | SEQ ID NO: 317 | CTGTCCTCCTAGCAGG | SEQ ID NO: 318 |
| ERBB2 | CATACCCTCTCAGCGT | SEQ ID NO: 319 | ATAGGGCATAAGCTGT | SEQ ID NO: 320 |
| ERBB2 | AGGTCTACATGGGTGC | SEQ ID NO: 321 | GCCCGAAGTCTGTAAT | SEQ ID NO: 322 |
| ERBB2 | CACACAGTTGGAGGAC | SEQ ID NO: 323 | TCACACACCATAACTC | SEQ ID NO: 324 |
| ERBB3 | CACTGTACAAGCTCTA | SEQ ID NO: 325 | AAAGAGGAGCAGGTTG | SEQ ID NO: 326 |
| ERBB3 | GTCACAGTGGATTCGA | SEQ ID NO: 327 | ATGACGAAGATGGCAA | SEQ ID NO: 328 |
| ERBB3 | ACACACGTAACATAAA | SEQ ID NO: 329 | GGGTTCCAGCTGGAAA | SEQ ID NO: 330 |
| ERBB3 | CACCAAGTATCAGTAT | SEQ ID NO: 331 | CAACTGGATTCTTTTT | SEQ ID NO: 332 |
| ERBB3 | CCATTGGTAGCTGGTG | SEQ ID NO: 333 | ATTTTTATCTACTTCC | SEQ ID NO: 334 |
| ERBB3 | TCCTCTCATCCTGTCT | SEQ ID NO: 335 | TATTGGCACTTATATA | SEQ ID NO: 336 |
| ERBB3 | AGAGCTAAGGAAGCTT | SEQ ID NO: 337 | AATCCTATGCAAAAAT | SEQ ID NO: 338 |
| ERBB3 | ACCTTGAGGAACATGG | SEQ ID NO: 339 | ATAGCAGCTGCTTATC | SEQ ID NO: 340 |
| ERBB3 | AAACCCTACAGATACC | SEQ ID NO: 341 | ATGTATCCAGATGATG | SEQ ID NO: 342 |
| ERBB4 | CTTACATTTGACCATG | SEQ ID NO: 343 | ATGACCTTTGGAGGAA | SEQ ID NO: 344 |
| ERBB4 | CCGATCTGGATCAGCA | SEQ ID NO: 345 | ACATTTCAGGGTCCTG | SEQ ID NO: 346 |
| ERBB4 | AGAGTGTTGTCCAGTT | SEQ ID NO: 347 | TGCTTATCCTCAAGCA | SEQ ID NO: 348 |
| ERBB4 | ACAAAAATTTAATACT | SEQ ID NO: 349 | GGCACAGGATCATTGA | SEQ ID NO: 350 |
| ERBB4 | TTTTCTTCTACTTCCA | SEQ ID NO: 351 | TGAGCTTGTTTGCTGA | SEQ ID NO: 352 |
| ERBB4 | AATCAAATAGGGAAGG | SEQ ID NO: 353 | GACCTTACGTCAGTGA | SEQ ID NO: 354 |
| ERBB4 | AATGTAACAAATATGA | SEQ ID NO: 355 | GGAAACTTTGGACTTC | SEQ ID NO: 356 |
| EZH2 | AAGCCCTTAGAGATCA | SEQ ID NO: 357 | CTTTGCAGTTATGATG | SEQ ID NO: 358 |
| EZH2 | GGGAGTTCCAATTCTC | SEQ ID NO: 359 | CTTTTTAGATTTTGTG | SEQ ID NO: 360 |
| EZH2 | TCTGAAACATACCATT | SEQ ID NO: 361 | TTATCCAAAAGAATTT | SEQ ID NO: 362 |
| EZH2 | ACATTAACGCTGACTT | SEQ ID NO: 363 | AACAGCTCTAGACAAC | SEQ ID NO: 364 |
| EZH2 | ACATTCAGGAGGAAGT | SEQ ID NO: 365 | CATGGAAACCTTTTAG | SEQ ID NO: 366 |
| EZH2 | TACATTGATTCCATTT | SEQ ID NO: 367 | TTCCTCAATGTTTCCA | SEQ ID NO: 368 |
| EZH2 | AGCCCTATTTCTACTC | SEQ ID NO: 369 | GATCCTGAAGAAAGAG | SEQ ID NO: 370 |
| EZH2 | GTCTCCATCATCATCA | SEQ ID NO: 371 | TTATTGCTTCTCCTGT | SEQ ID NO: 372 |
| EZH2 | TTATGTTAACCAACCT | SEQ ID NO: 373 | CAATCGTCAGAAAATT | SEQ ID NO: 374 |
| FBXW7 | TATATCGTCTACACAA | SEQ ID NO: 375 | AACACAAAGCTGGTGT | SEQ ID NO: 376 |
| FBXW7 | CTCTCCAATGTGACTA | SEQ ID NO: 377 | CAAGCATCAGAGTGCT | SEQ ID NO: 378 |
| FBXW7 | GTAAACACTGTCCTGT | SEQ ID NO: 379 | GGAATTGCATTCACAC | SEQ ID NO: 380 |
| FBXW7 | CATCAGGAGAGCATTT | SEQ ID NO: 381 | GCATATGATTTTATGG | SEQ ID NO: 382 |
| FBXW7 | AACCCTCCTGCCATCA | SEQ ID NO: 383 | CTCTGCAGAGTTGTTA | SEQ ID NO: 384 |
| FBXW7 | CAAATTCACCAATAAT | SEQ ID NO: 385 | GGAGAATGTATACACA | SEQ ID NO: 386 |
| FBXW7 | TCTCTGCATTCCACAC | SEQ ID NO: 387 | TCTTAAGTGTTTTTCC | SEQ ID NO: 388 |
| FBXW7 | TGCCAAGTGAAATAGT | SEQ ID NO: 389 | ACATCAGACAGCACAG | SEQ ID NO: 390 |
| FBXW7 | CAATTTTGAACCTTAC | SEQ ID NO: 391 | CTATGTGCTTTCATTC | SEQ ID NO: 392 |
| FBXW7 | ATCTTTACCTCTTTAG | SEQ ID NO: 393 | ACCAGAGAAATTGCTT | SEQ ID NO: 394 |
| FBXW7 | CACCTGAAACATTTTT | SEQ ID NO: 395 | GTACCATGTTCAGCAA | SEQ ID NO: 396 |
| FBXW7 | ACTATCATCAGACTGA | SEQ ID NO: 397 | GATGAGGACTCCTCAG | SEQ ID NO: 398 |
| FBXW7 | CCTCCTCTACCACACG | SEQ ID NO: 399 | GCTGGCTTTTGGAAAT | SEQ ID NO: 400 |
| FGFR1 | TCCTTGCTTCTCAGAT | SEQ ID NO: 401 | GGACAATGTGATGAAG | SEQ ID NO: 402 |
| FGFR1 | AGGCCTTGGGACTGAT | SEQ ID NO: 403 | AAGATGATCGGGAAGC | SEQ ID NO: 404 |
| FGFR2 | GTGTTACTGCCATCGA | SEQ ID NO: 405 | GATTTAGCAGCCAGAA | SEQ ID NO: 406 |
| FGFR2 | CAATCAAACTGCAGAG | SEQ ID NO: 407 | CTGGTGTCAGAGATGG | SEQ ID NO: 408 |
| FGFR2 | GACATGGCCAAGAGAA | SEQ ID NO: 409 | ATAACAACACGCCTCT | SEQ ID NO: 410 |
| FGFR2 | CAGAAGTCGATGGCAT | SEQ ID NO: 411 | AGCTGACCAAACGTAT | SEQ ID NO: 412 |
| FGFR2 | CGGCACAGGATGACTG | SEQ ID NO: 413 | TCCTGTGATCTGCAAT | SEQ ID NO: 414 |
| FGFR2 | GCGTCCTCAAAAGTTA | SEQ ID NO: 415 | CCACAATCATTCCTGT | SEQ ID NO: 416 |
| FGFR2 | CTGCCCTATATAATTG | SEQ ID NO: 417 | TATATTGTTCTCCTGT | SEQ ID NO: 418 |
| FGFR2 | AGATTCAGAAAGTCCT | SEQ ID NO: 419 | TTGTCTGCAAGGTTTA | SEQ ID NO: 420 |
| FGFR2 | ACGTCTCCTCCGACCA | SEQ ID NO: 421 | TTTATTGGTCTCTCAT | SEQ ID NO: 422 |
| FGFR2 | AAACTTATGGGAGAAA | SEQ ID NO: 423 | CATCAATCACACGTAC | SEQ ID NO: 424 |
| FGFR2 | GACCCGTATTCATTCT | SEQ ID NO: 425 | AGGATTGTTAAATAAC | SEQ ID NO: 426 |
| FGFR2 | ATGTTCTGAAAGCTTA | SEQ ID NO: 427 | CAACACTGTCAAGTTT | SEQ ID NO: 428 |
| FGFR2 | CCTGTGACATTCACCA | SEQ ID NO: 429 | CAATAGGACAGTGCTT | SEQ ID NO: 430 |
| FLT3 | CGACACAACACAAAAT | SEQ ID NO: 431 | GGGAAAGTGGTGAAGA | SEQ ID NO: 432 |
| FLT3 | TCTCTGTCCAAGTCCT | SEQ ID NO: 433 | TGTGTATGCCTATAAT | SEQ ID NO: 434 |
| FLT3 | TGGGTTACCTGACAGT | SEQ ID NO: 4 5 | CTTTCTTTGACAGAAA | SEQ ID NO: 436 |
| FLT3 | CTAAATTTTCTCTTGG | SEQ ID NO: 437 | AAGCAATTTAGGTATG | SEQ ID NO: 438 |
| FLT3 | AGTCAGTTAGGAATAG | SEQ ID NO: 439 | CAATTGGTGTTTGTCT | SEQ ID NO: 440 |
| FLT3 | TTACCTACGATGGTAA | SEQ ID NO: 441 | TTCAACAAACAGAACT | SEQ ID NO: 442 |
| GNA11 | CCTGACCGACGTTGA | SEQ ID NO: 443 | GTACCGGAAGATGATG | SEQ ID NO: 444 |
| GNA11 | CTGGGATTGCAGATTG | SEQ ID NO: 445 | GATGTCACGTTCTCAA | SEQ ID NO: 446 |
| GNAS | ACCAGTTCAGAGTGGA | SEQ ID NO: 447 | TCATGTTCCTATATGG | SEQ ID NO: 448 |
| GNAS | TCACTTTCAGGAATTC | SEQ ID NO: 449 | GGTGGCGGTTACTTAC | SEQ ID NO: 450 |
| GNAS | TTAGATTGGCAATTAT | SEQ ID NO: 451 | ACTTTGTCCACCTGGA | SEQ ID NO: 452 |
| GSTP1 | GGATGATACATGGTGG | SEQ ID NO: 453 | TCTCCCACAATGAAGG | SEQ ID NO: 454 |
| HNF1A | TGGTACGTCCGCAA | SEQ ID NO: 455 | TGGTGAAGCTTCCAGC | SEQ ID NO: 456 |
| HNF1A | GAAGAGCCCACAGGTG | SEQ ID NO: 457 | TCCTTGCTAGGGTTCT | SEQ ID NO: 458 |
| HRAS | TACTGGTGGATGTCCT | SEQ ID NO: 459 | GTTGGACATCCTGGAT | SEQ ID NO: 460 |
| HRAS | AGGCTCACCTCTATAG | SEQ ID NO: 461 | GCGATGACGGAATATA | SEQ ID NO: 462 |
| IDH2 | TTGTACTGCAGAGACA | SEQ ID NO: 463 | ACCAAGCCCATCACCA | SEQ ID NO: 464 |
| IDH2 | AGGCGTGGGATGTTTT | SEQ ID NO: 465 | GACCACTATTATCTCT | SEQ ID NO: 466 |
| JAK1 | GAGGTTCCTTAAGATC | SEQ ID NO: 467 | GTTGAGCTCTGCAGGT | SEQ ID NO: 468 |
| JAK1 | CCTAGACAGCACCGTA | SEQ ID NO: 469 | GGATAAAGACCTGGTC | SEQ ID NO: 470 |
| JAK1 | TTCTGGTGGGACCATT | SEQ ID NO: 471 | TCTGGATCTCTTCATG | SEQ ID NO: 472 |
| JAK1 | AAGAGAACACACTTAC | SEQ ID NO: 473 | GACATTCCTATGTCCT | SEQ ID NO: 474 |
| JAK2 | CTCTGTAAATTCTACC | SEQ ID NO: 475 | CTCGGCTTTCATTTGA | SEQ ID NO: 476 |
| JAK2 | TAACTCTAATAGGAAG | SEQ ID NO: 477 | AATACTAATGCCAGGA | SEQ ID NO: 478 |
| JAK3 | ACTGAGGTATCGCCTC | SEQ ID NO: 479 | CACATCATCCTTGGTT | SEQ ID NO: 480 |
| KDR | GTGGATGCTTCCTTTT | SEQ ID NO: 481 | CTCCAGTGAGGAAGCA | SEQ ID NO: 482 |
| KDR | CAAACCTGCTGAGCAT | SEQ ID NO: 483 | ATCAGTGTTTTGCTTC | SEQ ID NO: 484 |
| KDR | GCTGACACTGGACATC | SEQ ID NO: 485 | CATCTCATCTGTTACA | SEQ ID NO: 486 |
| KDR | TGAGAGCTCGATGCTC | SEQ ID NO: 487 | GAGGGTAAGTTGTATA | SEQ ID NO: 488 |
| KDR | TTTTGCACAGCCAAGA | SEQ ID NO: 489 | AATGATCGTTTTCTTC | SEQ ID NO: 490 |
| KDR | GTGCTCAAAAATTTCT | SEQ ID NO: 491 | ATTGGGTAATGTTATA | SEQ ID NO: 492 |
| KDR | ATTAATTTTTGCTTCA | SEQ ID NO: 493 | ACCCAGAGATACCCAG | SEQ ID NO: 494 |
| KIT | CATCCATCCAGGAAAA | SEQ ID NO: 495 | CATTCATTCTGCTTAT | SEQ ID NO: 496 |
| KIT | CTGTAGCAAAACCAGA | SEQ ID NO: 497 | AATCATCTCACCTCTG | SEQ ID NO: 498 |
| KIT | TGGATGTGCAGACACT | SEQ ID NO: 499 | CTTGCCCACATCGTTG | SEQ ID NO: 500 |
| KIT | CAGAAACCCATGTATG | SEQ ID NO: 501 | ACCAAAACTCAGCCTG | SEQ ID NO: 502 |
| KIT | AGTTGTGCTTTTTGCT | SEQ ID NO: 503 | CAAGTAGATTCACAAT | SEQ ID NO: 504 |
| KIT | TTCTTTCTAACCTTTT | SEQ ID NO: 505 | GCTTTGAACAAATAAA | SEQ ID NO: 506 |
| KIT | ACTCATGGTCGGATCA | SEQ ID NO: 507 | AAACTAAAAATCCTTT | SEQ ID NO: 508 |
| KIT | TGTTCAATTTTGTTGA | SEQ ID NO: 509 | GACGTCACTTTCAAAC | SEQ ID NO: 510 |
| KIT | GGTCCTATGGGATTTT | SEQ ID NO: 511 | AGCAGTGTTAATCACA | SEQ ID NO: 512 |
| KRAS | TGCTCATCTTTTCTTT | SEQ ID NO: 513 | AAATTTGTTACCTGTA | SEQ ID NO: 514 |
| KRAS | TCACACAGCCAGGAGT | SEQ ID NO: 515 | TGCAACAGACTTTAAA | SEQ ID NO: 516 |
| KRAS | TGATTTTGCAGAAAAC | SEQ ID NO: 517 | TCTAGAACAGTAGACA | SEQ ID NO: 518 |
| KRAS | TACTGGTCCCTCATTG | SEQ ID NO: 519 | TAATCCAGACTGTGTT | SEQ ID NO: 520 |
| KRAS | TCTATTGTTGGATCAT | SEQ ID NO: 521 | ATAAGGCCTGCTGAAA | SEQ ID NO: 522 |
| MED12 | GGCTCATTAAGATGAC | SEQ ID NO: 523 | TATCACTCCTTGAAGC | SEQ ID NO: 524 |
| MET | CAATCATACTGCTGAC | SEQ ID NO: 525 | AACCGGTCCTTTACAG | SEQ ID NO: 526 |
| MET | CACAAAGCAAGCCAGA | SEQ ID NO: 527 | CGTAAAAATGCTGGAG | SEQ ID NO: 528 |
| MET | TGTAATAACAAGTATT | SEQ ID NO: 529 | TTTTTAAAGTACATGT | SEQ ID NO: 530 |
| MET | GTAAGTGCCCGAAGTG | SEQ ID NO: 531 | ACCCACTGAGGTATAT | SEQ ID NO: 532 |
| MET | GTGCTAACCAAGTTCT | SEQ ID NO: 533 | GGTTAAATAAAATGCC | SEQ ID NO: 534 |
| MET | TGTTCCATAATGAAGT | SEQ ID NO: 535 | CAGGAGCGAGAGGACA | SEQ ID NO: 536 |
| MET | GTGGTCCTACCATACA | SEQ ID NO: 537 | AGCAGGCCTATTTTGA | SEQ ID NO: 538 |
| MET | TTTCTAACTCTCTTTG | SEQ ID NO: 539 | TACAGTTTCTTGCAGC | SEQ ID NO: 540 |
| MET | CACGGGTAATAATTTT | SEQ ID NO: 541 | CTTTGCACCTGTTTTG | SEQ ID NO: 542 |
| MPL | ATACAGCTGATTGCCA | SEQ ID NO: 543 | TCTGCTTTGGTCCATC | SEQ ID NO: 544 |
| MPL | AAGTCTGACCCTTTTT | SEQ ID NO: 545 | CCTGTAGTGTGCAGGA | SEQ ID NO: 546 |
| MTHFR | TTTGTGACCATTCCGG | SEQ ID NO: 547 | TTCTACCTGAAGAGCA | SEQ ID NO: 548 |
| MTHFR | TGTCAGCCTCAAAGAA | SEQ ID NO: 549 | CATCCCTATTGGCAGG | SEQ ID NO: 550 |
| NEUROG1 | AAGTAACAGTGTCTAC | SEQ ID NO: 551 | CCGAAGACTTCACCTA | SEQ ID NO: 552 |
| NEUROG1 | TGTTACTCTGTGCCAG | SEQ ID NO: 553 | GACATCACTCAGGA | SEQ ID NO: 554 |
| NFE2L2 | TTATTTTATACCTCAC | SEQ ID NO: 555 | TCCTTTGTGTCATTCC | SEQ ID NO: 556 |
| NFE2L2 | AGAACTGAGTACTCTG | SEQ ID NO: 557 | AGAAAGCCTTTTTCGC | SEQ ID NO: 558 |
| NFE2L2 | GTTCTTGTCTTTCCTT | SEQ ID NO: 559 | TGGATTTGATTGACAT | SEQ ID NO: 560 |
| NOTCH1 | GCTCATCATCTGGGAC | SEQ ID NO: 561 | AACCAATACAACCCTC | SEQ ID NO: 562 |
| NOTCH1 | GGCCTCGATCTTGTAG | SEQ ID NO: 563 | TACCTGGAGATTGACA | SEQ ID NO: 564 |
| NPM1 | TGTTTAGTGATGAAAA | SEQ ID NO: 565 | ATACCTACTAAGTGCT | SEQ ID NO: 566 |
| NQO1 | ATTCTCCAGGCGTTTC | SEQ ID NO: 567 | TATCCTCAGAGTGGCA | SEQ ID NO: 568 |
| NRAS | TACACAGAGGAAGCCT | SEQ ID NO: 569 | GATTCTTACAGAAAAC | SEQ ID NO: 570 |
| NRAS | ACCTCTATGGTGGGAT | SEQ ID NO: 571 | GTTCTTGCTGGTGTGA | SEQ ID NO: 572 |
| PAX5 | AAACATGGTGGGATTT | SEQ ID NO: 573 | TCTTTGGGTCCTAGGT | SEQ ID NO: 574 |
| PDGFRA | CTGTCAACCTGCATGA | SEQ ID NO: 575 | TCTTTTCCACATCAGT | SEQ ID NO: 576 |
| PDGFRA | TTTTGGCCAACAATGT | SEQ ID NO: 577 | CAAGGAGATTCTTAGC | SEQ ID NO: 578 |
| PDGFRA | TGTCTGCCAGGAAACT | SEQ ID NO: 579 | ATGACAACCAGGACAA | SEQ ID NO: 580 |
| PDGFRA | TTACCTGTCCTGGTCA | SEQ ID NO: 581 | ACTCCCATCTTGAGTC | SEQ ID NO: 582 |
| PDGFRA | AAAAACAAGCTCTCAT | SEQ ID NO: 583 | TGTCCAGTGAAAATCC | SEQ ID NO: 584 |
| PDGFRA | GTCTGCAGGACAATTC | SEQ ID NO: 585 | ATGCAAATAGTTGACC | SEQ ID NO: 586 |
| PDGFRA | AACAATGGTGACTACA | SEQ ID NO: 587 | CTTATATGAGGCTGGA | SEQ ID NO: 588 |
| PDGFRA | AAATTGTGAAGATCTG | SEQ ID NO: 589 | CTTTAGAGATTAAAGT | SEQ ID NO: 590 |
| PIK3CA | TATATCATTAAGCAAT | SEQ ID NO: 581 | TTCTAACATTTTGTTT | SEQ ID NO: 592 |
| PIK3CA | GTAGAATGTTTACTAC | SEQ ID NO: 593 | TCATCTTGAAGAAGTT | SEQ ID NO: 594 |
| PIK3CA | TGATGAAACAAGACGA | SEQ ID NO: 595 | AGGATATTGTATCATA | SEQ ID NO: 596 |
| PIK3CA | CAAATCTACAGAGTTC | SEQ ID NO: 597 | CATATCAAATTCACAC | SEQ ID NO: 598 |
| PIK3CA | GAGCAATGTATGTCTA | SEQ ID NO: 599 | CAGGTAGAAGACTGCA | SEQ ID NO: 600 |
| PIK3CA | TGATCTGGGTAATAGT | SEQ ID NO: 601 | CAGAGGATAGCAACAT | SEQ ID NO: 602 |
| PIK3CA | CTACACCATATATGAA | SEQ ID NO: 603 | CATTTGACTTTACCTT | SEQ ID NO: 604 |
| PIK3CA | TATGTTCGAACAGGTA | SEQ ID NO: 605 | CTAAACACTAATATAA | SEQ ID NO: 606 |
| PIK3CA | GTCTTCGTGATTTGTA | SEQ ID NO: 607 | CGAGGAAGATCAGGAA | SEQ ID NO: 608 |
| PIK3CA | AGAAAAGTGTTTTGAA | SEQ ID NO: 609 | TTTCCAGATACTAGAG | SEQ ID NO: 610 |
| PIK3CA | AATCTTTGGCCAGTAC | SEQ ID NO: 611 | AGAGAGAAGGTTTGAC | SEQ ID NO: 612 |
| PIK3CA | GCCAATTGGTCTGTAT | SEQ ID NO: 613 | CCTTTTCCATAGAGAA | SEQ ID NO: 614 |
| PIK3CA | GAGACAATGAATTAAG | SEQ ID NO: 615 | AGAATCTCCATTTTAG | SEQ ID NO: 616 |
| PIK3CA | ATGGCTCATTCACAAC | SEQ ID NO: 617 | TAATTACAGTCCAGAA | SEQ ID NO: 618 |
| PIK3CA | GATTCTTTTAGATCTG | SEQ ID NO: 619 | TTTCCATTGCCTCGAC | SEQ ID NO: 620 |
| PIK3CA | GCTCATTAACTTAACT | SEQ ID NO: 621 | GTATATACACTGGGCT | SEQ ID NO: 622 |
| PIK3CA | TTGTAGATATGATGCA | SEQ ID NO: 623 | ACCATTACTTGTCCAT | SEQ ID NO: 624 |
| PIK3CA | CTCTAATTTTGTGACA | SEQ ID NO: 625 | TGCTGTCGAATAGCTA | SEQ ID NO: 626 |
| PIK3CA | TGCCAATCTCTTCATA | SEQ ID NO: 627 | CTTGCTCAGTTTTATC | SEQ ID NO: 628 |
| PIK3CA | GCTTTGGAGTATTTCA | SEQ ID NO: 629 | TGAGCTTTCATTTTCT | SEQ ID NO: 630 |
| PPP2R1A | TCCATGTGTTCTGAGC | SEQ ID NO: 631 | AGGTTCCCAGCTGTTC | SEQ ID NO: 632 |
| PTCH1 | TCACAAAGTTTTTGCT | SEQ ID NO: 633 | ATCGGAATCAAGCTCA | SEQ ID NO: 634 |
| PTCH1 | AAGCTGAACACGCAAA | SEQ ID NO: 635 | TAACGTGAAGTATGTC | SEQ ID NO: 636 |
| PTCH1 | GTAGAAGCAATCTGAT | SEQ ID NO: 637 | TCATCTTTTGCTGAGA | SEQ ID NO: 638 |
| PTCH1 | GGGTGTCCTGTGTCAC | SEQ ID NO: 639 | AAACGCAGATTACCAT | SEQ ID NO: 640 |
| PTCH1 | CAGTGCATATACTTTC | SEQ ID NO: 641 | GGATTTTAACAAGGCA | SEQ ID NO: 642 |
| PTEN | GACATGACAGCCATCA | SEQ ID NO: 643 | TCTAAGAGAGTGACAG | SEQ ID NO: 644 |
| PTEN | TATTTCTTTCCTTAAC | SEQ ID NO: 645 | AATCAAAGCATTCTTA | SEQ ID NO: 646 |
| PTEN | ATGTTAGCTCATTTTT | SEQ ID NO: 647 | AGCATACAAATAAGAA | SEQ ID NO: 648 |
| PTEN | ATTCAGGCAATGTTTG | SEQ ID NO: 649 | CTCTGCAATTAAATTT | SEQ ID NO: 650 |
| PTEN | ATTCTGAGGTTATCTT | SEQ ID NO: 651 | CAACATGATTGTCATC | SEQ ID NO: 652 |
| PTEN | AATGATATGTGCATAT | SEQ ID NO: 653 | AGGAAGAGGAAAGGAA | SEQ ID NO: 654 |
| PTEN | TCTGTCCACCAGGGAG | SEQ ID NO: 655 | TGGAATAGTTTCAAAC | SEQ ID NO: 656 |
| PTEN | AAGTTCATGTACTTTG | SEQ ID NO: 657 | TTTTGGATATTTCTCC | SEQ ID NO: 658 |
| PTEN | TAGAGCGTGCAGATAA | SEQ ID NO: 659 | CAAAATGTTTAATTTA | SEQ ID NO: 660 |
| RAF1 | ATCACTTCACTGGCTT | SEQ ID NO: 661 | TCCTTTGATGCCCTCA | SEQ ID NO: 662 |
| RAF1 | CCTATTACCTCAATCA | SEQ ID NO: 663 | CTTCACCTTTAACACC | SEQ ID NO: 664 |
| RBI | AGGCTTGAGTTTGAAG | SEQ ID NO: 665 | TACCAATACTCCATCC | SEQ ID NO: 666 |
| RB1 | GGAAAACTTTCTTTCA | SEQ ID NO: 667 | TTAGCTAATAAAAATG | SEQ ID NO: 668 |
| RBI | TTTACAGAAACAGCTG | SEQ ID NO: 669 | GTTCTTTACAGAGAAC | SEQ ID NO: 670 |
| RBI | ATGTAAAGGATAATTG | SEQ ID NO: 671 | TCTGAAGAGTTTTATC | SEQ ID NO: 672 |
| RB1 | TCATTGCTTAACACAT | SEQ ID NO: 673 | CTTACGTTAAAATAGG | SEQ ID NO: 674 |
| RBI | CAGTGAATCCAAAAGA | SEQ ID NO: 675 | AATTACAATGAATTCA | SEQ ID NO: 676 |
| RB1 | AATTGTGATTTTCTAA | SEQ ID NO: 677 | TTTTTAACTTACTGAT | SEQ ID NO: 678 |
| RB1 | TATCAAAGCAGAAGGC | SEQ ID NO: 679 | TATGCACATGAATGAA | SEQ ID NO: 680 |
| RB1 | GAGAAGGACCAACTGA | SEQ ID NO: 681 | TCTATTTGCAGTTTGA | SEQ ID NO: 682 |
| RBI | GTACAACCTTGAAGTG | SEQ ID NO: 683 | TTTACACGCGTAGTTG | SEQ ID NO: 684 |
| RB1 | TGAACGCCTTCTGTCT | SEQ ID NO: 685 | GGTGAAGTGCTTGATT | SEQ ID NO: 686 |
| RBI | ATTATGATGTGTTCCA | SEQ ID NO: 687 | ATGGAAAATTACCTAC | SEQ ID NO: 688 |
| RB1 | TACTGTTCTTCCTCAG | SEQ ID NO: 689 | CCCTGGTGGAAGCATA | SEQ ID NO: 690 |
| RET | GGCTGTGTGGGACGTG | SEQ ID NO: 691 | GCATCGAAGACACGC | SEQ ID NO: 692 |
| RET | TCTGCCACCTGCAGAT | SEQ ID NO: 693 | TCCTTGCCTCCACTCA | SEQ ID NO: 694 |
| RET | AGTGGGCTACGTCT | SEQ ID NO: 695 | TCGGGCTCGCAGAA | SEQ ID NO: 696 |
| RET | TGCGACGAGCTGTG | SEQ ID NO: 697 | CAGCTGAGGAGATGGG | SEQ ID NO: 698 |
| RET | CCTGACCTGGTATGGT | SEQ ID NO: 699 | CTTCAGGACGTTGAAC | SEQ ID NO: 700 |
| RET | AACCACCCACATGTCA | SEQ ID NO: 701 | GGGAGAACAGGGCTGT | SEQ ID NO: 702 |
| RET | TCGTTCATCGGGACTT | SEQ ID NO: 703 | GGCTCCTCTTCACGTA | SEQ ID NO: 704 |
| RET | CTTCCTAGAGAGTTAG | SEQ ID NO: 705 | CACACTTACACATCAC | SEQ ID NO: 706 |
| RET | TTACACACACGCAAAA | SEQ ID NO: 707 | TTCCCAGTCCACTATA | SEQ ID NO: 708 |
| RHEB | GATGAGAACGCAATGC | SEQ ID NO: 709 | GGTGATCAGTTATGAA | SEQ ID NO: 710 |
| SF3B1 | TTCCATAAAGGCTTTA | SEQ ID NO: 711 | TGGTTTTGTAGGTCTT | SEQ ID NO: 712 |
| SLC19A1 | AAGCCTGGCACATAC | SEQ ID NO: 713 | TGGTCCTGTCTGTCCT | SEQ ID NO: 714 |
| SMAD4 | AGTGCAAGTGAAAGCC | SEQ ID NO: 715 | AACCTTAAATGTCTCT | SEQ ID NO: 716 |
| SMAD4 | CCTTCAAGCTGCCCTA | SEQ ID NO: 717 | TATACAATCAATACCT | SEQ ID NO: 718 |
| SMAD4 | CTAAGGTTGCACATAG | SEQ ID NO: 719 | AGCTTCTCTGTCTAAG | SEQ ID NO: 720 |
| SMAD4 | AAAGGTCTTTGATTTG | SEQ ID NO: 721 | CTATTCCACCTACTGA | SEQ ID NO: 722 |
| SMAD4 | ACCCAAGACAGAGCAT | SEQ ID NO: 723 | GTAAAAGACCTCAGTC | SEQ ID NO: 724 |
| SMARCB1 | GACCCTTATAATGAGC | SEQ ID NO: 725 | CTATTTTCTTCCTCTC | SEQ ID NO: 726 |
| SMARCB1 | GCTGTGATCCATGAGA | SEQ ID NO: 727 | CTGCCTTGTACCATTC | SEQ ID NO: 728 |
| SMO | GCAGAACATCAAGTTC | SEQ ID NO: 729 | TCAGCCTCTGTGAAGA | SEQ ID NO: 730 |
| SMO | GGTTTGTGGTCCTCAC | SEQ ID NO: 731 | TGCCACAGTGAGGACA | SEQ ID NO: 732 |
| SMO | GTAACCCACCTTCTGT | SEQ ID NO: 733 | AGCACCAGGCCGATT | SEQ ID NO: 734 |
| SMO | CCTCCACAGGCATTTT | SEQ ID NO: 735 | CACTCACAGCACATAG | SEQ ID NO: 736 |
| SMO | CCCTGACTGTGAGATC | SEQ ID NO: 737 | GTACGCCTCCAGATGA | SEQ ID NO: 738 |
| SMO | CCCTTCCCAAGATTTG | SEQ ID NO: 739 | AGGCCTTGGCAATCAT | SEQ ID NO: 740 |
| SMO | ATGAGCCCTCAGCTGA | SEQ ID NO: 741 | AAGCTTGAACTCTCAT | SEQ ID NO: 742 |
| SMO | GTCTCTCCTCCTGTCA | SEQ ID NO: 743 | ACCTCCTTCTTCCTCT | SEQ ID NO: 744 |
| SMO | GGTCTCCAACCCATT | SEQ ID NO: 745 | GGTGCGGGAGTGAATA | SEQ ID NO: 746 |
| STAT3 | ACAAAGTCTGTCAACC | SEQ ID NO: 747 | TGCAGCAATACCATTG | SEQ ID NO: 748 |
| STK11 | ATCACCACGGGTCTGT | SEQ ID NO: 749 | AGGCTCCCACCTTTCA | SEQ ID NO: 750 |
| SULT1A1 | GTGGTGTAGTTGGTCA | SEQ ID NO: 751 | GATTCAAAAGATCCTG | SEQ ID NO: 752 |
| SULT1A1 | CTGTGGGAATGAACAA | SEQ ID NO: 753 | TGCTGCACCAGGTTG | SEQ ID NO: 754 |
| TP53 | GAGTTCCAAGGCCTCA | SEQ ID NO: 755 | AACTTGAACCATCTTT | SEQ ID NO: 756 |
| TP53 | TTAGTACCTGAAGGGT | SEQ ID NO: 757 | GCAGTTATGCCTCAGA | SEQ ID NO: 758 |
| TP53 | TTCTTGCGGAGATTCT | SEQ ID NO: 759 | CTTACTGCCTCTTGCT | SEQ ID NO: 760 |
| TP53 | GAGTCTTCCAGTGTGA | SEQ ID NO: 761 | ATCTTGGGCCTGTGTT | SEQ ID NO: 762 |
| TP53 | AGGGCACCACCACACT | SEQ ID N0: 763 | TCTGATTCCTCACTGA | SEQ ID N0: 764 |
| TP53 | GCTCACCATCGCTATC | SEQ ID N0: 765 | TGTGGGTTGATTCCAC | SEQ ID NO: 766 |
| TP53 | GCATTGAAGTCTCATG | SEQ ID NO: 767 | TCTGTCCCTTCCCAGA | SEQ ID NO: 768 |
| TP53 | TTCTGGGAGCTTCATC | SEQ ID NO: 769 | CTGCTCTTTTCACCCA | SEQ ID NO: 770 |
| TPMT | CCTCAAAAACATGTCA | SEQ ID NO: 771 | ATGCTTTTGAAGAACG | SEQ ID NO: 772 |
| TPMT | CAACCTTCTCAAGACA | SEQ ID NO: 773 | CCAGCCAATTTTGAGT | SEQ ID NO: 774 |
| TPMT | CATTTGCGATCACCTG | SEQ ID NO: 775 | TCATCTTCTTAAAGAT | SEQ ID NO: 776 |
| TPMT | GTATCCCAAGTTCACT | SEQ ID NO: 777 | TTACTCTAATATAACC | SEQ ID NO: 778 |
| U2AF1 | GACCACGGTCTCTAGA | SEQ ID NO: 779 | AGAGAGTGGGTGTGGT | SEQ ID NO: 780 |
| U2AF1 | AGGCAAACAAACCTGG | SEQ ID NO: 781 | GCAAAATAATCAGCTC | SEQ ID NO: 782 |
| UGT1A1 | TCTGAAAGTGAACTCC | SEQ ID NO: 783 | TTCGCCCTCTCCTACT | SEQ ID NO: 784 |
| UGT1A1 | CATGAAATAGTTGTCC | SEQ ID NO: 785 | TTATGCCCGAGACTAA | SEQ ID NO: 786 |
| VHL | GCTTGTCCCGATAGGT | SEQ ID NO: 787 | GTGTGATATTGGCAAA | SEQ ID NO: 788 |

| | | SEQ ID | | SEQ ID |
|---|---|---|---|---|
| Amplicon_ID | P_forward | NO | R_reverse | NO |
| SGI_R4368001 | CCACTCTCACCTTCTCCATCTCT | SEQ ID NO: 789 | CAAGGTCCTACGCTTCCAAAAG | SEQ ID NO: 790 |
| SGI_R4556554 | GGGAAACGGTCGCACTCAA | SEQ ID NO: 791 | CCGTCTGCCTCGATGACCTA | SEQ ID NO: 792 |
| SGI_R4368743 | GATATAAAAAGTGGCTTAGGAGGAGCTT | SEQ ID NO: 793 | AGGAAGAACAGGATAGAAAGACTGCTTATA | SEQ ID NO: 794 |
| SGI_R4572858 | CTCACAGAAGTCGATGGCATGA | SEQ ID NO: 795 | CACAAGCTGACCAAACGTATCC | SEQ ID NO: 796 |
| SGI_R4368909 | CATGTACTGGTCCCTCATTGCA | SEQ ID NO: 797 | GTAATAATCCAGACTGTGTTTCTCCCTT | SEQ ID NO: 798 |
| SGI_R4642904 | CTCCCATACCCTCTCAGCGTA | SEQ ID NO: 799 | AGCCATAGGGCATAAGCTGTG | SEQ ID NO: 800 |
| SGI_R4369335 | GCATTCAGATTCCAAACAAGGAAAATATTTG | SEQ ID NO: 801 | GTTAAGACTTACACACAAAAGTAATATCACAAC | SEQ ID NO: 802 |
| SGI_R4644084 | ATGATCTGCTAGTGAATGAGATAAGTCA | SEQ ID NO: 803 | ACCTACTTACTGTACCTGGTGACA | SEQ ID NO: 804 |
| SGI_R4369401 | GCAAACAGAGATGAATTTCTGACTAAACC | SEQ ID NO: 805 | GACTGAATATCACACTTCTAAAAGGTACGT | SEQ ID NO: 806 |
| SGI_R4644094 | GCTGAGTGGGCTACGTCT | SEQ ID NO: 807 | GTCTTCGGGCTCGCAGAA | SEQ ID NO: 808 |
| SGI_R4369532 | GGGTACATTCAGGAGGAAGTGC | SEQ ID NO: 809 | AACACATGGAAACCTTTTAGAAACTGTTTT | SEQ ID NO: 810 |
| SGI_R4644109 | CCATCCCTGACTGTGAGATCAA | SEQ ID NO: 811 | CCAGGTACGCCTCCAGATGA | SEQ ID NO: 812 |
| SGI_R4369548 | AAAAGGGAGTTCCAATTCTCACGT | SEQ ID NO: 813 | TTTTCTTTTTAGATTTTGTGGTGGATGCAA | SEQ ID NO: 814 |
| SGI_R4644170 | GTCTCTCGGAGGAAGGACTTGA | SEQ ID NO: 815 | CCTCTCTGCTCTGCAGCAAATT | SEQ ID NO: 816 |
| SGI_R4370597 | CACCAAGCAGAAGTAAAACACCTC | SEQ ID NO: 817 | CCTCTGAACTGCAGCATTTACTG | SEQ ID NO: 818 |
| SGI_R4679056 | AGTTGTTCTTGTCTTTCCTTTTCAAGTTTT | SEQ ID NO: 819 | GACATGGATTTGATTGACATACTTTGGAG | SEQ ID NO: 820 |
| SGI_R4370599 | CCAGATGCTGATACTTTATTACATTTTGCC | SEQ ID NO: 821 | TGAACTGGAGGCATTATTCTTAATTCCAC | SEQ ID NO: 822 |
| SGI_R4679375 | ACTATTTTGGCCAACAATGTCTCAAAC | SEQ ID NO: 823 | GCTCCAAGGAGATTCTTAGCCA | SEQ ID NO: 824 |
| SGI_R4377365 | AGGGCATCTGGATCCCTGAT | SEQ ID NO: 825 | CTTCCTGTCCTCCTAGCAGGA | SEQ ID NO: 826 |
| SGI_R4679424 | CCCAAGAACTGAGTACTCTGTACCT | SEQ ID NO: 827 | CAAGAGAAAGCCTTTTTCGCTCA | SEQ ID NO: 828 |
| SGI_R4377371 | GCCAGGGTATGTGGCTACA | SEQ ID NO: 829 | ACTTCTCACACCGCTGTGTT | SEQ ID NO: 830 |
| SGI_R4746042 | CACCTCAGCCCATCTTGACAAA | SEQ ID NO: 831 | CACCGTGCGTTGCTTGTT | SEQ ID NO: 832 |
| SGI_R4377643 | TGATGGAGATGTGATAATTTCAGGAAACA | SEQ ID NO: 833 | CGGTGACTTACTGCAGCTGTTT | SEQ ID NO: 834 |
| SGI_R4746078 | AGGGTGAGAGGCATGGCTATTA | SEQ ID NO: 835 | GCTCCATCGAGTCTTCACTGTG | SEQ ID NO: 836 |
| SGI_R6596986 | CATGGCTGCGCTTCTACTTACT | SEQ ID NO: 837 | CTATGGGTGGTGTTGTGTTTTGTG | SEQ ID NO: 838 |
| SGI_R8190710 | CCGCTACATTGATTCCATTTGTAATAAACC | SEQ ID NO: 839 | CCATTTCCTCAATGTTTCCAGATAAGG | SEQ ID NO: 840 |
| SGI_R6596987 | AGAAGAGGAGCCTGGACAGTTA | SEQ ID NO: 841 | AAAGGAAGACTCAGAGGAGAGAGATAAG | SEQ ID NO: 842 |
| SGI_R8190712 | CATGTTATGTTAACCAACCTCCCTAGT | SEQ ID NO: 843 | GTTCCAATCGTCAGAAAATTTTGGAAAGAA | SEQ ID NO: 844 |
| SGI_R6597008 | CAGAAGGATTCGATGAATCACAAAATGG | SEQ ID NO: 845 | AGAACACCAAGCATCACTGGATG | SEQ ID NO: 846 |
| SGI_R8376036 | ACTCCCGATCTGGATCAGCATA | SEQ ID NO: 847 | TTTCACATTTCAGGGTCCTGACAA | SEQ ID NO: 848 |
| SGI_R6615135 | CCTCACCTCTATGGTGGGATCA | SEQ ID NO: 849 | ACAGGTTCTTGCTGGTGTGAAAT | SEQ ID NO: 850 |
| SGI_R8376053 | GAAGCTGTCAACCTGCATGAAG | SEQ ID NO: 851 | TGAATCTTTTCCACATCAGTGGTGATC | SEQ ID NO: 852 |
| SGI_R6615209 | GCAGAAGAAAAAGTCAGGATGTTTTCA | SEQ ID NO: 853 | GCCCTACTCAGGTTAAAATGATGTTTTG | SEQ ID NO: 854 |
| SGI_R8376054 | TGCATGTCTGCCAGGAAACTTT | SEQ ID NO: 855 | CCAAATGACAACCAGGACAATAAGTGA | SEQ ID NO: 856 |
| SGI_R6615295 | CTGGGACATGGCCAAGAGAAGT | SEQ ID NO: 857 | GAGGATAACAACACGCCTCTCTT | SEQ ID NO: 858 |
| SGI_R8376057 | AATGGCTGACACTGGACATCTT | SEQ ID NO: 859 | GGAGCATCTCATCTGTTACAGCTTC | SEQ ID NO: 860 |
| SGI_RG615296 | CATTCGGCACAGGATGACTGTTA | SEQ ID NO: 861 | CTCCTCCTGTGATCTGCAATCTAG | SEQ ID NO: 862 |
| SGI_R8376067 | ATCCTGTAATAACAAGTATTTCGCCGAA | SEQ ID NO: 863 | CACCTTTTTAAAGTACATGTTTTTCCACCA | SEQ ID NO: 864 |
| SGI_R6615297 | CCTGAAACTTATGGGAGAAACAGGA | SEQ ID NO: 865 | GGTCCATCAATCACACGTACCA | SEQ ID NO: 866 |
| SGI_R8376068 | GCTGGTGGTCCTACCATACATG | SEQ ID NO: 867 | TCAGAGCAGGCCTATTTTGAAGG | SEQ ID NO: 868 |
| SGI_R6615298 | GATGGACCCGTATTCATTCTCCA | SEQ ID NO: 869 | TGCTAGGATTGTTAAATAACCGCCTTT | SEQ ID NO: 870 |
| SGI_R8376092 | GATGAGTCAGTTAGGAATAGGCAGTTC | SEQ ID NO: 871 | GCAACAATTGGTGTTTGTCTCCT | SEQ ID NO: 872 |
| SGI_R6615320 | ACACTCTTGAGGGCCACAAAG | SEQ ID NO: 873 | TGTGATTGTAGGGTCTCCCTTGAT | SEQ ID NO: 874 |
| SGI_R8376150 | GAGAACCAGTTCAGAGTGGACTAC | SEQ ID NO: 875 | TCACTCATGTTCCTATATGGACACTGT | SEQ ID NO: 876 |
| SGI_R6624980 | ACAGCTACACCATATATGAATGGAGAAAC | SEQ ID NO: 877 | TCAGCATTTGACTTTACCTTATCAATGTCT | SEQ ID NO: 878 |
| SGI_R8376458 | GGGTACACACGTAACATAAATCTGATG | SEQ ID NO: 879 | GATTGGGTTCCAGCTGGAAAGTTA | SEQ ID NO: 880 |
| SGI_R6644435 | GCCAGGAACGTACTGGTGAAAA | SEQ ID NO: 881 | TGACCTAAAGCCACCTCCTTACTT | SEQ ID NO: 882 |
| SGI_R8376460 | TACTACCTTGAGGAACATGGTATGGT | SEQ ID NO: 883 | CTGTATAGCAGCTGCTTATCATCAGG | SEQ ID NO: 884 |
| SGI_R4389908 | GCACGGCCTCGATCTTGTAG | SEQ ID NO: 885 | CGTCTACCTGGAGATTGACAACC | SEQ ID NO: 886 |
| SGI_R4771018 | GGGTGTACAACCTTGAAGTGTATGT | SEQ ID NO: 887 | AGAATTTACACGCGTAGTTGAACCT | SEQ ID NO: 888 |
| SGI_R4390278 | TATCTCTGAAAGTGAACTCCCTGCTA | SEQ ID NO: 889 | GAGGTTCGCCCTCTCCTACTTA | SEQ ID NO: 890 |
| SGI_R4793611 | AGTAGAGCAATGTATGTCTATCCTCCA | SEQ ID NO: 891 | GACACAGGTAGAAGACTGCACTATAGTA | SEQ ID NO: 892 |
| SGI_R4390282 | TCTTGTATCCCAAGTTCACTGATTTCC | SEQ ID NO: 893 | ATGCTTACTCTAATATAACCCTCTATTTAGTCA | SEQ ID NO: 894 |
| SGI_R4800483 | AGAGGCTTTGGAGTATTTCATGAAACA | SEQ ID NO: 895 | AGAGTGAGCTTTCATTTTCTCAGTTATCTT | SEQ ID NO: 896 |
| SGI_R4390375 | GCCTCACGTTGGTCCACATC | SEQ ID NO: 897 | TCTCACCACCCGCACGTCT | SEQ ID NO: 898 |
| SGI_R4840700 | TCAGTGAAGAACTGTTCTACCAGATACT | SEQ ID NO: 899 | ATTAGTGGAGAGCTACTATTTTCAGAAACG | SEQ ID NO: 900 |
| SGI_R4395773 | GAATCAGAGCAGCCTAAAGAATCAAATG | SEQ ID NO: 901 | GGCATGGCAGAAATAATACATTCTTCTAGT | SEQ ID NO: 902 |
| SGI_R4856142 | CTCACGGCTTTGTCCAAGAGA | SEQ ID NO: 903 | GTGATGGGCAGAAGGGCACAAAG | SEQ ID NO: 904 |
| SGI_R4409014 | AGATATTTTTGGATTACTTACTCAAGTTGGTCA | SEQ ID NO: 905 | GAAAGCTGCTTTTCCAGGGTTTC | SEQ ID NO: 906 |
| SGI_R4883423 | GTGCCCTATTACCTCAATCATCCT | SEQ ID NO: 907 | ACGCCTTCACCTTTAACACCTC | SEQ ID NO: 908 |
| SGI_R4411427 | CTCTGTCACTGACTGCTGTGA | SEQ ID NO: 909 | GCTGACATTCCGGCAAGAGA | SEQ ID NO: 910 |
| SGI_R4975729 | CTAGTGTTCCTGGTCCTGACTTG | SEQ ID NO: 911 | GGTGTCAGTGACTGTGATCACAG | SEQ ID NO: 912 |
| SGI_R4411576 | CCTAGTAGAATGTTTACTACCAAATGGAATGA | SEQ ID NO: 913 | AGATTCATCTTGAAGAAGTTGATGGAGG | SEQ ID NO: 914 |
| SGI_R4975808 | CACTTTTACAGAAACAGCTGTTATACCC | SEQ ID NO: 915 | TCATGTTCTTTACAGAGAACTTCAATAATTCTT | SEQ ID NO: 916 |
| SGI_R4411583 | GCATGCCAATTGGTCTGTATCC | SEQ ID NO: 917 | GGATCCTTTTCCATAGAGAAAGTATCTACC | SEQ ID NO: 918 |
| SGI_R4978269 | AGGCAAGCCTGGCACATAC | SEQ ID NO: 919 | TCCATGGTCCTGTCTGTCCTT | SEQ ID NO: 920 |
| SGI_R4411602 | GCACGATTCTTTTAGATCTGAGATGCA | SEQ ID NO: 921 | AGCTTTTCCATTGCCTCGACTT | SEQ ID NO: 922 |
| SGI_R5012463 | AGACAGAGCTAAGGAAGCTTAAAGTG | SEQ ID NO: 923 | GGTCAATCCTATGCAAAAATCTTTCACC | SEQ ID NO: 924 |
| SGI_R4411606 | GATCTATGTTCGAACAGGTATCTACCATG | SEQ ID NO: 925 | ACTGCTAAACACTAATATAACCTTTGGAAATAT | SEQ ID NO: 926 |
| SGI_R5119473 | CTTGGTTCTTTGTTTGTCTTAATTGCAG | SEQ ID NO: 927 | GCAAAACACCAAGCATACTTACTAAACTTT | SEQ ID NO: 928 |
| SGI_R4411656 | GGAGTATATCGTCTACACAATTGGACA | SEQ ID NO: 929 | CTCAAACACAAAGCTGGTGTGT | SEQ ID NO: 930 |
| SGI_R5138044 | TCTCTCCTCTCATCCTGTCTCCTTA | SEQ ID NO: 931 | TGCCTATTGGCACTTATATAGATACGC | SEQ ID NO: 932 |
| SGI_R6644436 | TATTATGACTTGTCACAATGTCACCACAT | SEQ ID NO: 933 | GACTCGAGTGATGATTGGGAGATTC | SEQ ID NO: 934 |
| SGI_R8484603 | CGTGCCTGCCAATGGTGAT | SEQ ID NO: 935 | CTGAAGAAGATGTGGAAAAGTCCCA | SEQ ID NO: 936 |
| SGI_R6703639 | AATTCCTCAAAAACATGTCAGTGTGATTTTATT | SEQ ID NO: 937 | GTTGATGCTTTTGAAGAACGACATAAAAG | SEQ ID NO: 938 |
| SGI_R8520952 | GAAGTGGGTTACCTGACAGTGT | SEQ ID NO: 939 | GCTCCTTTCTTTGACAGAAAAAGCAG | SEQ ID NO: 940 |
| SGI_R6703642 | CCATCCAGCTTCAAAAGCTCTTC | SEQ ID NO: 941 | CCCTCTTTTACACTCCTATTGATCTGG | SEQ ID NO: 942 |
| SGI_R8525565 | TTTCCCTTGGAGATATCGATCTGTTAGA | SEQ ID NO: 943 | CTCTGAACAGGACGAACTGGAT | SEQ ID NO 944 |
| SGI_R6704094 | GACGGTGGTGTAGTTGGTCATA | SEQ ID NO: 945 | GGGAGATTCAAAAGATCCTGGAGTT | SEQ ID NO: 946 |
| SGI_R8529267 | TGCTTTTCTAACTCTCTTTGACTGCA | SEQ ID NO: 947 | TACATACAGTTTCTTGCAGCCAAGT | SEQ ID NO: 948 |
| SGI_R6713988 | ACTGTGCGACGAGCTGTG | SEQ ID NO: 949 | ATCTCAGCTGAGGAGATGGGT | SEQ ID NO: 950 |
| SGI_R5766725 | CCTTGAGTTCCAAGGCCTCATT | SEQ ID NO: 951 | TTGTAACTTGAACCATCTTTTAACTCAGGT | SEQ ID NO: 952 |
| SGI_R6734038 | GTTCTTCAGGGCAAAGAAGTCCA | SEQ ID NO: 953 | CTCTGTTTTCCAATCCAACCAGTT | SEQ ID NO: 954 |
| SGI_R8537020 | TTTCCTGTAGCAAAACCAGAAATCCT | SEQ ID NO: 955 | AAATAATCATCTCACCTCTGCTCAGTTC | SEQ ID NO: 956 |
| SGI_R6743722 | GATAGAGGTTCCTTAAGATCTCGATTTCC | SEQ ID NO: 957 | GAAGGTTGAGCTCTGCAGGTAT | SEQ ID NO: 958 |
| SGI_R8544191 | TTTCAGCATGAAATAGTGTATCAGTGGT | SEQ ID NO: 959 | CCTGGCTTTAAATCCTCGAACACAA | SEQ ID NO: 960 |
| SGI_R6743723 | TGGTTTCTGGTGGGACCATTATG | SEQ ID NO: 961 | GTCCTCTGGATCTCTTCATGCA | SEQ ID NO: 962 |
| SGI_R8562446 | AGGGCTTTTGTTTTCTTCCCTTTAGA | SEQ ID NO: 963 | GCCATTGTGCTTGAATGCACTA | SEQ ID NO: 964 |
| SGI_R6743993 | CTCTGTCACAGTGGATTCGAGA | SEQ ID NO: 965 | CAACATGACGAAGATGGCAAACTTC | SEQ ID NO: 966 |
| SGI_R8794357 | GTTGCTTTTGAACAGGGCAAAATC | SEQ ID NO: 967 | TTCCCTCCTTTACTTCATATCACTTACCT | SEQ ID NO: 968 |
| SGI_R6744095 | AAAAAGGCAAACAAACCTGGCTA | SEQ ID NO: 969 | CCCAGCAAAATAATCAGCTCTCATTTTC | SEQ ID NO: 970 |
| SGI_R8803260 | TCTGCGTGTACCTGTCGTAGTA | SEQ ID NO: 971 | CCTGACCACTTTCCCTCTCTTTTG | SEQ ID NO: 972 |
| SGI_R6758640 | GGGACATGAAATAGTTGTCCTAGCA | SEQ ID NO: 973 | AACATTATGCCCGAGACTAACAAAAGA | SEQ ID NO: 974 |
| SGI_R9094151 | GACTGATGAGAACGCAATGCAA | SEQ ID NO: 975 | TTAGGGTGATCAGTTATGAAGAAGGGA | SEQ ID NO: 976 |
| SGI_R6779848 | CTCTTCCCACAGCCACTGTTT | SEQ ID NO: 977 | TCAGTTCCTATATCCTGTGTCTGTGAAT | SEQ ID NO: 978 |
| SGI_R9039685 | CAAATACACAGAGGAAGCCTTCG | SEQ ID NO: 979 | CCAGCATTCTTACAGAAAACAAGTGGTTA | SEQ ID NO: 980 |
| SGI_R4411990 | TCACTGTTCCATAATGAAGTTAATGTCTCC | SEQ ID NO: 981 | TTCCCAGGAGCGAGAGGACATT | SEQ ID NO: 982 |
| SGI_R5237086 | TATGTTGGAGGAGGTCAGGCTTA | SEQ ID NO: 983 | CGTGAGCCCATCTGGGAAAC | SEQ ID NO: 984 |
| SGI_R4412562 | TTTTTGATGAAACAAGACGACTTTGTG | SEQ ID NO: 985 | GAATAGGATATTGTATCATACCAATTTCTCGAT | SEQ ID NO: 986 |
| SGI_R5243945 | TGAGTTTTCTGAGTGCTTTTATCAGAATGA | SEQ ID NO: 987 | CCTCAAGCAAAGTTTTAAGGCAATTTACT | SEQ ID NO: 988 |
| SGI_R4414038 | CTGTCCTCCACAGGCATTTTTG | SEQ ID NO: 989 | CCCTCACTCACAGCACATAGTC | SEQ ID NO: 990 |
| SGI_R5252171 | GTGAGGCAGTCTTTACTCACCT | SEQ ID NO: 991 | TAGGAAATGCATTTCCTTTCTTCCCA | SEQ ID NO: 992 |
| SGI_R4414904 | CCCTTCATTGCTTAACACATTTTCCTATT | SEQ ID NO: 993 | ATGGCTTACGTTAAAATAGGAAATCAGATTT | SEQ ID NO: 994 |
| SGI_R5266589 | CTTCTGTTCAATTTTGTTGAGCTTCTGA | SEQ ID NO: 995 | ACCAGACGTCACTTTCAAACGT | SEQ ID NO: 996 |
| SGI_R4414990 | GCTAGAGACAATGAATTAAGGGAAAATGACA | SEQ ID NO: 997 | ACAGAGAATCTCCATTTTAGCACTTACC | SEQ ID NO: 998 |
| SGI_R5287779 | CTCCACGCTCAGGTTGGAG | SEQ ID NO: 999 | CCACATGAGTGACTGCCTCTC | SEQ ID NO: 1000 |
| SGI_R4414994 | GAAGCCTACGTGATGGCCA | SEQ ID NO: 1001 | TTGTCTTTGTGTTCCCGGACAT | SEQ ID NO: 1002 |
| SGI_R5321351 | GGGAAATGTGAGCCCTTGAGAT | SEQ ID NO: 1003 | CCTGTGGCTGTCAGTATTTGGA | SEQ ID NO: 1004 |
| SGI_R4416985 | AATGTGTCAGCCTCAAAGAAAACC | SEQ ID NO: 1005 | CTGTCATCCCTATTGGCAGGTTAC | SEQ ID NO: 1006 |
| sGI_R5323020 | GGAGTCCATGTGTTCTGAGCT | SEQ ID NO: 1007 | GTGAAGGTTCCCAGCTGTTCT | SEQ ID NO: 1008 |
| SGI_R4416997 | TCACTTTGTGACCATTCCGGTT | SEQ ID NO: 1009 | CCTCTTCTACCTGAAGAGCAAGTC | SEQ ID NO: 1010 |
| SGI_R5438343 | GTTATGATTTTGCAGAAAACAGATCTGT | SEQ ID NO: 1011 | GCCTTCTAGAACAGTAGACACAAAACAG | SEQ ID NO: 1012 |
| SGI_R4417401 | TTGCAAGTCCTCTCAAGTCTAATAGC | SEQ ID NO: 1013 | AGATTATCCAATTCTGTTTCTTTCCTTCCA | SEQ ID NO: 1014 |
| SGI_R5456544 | CTGTTAAGGTCAATGACGCAGAGTA | SEQ ID NO: 1015 | CCTCACAACCTTGCGGAATTTTG | SEQ ID NO: 1016 |
| SGI_R4417471 | CCACATGACTGTCCTGTAGATTAAGAG | SEQ ID NO: 1017 | TTCACCGTGACCCAAAGTACTG | SEQ ID NO: 1018 |
| SGI_R5472183 | GCTATCAAAGAGATGATTGAGAACTGGT | SEQ ID NO: 1019 | TGGGCATGCGCTGTACAT | SEQ ID NO: 1020 |
| SGI_R4419217 | ATGCAATAATTTTCCCACTATCATTGATTATTT | SEQ ID NO: 1021 | CCCGAGGGTTGTTGATGTCC | SEQ ID NO: 1022 |
| SGI_R5490121 | CCAGACTGAGGTATCGCCTCAT | SEQ ID NO: 1023 | CACCCACATCATCCTTGGTTCA | SEQ ID NO: 1024 |
| SGI_R4421729 | TGCTCCCAGGCTGTTTATTTGAA | SEQ ID NO: 1025 | TGAGAACATTGCCTATGGAGACAAC | SEQ ID NO: 1026 |
| SGI_R5519595 | CTGGGTGCCCTCATTTACCTT | SEQ ID NO: 1027 | GTGCCACGACAGCGATGAGA | SEQ ID NO: 1028 |
| SGI_R6781922 | CTGAAGAGTGTTGTCCAGTTAATGGT | SEQ ID NO: 1029 | TCCTTGCTTATCCTCAAGCAACAG | SEQ ID NO: 1030 |
| SGI_R9471205 | ATTTTCACACAGCCAGGAGTCTT | SEQ ID NO: 1031 | CCAATGCAACAGACTTTAAAGAAGTTGTG | SEQ ID NO: 1032 |
| SGI_R6781937 | CACTGTGTTACTGCCATCGACTTA | SEQ ID NO: 1033 | TCGAGATTTAGCAGCCAGAAATGTTT | SEQ ID NO: 1034 |
| SGI_R9610154 | TGGGTGTTTTTGGAGAAGCACA | SEQ ID NO: 1035 | GTAGATTCTCGCCTCTATTGAGCTG | SEQ ID NO: 1036 |
| SGI_R6825663 | CATTCACCAACTTATGCCAATTCTCTTG | SEQ ID NO: 1037 | CTTTCTGAATATTGAGCTCATCAGTGAGA | SEQ ID NO: 1038 |
| SGI_R9772743 | AAAAATGATCTTGACAAAGCAAATAAAGACA | SEQ ID NO: 1039 | AGCTGTACTCCTAGAATTAAACACACATC | SEQ ID NO: 1040 |
| SGI_R6825987 | TTACCATTTGCGATCACCTGGATT | SEQ ID NO: 1041 | CTGCTCATCTTCTTAAAGATTTGATTTTTCTCC | SEQ ID NO: 1042 |
| SGI_R9803956 | CCCACACCAAGTATCAGTATGGAG | SEQ ID NO: 1043 | TCACCAACTGGATTCTTTTTCCCTT | SEQ ID NO: 1044 |
| SGI_R6826451 | AAATATTCTCCAGGCGTTTCTTCCA | SEQ ID NO: 1045 | TCTGTATCCTCAGAGTGGCATTCT | SEQ ID NO: 1046 |
| SGI_R9806482 | GTCGGAGATGCAGGTCTCAAG | SEQ ID NO: 1047 | CCAGGCTGTTGGGAACGTAAG | SEQ ID NO: 1048 |
| SGI_R6840334 | ACCCTTCCATAAAGGCTTTAACACA | SEQ ID NO: 1049 | TGTTTGGTTTTGTAGGTCTTGTGGA | SEQ ID NO: 1050 |
| SGI_R9936881 | ACTAAGCCCTATTTCTACTCTTTCTACTGT | SEQ ID NO: 1051 | AGACGATCCTGAAGAAAGAGAAGAAAAG | SEQ ID NO: 1052 |
| SGI_R6840335 | ATAACGACACAACACAAAATAGCCGT | SEQ ID NO: 1053 | CCACGGGAAAGTGGTGAAGATATG | SEQ ID NO: 1054 |
| SGI_R9964323 | AGGGACAAAGTCTGTCAACCAAAT | SEQ ID NO: 1055 | GACCTGCAGCAATACCATTGAC | SEQ ID NO: 1056 |
| SGI_R6848542 | AGTAGGATGATACATCGTGGTGTCT | SEQ ID NO: 1057 | CTGGTCTCCCACAATGAAGGTC | SEQ ID NO: 1058 |
| SGI_R9976754 | CCAGTCTCTGCATTCCACACTT | SEQ ID NO: 1059 | ATCATCTTAAGTGTTTTTCCAGTGTCTGA | SEQ ID NO: 1060 |
| SGI_R6851068 | GAGAAATATGAAGTCTTCATGGATGTTTGC | SEQ ID NO: 1061 | GAAGTAGCTACACTGCGCGTATAA | SEQ ID NO: 1062 |
| SGI_R0113144 | GGCCCAAATTCACCAATAATAGAGG | SEQ ID NO: 1063 | GACTGGAGAATGTATACACACCTTATATGG | SEQ ID NO: 1064 |
| SGI_R6905842 | CGCAGTGCTAACCAAGTTCTTTC | SEQ ID NO: 1065 | CCATGGTTAAATAAAATGCCACTTACTGTT | SEQ ID NO: 1066 |
| SGI_R0113198 | GAGAATCGAAGCGCTACCTGAT | SEQ ID NO: 1067 | CTGCCCAACGCACCGAATAGT | SEQ ID NO: 1068 |
| SGI_R6905843 | CAGCCACGGGTAATAATTTTTGTCC | SEQ ID NO 1069 | GCAGCTTTGCACCTGTTTTGTT | SEQ ID NO: 1070 |
| SGI_R0128157 | TTGCACAAAAATTTAATACTGACCCATGAA | SEQ ID NO: 1071 | CATTGGCACAGGATCATTGATGTC | SEQ ID NO: 1072 |
| SGI_R6905885 | TTTTTACCACAGCAATGTGTGTTCT | SEQ ID NO: 1073 | GTCCTTGAGCATCCCTTGTGTT | SEQ ID NO: 1074 |
| SGI_R0132838 | CAAGCCCACTGTCTATGGTGT | SEQ ID NO: 1075 | CCGTCAGGCTGTATTTCTTCCAC | SEQ ID NO: 1076 |
| SGI_R4424553 | CTTCCAAATCTACAGAGTTCCCTGTT | SEQ ID NO: 1077 | TAACCATATCAAATTCACACACTGGCAT | SEQ ID NO: 1078 |
| SGI_R5521127 | AACTCTAAATTTTCTCTTGGAAACTCCCAT | SEQ ID NO: 1079 | TCTGAAGCAATTTAGGTATGAAAGCCA | SEQ ID NO: 1080 |
| SGI_R4424786 | TTACAGAAACGCATCCAGCAAGA | SEQ ID NO: 1081 | CAATAGCGACAATGAAAAACTCCAAGATC | SEQ ID NO: 1082 |
| SGI_R5537174 | CAGCTCTGAAACATACCATTGTTCAA | SEQ ID NO: 1083 | ACCTTTATCCAAAAGAATTTTCTCCTGTGT | SEQ ID NO: 1084 |
| SGI_R4425775 | TATGGGCTGTGTGGGACGTG | SEQ ID NO: 1085 | GTCTGCATCGAAGACACGC | SEQ ID NO: 1086 |
| SGI_R5537613 | TATGCAATTTTGAACCTTACCCTCTTCT | SEQ ID NO: 1087 | CACTCTATGTGCTTTCATTCCTGGAA | SEQ ID NO: 1088 |
| SGI_R4425791 | CTTGTCTGCCACCTGCAGAT | SEQ ID NO: 1089 | CATCTCCTTGCCTCCACTCAC | SEQ ID NO: 1090 |
| SGI_R5537630 | TAACAACCCTCCTGCCATCATATTG | SEQ ID NO: 1091 | CTCCCTCTGCAGAGTTGTTAGC | SEQ ID NO: 1092 |
| SGI_R4426384 | TCAAGTGACACCTCACCTCTCT | SEQ ID NO: 1093 | GAAGGAAGTGTGCCAGGCATA | SEQ ID NO: 1094 |
| SGI_R5537631 | GATTCATCAGGAGAGCATTTAAGGGA | SEQ ID NO: 1095 | TGGAGCATATGATTTTATGGTAAAGGTGT | SEQ ID NO: 1096 |
| SGI_R4426396 | GCCAGTAACCCACCTTCTGT | SEQ ID NO: 1097 | GATGAGCACCAGGCCGATT | SEQ ID NO: 1098 |
| SGI_R5571881 | ATGGCTCTGTAAATTCTACCCGTTTT | SEQ ID NO: 1099 | ACAACTCGGCTTTCATTTGAACC | SEQ ID NO: 1100 |
| SGI_R4426405 | CCCAGTACCATTCCTCGACT | SEQ ID NO: 1101 | GCTCTGGGCAGAATGGGTTG | SEQ ID NO: 1102 |
| SGI_R5580373 | GCGGGTAGCTACGATGAGG | SEQ ID NO: 1103 | CCCAAAAGAAGCAAGATGGAAGTC | SEQ ID NO: 1104 |
| SGI_R4426519 | CTCTACGTCTCCTCCGACCA | SEQ ID NO: 1105 | CTTATTTATTGGTCTCTCATTCTCCCATCC | SEQ ID NO: 1106 |
| SGI_R5580375 | GAGCAGGGCCAACGTTAGAA | SEQ ID NO: 1107 | CCAGCCAATAGGAGCAGAGATG | SEQ ID NO: 1108 |
| SGI_R4426600 | CAAGGACCCAAACATCATCCATCT | SEQ ID NO: 1109 | CATCGCTGGAGGAAGAATTAGGG | SEQ ID NO: 1110 |
| SGI_R5631676 | CAGATATTTCTTTCCTTAACTAAAGTACTCAGA | SEQ ID NO: 1111 | AGAAAATCAAAGCATTCTTACCTTACTACATCA | SEQ ID NO: 1112 |
| SGI_R4426652 | GCTGGAGAAGAGATACGAAGAACC | SEQ ID NO: 1113 | GTGAGTGGTAGGTCTTGTAGGGA | SEQ ID NO: 1114 |
| SGI_R5635278 | ATAACTGGTGTACTTGATAGGCATTTGAAT | SEQ ID NO: 1115 | GATCTGTTGTCATCTTATAAATCTCCCAGA | SEQ ID NO: 1116 |
| SGI_R4426788 | TTGAAAGAGAACACACTTACTCTCCAC | SEQ ID NO: 1117 | CTGAGACATTCCTATGTCCTGCTC | SEQ ID NO: 1118 |
| SGI_R5678025 | GGTTCCACATAAGGTTCTCATGAGA | SEQ ID NO: 1119 | TGGACTGGCAGACTATGTTAATCTTTTTATTTT | SEQ ID NO: 1120 |
| SGI_R4426809 | CTTGCCTAGACAGCACCGTAAT | SEQ ID NO: 1121 | AGGAGGATAAAGACCTGGTCCAT | SEQ ID NO: 1122 |
| SGI_R5755718 | ACAACACACAGTTGGAGGACTT | SEQ ID NO: 1123 | CCCATCACACACCATAACTCCA | SEQ ID NO: 1124 |
| SGI_R6905907 | AGACTTAGTACCTGAAGGGTGAAATATTCT | SEQ ID NO: 1125 | GGGTGCAGTTATGCCTCAGATTC | SEQ ID NO: 1126 |
| SGI_R0135356 | TGAAAACAATGGTGACTACATGGACA | SEQ ID NO: 1127 | TCTTCTTATATGAGGCTGGACGATCATA | SEQ ID NO: 1128 |
| SGI_R6928815 | GACCGAGAAGGACCAACTGATC | SEQ ID NO: 1129 | AAAATCTATTTGCAGTTTGAATGGTCAACA | SEQ ID NO: 1130 |
| SGI_R0135381 | TGGTCTCAATGATATGGAGATGGTGA | SEQ ID NO: 1131 | TCACATTTCTTTGTACAGGAAAACACG | SEQ ID NO: 1132 |
| SGI_R6935268 | GTTGAAGCTGAACACGCAAAAGA | SEQ ID NO: 1133 | TCAGTAACGTGAAGTATGTCATGTTGG | SEQ ID NO: 1134 |
| SGI_R0135395 | CCCACACATGACAGCCATCATC | SEQ ID NO: 1135 | ACGTTCTAACAGAGTGACAGAAACGTAA | SEQ ID NO: 1136 |
| SGI_R7024618 | CTCACCTGTGACATTCACCATGA | SEQ ID NO: 1137 | CCAACAATAGGACAGTGCTTATTGG | SEQ ID NO: 1138 |
| SGI_R0143789 | CAGGTTATTTTATACCTCACCTCATTGTCA | SEQ ID NO: 1139 | GTTTTCCTTTGTGTCATTCCCTTTTATCAG | SEQ ID NO: 1140 |
| SGI_R7129863 | CCACTCCTTGCTTCTCAGATGA | SEQ ID NO: 1141 | CAGAGGACAATGTGATGAAGATAGCA | SEQ ID NO: 1142 |
| SGI_R0145558 | GCCTGGCTCATTAAGATGACCT | SEQ ID NO: 1143 | TCTCTATCACTCCTTGAAGCCATCA | SEQ ID NO: 1144 |
| SGI_R7129864 | AGAGAGGCCTTGGGACTGATAC | SEQ ID NO: 1145 | GATGAAGATGATCGGGAAGCATAAGA | SEQ ID NO: 1146 |
| SGI_R0218014 | AGGCAAACATGGTGGGATTTTG | SEQ ID NO: 1147 | TTTCTCTTTGGGTCCTAGGTATTATGAGA | SEQ ID NO: 1148 |
| SGI_R7129866 | TACTCAAACTATTGGGTGGATTTGTTTGT | SEQ ID NO: 1149 | AACATGTGTAGAAAGCAGATTTCTCCAT | SEQ ID NO: 1150 |
| SGI_R0231562 | CTCTCCAGGACGCACAGTTT | SEQ ID NO: 1151 | ACTCAGTCGGAGGTGAGGAA | SEQ ID NO: 1152 |
| SGI_R7129867 | TGCACAGTGAATCCAAAAGAAAGTATACT | SEQ ID NO: 1153 | CACGAATTACAATGAATTCAAGTTACCTGT | SEQ ID NO: 1154 |
| SGI_R0234257 | CGAGCAGCTCTCTCTTCAGGA | SEQ ID NO: 1155 | CTACGAGGCTGAGCACGAATA | SEQ ID NO: 1156 |
| SGI_R7165827 | GGTTTCATAACCCACAGATCCATTTC | SEQ ID NO: 1157 | CTCAGAAAAATGCCAACATACCTGATG | SEQ ID NO: 1158 |
| SGI_R0234264 | AAAAATGTACCACTACTCAACTGTGG | SEQ ID NO: 1159 | AGAGGAGGAGCTGGAGATCAG | SEQ ID NO: 1160 |
| SGI_R7168583 | CTTACACCATAGTAACCAGTACCCACTA | SEQ ID NO: 1161 | TGCACAAGCACTGAAACATAACAAAGA | SEQ ID NO: 1162 |
| SGI_R0234265 | AGTTAGTGTGGACGTCTCTGTACA | SEQ ID NO: 1163 | ATGGCGACTTGTGCGTTTTC | SEQ ID NO: 1164 |
| SGI_R7177284 | AGTTTGCCAAGTGAAATAGTACACTAGG | SEQ ID NO: 1165 | GCATACATCAGACAGCACAGAATTGATA | SEQ ID NO: 1166 |
| SGI_R0234279 | AATCCCTGGAAAAGGCAATCGA | SEQ ID NO: 1167 | CCCTCCTCGCTTTATTTTTGGGA | SEQ ID NO: 1168 |
| SGI_R7191721 | TGTTCCTCCTCTACCACACGAT | SEQ ID NO: 1169 | GCAAGCTGGCTTTTGGAAATGAAT | SEQ ID NO: 1170 |
| SGI_R0234295 | TAACACTTGAGAAAACCCAGGCTAAAA | SEQ ID NO: 1171 | TTGCTGGAGGATAGAAAGTAAGTGC | SEQ ID NO: 1172 |
| SGI_R4427102 | GGAAAAATTGTGAAGATCTGTGACTTTGG | SEQ ID NO: 1173 | CTGACTTTAGAGATTAAAGTGAAGGAGGAT | SEQ ID NO: 1174 |
| SGI_R5756039 | GACACCCAAAAGTCCACCTGAA | SEQ ID NO: 1175 | CCATTCCACTGCATGGTTCACT | SEQ ID NO: 1176 |
| SGI_R4427840 | TCATAGGGCACCACCACACTAT | SEQ ID NO: 1177 | GGCCTCTGATTCCTCACTGATTG | SEQ ID NO: 1178 |
| SGI_R5778387 | TTCCTTCTTCAATTTTTGTTGTTTCCATGT | SEQ ID NO: 1179 | TGCAATTTACCTAGTAATGGGTTGTAACA | SEQ ID NO: 1180 |
| SGI_R4427854 | CCCTTTCTTGCGGAGATTCTCT | SEQ ID NO: 1181 | TTTCCTTACTGCCTCTTGCTTCTC | SEQ ID NO: 1182 |
| SGI_R5781852 | GTCTTGCATTTGAAGAAGGAAGCC | SEQ ID NO: 1183 | AACCCAAAGTATGAGATAAATACTGTCATAAAT | SEQ ID NO: 1184 |
| SGI_R4428652 | TTCAGATGCATCTGTTACTATCTTTTGCT | SEQ ID NO: 1185 | TGCCACTCCCTCTAGGATCAAA | SEQ ID NO: 1186 |
| SGI_R5781893 | CCATGTATGAAGTACACTCGAAGCT | SEQ ID NO: 1187 | CCCTGTTTCATACTCACCAAAACTCA | SEQ ID NO: 1188 |
| SGI_R4430743 | CGCCAGGCTCACCTCTATAG | SEQ ID NO: 1189 | AGGAGCGATGACGGAATATAAGC | SEQ ID NO: 1190 |
| SGI_R5782149 | TGATGCTTTCTGGCTGGATTTAAATTATCT | SEQ ID NO: 1191 | CCATTACCTTTTCTCTTGATCATCCATACT | SEQ ID NO: 1192 |
| SGI_R4433393 | CCTGGAGTCTTCCAGTGTGATG | SEQ ID NO: 1193 | CCTCATCTTGGGCCTGTGTTAT | SEQ ID NO: 1194 |
| SGI_R5782161 | GGTAGCTCATCATCTGGGACAG | SEQ ID NO: 1195 | GCCGAACCAATACAACCCTCT | SEQ ID NO: 1196 |
| SGI_R4484197 | CTAGATTATGATGTGTTCCATGTATGGCA | SEQ ID NO: 1197 | TACTATGGAAAATTACCTACCTCCTGAACA | SEQ ID NO: 1198 |
| SGI_R5782166 | TACCTCTATTGTTGGATCATATTCGTCCA | SEQ ID NO: 1199 | TATTATAAGGCCTGCTGAAAATGACTGAAT | SEQ ID NO: 1200 |
| SGI_R4484576 | GCCGAAGTCTGACCCTTTTTGT | SEQ ID NO: 1201 | GGTACCTGTAGTGTGCAGGAAA | SEQ ID NO: 1202 |
| SGI_R5872534 | CTTCCTAAGGTTGCACATAGGCA | SEQ ID NO: 1203 | GCCCAGCTTCTCTGTCTAAGTAGTAA | SEQ ID NO: 1204 |
| SGI_R4486235 | GGGAAGAAAAGTGTTTTGAAATGTGTTT | SEQ ID NO: 1205 | CATTTTTCCAGATACTAGAGTGTCTGTGTA | SEQ ID NO: 1206 |
| SGI_R6043242 | TCTTATTCTGAGGTTATCTTTTTACCACAGTTG | SEQ ID NO: 1207 | GCTGCAACATGATTGTCATCTTCA | SEQ ID NO: 1208 |
| SGI_R4502373 | GTCAGGTGGTGTGATGGTGAT | SEQ ID NO: 1209 | GGAGCGAAGCTCATGACTGTC | SEQ ID NO: 1210 |
| SGI_R6052482 | GCTTGGATCTGGCGCTTTT | SEQ ID NO: 1211 | AAACACTGCCTCCAGCTCTT | SEQ ID NO: 1212 |
| SGI_R4502383 | ATCGAAGGTGCGTTCGATCA | SEQ ID NO: 1213 | ATGCACGCAGACAGAGGCTCT | SEQ ID NO: 1214 |
| SGI_R6066373 | AGCTGCTCACCATCGCTATC | SEQ ID NO: 1215 | CAGCTGTGGGTTGATTCCAC | SEQ ID NO: 1216 |
| SGI_R4506663 | CCTGAATCAAATAGGGAAGGAAAGGA | SEQ ID NO: 1217 | TACGGACCTTACGTCAGTGACT | SEQ ID NO: 1218 |
| SGI_R6070401 | AGCAAATGTGTCTTCACTTTTTCATGA | SEQ ID NO: 1219 | CTGCTGGGCACAGATGATTTTG | SEQ ID NO: 1220 |
| SGI_R7230300 | GATTCAATCAAACTGCAGAGTATTTGGG | SEQ ID NO: 1221 | TGATCTGGTGTCAGAGATGGAGAT | SEQ ID NO: 1222 |
| SGI_R0234296 | GTGTCAGTAATGGGAAATCTGCAAG | SEQ ID NO: 1223 | CCAAGAACTCCGCACTTTCTCTC | SEQ ID NO: 1224 |
| SGI_R7252344 | CACATGTTTAGTGATGAAAAATTTCTCCCT | SEQ ID NO: 1225 | TAACATACCTACTAAGTGCTGTCCACTAAT | SEQ ID NO: 1226 |
| SGI_R0234307 | GGAGATCCGCTGGGACAAAT | SEQ ID NO: 1227 | GGCTAGACCAAACCGCAATTCT | SEQ ID NO: 1228 |
| SGI_R7311943 | TTTGTGAACGCCTTCTGTCTGA | SEQ ID NO: 1229 | AGAAGGTGAAGTGCTTGATTTTCTTACTT | SEQ ID NO: 1230 |
| SGI_R0234308 | GGGATGACCTGGAAACTTCGG | SEQ ID NO: 1231 | CAAACTTTTCTCTCTGGACACTCG | SEQ ID NO: 1232 |
| SGI_R7344281 | TCATAATTGTGATTTTCTAAAATAGCAGGCTCT | SEQ ID NO: 1233 | ATTGTTTTTAACTTACTGATTTAAGCATGGATT | SEQ ID NO: 1234 |
| SGI_R0234309 | CGGAACGCGTCCGAAAATG | SEQ ID NO: 1235 | GCACTCCCGTGTAACTCCTATGA | SEQ ID NO: 1236 |
| SGI_R73S3860 | GGTTCCATTGGTAGCTGGTGAT | SEQ ID NO: 1237 | GCCCATTTTTATCTACTTCCATCTTGTCA | SEQ ID NO: 1238 |
| SGI_R0234359 | CATCCGACTCGCATCTTCG | SEQ ID NO: 1239 | GCCAAACAAAGTTCTCTCTCACC | SEQ ID NO: 1240 |
| SGI_R7484042 | GTTGCAGCAATTCACTGTAAAGCT | SEQ ID NO: 1241 | ACCTTTTTGTCTCTGGTCCTTACTTC | SEQ ID NO: 1242 |
| SGI_R0234360 | GTCTCTGAGCCTGTGAGTGC | SEQ ID NO: 1243 | CAGAGCGCTGGAGACCATT | SEQ ID NO: 1244 |
| SGI_R7645798 | CACCTTCTTTCTAACCTTTTCTTATGTGC | SEQ ID NO: 1245 | TCCTGCTTTGAACAAATAAATGAATCACG | SEQ ID NO: 1246 |
| SGI_R0276351 | TTGAAGAACACGAATCTCCGCA | SEQ ID NO: 1247 | AGGATGATGCCACAGTCGTC | SEQ ID NO: 1248 |
| SGI_R7648155 | GCTCAAGTTCTTGTGTTTGTGTGT | SEQ ID NO: 1249 | CCATATGCAGGTGGAGGGATTTG | SEQ ID NO: 1250 |
| SGI_R0276354 | GAGAGACCGAAGCCACCTTT | SEQ ID NO: 1251 | TAGAGCCGCAGCATGTGTT | SEQ ID NO: 1252 |
| SGI_R7743764 | TAGGACACTACCCAATGCCTCA | SEQ ID NO: 1253 | CCAAAATAATGTGATGGAATGATAAACCAAGAT | SEQ ID NO: 1254 |
| SGI_R0276358 | GTGCTACCTGTTTGTGTGCG | SEQ ID NO: 1255 | TAATCCGAGCTCCGCTGGTCA | SEQ ID NO: 1256 |
| SGI_R7743795 | TAACGTCTTCCTTCTCTCTCTGTCAT | SEQ ID NO: 1257 | AGCAGAAACTCACATCGAGGATTTC | SEQ ID NO: 1258 |
| SGI_R0283579 | GTGGTGATCTGGGTAATAGTTTCTCC | SEQ ID NO: 1259 | TGTTCAGAGGATAGCAACATACTTCG | SEQ ID NO: 1260 |
| SGI_R7743853 | AATCTACAGGAATAGCCACATACAGAATG | SEQ ID NO: 1261 | CTTTCTGTGTAGTACCTTCATGAAAACG | SEQ ID NO: 1262 |
| SGI_R0283581 | TATGGTCTGCAGGACAATTCATGG | SEQ ID NO: 1263 | TCTTATGCAAATAGTTGACCAAATCTCCAT | SEQ ID NO: 1264 |
| SGI_R7746037 | CCCAGCGTCCTCAAAAGTTACA | SEQ ID NO: 1265 | CCCTCCACAATCATTCCTGTGT | SEQ ID NO: 1266 |
| SGI_R0283582 | CCACTTTTGCACAGCCAAGAAC | SEQ ID NO: 1267 | TGAGAATGATCGTTTTCTTCCTCTGTTAG | SEQ ID NO: 1268 |
| SGI_R4508122 | CCAGGCATTGAAGTCTCATGGA | SEQ ID NO: 1269 | ATCTTCTGTCCCTTCCCAGAAAAC | SEQ ID NO: 1270 |
| SGI_R6070426 | GCAGTTGGGCACTTTTGAAGAT | SEQ ID NO: 1271 | AATCAAAGTCACCAACCTTTAAGAAGGA | SEQ ID NO: 1272 |
| SGI_R4509347 | GGCATTCTGGGAGCTTCATCTG | SEQ ID NO: 1273 | CTGACTGCTCTTTTCACCCATCT | SEQ ID NO: 1274 |
| SGI_R6282741 | GGCCAGGGTCAAAGATATTTGGA | SEQ ID NO: 1275 | ACTTCTCCTCACTTCTGGACTTCTTTATA | SEQ ID NO: 1276 |
| SGI_R4509463 | AGAAGCCTTCCGGCACAAG | SEQ ID NO: 1277 | CTTACCGTGGACCTTACTGGG | SEQ ID NO: 1278 |
| SGI_R6282773 | GTATGGTGTGTTCTGGAAGTCCA | SEQ ID NO: 1279 | CGTGATAGTGGCCATCTTCCT | SEQ ID NO: 1280 |
| SGI_R4509515 | CACCTGGTACGTCCGCAA | SEQ ID NO: 1281 | GGGATGGTGAAGCTTCCAGC | SEQ ID NO: 1282 |
| SGI_R6306375 | TTTTCTTAACACATTGACTTTTTGGTTCGT | SEQ ID NO: 1283 | GTATCTTGAAGATTTAGCCATTCCAAAACC | SEQ ID NO: 1284 |
| SGI_R4519384 | CGACCGGAAGTCCATCTCCT | SEQ ID NO: 1285 | TGGAGCTCCTGATCTGGTACAG | SEQ ID NO: 1286 |
| SGI_R6326495 | GAATGCAAAACAGAGCCTCGT | SEQ ID NO: 1287 | CCAGACGTCCTGTCACTCG | SEQ ID NO: 1288 |
| SGI_R4521086 | GAGTAAATGTTGACCAAAGGGAGAAAATG | SEQ ID NO: 1289 | GCTTCTTCTTTTAGATACCGGATAATGACT | SEQ ID NO: 1290 |
| SGI_R6564300 | TGACCACCAGTATAGTTCCAGGA | SEQ ID NO: 1291 | ACCCTCTAACTGATACAATAACACCCATTT | SEQ ID NO: 1292 |
| SGI_R4534171 | TTGACAGAACGGGAAGCCCTCAT | SEQ ID NO: 1293 | CCTGACAGACAATAAAAGGCAGCTT | SEQ ID NO: 1294 |
| SGI_R6576266 | CAGCTCGTTCATCGGGACTT | SEQ ID NO: 1295 | ACCTGGCTCCTCTTCACGTA | SEQ ID NO: 1296 |
| SGI_R4534172 | AGTGAAAAACAAGCTCTCATGTCTGA | SEQ ID NO: 1297 | CATGTGTCCAGTGAAAATCCTCACT | SEQ ID NO: 1298 |
| SGI_R6584115 | CTCAAGAGTGAGCCACTTCTTACC | SEQ ID NO: 1299 | CTCCTCTTGTCTTCTCCTTTGCA | SEQ ID NO: 1300 |
| SGI_R4534197 | CCTTACTCATGGTCGGATCACAA | SEQ ID NO: 1301 | GTTGAAACTAAAAATCCTTTGCAGGACT | SEQ ID NO 1302 |
| SGI_RG584116 | GAGCTTGCTCAGCTTGTACTCA | SEQ ID NO: 1303 | GCCTGTGTAGTGCTTCAAGGG | SEQ ID NO: 1304 |
| SGI_R4534206 | CAACATCACCACGGGTCTGTA | SEQ ID NO: 1305 | GATGAGGCTCCCACCTTTCAG | SEQ ID NO: 1306 |
| SGI_R6584134 | CCCATTTTCTTCTACTTCCATCTTGGA | SEQ ID NO: 1307 | GTTTTGAGCTTGTTTGCTGAATGTTAAC | SEQ ID NO: 1308 |
| SGI_R4534211 | CGTCCTGGGATTGCAGATTGG | SEQ ID NO: 1309 | GATGGATGTCACGTTCTCAAAGC | SEQ ID NO: 1310 |
| SGI_R6584137 | CCTCAATGTAACAAATATGACAGTAACCCT | SEQ ID NO: 1311 | AGATGGAAACTTTGGACTTCAAGAACTT | SEQ ID NO: 1312 |
| SGI_R4534216 | CTTAAAAGGTCTTTGATTTGCGTCAGT | SEQ ID NO: 1313 | GGAGCTATTCCACCTACTGATCCT | SEQ ID NO: 1314 |
| SGI_R6584187 | TTTGAATCTTTGGCCAGTACCTCA | SEQ ID NO: 1315 | CATAAGAGAGAAGGTTTGACTGCCATAAA | SEQ ID NO: 1316 |
| SGI_R7774641 | GAACCTCATGACCTGAAGGAGT | SEQ ID NO: 1317 | TCCCGACTGTAATTGATCTTCTACATG | SEQ ID NO: 1318 |
| SGI_R0283583 | GTCCAGAGTGAGTTAACTTTTTCCAAC | SEQ ID NO: 1319 | CATCACTCTGGTGGGTATAGATTCTG | SEQ ID NO: 1320 |
| SGI_R7774649 | CTGGCCCTTCCCAAGATTTGAT | SEQ ID NO: 1321 | GAGAAGGCCTTGGCAATCATCT | SEQ ID NO: 1322 |
| SGI_R0283584 | AAAAGTAGAAGCAATCTGATGAACTCCA | SEQ ID NO: 1323 | ACTCTCATCTTTTGCTGAGAAGCA | SEQ ID NO: 1324 |
| SGI_R7775787 | CAATCCCTGACCCTGGCTT | SEQ ID NO: 1325 | GTGTACTTCCGGATCTTCTGCTG | SEQ ID NO: 1326 |
| SGI_R5453528 | TTTTTACTGTTCTTCCTCAGACATTCAAAC | SEQ ID NO 1327 | CCTACCCTGGTGGAAGCATACT | SEQ ID NO: 1328 |
| SGI_R7006681 | GGAACCTCCTGGACTACCTGA | SEQ ID NO: 1329 | CCCTACCTGTGGATGAAGTTTTTCTTC | SEQ ID NO: 1330 |
| SGI_R6594735 | TTGGAAGTTGTTTTGTTTTGCTAAAACAAAG | SEQ ID NO: 1331 | GGATTTGAGCTGAGGTCTTCTGATG | SEQ ID NO: 1332 |
| SGI_R7817487 | CAGACACTGTACAAGCTCTACGA | SEQ ID NO: 1333 | GAATAAAGAGGAGCAGGTTGAGGAA | SEQ ID NO: 1334 |
| SGI_R6758860 | GCTGCTGTGGGAATGAACAAA | SEQ ID NO: 1335 | GCAATGCTGCACCAGGTTG | SEQ ID NO: 1336 |
| SGI_R7848528 | ACTCCTCCATATGTAGTTCGCTTTG | SEQ ID NO: 1337 | GAAAATGTTGATGTGTCTTGCATAGGT | SEQ ID NO: 1338 |
| SGI_R6848743 | AAAAGCTCATTAACTTAACTGACATTCTCA | SEQ ID NO: 1339 | ATCTGTATATACACTGGGCTTCTAAACAAC | SEQ ID NO: 1340 |
| SGI_R7851848 | TGGTAGGCTTGAGTTTGAAGAAACA | SEQ ID NO: 1341 | TCCTTACCAATACTCCATCCACAGA | SEQ ID NO: 1342 |
| SGI_R7251681 | GCATCAACCTTCTCAAGACAACCT | SEQ ID NO: 1343 | GCACCCAGCCAATTTTGAGTATTTTTAAAA | SEQ ID NO: 1344 |
| SGI_R7851854 | TGACATGTAAAGGATAATTGTCAGTGACTTT | SEQ ID NO: 1345 | TCAGTCTGAAGAGTTTTATCATGATCCAAAAAT | SEQ ID NO: 1346 |
| SGI_R6181676 | AAAGATTCAGGCAATGTTTGTTAGTATTAGT | SEQ ID NO: 1347 | CTACCTCTGCAATTAAATTTGGCGG | SEQ ID NO: 1348 |
| SGI_R7867605 | TCCTACCTGGTCTTCTAGGAAGC | SEQ ID NO: 1349 | GAGGGTTTTCGTGGTTCACATC | SEQ ID NO: 1350 |
| SGI_R8529102 | CTTTGTCTTCGTGATTTGTAGGAGTCA | SEQ ID NO: 1351 | AGCACGAGGAAGATCAGGAATG | SEQ ID NO: 1352 |
| SGI_R7911141 | CGTGAAGAACAGCACGTACACA | SEQ ID NO: 1353 | AGAATGAACTCTTCCCTCCAAAAGAAG | SEQ ID NO: 1354 |
| SGI_R0135391 | CTGCCAGTGCATATACTTTCTGGA | SEQ ID NO: 1355 | CACTGGATTTTAACAAGGCATGTGA | SEQ ID NO: 1356 |
| SGI_R7975413 | CTCAAGTTATTTGGAATTTTGAAGAGGTGA | SEQ ID NO: 1357 | GGCACTGTATGCACTCAGAGTTC | SEQ ID NO: 1358 |
| SGI_R0317010 | AGATGCATAGAGCCTACCTGTCA | SEQ ID NO: 1359 | CTTGGTGCTAGTGGAGAACAAAAC | SEQ ID NO: 1360 |
| SGI_R7986175 | TCCTGCTCGTCGTCCTGTG | SEQ ID NO: 1361 | CTTCCTCACCGACGAGGAAG | SEQ ID NO: 1362 |
| SGI_R0317014 | CAGCATCACTTCACTGGCTTCT | SEQ ID NO: 1363 | TTGATCCTTTGATGCCCTCATTATCAA | SEQ ID NO: 1364 |
| SGI_R4534229 | TGCTTACTTTGAAATGGATGTTCAGGT | SEQ ID NO: 1365 | TCCTGTGGACATTGGAGAGTTG | SEQ ID NO: 1366 |
| SGI_R6584196 | CATCCATCCATCCAGGAAAATCAGA | SEQ ID NO: 1367 | GATCCATTCATTCTGCTTATTCTCATTCG | SEQ ID NO: 1368 |
| SGI_R4534256 | GTTTTATCAAAGCACAACGCAACTTGA | SEQ ID NO: 1369 | CCCATATGCACATGAATCAATTTCTTCAAT | SEQ ID NO: 1370 |
| SGI_R6584201 | GACATGAGAGCTCGATGCTCA | SEQ ID NO: 1371 | CCCGGAGGGTAAGTTGTATAGTG | SEQ ID NO: 1372 |
| SGI_R4534273 | CATGCATGAACATTTTCTCCACCTT | SEQ ID NO: 1373 | CTTCCAGACCAGGGTGTTGTTT | SEQ ID NO: 1374 |
| SGI_R6584203 | TAAGGTGCTCAAAAATTTCTTCATCTCACT | SEQ ID NO: 1375 | AGTTATTGGGTAATGTTATATGCTGTGCTT | SEQ ID NO: 1376 |
| SGI_R4534279 | CGAGGGCAAATACAGCTTTGGT | SEQ ID NO: 1377 | GACTCTCCAAGATGGGATACTCCA | SEQ ID NO: 1378 |
| SGI_R6584224 | GTTTGTAAACACTGTCCTGTTTTGATATCC | SEQ ID NO: 1379 | ACAGGGAATTGCATTCACACGTTA | SEQ ID NO: 1380 |
| SGI_R4534297 | TTCACCTCACTGAAACCTTTGTGT | SEQ ID NO: 1381 | GTCCACCAACACTGAGCACAGT | SEQ ID NO: 1382 |
| SGI_R6584227 | GATAATCTTTACCTCTTTAGGGAGCAATGA | SEQ ID NO: 1383 | GTGGACCAGAGAAATTGCTTGC | SEQ ID NO: 1384 |
| SGI_R4534307 | CCATCCTGACCTGGTATGGTCA | SEQ ID NO: 1385 | CCTGCTTCAGGACGTTGAACTC | SEQ ID NO: 1386 |
| SGI_R6584305 | GTTATGTCCTCATTGCCCTCAACA | SEQ ID NO: 1387 | CTTCAGTCCGGTTTTATTTGCATCATAG | SEQ ID NO: 1388 |
| SGI_R4534312 | CTCCACCATGACTTTGAGGTTGA | SEQ ID NO: 1389 | ACAAGGACATCTTCCCACTAATGC | SEQ ID NO: 1390 |
| SGI_R6584316 | CCCACAATCATACTGCTGACATACA | SEQ ID NO: 1391 | GATGAACCGGTCCTTTACAGATGAAA | SEQ ID NO: 1392 |
| SGI_R4534365 | ATGGCCATGGAACCAGACAGAA | SEQ ID NO: 1393 | TCCACATCCTCTTCCTCAGGATT | SEQ ID NO: 1394 |
| SGI_R6584317 | GTTCGCACAAAGCAAGCCAGAT | SEQ ID NO: 1395 | GTCCGTAAAAATGCTGGAGACATC | SEQ ID NO: 1396 |
| SGI_R4534376 | CCCAGCTGTGATCCATGAGAAC | SEQ ID NO: 1397 | CCGACTGCCTTGTACCATTCAT | SEQ ID NO: 1398 |
| SGI_R6584320 | GCTTGTAAGTGCCCGAAGTGTA | SEQ ID NO: 1399 | CACAACCCACTGAGGTATATGTATAGGTAT | SEQ ID NO: 1400 |
| SGI_R4534392 | TCAAATGTTAGCTCATTTTTGTTAATGGTGG | SEQ ID NO: 1401 | TGCAAGCATACAAATAAGAAAACATACTTACAG | SEQ ID NO: 1402 |
| SGI_R6584323 | CTCAATGAGCCCTCAGCTGAT | SEQ ID NO: 1403 | CCAGAAGCTTGAACTCTCATACCTG | SEQ ID NO: 1404 |
| SGI_R4534420 | GCATTTCCTGTGAAATAATACTGGTATGTATTT | SEQ ID NO: 1405 | GGGAACTCAAAGTACATGAACTTGTCT | SEQ ID NO: 1406 |
| SGI_R6584395 | TTTTTCACAAAGTTTTTGCTTCAAATGTCT | SEQ ID NO: 1407 | CCTCATCGGAATCAAGCTCAGT | SEQ ID NO: 1408 |
| SGI_R4534459 | CTTTGCTTGTCCCGATAGGTCA | SEQ ID NO: 1409 | GGCAGTGTGATATTGGCAAAAATAGG | SEQ ID NO: 1410 |
| SGI_R6584418 | CCACTTGGTGAAGGTAGCTGAT | SEQ ID NO: 1411 | CGGACTTGATGGAGAACTTGTTGTAG | SEQ ID NO: 1412 |
| SGI_R7997270 | CAGCTTTCGACAAAAGTCACAAAATG | SEQ ID NO: 1413 | TTAAACAAGAGAGTAGATACGTCAGTTTCTAGA | SEQ ID NO: 1414 |
| SGI_R0317019 | TTAGATGGCTCATTCACAACTATCTTTCC | SEQ ID NO: 1415 | TGGGTAATTACAGTCCAGAAGTTCCATA | SEQ ID NO: 1416 |
| SGI_R8002155 | GAGCACAGGAACTTCTTGGTGT | SEQ ID NO: 1417 | ACGGCATCGAATACCAGAACAT | SEQ ID NO: 1418 |
| SGI_R0317024 | AGGCAAATCCTAAGAGAGAACAACTG | SEQ ID NO: 1419 | CATAATGCTTCCTGGTCTTTAGGATTTCT | SEQ ID NO: 1420 |
| SGI_R8153189 | CCCACTCTCCAATGTGACTAGGT | SEQ ID NO: 1421 | CCAACAAGCATCAGAGTGCTGT | SEQ ID NO: 1422 |
| SGI_R0317029 | GAAAAAGCCCTTAGAGATCATGCTAGA | SEQ ID NO: 1423 | GTCTCTTTGCAGTTATGATGGTTAACG | SEQ ID NO: 1424 |
| SGI_R8153197 | ATGTCACCTGAAACATTTTTAGCCATTC | SEQ ID NO: 1425 | GCTTGTACCATGTTCAGCAACAC | SEQ ID NO: 1426 |
| SGI_R0317030 | GACAACATTAACGCTGACTTGATCAC | SEQ ID NO: 1427 | CAGAAACAGCTCTAGACAACAAACCT | SEQ ID NO: 1428 |
| SGI_R8153431 | CTGAGGGTGTCCTGTGTCAC | SEQ ID NO: 1429 | CATGAAACGCAGATTACCATGCAG | SEQ ID NO: 1430 |
| SGI_R0317033 | TGGCCTGCCCTATATAATTGGAGA | SEQ ID NO: 1431 | CCGTTATATTGTTCTCCTGTGTCTGT | SEQ ID NO: 1432 |
| SGI_R8179347 | GGGAGTGAGGATGGCTACAG | SEQ ID NO: 1433 | CCTTCCATGTGGAGACTCCTG | SEQ ID NO: 1434 |
| SGI_R0317034 | AAGGCAGTAGAAGTTGCTGGAAA | SEQ ID NO: 1435 | TCCGATGATTTCATGTAGTTTTCAATTCTTTG | SEQ ID NO: 1436 |
| SGI_R8179895 | AGCATGCCAATCTCTTCATAAATCTTTTC | SEQ ID NO: 1437 | GCCTCTTGCTCAGTTTTATCTAAGGC | SEQ ID NO: 1438 |
| SGI_R0317035 | CGGAATTTGAAAACAAGCAAGCTCT | SEQ ID NO: 1439 | CACTCACTCAGTTAACTGGTGAACATAAA | SEQ ID NO: 1440 |
| SGI_R8180002 | GGTCATACAGCTGATTGCCACA | SEQ ID NO: 1441 | GAGGTCTGCTTTGGTCCATCTT | SEQ ID NO: 1442 |
| SGI_R0317036 | GAATGGAGAAACTCCCAGATTCCAT | SEQ ID NO: 1443 | TAAGCCAGTCAGATCAGGATTCTGAT | SEQ ID NO: 1444 |
| SGI_R8180033 | GGTCAACCACCCACATGTCA | SEQ ID NO: 1445 | AAGAGGGAGAACAGGGCTGTA | SEQ ID NO: 1446 |
| SGI_R0317037 | AAAGGAACAATATGAATTATACTGTGAGATGG | SEQ ID NO: 1447 | GTACCTGCCAGGATGTAAGACAG | SEQ ID NO: 1448 |
| SGI_R8180044 | CTTTAGATTCAGAAAGTCCTCACCTTGA | SEQ ID NO: 1449 | GAGTTTGTCTGCAAGGTTTACAGTG | SEQ ID NO: 1450 |
| SGI_R0317038 | TCACAAACCCTACAGATACCCAGA | SEQ ID NO: 1451 | GGGCATGTATCCAGATGATGGA | SEQ ID NO: 1452 |
| SGI_R8180046 | TGTGATGTTCTGAAAGCTTAATTCTACCTT | SEQ ID NO: 1453 | CGGCCAACACTGTCAAGTTTC | SEQ ID NO: 1454 |
| SGI_R0317041 | ATCTGGAAAACTTTCTTTCAGTGATACA | SEQ ID NO: 1455 | ACCTTTAGCTAATAAAAATGTGATCCAAGAAAC | SEQ ID NO: 1456 |
| SGI_R8180051 | GGAGCACCTAGGCTAAAATGTCA | SEQ ID NO: 1457 | CACCAGTATTTTCTCACAGAAAGAATGTC | SEQ ID NO: 1458 |
| SGI_R0317042 | GTTTAACCTTTCTACTGTTTTCTTTGTCTGA | SEQ ID NO: 1459 | ATCTGTTCCAGAATCAAGATTCTGAGATG | SEQ ID NO: 1460 |
| SGI_R4534501 | CAGTCTTACATTTGACCATGACCATG | SEQ ID NO: 1461 | ACTGATGACCTTTGGAGGAAAACC | SEQ ID NO: 1462 |
| SGI_R6584429 | CCTCCTTCCTAGAGAGTTAGAGTAACT | SEQ ID NO: 1463 | CACCCACACTTACACATCACTTTG | SEQ ID NO: 1464 |
| SGI_R4534523 | CCAGTTACCTGTCCTGGTCATT | SEQ ID NO: 1465 | GGAAACTCCCATCTTGAGTCATAAGG | SEQ ID NO: 1466 |
| SGI_R6584437 | TTTTTCTGTCCACCAGGGAGTA | SEQ ID NO: 1467 | ACATTGGAATAGTTTCAAACATCATCTTGTG | SEQ ID NO: 1468 |
| SGI_R4534528 | AGACGACACAGGAAGCAGATTC | SEQ ID NO: 1469 | CAGTCTGCTGGATTTGGTTCTAGG | SEQ ID NO: 1470 |
| SGI_R6584464 | AAGATCACCTTCAGAAGTCACAGAATG | SEQ ID NO: 1471 | CTGGTTGAGATGAAAGGATTCCACT | SEQ ID NO: 1472 |
| SGI_R4534540 | TGGACCACACAGGAGAATATGGA | SEQ ID NO: 1473 | CTTAACAAGCTGTCTCCTCTCCTT | SEQ ID NO: 1474 |
| SGI_R6584466 | GTTCTGTTAAAGTTCATGGCTTTTGTGT | SEQ ID NO: 1475 | TTTACATAAGAAGCGTTTACGATCCTCTTT | SEQ ID NO: 1476 |
| SGI_R4534548 | AGGTGCAGAACATCAAGTTCAACA | SEQ ID NO: 1477 | GTGCTCAGCCTCTGTGAAGAG | SEQ ID NO: 1478 |
| SGI_R6584608 | CAGAAGGTCTACATGGGTGCTT | SEQ ID NO: 1479 | GCCAGCCCGAAGTCTGTAATTTT | SEQ ID NO: 1480 |
| SGI_R4534583 | TCTATATGTAGAGGCTGTTGGAAGCT | SEQ ID NO: 1481 | TCCACTGAAGTTCTTTATCTTCAAATAACT | SEQ ID NO: 1482 |
| SGI_R6584668 | TGCTTTAGATTGGCAATTATTACTGTTTCG | SEQ ID NO: 1483 | GTTGACTTTGTCCACCTGGAACT | SEQ ID NO: 1484 |
| SGI_R4534G15 | AAGGCTTTTTCTTTAGACAGTTGTTTGTT | SEQ ID NO: 1485 | GAGGTTCCCGTAGGTCATGAAC | SEQ ID NO: 1486 |
| SGI_R6684680 | CTGCGACCCTTATAATGAGCCT | SEQ ID NO: 1487 | GCAACTATTTTCTTCCTCTCTTCCACA | SEQ ID NO: 1488 |
| SGI_R4534646 | GGCACGGTTGAATGTAAGGCTTA | SEQ ID NO: 1489 | ACTGATATGGTAGACAGAGCCTAAACAT | SEQ ID NO: 1490 |
| SGI_R6S94733 | AGGCTTCATATGATGAAGGGTAATGTG | SEQ ID NO: 1491 | TAGGAGATACCCACGTATGTACCAC | SEQ ID NO: 1492 |
| SGI_R4534796 | CCACTCCATCGAGATTTCACTGTA | SEQ ID NO: 1493 | TCATAATGCTTGCTCTGATAGGAAAATGA | SEQ ID NO: 1494 |
| SGI_R6594734 | AAAAATCAAATCTTAAAAGCTTCTTGGTGT | SEQ ID NO: 1495 | TCTTTCTCCACTCAGCGTCTTTG | SEQ ID NO: 1496 |
| SGI_R4534799 | GATTGAAGAGCCCACAGGTGAT | SEQ ID NO: 1497 | CTCCTCCTTGCTAGGGTTCTTC | SEQ ID NO: 1498 |
| SGI_R6594736 | CAGAAACGTTTCGATTATAAAGATCAGCA | SEQ ID NO: 1499 | AAAAAGACTGTAAGTGGTTTCTCAGGAA | SEQ ID NO: 1500 |
| SGI_R4534814 | GGACTTGGTGATAGACATGTACAGAAT | SEQ ID NO: 1501 | GCAAACAACATTCCATGATGACCAAATATT | SEQ ID NO: 1502 |
| SGI_R6594741 | CTGCACATCGGGATGTAGGATC | SEQ ID NO: 1503 | GAACCCTGAGAGCAGCTTCAAT | SEQ ID NO: 1504 |
| SGI_R4534847 | TTCTTTGTAGATATGATGCAGCCATTGA | SEQ ID NO: 1505 | GAAAACCATTACTTGTCCATCGTCTTTC | SEQ ID NO: 1506 |
| SGI_R6596984 | AGAAAATTGACTAACCTGTGTTTCTTTACA | SEQ ID NO: 1507 | CCTTTGGAAGTGGACCCAGAAAC | SEQ ID NO: 1508 |
| SGI_R8180064 | CCATTTTCTCTCAGTAAGTGTTTATGATGC | SEQ ID NO: 1509 | ATTTAAAATTAGCACCCTGAGAAGCTCT | SEQ ID NO: 1510 |
| SGI_R0317049 | GAACAGGCCCTCAGTTCAAGAT | SEQ ID NO: 1511 | ACTCTCCCTTCACAGGTGGTATT | SEQ ID NO: 1512 |
| SGI_R8180066 | TTTGTTTGTCAGAGTCAGAGCACT | SEQ ID NO: 1513 | TCTAGATCCTAAACGTAAGAAGCAACAC | SEQ ID NO: 1514 |
| SGI_R0326962 | GTGACAAACCTGCTGAGCATTAG | SEQ ID NO: 1515 | TGAAATCAGTGTTTTGCTTCTCTAGGTAC | SEQ ID NO: 1516 |
| SGI_R8180067 | CCTGTTTAGGCCTTGCAGAATTTG | SEQ ID NO: 1517 | TCCCACTGCATATTCCTCCATG | SEQ ID NO: 1518 |
| SGI_R0234302 | GCATAGAGGAGAGAGGAAAAGTGG | SEQ ID NO: 1519 | ATTGGCAGCTCCGAGGACCA | SEQ ID NO: 1520 |
| SGI_R8180075 | TGGTGGACAAGTGAATTTGCTCA | SEQ ID NO: 1521 | TTCTAAAGGCTGAATGAAAGGGTAATTCAT | SEQ ID NO: 1522 |
| SGI_R0234303 | CTGCCAATCGGCGTGTAA | SEQ ID NO: 1523 | CTCCTCTTCTTTTCCTCTGGCT | SEQ ID NO: 1524 |
| SGI_R8180076 | TCTTTGCTCATCTTTTCTTTATGTTTTCGAATT | SEQ ID NO: 1525 | AATGAAATTTGTTACCTGTACACATGAAGC | SEQ ID NO: 1526 |
| SGI_R0327759 | GTTCTTTTGTCCTACTCCTTCTTTCCA | SEQ ID NO: 1527 | TTACTTCAGTGTTTCTCCATCATCACAG | SEQ ID NO: 1528 |
| SGI_R8180094 | AAAATCTCTGTCCAAGTCCTGTGAAA | SEQ ID NO: 1529 | GCTTTGTGTATGCCTATAATTGAAACTGT | SEQ ID NO: 1530 |
| SGI_R0333112 | TCTTACACCCAGTGGAGAAGCT | SEQ ID NO: 1531 | TGTGCCAGGGACCTTACCTTATA | SEQ ID NO: 1532 |
| SGI_R8180099 | TGCATTACCTACGATGGTAACCAAAG | SEQ ID NO: 1533 | CCTATTCAACAAACAGAACTATGATACGGA | SEQ ID NO: 1534 |
| SGI_R0333114 | GCATTAACTAGTCAAGTACTTACCCACT | SEQ ID NO: 1535 | ATCTCTTTCATGACTGCAGCTTCTT | SEQ ID NO: 1536 |
| SGI_R8180128 | GTGTTCACTTTCAGGAATTCTATGAGC | SEQ ID NO: 1537 | GTTGGGTGGCGGTTACTTACTA | SEQ ID NO: 1538 |
| SGI_R0333115 | AAAGAGATCAAACACCCTAACCTGG | SEQ ID NO: 1539 | CGAGGTTTTGTGCAGTGAGC | SEQ ID NO: 1540 |
| SGI_R8190610 | GCCTCTCTAATTTTGTGACATTTGAGC | SEQ ID NO: 1541 | GGCATGCTGTCGAATAGCTAGA | SEQ ID NO: 1542 |
| SGI_R0333116 | CTCCTGAAAAGAGAGTGGAAGTGT | SEQ ID NO: 1543 | AGTTGCTGCAAGTCAGTTGAAAAATC | SEQ ID NO: 1544 |
| SGI_R8190626 | GGGTGTGGATGCTTCCTTTTAAAC | SEQ ID NO: 1545 | TGTACTCCAGTGAGGAAGCAGAA | SEQ ID NO: 1546 |
| SGI_R4679131 | GATCGTCTCCATCATCATCATCGT | SEQ ID NO: 1547 | GACATTATTGCTTCTCCTGTGTGTTTC | SEQ ID NO: 1548 |
| SGI_R8190643 | CATCATTAATTTTTGCTTCACAGAAGACCA | SEQ ID NO: 1549 | TATTACCCAGAGATACCCAGAAAAGAGATT | SEQ ID NO: 1550 |
| SGI_R8180058 | TTTGTGGTTTACTTTAAGATTACAAATTCAGAA | SEQ ID NO: 1551 | GCTTTCTGGAATAATTCTGACTTATATGCTTC | SEQ ID NO: 1552 |
| SGI_R8190649 | TGCTACTATCATCAGACTGATCAAAATCG | SEQ ID NO: 1553 | GGTAGATGAGGACTCCTCAGGAAA | SEQ ID NO: 1554 |
| SGI_R0317048 | CGACGACCACGGTCTCTAGA | SEQ ID NO: 1555 | GTTGAGAGAGTGGGTGTGGTT | SEQ ID NO: 1556 |

Table 3 lists the chromosome location and starting and ending positions of the genes for methylation analysis and variant detection.

| Chromosome | Chr_start | Chr_end | Gene | Tag |
|---|---|---|---|---|
| chr16 | 58498542 | 58498671 | mC_NDRG4 | met |
| chr17 | 75368916 | 75369044 | mC_SEPT | met |
| chr17 | 75370019 | 75370139 | mC_SEPT | met |
| chr17 | 75370467 | 75370591 | mC_SEPT | met |
| chr3 | 37034313 | 37034427 | mC_MLH1 | met |
| chr3 | 37034457 | 37034582 | mC_MLH1 | met |
| chr3 | 37034709 | 37034833 | mC_MLH1 | met |
| chr3 | 37035176 | 37035300 | mC_MLH1 | met |
| chr3 | 37053566 | 37053681 | mC_MLH1 | met |
| chr3 | 37083802 | 37083912 | mC_MLH1 | met |
| chr3 | 55520233 | 55520354 | mC_WNT5A | met |
| chr3 | 55520384 | 55520510 | mC_WNT5A | met |
| chr3 | 55520568 | 55520684 | mC_WNT5A | met |
| chr3 | 55520846 | 55520969 | mC_WNT5A | met |
| chr3 | 55521518 | 55521641 | mC_WNT5A | met |
| chr3 | 55521707 | 55521833 | mC_WNT5A | met |
| chr3 | 148415435 | 148415563 | mC_AGTR1 | met |
| chr3 | 148415646 | 148415775 | mC_AGTR1 | met |
| chr4 | 81952009 | 81952134 | mC_BMP3 | met |
| chr4 | 81952545 | 81952673 | mC_BMP3 | met |
| chr4 | 154709589 | 154709716 | mC_SFRP2 | met |
| chr4 | 154709739 | 154709864 | mC_SFRP2 | met |
| chr5 | 134871210 | 134871339 | mC_NEUROG1 | met |
| chr5 | 134871388 | 134871515 | mC_NEUROG1 | met |
| chr7 | 93519372 | 93519490 | mC_TFPI2 | met |
| chr7 | 93519583 | 93519704 | mC_TFPI2 | met |
| chr7 | 93520337 | 93520459 | mC_TFPI2 | met |
| chr8 | 97505718 | 97505844 | mC_SDC2 | met |
| chr8 | 97505844 | 97505974 | mC_SDC2 | met |
| chr8 | 97506065 | 97506174 | mC_SDC2 | met |
| chr8 | 97506191 | 97506311 | mC_SDC2 | met |
| chr8 | 97506430 | 97506560 | mC_SDC2 | met |
| chr8 | 97506626 | 97506741 | mC_SDC2 | met |
| chr8 | 97507003 | 97507128 | mC_SDC2 | met |
| chr8 | 97507242 | 97507370 | mC_SDC2 | met |
| chr1 | 43805140 | 43805255 | MPL | Onco |
| chr1 | 43814946 | 43815063 | MPL | Onco |
| chr1 | 65305376 | 65305495 | JAK1 | Onco |
| chr1 | 65310478 | 65310601 | JAK1 | Onco |
| chr1 | 65311196 | 65311321 | JAK1 | Onco |
| chr1 | 65312358 | 65312477 | JAK1 | Onco |
| chr1 | 115256506 | 115256624 | NRAS | Onco |
| chr1 | 115258706 | 115258829 | NRAS | Onco |
| chr1 | 162724504 | 162724625 | DDR2 | Onco |
| chr1 | 162745524 | 162745647 | DDR2 | Onco |
| chr1 | 162750003 | 162750125 | DDR2 | Onco |
| chr10 | 43601762 | 43601893 | RET | Onco |
| chr10 | 43607568 | 43607695 | RET | Onco |
| chr10 | 43609015 | 43609148 | RET | Onco |
| chr10 | 43609969 | 43610098 | RET | Onco |
| chr10 | 43613786 | 43613908 | RET | Onco |
| chr10 | 43613918 | 43614034 | RET | Onco |
| chr10 | 43615565 | 43615683 | RET | Onco |
| chr10 | 43617384 | 43617503 | RET | Onco |
| chr10 | 89624261 | 89624381 | PTEN | Onco |
| chr10 | 89653802 | 89653904 | PTEN | Onco |
| chr10 | 89685262 | 89685362 | PTEN | Onco |
| chr10 | 89690761 | 89690875 | PTEN | Onco |
| chr10 | 89692792 | 89692904 | PTEN | Onco |
| chr10 | 89692962 | 89693067 | PTEN | Onco |
| chr10 | 89711900 | 89712017 | PTEN | Onco |
| chr10 | 89717726 | 89717834 | PTEN | Onco |
| chr10 | 89720808 | 89720923 | PTEN | Onco |
| chr10 | 123247523 | 123247643 | FGFR2 | Onco |
| chr10 | 123258002 | 123258120 | FGFR2 | Onco |
| chr10 | 123263317 | 123263435 | FGFR2 | Onco |
| chr10 | 123274574 | 123274700 | FGFR2 | Onco |
| chr10 | 123274760 | 123274883 | FGFR2 | Onco |
| chr10 | 123276944 | 123277063 | FGFR2 | Onco |
| chr10 | 123278278 | 123278398 | FGFR2 | Onco |
| chr10 | 123279517 | 123279634 | FGFR2 | Onco |
| chr10 | 123279646 | 123279764 | FGFR2 | Onco |
| chr10 | 123298047 | 123298169 | FGFR2 | Onco |
| chr10 | 123298176 | 123298295 | FGFR2 | Onco |
| chr10 | 123310826 | 123310945 | FGFR2 | Onco |
| chr10 | 123324989 | 123325111 | FGFR2 | Onco |
| chr11 | 533806 | 533932 | HRAS | Onco |
| chr11 | 534239 | 534356 | HRAS | Onco |
| chr11 | 108098615 | 108098721 | ATM | Onco |
| chr11 | 108106438 | 108106556 | ATM | Onco |
| chr11 | 108117783 | 108117895 | ATM | Onco |
| chr11 | 108119830 | 108119948 | ATM | Onco |
| chr11 | 108122635 | 108122737 | ATM | Onco |
| chr11 | 108126976 | 108127081 | ATM | Onco |
| chr11 | 108129732 | 108129844 | ATM | Onco |
| chr11 | 108139241 | 108139364 | ATM | Onco |
| chr11 | 108142010 | 108142133 | ATM | Onco |
| chr11 | 108143245 | 108143356 | ATM | Onco |
| chr11 | 108153452 | 108153560 | ATM | Onco |
| chr11 | 108160493 | 108160602 | ATM | Onco |
| chr11 | 108165711 | 108165823 | ATM | Onco |
| chr11 | 108170475 | 108170586 | ATM | Onco |
| chr11 | 108172382 | 108172492 | ATM | Onco |
| chr11 | 108175412 | 108175525 | ATM | Onco |
| chr11 | 108178655 | 108178773 | ATM | Onco |
| chr11 | 108180960 | 108181069 | ATM | Onco |
| chr11 | 108183183 | 108183296 | ATM | Onco |
| chr11 | 108186563 | 108186669 | ATM | Onco |
| chr11 | 108188134 | 108188258 | ATM | Onco |
| chr11 | 108199787 | 108199902 | ATM | Onco |
| chr11 | 108199925 | 108200041 | ATM | Onco |
| chr11 | 108200936 | 108201048 | ATM | Onco |
| chr11 | 108202720 | 108202831 | ATM | Onco |
| chr11 | 108205739 | 108205862 | ATM | Onco |
| chr11 | 108216543 | 108216653 | ATM | Onco |
| chr11 | 108218066 | 108218179 | ATM | Onco |
| chr11 | 108224538 | 108224655 | ATM | Onco |
| chr11 | 108236059 | 108236183 | ATM | Onco |
| chr11 | 108236190 | 108236295 | ATM | Onco |
| chr11 | 119148420 | 119148539 | CBL | Onco |
| chr11 | 119148923 | 119149038 | CBL | Onco |
| chr11 | 119149229 | 119149341 | CBL | Onco |
| chr12 | 25362830 | 25362937 | KRAS | Onco |
| chr12 | 25368439 | 25368557 | KRAS | Onco |
| chr12 | 25378546 | 25378660 | KRAS | Onco |
| chr12 | 25380283 | 25380401 | KRAS | Onco |
| chr12 | 25398253 | 25398358 | KRAS | Onco |
| chr12 | 56477633 | 56477755 | ERBB3 | Onco |
| chr12 | 56478809 | 56478932 | ERBB3 | Onco |
| chr12 | 56481806 | 56481924 | ERBB3 | Onco |
| chr12 | 56481942 | 56482063 | ERBB3 | Onco |
| chr12 | 56482303 | 56482422 | ERBB3 | Onco |
| chr12 | 56487141 | 56487259 | ERBB3 | Onco |
| chr12 | 56490393 | 56490509 | ERBB3 | Onco |
| chr12 | 56491620 | 56491738 | ERBB3 | Onco |
| chr12 | 56493900 | 56494024 | ERBB3 | Onco |
| chr12 | 58145431 | 58145556 | CDK4 | Onco |
| chr12 | 121426835 | 121426954 | HNF1A | Onco |
| chr12 | 121431392 | 121431508 | HNF1A | Onco |
| chr13 | 28592593 | 28592711 | FLT3 | Onco |
| chr13 | 28601324 | 28601439 | FLT3 | Onco |
| chr13 | 28602344 | 28602466 | FLT3 | Onco |
| chr13 | 28608270 | 28608381 | FLT3 | Onco |
| chr13 | 28608413 | 28608533 | FLT3 | Onco |
| chr13 | 28623558 | 28623672 | FLT3 | Onco |
| chr13 | 48881454 | 48881574 | RB1 | Onco |
| chr13 | 48923072 | 48923178 | RB1 | Onco |
| chr13 | 48936987 | 48937094 | RB1 | Onco |
| chr13 | 48941638 | 48941744 | RB1 | Onco |
| chr13 | 48947546 | 48947656 | RB1 | Onco |
| chr13 | 48951105 | 48951216 | RB1 | Onco |
| chr13 | 48953724 | 48953819 | RB1 | Onco |
| chr13 | 48955328 | 48955438 | RB1 | Onco |
| chr13 | 48955531 | 48955644 | RB1 | Onco |
| chr13 | 49027206 | 49027316 | RB1 | Onco |
| chr13 | 49030302 | 49030422 | RB1 | Onco |
| chr13 | 49033898 | 49034017 | RB1 | Onco |
| chr13 | 49037911 | 49038011 | RB1 | Onco |
| chr13 | 49039163 | 49039280 | RB1 | Onco |
| chr14 | 105237126 | 105237254 | AKT1 | Onco |
| chr14 | 105242097 | 105242214 | AKT1 | Onco |
| chr14 | 105242926 | 105243052 | AKT1 | Onco |
| chr14 | 105243055 | 105243169 | AKT1 | Onco |
| chr14 | 105246490 | 105246607 | AKT1 | Onco |
| chr15 | 90631766 | 90631893 | IDH2 | Onco |
| chr15 | 90631911 | 90632034 | IDH2 | Onco |
| chr16 | 68835723 | 68835840 | CDH1 | Onco |
| chr16 | 68846036 | 68846160 | CDH1 | Onco |
| chr16 | 68849603 | 68849723 | CDH1 | Onco |
| chr16 | 68853323 | 68853444 | CDH1 | Onco |
| chr17 | 7574014 | 7574125 | TP53 | Onco |
| chr17 | 7576891 | 7577008 | TP53 | Onco |
| chr17 | 7577100 | 7577223 | TP53 | Onco |
| chr17 | 7577539 | 7577665 | TP53 | Onco |
| chr17 | 7578228 | 7578346 | TP53 | Onco |
| chr17 | 7578400 | 7578530 | TP53 | Onco |
| chr17 | 7579307 | 7579431 | TP53 | Onco |
| chr17 | 7579528 | 7579644 | TP53 | Onco |
| chr17 | 37868182 | 37868309 | ERBB2 | Onco |
| chr17 | 37879581 | 37879709 | ERBB2 | Onco |
| chr17 | 37879918 | 37880049 | ERBB2 | Onco |
| chr17 | 37880202 | 37880331 | ERBB2 | Onco |
| chr17 | 37880985 | 37881113 | ERBB2 | Onco |
| chr17 | 37881311 | 37881435 | ERBB2 | Onco |
| chr17 | 37881581 | 37881695 | ERBB2 | Onco |
| chr17 | 40468820 | 40468944 | STAT3 | Onco |
| chr18 | 48591769 | 48591887 | SMAD4 | Onco |
| chr18 | 48591898 | 48592014 | SMAD4 | Onco |
| chr18 | 48593422 | 48593531 | SMAD4 | Onco |
| chr18 | 48603046 | 48603164 | SMAD4 | Onco |
| chr18 | 48604757 | 48604875 | SMAD4 | Onco |
| chr19 | 1221255 | 1221382 | STK11 | Onco |
| chr19 | 3114979 | 3115108 | GNA11 | Onco |
| chr19 | 3118895 | 3119021 | GNA11 | Onco |
| chr19 | 17949074 | 17949188 | JAK3 | Onco |
| chr19 | 52709179 | 52709305 | PPP2R1A | Onco |
| chr2 | 25457187 | 25457309 | RET | Onco |
| chr2 | 25469511 | 25469640 | DNMT3A | Onco |
| chr2 | 29419649 | 29419760 | ALK | Onco |
| chr2 | 29432673 | 29432795 | ALK | Onco |
| chr2 | 29436807 | 29436920 | ALK | Onco |
| chr2 | 29443626 | 29443745 | ALK | Onco |
| chr2 | 29445165 | 29445285 | ALK | Onco |
| chr2 | 29445403 | 29445526 | ALK | Onco |
| chr2 | 29446359 | 29446486 | ALK | Onco |
| chr2 | 29474074 | 29474197 | ALK | Onco |
| chr2 | 29519779 | 29519902 | ALK | Onco |
| chr2 | 29606650 | 29606773 | ALK | Onco |
| chr2 | 178098007 | 178098117 | NFE2L2 | Onco |
| chr2 | 178098754 | 178098876 | NFE2L2 | Onco |
| chr2 | 178098909 | 178099020 | NFE2L2 | Onco |
| chr2 | 198266774 | 198266894 | SF3B1 | Onco |
| chr2 | 198285812 | 198285922 | PIK3CA | Onco |
| chr2 | 212288916 | 212289036 | ERBB4 | Onco |
| chr2 | 212530120 | 212530241 | ERBB4 | Onco |
| chr2 | 212566790 | 212566910 | ERBB4 | Onco |
| chr2 | 212576801 | 212576917 | ERBB4 | Onco |
| chr2 | 212578346 | 212578461 | ERBB4 | Onco |
| chr2 | 212589784 | 212589906 | ERBB4 | Onco |
| chr2 | 212812111 | 212812223 | ERBB4 | Onco |
| chr20 | 57478824 | 57478943 | GNAS | Onco |
| chr20 | 57480470 | 57480583 | GNAS | Onco |
| chr20 | 57484383 | 57484500 | GNAS | Onco |
| chr21 | 44513339 | 44513466 | U2AF1 | Onco |
| chr21 | 44515790 | 44515905 | U2AF1 | Onco |
| chr21 | 44524451 | 44524570 | U2AF1 | Onco |
| chr21 | 44527602 | 44527735 | U2AF1 | Onco |
| chr21 | 46934829 | 46934959 | SLC19A1 | Onco |
| chr22 | 24133945 | 24134066 | SMARCB1 | Onco |
| chr22 | 24145538 | 24145652 | SMARCB1 | Onco |
| chr22 | 29091840 | 29091952 | CHEK2 | Onco |
| chr22 | 29092896 | 29093009 | CHEK2 | Onco |
| chr3 | 10188221 | 10188342 | VHL | Onco |
| chr3 | 12641286 | 12641407 | RAF1 | Onco |
| chr3 | 12645666 | 12645790 | RAF1 | Onco |
| chr3 | 41266078 | 41266203 | CTNNB1 | Onco |
| chr3 | 178916724 | 178916833 | PIK3CA | Onco |
| chr3 | 178916904 | 178917003 | PIK3CA | Onco |
| chr3 | 178917429 | 178917541 | PIK3CA | Onco |
| chr3 | 178917652 | 178917767 | PIK3CA | Onco |
| chr3 | 178919134 | 178919252 | PIK3CA | Onco |
| chr3 | 178921503 | 178921614 | PIK3CA | Onco |
| chr3 | 178922338 | 178922446 | PIK3CA | Onco |
| chr3 | 178927341 | 178927462 | PIK3CA | Onco |
| chr3 | 178927953 | 178928065 | PIK3CA | Onco |
| chr3 | 178928091 | 178928208 | PIK3CA | Onco |
| chr3 | 178928337 | 178928454 | PIK3CA | Onco |
| chr3 | 178936060 | 178936171 | PIK3CA | Onco |
| chr3 | 178937342 | 178937455 | PIK3CA | Onco |
| chr3 | 178938805 | 178938921 | PIK3CA | Onco |
| chr3 | 178938936 | 178939046 | PIK3CA | Onco |
| chr3 | 178947829 | 178947943 | PIK3CA | Onco |
| chr3 | 178951838 | 178951958 | PIK3CA | Onco |
| chr3 | 178951971 | 178952073 | PIK3CA | Onco |
| chr3 | 178952090 | 178952203 | PIK3CA | Onco |
| chr4 | 55133801 | 55133922 | PDGFRA | Onco |
| chr4 | 55139772 | 55139893 | PDGFRA | Onco |
| chr4 | 55140688 | 55140809 | PDGFRA | Onco |
| chr4 | 55141022 | 55141144 | PDGFRA | Onco |
| chr4 | 55144122 | 55144241 | PDGFRA | Onco |
| chr4 | 55144495 | 55144611 | PDGFRA | Onco |
| chr4 | 55146546 | 55146659 | PDGFRA | Onco |
| chr4 | 55152101 | 55152212 | PDGFRA | Onco |
| chr4 | 55561764 | 55561880 | KIT | Onco |
| chr4 | 55589785 | 55589901 | KIT | Onco |
| chr4 | 55592083 | 55592205 | KIT | Onco |
| chr4 | 55593618 | 55593742 | KIT | Onco |
| chr4 | 55594177 | 55594293 | KIT | Onco |
| chr4 | 55594336 | 55594454 | KIT | Onco |
| chr4 | 55595514 | 55595615 | KIT | Onco |
| chr4 | 55599313 | 55599432 | KIT | Onco |
| chr4 | 55602647 | 55602767 | KIT | Onco |
| chr4 | 55602778 | 55602896 | KIT | Onco |
| chr4 | 55946133 | 55946253 | KDR | Onco |
| chr4 | 55955068 | 55955186 | KDR | Onco |
| chr4 | 55958749 | 55958872 | KDR | Onco |
| chr4 | 55962513 | 55962638 | KDR | Onco |
| chr4 | 55968126 | 55968245 | KDR | Onco |
| chr4 | 55979620 | 55979726 | KDR | Onco |
| chr4 | 55981129 | 55981239 | KDR | Onco |
| chr4 | 153244033 | 153244154 | FBXW7 | Onco |
| chr4 | 153244201 | 153244326 | FBXW7 | Onco |
| chr4 | 153245393 | 153245509 | FBXW7 | Onco |
| chr4 | 153247160 | 153247275 | FBXW7 | Onco |
| chr4 | 153247300 | 153247423 | FBXW7 | Onco |
| chr4 | 153249345 | 153249451 | FBXW7 | Onco |
| chr4 | 153249467 | 153249584 | FBXW7 | Onco |
| chr4 | 153251854 | 153251968 | FBXW7 | Onco |
| chr4 | 153253775 | 153253891 | FBXW7 | Onco |
| chr4 | 153258991 | 153259109 | FBXW7 | Onco |
| chr4 | 153268122 | 153268241 | FBXW7 | Onco |
| chr4 | 153332607 | 153332724 | FBXW7 | Onco |
| chr4 | 153332875 | 153332999 | FBXW7 | Onco |
| chr5 | 112173293 | 112173408 | APC | Onco |
| chr5 | 112175206 | 112175329 | APC | Onco |
| chr5 | 112175433 | 112175559 | APC | Onco |
| chr5 | 112175629 | 112175752 | APC | Onco |
| chr5 | 112175787 | 112175898 | APC | Onco |
| chr5 | 112175950 | 112176062 | APC | Onco |
| chr5 | 134870684 | 134870800 | NEUROG1 | Onco |
| chr5 | 134871527 | 134871650 | NEUROG1 | Onco |
| chr5 | 149453010 | 149453133 | CSF1R | Onco |
| chr5 | 170818724 | 170818831 | NPM1 | Onco |
| chr6 | 18130903 | 18131000 | TPMT | Onco |
| chr6 | 18131015 | 18131117 | TPMT | Onco |
| chr6 | 18139233 | 18139346 | TPMT | Onco |
| chr6 | 18143946 | 18144051 | TPMT | Onco |
| chr7 | 55210048 | 55210168 | EGFR | Onco |
| chr7 | 55211060 | 55211178 | EGFR | Onco |
| chr7 | 55220172 | 55220292 | EGFR | Onco |
| chr7 | 55221840 | 55221964 | EGFR | Onco |
| chr7 | 55227952 | 55228070 | EGFR | Onco |
| chr7 | 55229193 | 55229313 | EGFR | Onco |
| chr7 | 55231384 | 55231496 | EGFR | Onco |
| chr7 | 55232985 | 55233105 | EGFR | Onco |
| chr7 | 55241666 | 55241780 | EGFR | Onco |
| chr7 | 55242432 | 55242551 | EGFR | Onco |
| chr7 | 55249024 | 55249153 | EGFR | Onco |
| chr7 | 55259501 | 55259615 | EGFR | Onco |
| chr7 | 55260429 | 55260546 | EGFR | Onco |
| chr7 | 55273564 | 55273682 | EGFR | Onco |
| chr7 | 116339622 | 116339741 | MET | Onco |
| chr7 | 116340215 | 116340339 | MET | Onco |
| chr7 | 116397740 | 116397851 | MET | Onco |
| chr7 | 116412002 | 116412120 | MET | Onco |
| chr7 | 116417452 | 116417569 | MET | Onco |
| chr7 | 116418832 | 116418949 | MET | Onco |
| chr7 | 116418989 | 116419114 | MET | Onco |
| chr7 | 116422060 | 116422179 | MET | Onco |
| chr7 | 116423368 | 116423489 | MET | Onco |
| chr7 | 128845091 | 128845216 | SMO | Onco |
| chr7 | 128846100 | 128846224 | SMO | Onco |
| chr7 | 128846304 | 128846434 | SMO | Onco |
| chr7 | 128849158 | 128849277 | SMO | Onco |
| chr7 | 128850286 | 128850414 | SMO | Onco |
| chr7 | 128850776 | 128850902 | SMO | Onco |
| chr7 | 128851534 | 128851658 | SMO | Onco |
| chr7 | 128851885 | 128852005 | SMO | Onco |
| chr7 | 128852158 | 128852280 | SMO | Onco |
| chr7 | 140434476 | 140434599 | BRAF | Onco |
| chr7 | 140453095 | 140453205 | BRAF | Onco |
| chr7 | 140453976 | 140454091 | BRAF | Onco |
| chr7 | 140476812 | 140476929 | BRAF | Onco |
| chr7 | 140481384 | 140481500 | BRAF | Onco |
| chr7 | 140501243 | 140501344 | BRAF | Onco |
| chr7 | 140501355 | 140501458 | BRAF | Onco |
| chr7 | 148506166 | 148506282 | EZH2 | Onco |
| chr7 | 148506408 | 148506514 | EZH2 | Onco |
| chr7 | 148507454 | 148507568 | EZH2 | Onco |
| chr7 | 148516646 | 148516764 | EZH2 | Onco |
| chr7 | 148523710 | 148523828 | EZH2 | Onco |
| chr7 | 148524217 | 148524330 | EZH2 | Onco |
| chr7 | 148525800 | 148525909 | EZH2 | Onco |
| chr7 | 148525923 | 148526042 | EZH2 | Onco |
| chr7 | 148543590 | 148543700 | EZH2 | Onco |
| chr7 | 151167652 | 151167765 | RHEB | Onco |
| chr8 | 38272281 | 38272403 | FGFR1 | Onco |
| chr8 | 38274787 | 38274909 | FGFR1 | Onco |
| chr9 | 5055663 | 5055784 | JAK2 | Onco |
| chr9 | 5078339 | 5078449 | JAK2 | Onco |
| chr9 | 21974622 | 21974747 | CDKN2A | Onco |
| chr9 | 21994174 | 21994299 | CDKN2A | Onco |
| chr9 | 37015111 | 37015230 | PAX5 | Onco |
| chr9 | 98218570 | 98218676 | PTCH1 | Onco |
| chr9 | 98229384 | 98229504 | PTCH1 | Onco |
| chr9 | 98230998 | 98231116 | PTCH1 | Onco |
| chr9 | 98231229 | 98231355 | PTCH1 | Onco |
| chr9 | 98242347 | 98242468 | PTCH1 | Onco |
| chr9 | 133738303 | 133738429 | ABL1 | Onco |
| chr9 | 133747486 | 133747596 | ABL1 | Onco |
| chr9 | 133747608 | 133747732 | ABL1 | Onco |
| chr9 | 133748217 | 133748336 | ABL1 | Onco |
| chr9 | 133748341 | 133748453 | ABL1 | Onco |
| chr9 | 133750332 | 133750454 | ABL1 | Onco |
| chr9 | 139391136 | 139391263 | NOTCH1 | Onco |
| chr9 | 139397676 | 139397803 | NOTCH1 | Onco |
| chrX | 47422374 | 47422494 | ARAF | Onco |
| chrX | 47428925 | 47429039 | ARAF | Onco |
| chrX | 70339977 | 70340100 | MED12 | Onco |
| chrX | 100614252 | 100614377 | BTK | Onco |

To demonstrate the feasibility of quantifying DNA methylation and identifying genetic variants on tumor samples, MSA-seq was applied to 10 pairs of tumor and adjacent normal tissues from colorectal cancer (CRC) patients.

With 20 ng of FFPE input DNA per sample, the DNA methylation levels of the 24 promoter CpG sites on the ten genes were quantified, and classified the ten tumor samples into two distinct groups, one is highly methylated for SEPT, AGTR1, SDC2, SFRP2 and TFPI2, whereas the second group is also highly methylated on additional genes such as WNT5A, MLH1 and BMP3. With the same data set, 0-12 somatic mutations in each of the 10 tumor samples were also identified (Table 4).

All 28 mutations were detected in a single reaction on the HpaII digested DNA, without the need for a separate reaction on undigested DNA.

TABLE 4
Somatic mutation identified in 10 CRC tumor samples.

| Sample_ID | Mutation_freq | Gene | AA_change |
|---|---|---|---|
| Tumor-1LCS | 28.6% | ARC | p.E1309* |
| Tumor-2YMH | 18.1% | PIK3CA | p.E545K |
| Tumor-3SXN | 52.6% | TP53 | p.V122fs*26 |
| Tumor-4WXH | 32.8% | KRAS | p.G12V |
| Tumor-5CYJ | 43.3% | KRAS | p.G12V |
| Tumor-5CYJ | 40.2% | TP53 | p.R248P |
| Tumor-6YWZ | no mutation found | | |
| Tumor-7FHG | 77.0% | TP53 | p.R213* |
| Tumor-7FHG | 57.7% | APC | p.E1552* |
| Tumor-7FHG | 54.1% | EGFR | p.P753P |
| Tumor-7FHG | 44.6% | NRAS | p.Q61L |
| Tumor-8XXH | 10.7% | APC | p.E1309* |
| Tumor-8XXH | 30.6% | TP53 | p.R213* |
| Tumor-8XXH | 9.5% | EGFR | p.P753P |
| Tumor-8XXH | 32.5% | NRAS | p.Q61L |
| Tumor-8XXH | 14.9% | KRAS | p.G12V |
| Tumor-8XXH | 10.7% | APC | p.E1309* |
| Tumor-8XXH | 23.3% | ATM | p.G2382R |
| Tumor-8XXH | 11.5% | PIK3CA | p.W1057* |
| Tumor-8XXH | 9.1% | TP53 | p.P250L |
| Tumor-8XXH | 8.6% | SMAD4 | p.M331I |
| Tumor-8XXH | 5.8% | ATM | p.R805* |
| Tumor-8XXH | 5.8% | CTNNB1 | p.S45F |
| Tumor-9PXL | 5.6% | PIK3CA | p.H1047R |
| Tumor-9PXL | 24.2% | ERBB2 | p.V842I |
| Tumor-9PXL | 23.4% | PIK3CA | p.C378R |
| Tumor-9PXL | 21.6% | ATM | p.R2443* |
| Tumor-9PXL | 20.4% | MLH1 | p.S556fs*14 |
| Tumor-10XYM | 22.8% | KRAS | p.G12V |

A customized AmpliSeq primer panel was designed using the Ion AmpliSeq Designer tool available at ampliseq.com, and purchased from ThermoFisher Scientific. For the purpose of method calibration, genomic DNAs from the cell lines HCT116 and NA12878 were fragmented by Bioruptor. A series of synthetic DNA mixtures was prepared that contain HCT116 at 0%, 1%, 5%, 10%, 20% and 50%. In each reaction, 10 ng of DNA mixture was digested with NEB MspI/HpaII at 37° C. for 4 hours, purified with AmPure beads, and processed with the AmpliSeq amplification and Ion library preparation protocol with slight modification in volume. Ten tumor samples derived from colon rectal cancer patients underwent the same procedure in a pair of digested and undigested to calibrate the background. The resulting sequencing libraries were sequenced on Ion pgm/S5 sequencer. Mutation calling was performed with Torrent Suite. CpG methylation levels were calculated from the amplicon read depth data using customized Perl/Python scripts.

1. A method for analyzing a first target polynucleotide sequence and a methylation status of a second target polynucleotide sequence in a sample, comprising:
1) contacting a sample comprising a polynucleotide with a methylation-sensitive restriction enzyme (MSRE), wherein the MSRE selectively cleaves the polynucleotide at a residue when it is unmethylated or selectively cleaves the polynucleotide at the residue when it is methylated;
2) subjecting the sample from step 1) to polynucleotide amplification, using a mixture of:
i) a first primer set for amplifying a first target polynucleotide sequence in the sample, and
ii) a second primer set for analyzing a methylation status of a second target polynucleotide sequence in the sample, wherein the methylation status is of a residue in the second target polynucleotide sequence, and one primer of the second primer set hybridizes to the uncleaved second target polynucleotide sequence and together with another primer in the set, amplifies the uncleaved sequence but not the second target polynucleotide sequence cleaved at the residue by the MSRE; and
3) sequencing polynucleotides amplified in step 2),
wherein the first target polynucleotide sequence is analyzed using sequencing reads from the amplified first target polynucleotide sequence, and the methylation status of the residue of the second target polynucleotide sequence is analyzed by comparing the observed number of sequencing reads (No) from the amplified second target polynucleotide sequence to a reference number.

2. The method of claim 1, wherein the MSRE cleaves the polynucleotide at a residue when it is unmethylated and does not cleave at the residue when it is methylated.

3. The method of claim 1, wherein the method comprises amplification and sequencing of a polynucleotide from a sample that is not contacted with the MSRE.

4. The method of claim 1, wherein the MSRE is selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and any combination thereof.

5. The method of claim 1, wherein the first target polynucleotide sequence comprises a genetic or epigenetic information, such as a mutation, a single nucleotide polymorphism (SNP), a copy number variation (CNV), a DNA modification such as DNA methylation, and/or a histone modification.

6-7. (canceled)

8. The method of claim 1, wherein the second target polynucleotide sequence comprises one or more CpG sites within the recognition site of the MSRE, wherein at each CpG site the cytosine (C) comprises a 5-methyl moiety or a 5-hydrogen moiety.

9-10. (canceled)

11. The method of claim 1, wherein the sample is a biological sample.

12-13. (canceled)

14. The method of claim 1, wherein the polynucleotide in the sample is or comprises a double-stranded sequence or a single-stranded sequence.

15. (canceled)

16. The method of claim 1, wherein the first and second target polynucleotide sequences are on the same molecule or on different molecules, for example, two different DNA fragments, in the sample.

17. The method of claim 1, wherein the first and second target polynucleotide sequences are on the same gene, optionally wherein the first target polynucleotide sequence is in a coding region of the gene whereas the second target polynucleotide sequence is in a non-coding, regulatory region of the gene.

18. The method of claim 1, wherein the first and second target polynucleotide sequences are on different genes, optionally wherein the genes function in the same biological pathway or network.

19. The method of claim 1, wherein the first and second target polynucleotide sequences are on the same or different chromosomes, or on the same or different extrachromosomal DNA molecules (such as mitochondria DNA), or one on a chromosome and the other on an extrachromosomal DNA molecule.

20. The method of claim 1, wherein the amplification step comprises a polymerase chain reaction (PCR), reverse-transcription PCR amplification, allele-specific PCR (ASPCR), single-base extension (SBE), allele specific primer extension (ASPE), strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), the use of Q Beta replicase, nick translation, or loop-mediated isothermal amplification (LAMP), or any combination thereof.

21-22. (canceled)

23. The method of claim 1, wherein the second set of primers comprise a common primer and at least two primers each for a different CpG site in the second target polynucleotide sequence.

24. The method of claim 1, further comprising purifying polynucleotides from the sample in step 1), purifying polynucleotides from the sample in step 2), and/or purifying polynucleotides during the sequencing step 3).

25. The method of claim 1, wherein the sequencing step comprises attaching a sequencing adapter and/or a sample-specific barcode to each polynucleotide.

26. (canceled)

27. The method of claim 1, wherein the sequencing is a high-throughput sequencing, a digital sequencing, or a next-generating sequencing (NGS) such as Illumina (Solexa) sequencing, Roche 454 sequencing, Ion torrent: Proton/PGM sequencing, and SOLiD sequencing.

28. The method of claim 1, wherein the reference number is determined in parallel as the analysis of the first and second target polynucleotide sequences, as the expected number of sequencing reads (Nₑ) based on a control locus and/or a reference sample, with or without a control reaction using an isoschizomer of the MSRS that is methylation insensitive.

29. (canceled)

30. The method of claim 1, wherein the first primer set and/or the second primer set comprise one or more primers listed in Table 1 and/or Table 2, in any suitable combination.

31. The method of claim 1, wherein the first primer set comprises one or more primers for a gene selected from the group consisting of ABCB1, CYP2C19, CYP2C8, CYP2D6, CYP3A4, CYP3A5, DPYD, GSTP1, MTHFR, NQO1, RHEB, SULT1A1, UGT1A1, MPL, JAK1, NRAS, DDR2, PTEN, FGFR2, HRAS, ATM, CBL, KRAS, ERBB3, CDK4, HNF1A, FLT3, RB1, AKT1, IDH2, CDH1, TR53, ERBB2, STAT3, SMAD4, STK11, GNA11, JAK3, PPP2R1A, RET, DNMT3A, ALK, NFE2L2, SF3B1, PIK3CA, ERBB4, GNAS, U2AF1, SLC19A1, SMARCB1, CHEK2, VHL, RAF1, CTNNB1, PDGFRA, KIT, KDR, FBXW7, APC, NEUROG1, CSF1R, NPM1, TPMT, EGFR, MET, SMO, BRAF, EZH2, FGFR1, JAK2, CDKN2A, PAX5, PTCH1, ABL, NOTCH1, ARAF, MED12, BTK, and any combination thereof.

32. (canceled)

33. The method of claim 1, wherein the second primer set comprises one or more primers for a gene selected from the group consisting of NDRG4, SEPT, MLH1, WTN5A, AGTR1, BMP3, SFRP2, NEUROG1, TFPI2, SDC2, and any combination thereof.

34. (canceled)

35. The method of claim 1, wherein the amplification is multiplexed.

36. The method of claim 1, wherein the analysis of the first target polynucleotide sequence and the analysis of the methylation status of the second target polynucleotide sequence are conducted simultaneously in a single reaction.

37. The method of claim 1, wherein the polynucleotide concentration in the sample is less than about 0.1 ng/mL, less than about 1 ng/mL, less than about 3 ng/mL, less than about 5 ng/mL, less than about 10 ng/mL, less than about 20 ng/mL, or less than about 100 ng/mL.

38. The method of claim 1, which is used for the diagnosis and/or prognosis of a disease or condition in a subject, predicting the responsiveness of a subject to a treatment, identifying a pharmacogenetics marker for the disease/condition or treatment, and/or screening a population for a genetic information.

39. (canceled)

40. A kit, comprising:
a methylation-sensitive restriction enzyme (MSRE), wherein the MSRE selectively cleaves at a residue when it is unmethylated or selectively cleaves at the residue when it is methylated;
a first primer set for amplifying a first target polynucleotide sequence in a sample; and/or
a second primer set for analyzing a methylation status of a second target polynucleotide sequence in the sample, wherein the methylation status is of a residue in the second target polynucleotide sequence, and one primer of the second primer set hybridizes to the uncleaved second target polynucleotide sequence and together with another primer in the set, amplifies the uncleaved sequence but not the second target polynucleotide sequence cleaved at the residue by the MSRE.

41-54. (canceled)
